SEQUENCE LISTING (271 sequences)

SEQ ID NO: 1 - RNA, 23 nt, Homo sapiens
agcagcauuguacagggcuauga

SEQ ID NO: 2 - RNA, 23 nt, Homo sapiens
ucauagcccuguacaaugcugcu

SEQ ID NO: 3 - RNA, 23 nt, Homo sapiens
agcuucuuuacagugcugccuug

SEQ ID NO: 4 - RNA, 7 nt, Homo sapiens
gcagcau

SEQ ID NO: 5 - DNA, 5 bp, Artificial Sequence
  Description of Artificial Sequence: Synthetic oligonucleotide
tgtgc

SEQ ID NO: 6 - DNA, 9 bp, Homo sapiens
ctctgctgc

SEQ ID NO: 7 - RNA, 23 nt, Homo sapiens
ucaaaugcucagacuccuguggu

SEQ ID NO: 8 - RNA, 22 nt, Homo sapiens
acggauguuugagcaugugcua

SEQ ID NO: 9 - RNA, 7 nt, Homo sapiens
caaaugc

SEQ ID NO: 10 - RNA, 11 nt, Homo sapiens
ugcugcauuug

SEQ ID NO: 11 - RNA, 23 nt, Homo sapiens
agcagcauuguacagggcuauca

SEQ ID NO: 12 - RNA, 23 nt, Homo sapiens
uuaaugcuaaucgugauaggggu

SEQ ID NO: 13 - RNA, 23 nt, Homo sapiens
ucauagcccuguacaaugcugcu

SEQ ID NO: 14 - RNA, 6 nt, Homo sapiens
aaugcu

SEQ ID NO: 15 - RNA, 23 nt, Unknown
  Description of Unknown: miRNA-425 oligonucleotide
aaugacacgaucacucccguuga

SEQ ID NO: 16 - RNA, 6 nt, Unknown
  Description of Unknown: miR-425 seed oligonucleotide
augaca

SEQ ID NO: 17 - DNA, 23 bp, Unknown
  Description of Unknown: anti-miR-425 oligonucleotide
ttactgtgctagtgagggcaact

SEQ ID NO: 18 - DNA, 7 bp, Unknown
  Description of Unknown: miR-425 “A” allele miRNA TARGET sequence
atgacac

SEQ ID NO: 19 - DNA, 7 bp, Unknown
  Description of Unknown: miR-425 “G” allele miRNA TARGET sequence
gtgacac

SEQ ID NO: 20 - RNA, 297 nt, Homo sapiens
agauaacagccagggaggacaagcagggcugggccuagggacagacugcaagaggcuccuguccccuggggucucugcugcauuugugucaucuuguugccauggaguugugaucaucccaucuaagcugcagcuuccugucaacacuucucacaucuuaugcuaacuguagauaaagugguuugauggugacuuccucgccucucccaccccaugcauuaaauuuuaagguagaaccucaccuguuacugaaagugguuugaaagugaauaaacuucagcaccauggacagaagac

SEQ ID NO: 21 - RNA, 78 nt, Homo sapiens
uacugcccucggcuucuuuacagugcugccuuguugcauauggaucaagcagcauuguacagggcuaugaaggcauug

SEQ ID NO: 22 - RNA, 78 nt, Homo sapiens
uugugcuuucagcuucuuuacagugcugccuuguagcauucaggucaagcagcauuguacagggcuaugaaagaacca

SEQ ID NO: 23 - RNA, 62 nt, Homo sapiens
ucauagcccuguacaaugcugcuugauccauaugcaacaaggcagcacuguaaagaagccga

SEQ ID NO: 24 - RNA, 62 nt, Homo sapiens
ucauagcccuguacaaugcugcuugaccugaaugcuacaaggcagcacuguaaagaagcuga

SEQ ID NO: 25 - RNA, 81 nt, Homo sapiens
ugugcaucguggucaaaugcucagacuccugugguggcugcucaugcaccacggauguuugagcaugugcuacggugucua

SEQ ID NO: 26 - RNA, 81 nt, Homo sapiens
ugugcaucguggucaaaugcucagacuccugugguggcugcuuaugcaccacggauguuugagcaugugcuauggugucua

SEQ ID NO: 27 - RNA, 81 nt, Homo sapiens
cucucugcuuucagcuucuuuacaguguugccuuguggcauggaguucaagcagcauuguacagggcuaucaaagcacaga

SEQ ID NO: 28 - RNA, 87 nt, Unknown
  Description of Unknown: pre-miRNA-425 oligonucleotide
gaaagcgcuuuggaaugacacgaucacucccguugagugggcacccgagaagccaucgggaaugucguguccgcccagugcucuuuc

SEQ ID NO: 29 - RNA, 23 nt, Ophiophagus hannah
agcagcauuguacagggcuauga

SEQ ID NO: 30 - RNA, 25 nt, Eptesicus fuscus
agcagcauuguacagggcuaugaag

SEQ ID NO: 31 - RNA, 23 nt, Sus scrofa
agcagcauuguacagggcuauga

SEQ ID NO: 32 - RNA, 23 nt, Mus musculus
agcagcauuguacagggcuauga

SEQ ID NO: 33 - RNA, 23 nt, Monodelphis domestica
agcagcauuguacagggcuauga

SEQ ID NO: 34 - RNA, 23 nt, Macaca nemestrina
agcagcauuguacagggcuauga

SEQ ID NO: 35 - RNA, 23 nt, Macaca mulatta
agcagcauuguacagggcuauga

SEQ ID NO: 36 - RNA, 23 nt, Pan paniscus
agcagcauuguacagggcuauga

SEQ ID NO: 37 - RNA, 23 nt, Pan troglodytes
agcagcauuguacagggcuauga

SEQ ID NO: 38 - RNA, 23 nt, Tupaia chinensis
agcagcauuguacagggcuauga

SEQ ID NO: 39 - RNA, 23 nt, Equus caballus
agcagcauuguacagggcuauga

SEQ ID NO: 40 - RNA, 23 nt, Equus caballus
agcagcauuguacagggcuauga

SEQ ID NO: 41 - RNA, 23 nt, Lagothrix lagotricha
agcagcauuguacagggcuauga

SEQ ID NO: 42 - RNA, 23 nt, Petromyzon marinus
agcagcauuguacagggcuauga

SEQ ID NO: 43 - RNA, 23 nt, Ornithorhynchus anatinus
agcagcauuguacagggcuauga

SEQ ID NO: 44 - RNA, 23 nt, Capra hircus
agcagcauuguacagggcuauga

SEQ ID NO: 45 - RNA, 25 nt, Eptesicus fuscus
agcagcauuguacagggcuaugaaa

SEQ ID NO: 46 - RNA, 23 nt, Canis familiaris
agcagcauuguacagggcuauga

SEQ ID NO: 47 - RNA, 23 nt, Salmo salar
agcagcauuguacagggcuauga

SEQ ID NO: 48 - RNA, 23 nt, Bos taurus
agcagcauuguacagggcuauga

SEQ ID NO: 49 - RNA, 23 nt, Gallus gallus
agcagcauuguacagggcuauga

SEQ ID NO: 50 - RNA, 23 nt, Ateles geoffroyi
agcagcauuguacagggcuauga

SEQ ID NO: 51 - RNA, 23 nt, Danio rerio
agcagcauuguacagggcuauga

SEQ ID NO: 52 - RNA, 23 nt, Gorilla gorilla
agcagcauuguacagggcuauga

SEQ ID NO: 53 - RNA, 23 nt, Cricetulus griseus
agcagcauuguacagggcuauga

SEQ ID NO: 54 - RNA, 23 nt, Xenopus tropicalis
agcagcauuguacagggcuauga

SEQ ID NO: 55 - RNA, 23 nt, Rattus norvegicus
agcagcauuguacagggcuauga

SEQ ID NO: 56 - RNA, 23 nt, Tetraodon nigroviridis
agcagcauuguacagggcuauga

SEQ ID NO: 57 - RNA, 23 nt, Ictalurus punctatus
agcagcauuguacagggcuauga

SEQ ID NO: 58 - RNA, 23 nt, Fugu rubripes
agcagcauuguacagggcuauga

SEQ ID NO: 59 - RNA, 23 nt, Pongo pygmaeus
agcagcauuguacagggcuauga

SEQ ID NO: 60 - RNA, 23 nt, Taeniopygia guttata
agcagcauuguacagggcuauga

SEQ ID NO: 61 - RNA, 23 nt, Fugu rubripes
agcagcauuguacagggcuauga

SEQ ID NO: 62 - RNA, 23 nt, Ophiophagus hannah
agcagcauuguacagggcuauga

SEQ ID NO: 63 - RNA, 23 nt, Cyprinus carpio
agcagcauuguacagggcuauga

SEQ ID NO: 64 - RNA, 22 nt, Anolis carolinensis
agcagcauuguacagggcuaug

SEQ ID NO: 65 - RNA, 22 nt, Ovis aries
agcagcauuguacagggcuaug

SEQ ID NO: 66 - RNA, 21 nt, Canis familiaris
agcagcauuguacagggcuau

SEQ ID NO: 67 - RNA, 21 nt, Tupaia chinensis
agcagcauuguacagggcuau

SEQ ID NO: 68 - RNA, 21 nt, Oryzias latipes
agcagcauuguacagggcuau

SEQ ID NO: 69 - RNA, 21 nt, Oryzias latipes
agcagcauuguacagggcuau

SEQ ID NO: 70 - RNA, 22 nt, Salmo salar
agcagcauuguacagggcuauc

SEQ ID NO: 71 - RNA, 22 nt, Ictalurus punctatus
agcagcauuguacagggcuauc

SEQ ID NO: 72 - RNA, 22 nt, Ovis aries
agcagcauuguacagggcuauc

SEQ ID NO: 73 - RNA, 22 nt, Cyprinus carpio
agcagcauuguacagggcuauc

SEQ ID NO: 74 - RNA, 22 nt, Cricetulus griseus
agcagcauuguacagggcuauc

SEQ ID NO: 75 - RNA, 22 nt, Ornithorhynchus anatinus
agcagcauuguacagggcuauc

SEQ ID NO: 76 - RNA, 22 nt, Bos taurus
agcagcauuguacagggcuauc

SEQ ID NO: 77 - skipped

SEQ ID NO: 78 - RNA, 22 nt, Anolis carolinensis
agcagcauuguacagggcuauc

SEQ ID NO: 79 - RNA, 25 nt, Eptesicus fuscus
agcagcauuguacagggcuaucaaa

SEQ ID NO: 80 - RNA, 23 nt, Lagothrix lagotricha
agcagcauuguacagggcuauca

SEQ ID NO: 81 - RNA, 23 nt, Danio rerio
agcagcauuguacagggcuauca

SEQ ID NO: 82 - RNA, 23 nt, Macaca nemestrina
agcagcauuguacagggcuauca

SEQ ID NO: 83 - RNA, 23 nt, Rattus norvegicus
agcagcauuguacagggcuauca

SEQ ID NO: 84 - RNA, 23 nt, Sus scrofa
agcagcauuguacagggcuauca

SEQ ID NO: 85 - RNA, 23 nt, Gallus gallus
agcagcauuguacagggcuauca

SEQ ID NO: 86 - RNA, 23 nt, Monodelphis domestica
agcagcauuguacagggcuauca

SEQ ID NO: 87 - RNA, 23 nt, Ophiophagus hannah
agcagcauuguacagggcuauca

SEQ ID NO: 88 - RNA, 23 nt, Macaca mulatta
agcagcauuguacagggcuauca

SEQ ID NO: 89 - RNA, 23 nt, Homo sapiens
agcagcauuguacagggcuauca

SEQ ID NO: 90 - RNA, 23 nt, Equus caballus
agcagcauuguacagggcuauca

SEQ ID NO: 91 - RNA, 23 nt, Taeniopygia guttata
agcagcauuguacagggcuauca

SEQ ID NO: 92 - RNA, 23 nt, Tetraodon nigroviridis
agcagcauuguacagggcuauca

SEQ ID NO: 93 - RNA, 23 nt, Gorilla gorilla
agcagcauuguacagggcuauca

SEQ ID NO: 94 - RNA, 23 nt, Pan troglodytes
agcagcauuguacagggcuauca

SEQ ID NO: 95 - RNA, 23 nt, Xenopus tropicalis
agcagcauuguacagggcuauca

SEQ ID NO: 96 - RNA, 23 nt, Pan paniscus
agcagcauuguacagggcuauca

SEQ ID NO: 97 - RNA, 23 nt, Mus musculus
agcagcauuguacagggcuauca

SEQ ID NO: 98 - RNA, 23 nt, Pongo pygmaeus
agcagcauuguacagggcuauca

SEQ ID NO: 99 - RNA, 21 nt, Capra hircus
agcagcauuguacagggcuau

SEQ ID NO: 100 - RNA, 23 nt, Petromyzon marinus
agcagcaucguacagggcuugga

SEQ ID NO: 101 - RNA, 21 nt, Danio rerio
agcagcauuguacagggcuuu

SEQ ID NO: 102 - RNA, 19 nt, Ictalurus punctatus
agcagcauuguacagggcu

SEQ ID NO: 103 - RNA, 30 nt, Capra hircus
agcuucuuuacagugcugccuuguagcauu

SEQ ID NO: 104 - RNA, 24 nt, Taeniopygia guttata
agcuucuuuacagugcugccuugu

SEQ ID NO: 105 - RNA, 24 nt, Cricetulus griseus
agcuucuuuacagugcugccuugu

SEQ ID NO: 106 - RNA, 23 nt, Ophiophagus hannah
agcuucuuuacagugcugccuug

SEQ ID NO: 107 - RNA, 23 nt, Mus musculus
agcuucuuuacagugcugccuug

SEQ ID NO: 108 - RNA, 23 nt, Gallus gallus
agcuucuuuacagugcugccuug

SEQ ID NO: 109 - RNA, 23 nt, Monodelphis domestica
agcuucuuuacagugcugccuug

SEQ ID NO: 110 - RNA, 23 nt, Ornithorhynchus anatinus
agcuucuuuacagugcugccuug

SEQ ID NO: 111 - RNA, 21 nt, Anolis carolinensis
agcuucuuuacagugcugccu

SEQ ID NO: 112 - RNA, 25 nt, Monodelphis domestica
ucggcuucuuuacagugcugccuug

SEQ ID NO: 113 - RNA, 25 nt, Gallus gallus
ucggcuucuuuacagugcugccuug

SEQ ID NO: 114 - RNA, 25 nt, Ornithorhynchus anatinus
ucggcuucuuuacagugcugccuug

SEQ ID NO: 115 - RNA, 25 nt, Macaca mulatta
ucggcuucuuuacagugcugccuug

SEQ ID NO: 116 - RNA, 23 nt, Mus musculus
ggcuucuuuacagugcugccuug

SEQ ID NO: 117 - RNA, 24 nt, Rattus norvegicus
ggcuucuuuacagugcugccuugu

SEQ ID NO: 118 - RNA, 24 nt, Ophiophagus hannah
ggcuucuuuacagugcugccuugu

SEQ ID NO: 119 - RNA, 24 nt, Ophiophagus hannah
agcuucuuuacaguguugccuugu

SEQ ID NO: 120 - RNA, 24 nt, Rattus norvegicus
agcuucuuuacaguguugccuugu

SEQ ID NO: 121 - RNA, 23 nt, Macaca mulatta
agcuucuuuacaguguugccuug

SEQ ID NO: 122 - RNA, 23 nt, Mus musculus
agcuucuuuacaguguugccuug

SEQ ID NO: 123 - RNA, 23 nt, Gallus gallus
agcuucuuuacaguguugccuug

SEQ ID NO: 124 - RNA, 23 nt, Salmo salar
agcuucuuuacaguguugccuug

SEQ ID NO: 125 - RNA, 21 nt, Ornithorhynchus anatinus
agcuucuuuacaguguugccu

SEQ ID NO: 126 - RNA, 20 nt, Anolis carolinensis
agcuucuuuacaguguugcc

SEQ ID NO: 127 - RNA, 23 nt, Danio rerio
agcuucuuuacaguguugucuug

SEQ ID NO: 128 - RNA, 17 nt, Rattus norvegicus
agcuucuuuacagugcu

SEQ ID NO: 129 - RNA, 24 nt, Salmo salar
agccucuuuaugaugcugccuugu

SEQ ID NO: 130 - RNA, 23 nt, Petromyzon marinus
ggccucuuuacagugcuaucuug

SEQ ID NO: 131 - RNA, 22 nt, Capra hircus
ucaaaugcucagacuccugugg

SEQ ID NO: 132 - RNA, 20 nt, Gorilla gorilla
ucaaaugcucagacuccugu

SEQ ID NO: 133 - RNA, 20 nt, Saguinus labiatus
ucaaaugcucagacuccugu

SEQ ID NO: 134 - RNA, 20 nt, Macaca nemestrina
ucaaaugcucagacuccugu

SEQ ID NO: 135 - RNA, 20 nt, Lagothrix lagotricha
ucaaaugcucagacuccugu

SEQ ID NO: 136 - RNA, 20 nt, Pan paniscus
ucaaaugcucagacuccugu

SEQ ID NO: 137 - RNA, 20 nt, Canis familiaris
ucaaaugcucagacuccugu

SEQ ID NO: 138 - RNA, 20 nt, Pan troglodytes
ucaaaugcucagacuccugu

SEQ ID NO: 139 - RNA, 20 nt, Sus scrofa
ucaaaugcucagacuccugu

SEQ ID NO: 140 - RNA, 23 nt, Pongo pygmaeus
ucaaaugcucagacuccuguggu

SEQ ID NO: 141 - RNA, 20 nt, Canis familiaris
ucaaaugcucagacuccuug

SEQ ID NO: 142 - RNA, 20 nt, Sus scrofa
ucaaaugcucagacuccuug

SEQ ID NO: 143 - RNA, 23 nt, Capra hircus
ucaaaugcucagacuccuuggug

SEQ ID NO: 144 - RNA, 22 nt, Bos taurus
ucaaaugcucagacuccuuggu

SEQ ID NO: 145 - RNA, 23 nt, Bos taurus
ucaaaugcucagacuccuguggu

SEQ ID NO: 146 - RNA, 23 nt, Macaca mulatta
ucaaaugcucagacuccuguggu

SEQ ID NO: 147 - RNA, 23 nt, Equus caballus
ucaaaugcucagacuccuguggu

SEQ ID NO: 148 - RNA, 23 nt, Mus musculus
ccaagugcucagaugcuuguggu

SEQ ID NO: 149 - RNA, 22 nt, Rattus norvegicus
caagugcucagaugucuguggu

SEQ ID NO: 150 - RNA, 22 nt, Macaca mulatta
acggauguuugagcaugugcua

SEQ ID NO: 151 - RNA, 22 nt, Capra hircus
ccacggauguuugagcaugugc

SEQ ID NO: 152 - DNA, 858 bp, Homo sapiens
gagacagggacagacgtaggccaagagaggggaaccagagaggaaccagaggggagagacagagcagcaagcagtggattgctccttgacgacgccagcatgagctccttctccaccaccaccgtgagcttcctccttttactggcattccagctcctaggtcagaccagagctaatcccatgtacaatgccgtgtccaacgcagacctgatggatttcaagaatttgctggaccatttggaagaaaagatgcctttagaagatgaggtcgtgcccccacaagtgctcagtgagccgaatgaagaagcgggggctgctctcagccccctccctgaggtgcctccctggaccggggaagtcagcccagcccagagagatggaggtgccctcgggcggggcccctgggactcctctgatcgatctgccctcctaaaaagcaagctgagggcgctgctcactgcccctcggagcctgcggagatccagctgcttcgggggcaggatggacaggattggagcccagagcggactgggctgtaacagcttccggtactgaagataacagccagggaggacaagcagggctgggcctagggacagactgcaagaggctcctgtcccctggggtctctgctgcatttgtgtcatcttgttgccatggagttgtgatcatcccatctaagctgcagcttcctgtcaacacttctcacatcttatgctaactgtagataaagtggtttgatggtgacttcctcgcctctcccaccccatgcattaaattttaaggtagaacctcacctgttactgaaagtggtttgaaagtgaataaacttcagcaccatggacagaagacaaaaaa

SEQ ID NO: 153 - protein, 30 aa, Artificial Sequence
  Description of Artificial Sequence: Synthetic polypeptide
AALAEALAEALAEALAEALAEALAAAAGGC

SEQ ID NO: 154 - protein, 15 aa, Artificial Sequence
  Description of Artificial Sequence: Synthetic peptide
ALEALAEALEALAEA

SEQ ID NO: 155 - protein, 22 aa, Artificial Sequence
  Description of Artificial Sequence: Synthetic peptide
GLFEAIEGFIENGWEGMIWDYG

SEQ ID NO: 156 - protein, 23 aa, Artificial Sequence
  Description of Artificial Sequence: Synthetic peptide
GLFGAIAGFIENGWEGMIDGWYG

SEQ ID NO: 157 - protein, 48 aa, Artificial Sequence
  Description of Artificial Sequence: Synthetic polypeptide
GLFEAIEGFIENGWEGMIDGWYGCGLFEAIEGFIENGWEGMIDGWYGC

SEQ ID NO: 158 - protein, 44 aa, Artificial Sequence
  Description of Artificial Sequence: Synthetic polypeptide
GLFEAIEGFIENGWEGMIDGGCGLFEAIEGFIENGWEGMIDGGC

SEQ ID NO: 159 - protein, 35 aa, Artificial Sequence
  Description of Artificial Sequence: Synthetic polypeptide
GLFGALAEALAEALAEHLAEALAEALEALAAGGSC

SEQ ID NO: 160 - protein, 34 aa, Artificial Sequence
  Description of Artificial Sequence: Synthetic polypeptide
GLFEAIEGFIENGWEGLAEALAEALEALAAGGSC

SEQ ID NO: 161 - protein, 41 aa, Artificial Sequence
  Description of Artificial Sequence: Synthetic polypeptide
  MOD_RES (17)..(17): Norleucine
  MOD_RES (38)..(38): Norleucine
GLFEAIEGFIENGWEGXIDGKGLFEAIEGFIENGWEGXIDG

SEQ ID NO: 162 - protein, 19 aa, Artificial Sequence
  Description of Artificial Sequence: Synthetic peptide
LFEALLELLESLWELLLEA

SEQ ID NO: 163 - protein, 20 aa, Artificial Sequence
  Description of Artificial Sequence: Synthetic peptide
GLFKALLKLLKSLWKLLLKA

SEQ ID NO: 164 - protein, 20 aa, Artificial Sequence
  Description of Artificial Sequence: Synthetic peptide
GLFRALLRLLRSLWRLLLRA

SEQ ID NO: 165 - protein, 30 aa, Artificial Sequence
  Description of Artificial Sequence: Synthetic polypeptide
WEAKLAKALAKALAKHLAKALAKALKACEA

SEQ ID NO: 166 - protein, 22 aa, Artificial Sequence
  Description of Artificial Sequence: Synthetic peptide
GLFFEAIAEFIEGGWEGLIEGC

SEQ ID NO: 167 - protein, 26 aa, Artificial Sequence
  Description of Artificial Sequence: Synthetic peptide
GIGAVLKVLTTGLPALISWIKRKRQQ

SEQ ID NO: 168 - protein, 8 aa, Artificial Sequence
  Description of Artificial Sequence: Synthetic peptide
HHHHHWYG

SEQ ID NO: 169 - protein, 10 aa, Artificial Sequence
  Description of Artificial Sequence: Synthetic peptide
CHKKKKKKHC

SEQ ID NO: 170 - protein, 16 aa, Artificial Sequence
  Description of Artificial Sequence: Synthetic peptide
RQIKIWFQNRRMKWKK

SEQ ID NO: 171 - protein, 14 aa, Artificial Sequence
  Description of Artificial Sequence: Synthetic peptide
GRKKRRQRRRPPQC

SEQ ID NO: 172 - protein, 27 aa, Artificial Sequence
  Description of Artificial Sequence: Synthetic peptide
GALFLGWLGAAGSTMGAWSQPKKKRKV

SEQ ID NO: 173 - protein, 18 aa, Artificial Sequence
  Description of Artificial Sequence: Synthetic peptide
LLIILRRRIRKQAHAHSK

SEQ ID NO: 174 - protein, 26 aa, Artificial Sequence
  Description of Artificial Sequence: Synthetic peptide
GWTLNSAGYLLKINLKALAALAKKIL

SEQ ID NO: 175 - protein, 18 aa, Artificial Sequence
  Description of Artificial Sequence: Synthetic peptide
KLALKLALKALKAALKLA

SEQ ID NO: 176 - protein, 9 aa, Artificial Sequence
  Description of Artificial Sequence: Synthetic peptide
RRRRRRRRR

SEQ ID NO: 177 - protein, 10 aa, Artificial Sequence
  Description of Artificial Sequence: Synthetic peptide
KFFKFFKFFK

SEQ ID NO: 178 - protein, 37 aa, Artificial Sequence
  Description of Artificial Sequence: Synthetic polypeptide
LLGDFFRKSKEKIGKEFKRIVQRIKDFLRNLVPRTES

SEQ ID NO: 179 - protein, 31 aa, Artificial Sequence
  Description of Artificial Sequence: Synthetic polypeptide
SWLSKTAKKLENSAKKRISEGIAIAIQGGPR

SEQ ID NO: 180 - protein, 30 aa, Artificial Sequence
  Description of Artificial Sequence: Synthetic polypeptide
ACYCRIPACIAGERRYGTCIYQGRLWAFCC

SEQ ID NO: 181 - protein, 36 aa, Artificial Sequence
  Description of Artificial Sequence: Synthetic polypeptide
DHYNCVSSGGQCLYSACPIFTKIQGTCYRGKAKCCK

SEQ ID NO: 182 - protein, 42 aa, Artificial Sequence
  Description of Artificial Sequence: Synthetic polypeptide
RRRPRPPYLPRPRPPPFFPPRLPPRIPPGFPPRFPPRFPGKR

SEQ ID NO: 183 - protein, 13 aa, Artificial Sequence
  Description of Artificial Sequence: Synthetic peptide
ILPWKWPWWPWRR

SEQ ID NO: 184 - protein, 16 aa, Artificial Sequence
  Description of Artificial Sequence: Synthetic peptide
AAVALLPAVLLALLAP

SEQ ID NO: 185 - protein, 11 aa, Artificial Sequence
  Description of Artificial Sequence: Synthetic peptide
AALLPVLLAAP

SEQ ID NO: 186 - protein, 12 aa, Artificial Sequence
  Description of Artificial Sequence: Synthetic peptide
RKCRIVVIRVCR

SEQ ID NO: 187 - RNA, 23 nt, Homo sapiens
ugauagcccuguacaaugcugcu

SEQ ID NO: 188 - RNA, 8 nt, Artificial Sequence
  Description of Artificial Sequence: Synthetic oligonucleotide
augcugcu

SEQ ID NO: 189 - RNA, 7 nt, Artificial Sequence
  Description of Artificial Sequence: Synthetic oligonucleotide
augcugc

SEQ ID NO: 190 - RNA, 7 nt, Artificial Sequence
  Description of Artificial Sequence: Synthetic oligonucleotide
ugcugcu

SEQ ID NO: 191 - RNA, 7 nt, Artificial Sequence
  Description of Artificial Sequence: Synthetic oligonucleotide
augcugc

SEQ ID NO: 192 - RNA, 6 nt, Artificial Sequence
  Description of Artificial Sequence: Synthetic oligonucleotide
gcugcu

SEQ ID NO: 193 - RNA, 6 nt, Artificial Sequence
  Description of Artificial Sequence: Synthetic oligonucleotide
ugcugc

SEQ ID NO: 194 - RNA, 6 nt, Artificial Sequence
  Description of Artificial Sequence: Synthetic oligonucleotide
augcug

SEQ ID NO: 195 - protein, 29 aa, Artificial Sequence
  Description of Artificial Sequence: Synthetic peptide
AALEALAEALEALAEALEALAEAAAAGGC

SEQ ID NO: 196 - RNA, 23 nt, Homo sapiens
ucgucguaacaugucccgauacu

SEQ ID NO: 197 - RNA, 23 nt, Homo sapiens
aguuuacgagucugaggacacca

SEQ ID NO: 198 - RNA, 23 nt, Homo sapiens
ucgucguaacaugucccgauagu

SEQ ID NO: 199 - RNA, 21 nt, Homo sapiens
uaaugcauggggugggagagg

SEQ ID NO: 200 - RNA, 21 nt, Homo sapiens
uaaugcguggggugggagagg

SEQ ID NO: 201 - DNA, 23 bp, Homo sapiens
tgtcccctggggtctctgctgca

SEQ ID NO: 202 - RNA, 26 nt, Homo sapiens
uccccuggggucucugcugcauuugu

SEQ ID NO: 203 - RNA, 65 nt, Homo sapiens
cuguuaaugcuaaucgugauagggguuuuugccuccaacugacuccuacauauuagcauuaacag

SEQ ID NO: 204 - DNA, 22 bp, Artificial Sequence
  Description of Artificial Sequence: Synthetic oligonucleotide
cccctatcacgattagcattaa

SEQ ID NO: 205 - DNA, 22 bp, Artificial Sequence
  Description of Artificial Sequence: Synthetic oligonucleotide
cccctatcacgattagcattaa

SEQ ID NO: 206 - DNA, 22 bp, Artificial Sequence
  Description of Artificial Sequence: Synthetic oligonucleotide
cccctatcacgattagcattaa

SEQ ID NO: 207 - RNA, 21 nt, Unknown
  Description of Unknown: target site for miR-105 of the major allele of rs5067
caaaugaagcagagaccccag

SEQ ID NO: 208 - RNA, 21 nt, Unknown
  Description of Unknown: target site for miR-105 of the minor allele of rs5067
cgaaugaagcagagaccccag

SEQ ID NO: 209 - RNA, 22 nt, Homo sapiens
cuccuacauauuagcauuaaca

SEQ ID NO: 210 - RNA, 6 nt, Homo sapiens
uccuac

SEQ ID NO: 211 - RNA, 65 nt, Mus musculus
cuguuaaugcuaaucgugauagggguuuuuaccuccaacugacuccuacauguuagcauuaacag

SEQ ID NO: 212 - RNA, 23 nt, Mus musculus
uuaaugcuaauugugauaggggu

SEQ ID NO: 213 - RNA, 80 nt, Anolis carolinensis
cacacguuguuaaugcuaaucgugauagggguuuuuaucucugacugacuccuacauguuagcauuaagaauguguagug

SEQ ID NO: 214 - RNA, 63 nt, Bos taurus
uguuaaugcuaaucgugauagggguuuuuaccucggacugacuccuacauguuagcauuaaca

SEQ ID NO: 215 - RNA, 71 nt, Cyprinus carpio
ucagguuuaaugcuaaucgugauagggguuuuguacuacugaacaccuaugcuguuagcauuaguccugcg

SEQ ID NO: 216 - RNA, 61 nt, Canis familiaris
uuaaugcuaaucgugauagggguuuuuaccuccgacugacuccuacauguuagcauuaaca

SEQ ID NO: 217 - RNA, 90 nt, Cricetulus griseus
cuguaugcuguuaaugcuaaucgugauagggguuuuugcgucugacugacuccuacauguuagcauuaacaggguacgaugccugccugc

SEQ ID NO: 218 - RNA, 99 nt, Capra hircus
uugcugugggcugugugcuauuaaugcuaaucgugauagggguuuuuaccucggacugacuccuacauguuagcauuaacagggcauggcgccuguuac

SEQ ID NO: 219 - RNA, 78 nt, Danio rerio
uggugcagguuuaaugcuaaucgugauagggguuuagugcugaugaacaccuaugcuguuagcauuaaucuugcgcua

SEQ ID NO: 220 - RNA, 65 nt, Equus caballus
cuguuaaugcuaaucgugauagggguuuuuaccucggacugacuccuacauguuagcauuaacag

SEQ ID NO: 221 - RNA, 135 nt, Eptesicus fuscus
cugaaagccggaggccugcuggaggcuguaugcuguuaaugcuaaucgugauagggguuuuuaccuccgacugacuccuacauguuagcauuaacaggguacggugccugauacuagcauucacacggaacagau

SEQ ID NO: 222 - RNA, 63 nt, Gallus gallus
uguuaaugcuaaucgugauagggguuuuuaccucugaaugacuccuacauguuagcauuaaca

SEQ ID NO: 223 - RNA, 111 nt, Gorilla gorilla
uugcuguaggcuguaugcuguuaaugcuaaucgugauagggguuuuugccuccaacugacuccuacauauuagcauuaacaggguaugaugccuguuacuagcauucacau

SEQ ID NO: 224 - RNA, 60 nt, Ictalurus punctatus
uuaaugcuaaucgugauagggguuuagugcugaugaacaccuaugcuguuagcauuaauc

SEQ ID NO: 225 - RNA, 65 nt, Mus musculus
cuguuaaugcuaauugugauagggguuuuggccucugacugacuccuaccuguuagcauuaacag

SEQ ID NO: 226 - RNA, 105 nt, Ornithorhynchus anatinus
gggacgccgugggccgcaggucguuaaugcuaaucgugauagggguuuucucugucugacugaccccugcccguuagcauuagcaacgugcggccccucgaaccc

SEQ ID NO: 227 - RNA, 65 nt, Pongo pygmaeus; identical to SEQ ID NO: 211

SEQ ID NO: 228 - RNA, 64 nt, Pan troglodytes
uguuaaugcuaaucgugauagggguuuuugccuccaaaugacuccuacauauuagcauuaacag

SEQ ID NO: 229 - RNA, 65 nt, Rattus norvegicus; identical to SEQ ID NO: 225

SEQ ID NO: 230 - RNA, 65 nt, Salmo salar
uuaaugcuaaucgugauagggguucuguuguuuaaaaaaaagacaccuagcauguaagcauuagc

SEQ ID NO: 231 - RNA, 65 nt, Salmo salar
uuaaugcuaaucgugauagggguucuguuguuaaaaaaaaagacaccuagcauguaagcauuagc

SEQ ID NO: 232 - RNA, 81 nt, Sus scrofa
cuauaugcuguuaaugcuaauugugauagggguuuuuaccuccgucugacuccuacauguuagcauuaacaggguaugaug

SEQ ID NO: 233 - RNA, 61 nt, Tupaia chinensis
uuaaugcuaaucgugauagggguuuuugccuccgacugacuccuacauguuagcauuaaca

SEQ ID NO: 234 - RNA, 71 nt, Taeniopygia guttata
cuguauguuguuaaugcuaaucgugauagggguuuuuaccuaugacugacuccuacacguuagcauuaaca

SEQ ID NO: 235 - RNA, 69 nt, Xenopus tropicalis
uauuaaugcuaaucgugauagggguuuuuuaauacauauugacuccuacauguuagcauuauuaucaug

SEQ ID NO: 236 - RNA, 7 nt, Homo sapiens
uaaugca

SEQ ID NO: 237 - RNA, 7 nt, Homo sapiens
uaaugcg

SEQ ID NO: 238 - DNA, 23 bp, Homo sapiens
accacaggagtctgagcatttga

SEQ ID NO: 239 - DNA, 22 bp, Artificial Sequence
  Description of Artificial Sequence: Synthetic oligonucleotide
  Description of Combined DNA/RNA Molecule: Synthetic oligonucleotide
ccccuaucacaatuagcauuaa

SEQ ID NO: 240 - DNA, 15 bp, Artificial Sequence
  Description of Artificial Sequence: Synthetic oligonucleotide
  modified_base (1)..(1): Locked nucleic acid moiety
  modified_base (3)..(3): Locked nucleic acid moiety
  modified_base (5)..(7): Locked nucleic acid moiety
  modified_base (10)..(12): Locked nucleic acid moiety
  modified_base (14)..(15): Locked nucleic acid moiety
tcacaattagcatta

SEQ ID NO: 241 - DNA, 44 bp, Artificial Sequence
  Description of Artificial Sequence: Synthetic oligonucleotide
ctcgcctctcccaccccatcgtggcaattttaaggtagaacctc

SEQ ID NO: 242 - DNA, 45 bp, Artificial Sequence
  Description of Artificial Sequence: Synthetic oligonucleotide
gaggttctaccttaaaattgccacgatggggtgggagaggcgagg

SEQ ID NO: 243 - DNA, 31 bp, Artificial Sequence
  Description of Artificial Sequence: Synthetic oligonucleotide
gcctctcccaccccacgcattaaattttaag

SEQ ID NO: 244 - DNA, 31 bp, Artificial Sequence
  Description of Artificial Sequence: Synthetic oligonucleotide
cttaaaatttaatgcgtggggtgggagaggc

SEQ ID NO: 245 - DNA, 31 bp, Artificial Sequence
  Description of Artificial Sequence: Synthetic oligonucleotide
ggtctctgctgcattcgtgtcatcttgttgc

SEQ ID NO: 246 - DNA, 31 bp, Artificial Sequence
  Description of Artificial Sequence: Synthetic oligonucleotide
gcaacaagatgacacgaatgcagcagagacc

SEQ ID NO: 247 - DNA, 45 bp, Artificial Sequence
  Description of Artificial Sequence: Synthetic oligonucleotide
ctcctgtcccctggggtctcaacgcgatttgtgtcatcttgttgc

SEQ ID NO: 248 - DNA, 45 bp, Artificial Sequence
  Description of Artificial Sequence: Synthetic oligonucleotide
gcaacaagatgacacaaatcgcgttgagaccccaggggacaggag

SEQ ID NO: 249 - RNA, 87 nt, Unknown; identical to SEQ ID NO: 28
  Description of Unknown: pre-miRNA-425 oligonucleotide

SEQ ID NO: 250 - RNA, 54 nt, Homo sapiens
aagaggcuccuguccccuggggucucugcugcauuugugucaucuuguugccau

SEQ ID NO: 251 - RNA, 54 nt, Pan troglodytes
aagaggcuccuguccccuggggucucugcugcauucgugucaucuuguugccau

SEQ ID NO: 252 - RNA, 54 nt, Macaca mulatta
aagagguuccugcccgcuggggucucuccugcauucgugucaucucguugucau

SEQ ID NO: 253 - RNA, 45 nt, Otolemur garnetti
cccauccccugggcucucucauacaacuccgucucaucuugccau

SEQ ID NO: 254 - RNA, 39 nt, Tupaia belangeri
augagacccacaugccacuuggucucaucucguuggcau

SEQ ID NO: 255 - RNA, 51 nt, Mus musculus
aaaagauccuaagcccuuguggugugucacgcagcuuggucacauugccac

SEQ ID NO: 256 - RNA, 51 nt, Rattus norvegicus
aaaagaucccaagcccuugcggugugucacacagcuuggucgcauugccac

SEQ ID NO: 257 - RNA, 56 nt, Cavia porcellus
cagagccccuccuccccuggagucucuccugcagcgaggccucacgucacggucau

SEQ ID NO: 258 - RNA, 54 nt, Oryctolagus cuniculus
aggaacccuccuccuccugcgucucucaagucacuuugucucaucucauugcau

SEQ ID NO: 259 - RNA, 56 nt, Canis familiaris
gagagacccucgucccuuggggucucucacucaacuuugucccagcucauugccau

SEQ ID NO: 260 - RNA, 56 nt, Felis catus
gagagacccucauccuuuggggucucucacucaacuuugucccaucucauugccau

SEQ ID NO: 261 - RNA, 55 nt, Equus caballus
gagagaccauauccccuggggucucucacucaacuuuguuccaucucauugcucu

SEQ ID NO: 262 - RNA, 51 nt, Bos taurus
aagagaauccccuggggucucucacucaacuuugucgcaucugguugccau

SEQ ID NO: 263 - RNA, 55 nt, Dasypus novemcinctus
aagagaccuucagcccuggggucucucaugcaaccuugucccaucucauucucaa

SEQ ID NO: 264 - RNA, 54 nt, Loxodonta africana
gagacccucacccccugggaucucucacucaaccuuguccuaucccauggccau

SEQ ID NO: 265 - RNA, 53 nt, Echinops telfairi
gacacccucgccggcugggacuuccacccagccucgucccaucgcgcagcugu

SEQ ID NO: 266 - RNA, 53 nt, Monodelphis domestica
agaaguuucagauccuuugggacguuggcuuucuuuucuccacucauuguccg

SEQ ID NO: 267 - RNA, 47 nt, Ornithorhynchus anatinus
agaggcuucauauccucgguccggcccgagacuugaacccacgguuc

SEQ ID NO: 268 - RNA, 45 nt, Homo sapiens
guucuaccuuaaaauuuaaugcauggggugggagaggcgaggaag

SEQ ID NO: 269 - RNA, 45 nt, Homo sapiens
guucuaccuuaaaauuuaaugcguggggugggagaggcgaggaag

SEQ ID NO: 270 - RNA, 45 nt, Homo sapiens
cuccauggcaacaagaugacacaaaugcagcagagaccccagggg

SEQ ID NO: 271 - RNA, 45 nt, Homo sapiens
cuccauggcaacaagaugacacgaaugcagcagagaccccagggg

(19) United States
(12) Patent Application Publication
(10) Pub. No.: US 2018/0291374 A1
(43) Pub. Date: Oct. 11, 2018

(54) INHIBITORS OF MICRORNAs miR-155, miR-103, miR-105 and miR-107 THAT REGULATE PRODUCTION OF ATRIAL NATRIURETIC PEPTIDE (ANP) AS THERAPEUTICS AND USES THEREOF

(71) Applicant: THE GENERAL HOSPITAL CORPORATION, Boston, MA (US)

(72) Inventors: Kenneth D. BLOCH, Newton, MA (US); Connie WU, West Roxbury, MA (US); Pankaj ARORA, Birmingham, AL (US); Christopher NEWTON-CHEH, Lexington, MA (US); Thomas J. WANG, Lexington, MA (US)

(73) Assignee: The General Hospital Corporation, Boston, MA (US)

(21) Appl. No.: 15/526,428

(22) PCT Filed: Nov. 12, 2015
(86) PCT No.: PCT/US2015/060362

§ 371 Date: May 12, 2017

Related U.S. Application Data
(60) Provisional application No. 62/078,734, filed on Nov. 12, 2014.

(51) Int. Cl. C12N 15/113

(52) U.S. Cl. CPC C12N 15/113; C12N 2310/141; C12N 2310/113

(57) ABSTRACT

Provided herein are methods, kits and compositions to increase ANP levels in a subject for the treatment of hypertension and/or cardiovascular diseases, comprising at least one anti-miR agent that inhibits at least one of, or a combination of miR-103, miR-105, miR-107 and/or miR-155, alone, or in combination with an inhibitor of miR-425. Anti-miR agents can be small molecules or an oligonucleotide complementary to at least part of the sequence of hsa-miR-103, hsa-miR-105, hsa-miR-107 and/or hsa-miR-155, or their respective seed sequences or.their binding site in the 3′UTR of NPPA gene. Also provided herein are methods, kits and compositions to decrease ANP levels in a subject for the treatment of low blood pressure, comprising at least one or a combination of agonists of miR-103, miR-105, miR-107 and/or miR-155, alone or in combination with a miR-425 agonist.

### CROSS REFERENCE TO RELATED APPLICATIONS

The present application claims benefit under 35 U.S.C. § 119(e) of U.S. Provisional Application No. 62/078,734 filed Nov. 12, 2014, the contents of which is fully incorporated herein by reference in its entirety.

### GOVERNMENT SUPPORT

The invention was made with Government Support under National Institute of Health Grant Numbers: R01-HL098283, R01-HL113933 and R01-HL124262. The government has certain rights in the invention.

### FIELD OF THE INVENTION

The present invention relates generally to methods to increase atrial natriuric peptide (ANP) levels in a subject, e.g., to treat a cardiovascular disease by using an antagonist or inhibitor to at least one of miR-155, miR-103, miR-105, miR-107, alone or in combination with an antagonist to miR-425. Alternative embodiments relate to treating a subject with low blood pressure by administering at least one miRNA agent selected from a miR-155, miR-103, miR-105, miR-107 agent. Other aspects relates to compositions, methods and kits comprising inhibitors of miR-155, miR-103, miR-105, miR-107, and assays and arrays to assess a subject amenable to treatment with an inhibitor of miR-155, miR-103, miR-105, miR-107, alone or in combination with an inhibitor of miR-425 for use in a method to inhibit metastasis of cancer cells in a subject.

### BACKGROUND OF THE INVENTION

The average U.S. adult consumes nearly 10 g of salt per day, far in excess of the limit recommended by the Departments of Agriculture and Health and Human Services.^{,2} High salt intake has been linked to hypertension and cardiovascular disease.^{3, 4} Randomized clinical trials demonstrate that reducing salt intake substantially lowers this risk.⁵ It has been estimated that a 3 g reduction of daily salt intake would save an estimated 44,000 to 92,000 lives annually in the U.S.⁶

High salt consumption can significantly contribute to high blood pressure, hypertension, and cardiovascular disease. High blood pressure represents a potent risk factor for cardiovascular disease, stroke, and kidney disease. Although blood pressure is highly heritable, the genetic causes of variation in blood pressure in the general population have been ill-defined. An important challenge to the study of the genetics of blood pressure is the multifactorial nature of hypertension. Multiple approaches to sub-dividing hypertension by etiology have been proposed. Defining abnormalities of salt-handling has generated particular attention, given the potential implications of “salt-sensitivity” for non-pharmacologic and pharmacologic therapies.

The natriuretic peptide system plays a central role in the response to salt intake. Synthesized by the heart, atrial natriuretic peptide (ANP) and B-type natriuretic peptide (BNP) promote natriuresis, diuresis, and vasodilation. In animals, deficient ANP signaling results in salt-sensitive hypertension, adverse cardiac remodeling, and premature mortality.^{7, 8} Evidence from population genetic studies suggests that variation in plasma natriuretic peptides may alter susceptibility to cardiovascular disease in humans as well. Common genetic polymorphisms in the chromosome 1 region containing NPPA and NPPB, which encode the ANP and BNP propeptides, respectively, are associated with circulating natriuretic peptide levels.⁹ Large-scale epidemiologic studies have established that the same variants are associated with blood pressure with, for example, a 15% lower risk of hypertension in carriers of the rs5068 minor allele.^{9, 10}

Newton-Cheh and colleagues have previously reported several common genetic variants in the locus containing the genes encoding atrial natriuretic peptide (ANP) and B-type natriuretic peptide (BNP), NPPA and NPPB, respectively, that are associated with resting natriuretic peptide (NP) concentrations and blood pressure (C Newton-Cheh et al. Nature Genetics 2009, 41:348-353). Using data from up to 30,000 individuals, they reported that genetically-determined lower NP levels were associated with higher blood pressure and a greater risk of hypertension, directly implicating the NP system in blood pressure regulation.

More than 30% of adults in the US have hypertension, and hypertension is an important risk factor for myocardial infarction, stroke, and heart failure (Go, et al., Circulation 127:143-152.5). Seventy-five percent of adults with hypertension are taking antihypertensive medications, but target blood pressures are attained in only 53%. It is estimated that, in 2010, ˜3% of Americans over the age of 18 had heart failure, and about 50% of patients diagnosed with heart failure die within 5 years (Roger et al., Circulation 125:e2-e2206). The 2010 health care costs associated with treating hypertension and heart failure (including those related to lost productivity) were estimated to be 93.5 and 34.4 billion dollars, respectively (Heidenreich, et al., Circulation 123:933-944.). These data strongly indicate that it is necessary to elucidate the mechanisms responsible for hypertension and demonstrate the need to develop novel treatment strategies for the effective treatment of hypertension and heart failure (HF).

### SUMMARY OF THE INVENTION

The present invention relates to agonists and antagonists to miR-155, miR-103, miR-105 and miR-107 to modulate ANP (atrial natriuretic peptide) in a subject. More specifically, inhibitors to one or more of miR-155, miR-103, miR-105 and miR-107 can be used to increase ANP in a subject in method, kits and compositions for the treatment or prevention of hypertension, high blood pressure and/or a cardiovascular disease or disorder in a subject, including, but not limited to, hypertrophy, heart failure (HF), metabolic syndrome, stroke or renal failure, or any combination thereof. Importantly, as miR-155, miR-103, miR-105 and miR-107 have target binding sites on a variety of different genes and therefore regulate the expression of a multitude of genes, inhibiting one of these miRs alone may increase the expression of an off target gene in addition to increasing NPPA gene expression. Since antagonism of one miR by its self may require a higher dose of the anti-miR to increase NPPA gene expression, and thus may be associated with undesirable side effect, the inventors have discovered a method to inhibit a number of different miRs, therefore reducing the effect of off-target effects, and envision a combination of anti-miRs which can be used at lower doses to maximize the effect on increasing NPPA expression and simultaneously having reduced off-target effects or undesirable side effects.

Using a multidimensional approach, the inventors herein discovered two novel functional miRNAs (miR-155 and miR-105) that target NPPA mRNA and modulate ANP levels in an allele-specific manner. miR-155 and miR-105 repress NPPA mRNA containing the major alleles of rs61764044 and rs5067, respectively, while the minor alleles of each respective variant confer resistance to the miRNAs. Population genetic studies have demonstrated that the minor alleles of rs61764044 and rs5067 are associated with increased plasma ANP levels in humans. Accordingly, the present invention discovers a mechanistic explanationsby which these variants are associated with circulating ANP levels in the human population.

In particular, the inventors have identified multiple miRNAs can regulate ANP production. Anti-miRs targeting miR-425, miR-155, or miR-105 that increase endogenous NPPA mRNA and ANP protein levels could have clinical applications for the treatment of hypertension or heart failure. The biological relevance of targeting these miRNAs is highlighted by the interaction of these miRNAs with genetic variants that have been identified in our human genetic association studies to be associated with circulating human ANP levels. Further studies on the in vivo effects of these anti-miRs in de-repressing ANP production would help characterize phenotypic responses that will further expand our understanding of the clinical applications of such treatments. Moreover, additional studies investigating optimal dosage and combinatorial administration of multiple anti-miRs are needed.

Accordingly, one aspect of the present invention is to use a combination anti-miRs (e.g., a combination of anti-miRs to any one of miR-155, miR-103, miR-107 and miR-105, as well as in some embodiments, in combination with an anti-miR-425 agent) to enable a lower dose of each anti-miR to be used, thereby reducing off-target effects on genes other than ANP. Additionally, a combination anti-miRs (e.g., a combination of anti-miRs to any one of miR-155, miR-103, miR-107 and miR-105, as well as in some embodiments, in combination with an anti-miR-425 agent) also enables efficient increase in expression of NPPA gene and thus potent ANP expression because the effect of the combination of the miRs is deemed to be additive. In some embodiments, a combination of anti-miRs for use in increasing NPPA gene expression is selected from the group consisting of miR-155, miR-103, miR-107 and miR-105 in combination with an anti-miR-425 agent. In some embodiments, an anti-miR-105 is used in combination with an anti-miR-245 agent in the methods, compositions, assays and kits as disclosed herein to increase NPPA gene expression.

In some embodiments, an anti-miR-155 agent is used alone, or in combination with an anti-miR-245 agent in the methods, compositions, assays and kits as disclosed herein to increase NPPA gene expression. In an alternative embodiments, an anti-miR-105 agent is used alone, or in combination with an anti-miR-245 agent in the methods, compositions, assays and kits as disclosed herein to increase NPPA gene expression.

In some embodiments, an anti-miR-103 or anti-miR-107 agent, or both an anti-miR-103 and anti-miR-107 agent are used in combination with an anti-miR-245 agent in the methods, compositions, assays and kits as disclosed herein to increase NPPA gene expression.

Accordingly, in some embodiments, the present invention relates to at least one anti-miR agent, or at least 2 agents, or at least 3 agents, selected from the group of: anti-miR-155, anti-miR-103, anti-miR-107 or anti-miR-105 to specifically inhibit the miR-mediated repression of the NPPA gene. In some embodiments, at least one, or a combination of anti-miRs to any one of miR-155, miR-103, miR-107 and miR-105 can be used in combination with an anti-miR-425 agent, to specifically inhibit the miR-mediated repression of the NPPA gene.

Because miR-155, miR-103, miR-107, miR-105 and miR-425 each target the 3′UTR of the NPPA gene, using a combination of anti-miRs (e.g., to one or more of miR-155, miR-103/miR-107, miR-105 or miR-425) will inhibit the miR-mediated repression of NPPA gene but minimize the inhibition of miR-mediated repression of off-target genes (e.g., genes other than NPPA that are repressed by the miRs) and therefpre minimize any unwanted or adverse side effects. Accordingly, the present invention encompasses inhibiting at least 2, and possibly 3, or 4 or more miRs of any combination of miR-155, miR-103, miR-107 and miR-105 and miR-425, so that inhibition of miR-mediated repression of off-target genes is reduced.

In some embodiments, an anti-miR-155 is used alone in the methods, kits and compositions as disclosed herein to increase expression from the NPPA gene. In some embodiments, an anti-miR-155 is used in combination with an anti-miR-425 in the methods, kits and compositions as disclosed herein to increase expression from the NPPA gene.

In some embodiments, an anti-miR-105 is used alone in the methods, kits and compositions as disclosed herein to increase expression from the NPPA gene. In some embodiments, an anti-miR-105 is used in combination with an anti-miR-425 in the methods, kits and compositions as disclosed herein to increase expression from the NPPA gene.

Another aspect of the present invention relates to agonists to miR-155, miR-103, miR-107 and/or miR-105 to decrease ANP in a subject, for example, for the treatment of hypotension or where it is desirable to increase blood pressure in a subject. In some embodiments, miR-155 or a miR-155 agonist or miR-155 activating agent is used alone in the methods, kits and compositions as disclosed herein to decrease expression from the NPPA gene and decrease ANP in a subject. In some embodiments, a miR-155 or a miR-155 agonist or miR-155 activating agentis used in combination with an anti-miR-425 in the methods, kits and compositions as disclosed herein to decrease expression from the NPPA gene and decrease ANP in a subject. In some embodiments, miR-105 or a miR-105 agonist or miR-105 activating agent is used alone in the methods, kits and compositions as disclosed herein to decrease expression from the NPPA gene and decrease ANP in a subject. In some embodiments, a miR-105 or a miR-105 agonist or miR-105 activating agentis used in combination with an anti-miR-425 in the methods, kits and compositions as disclosed herein to decrease expression from the NPPA gene and decrease ANP in a subject.

The inventors previously identified a microRNA, miR-425, which is expressed in the human heart, that binds to a sequence spanning rs5068 for the major allele (A) (but not the minor allele (G)) and represses ANP expression, which is discussed in P Arora et al. Journal of Clinical Investigation 2013, 123:3378-3382, and International Application WO2013/188787, which is incorporated herein in its entirety. In particular, in International Application WO2013/188787, the inventors previously reported use of an anti-miR to miR-425 to decrease miR-425 function and thus elevate ANP levels in subjects who are AA (major homozygotes) or AG (heterozygotes) for the rs5068 variant of the 3′ untranslated region (3′UTR) of the NPPA gene. Herein, using bioinformatics methods, the inventors have discovered at least 4 additional miRs, miR-155, miR-103, miR-105 and miR-107 that bind to the 3′ untranslated region (3′UTR) of NPPA. While there are some single nucleotide polymorphisms (SNPs) at some of the miR binding sites on NPPA (e.g., variant rs5067 is located in the 3′UTR of NPPA gene that is also part of the binding site for miR-105, and rs5066 is located in the 3′UTR of NPPA gene in a region which is also part of the binding site for miR-103 and miR-107), the majority of the population have a genotype with at least one allele that comprises the binding site for miR-103/miR-107 or miR-105. Furthermore, because these SNPs (i.e., rs5067, rs5066 and rs5068) are not associated or correlated with each other, the presence of a non-susceptible genotype at one miR binding site would still be associated with susceptible genotypes at other miR binding sites, supporting the value of combination therapy.

However, as disclosed herein, the miR-155 seed sequence binds to the 3′UTR of NPPA gene that contains the major allele (A) of the rs61764044 variant. The inventors have discovered that this rs61764044 variant is perfectly assopciated (e.g., linked) with the rs5068 variant, which lies 123 nucleotides upstream in the 3′ UTR of the NPPA gene, indicating that the minor alleles of rs61764044 and rs5068 are inherted together (perfect linkage disequlibrium, r²=1). Although the inventors have previously discovered that the minor allele (G) of the rs5068 variant disrupts the binding of miR-425 and therefore avoids miR-425 repression of NPPA gene, resulting in increased NPPA mRNA and ANP production, (and increased plasma ANP levels, lower blood pressure (BP) and reduced risk of hypertension, the inventors herein have extended this discovery, and demonstrate that the minor allele (G) of the rs61764044 variant results in a G:U wobble base pairing of the miR-155 seed sequence to the 3′UTR of the NPPA gene, therefore disrupts the binding of miR-155 preventing miR-155-mediated repression of NPPA gene, resulting in increased NPPA mRNA and ANP production. As a result, human subjects with the minor allele (G) of the rs61764044 variant have increased plasma ANP levels, lower blood pressure (BP) and reduced risk of hypertension.

Accordingly, anti-miRs to at least one or a combination of miR-155, miR-103, miR-105 and/or miR-107, alone or in combination with an anti-miR-425 agent, can be used to decrease the function of their respective miRs (e.g., decrease the function of miR-155, miR-103, miR-105 and/or miR-107 and/or miR-425), preventing their binding to the 3′ untranslated region (3′UTR) of the NPPA gene, they are useful for elevating ANP levels in virtually all subjects. In some embodiments, an anti-miR to at least one or a combination of miR-155, miR-103, miR-105 and/or miR-107 can be used in combination with an anti-miR-425 agent as disclosed in WO2013/188787 preventing their binding to the 3′ untranslated region (3′UTR) of the NPPA gene, resulting in inhibiting the miR-155, miR-103, miR-105 and/or miR-107 mediated repression of ANP expression and elevating ANP levels in virtually all subjects, as well as, subject who are AA (major homozygotes) or AG (heterozygotes) for the rs5068 variant or rs61764044 variant of the 3′ untranslated region (3′UTR) of the NPPA gene.

Accordingly, the present invention generally relates to a method to treat a cardiovascular disease or disorder, hypertension, or a volume overload state (e.g., congestive heart failure) comprising administering a pharmaceutical composition comprising at least one anti-miR agent which inhibits the function of at least one of, or any combination of, miR-155, miR-103, miR-105 and/or miR-107 to increase ANP expression and biosynthesis. In some embodiments, such a pharmaceutical composition can comprise an additional agent, for example an anti-miR agent that inhibits miR-425 for the treatment of subjects who are AA (major homozygotes) or AG (heterozygotes) for the rs5068 variant, and/or subjects who are AA (major homozygotes) or AG (heterozygotes) for the rs61764044 variant.

In some embodiments, a pharmaceutical composition comprising at least one anti-miR agent which inhibits the function at least one of, or a combination of miR-155, miR-103, miR-105 or miR-107, alone or in combination with an anti-miR-425 agent can be administered to a subject to increase ANP biosynthesis to prevent adverse left ventricular remodeling, which occurs in some patients after myocardial infarction or other myocardial injury.

In alternative embodiments, co-administration of low doses of anti-miRs directed against miR-425 and either miR-155, miR-103, miR-105, or miR-107, or alternatively, low doses of anti-miRs directed against at least 2, or at least 3, or at least 4 or more of the miRs, can be used in a method to augment ANP production for the treatment of hypertension and congestive heart failure, while minimizing adverse “off-target” effects.

Accordingly, one aspect of the present invention relates to a method for increasing ANP levels in a subject, comprising administering to the subject an anti-miR agent that inhibits the function of at least one of: miR-103, miR-105, miR-107 or miR-155, wherein the anti-miR agent prevents miR-103, miR-105, miR-107 or miR-155 mediated repression of NPPA gene expression. In some embodiments, an anti-miR agent is at least 90% complementary to the nucleobase sequence of any one of miR-103, miR-105, miR-107 or miR-155. In some embodiments, an anti-miR agent specifically binds to the seed sequence of any one or a combination of miR-103, miR-105, miR-107 or miR-155, wherein the seed sequence of miR-103 or miR-107 is SEQ ID NO: 4 and the seed sequence of miR-105 is SEQ ID NO: 9, and the seed sequence of miR-155 is 14. In some embodiments, an anti-miR agent binds to the target sequence (e.g., the binding sequence) of miR-103, miR-105, miR-107 or miR-155 in the 3′UTR of the NPPA gene, thereby preventing the binding (and therefore repression of gene expression) by miR-103, miR-105, miR-107 or miR-155. In some embodiments, the method is applicable to a subject that has been identified to have hypertension and has been selected for administration with an anti-miR agent as disclosed herein.

In some embodiments, a composition comprises at least two anti-miR agents selected from the groups of; anti-miR-103 and anti-miR-107; anti-miR-103 and anti-miR-105, anti-miR-105 and anti-miR-107, anti-miR-105 and anti-miR-155, anti-miR-103 and anti-miR-155, or anti-miR-107 and anti-miR-155. In some embodiments, a composition comprises at least one of anti-miR-103, anti-miR-105, anti-miR-107 or anti-miR-155 and can optionally comprise an anti-miR agent that inhibits the function of miR-425. In some embodiments, an anti-miR that inhibits the function of miR-103 inhibits the function of hsa-miR-103a-3p (SEQ ID NO: 1). In some embodiments, an anti-miR that inhibits the function of miR-105 inhibits the function of hsa-miR-105-5p (SEQ ID NO: 7).

In some embodiments, an anti-miR that inhibits the function of miR-105 inhibits the function of hsa-miR 105-5p (SEQ ID NO: 7). In some embodiments, an anti-miR that inhibits the function of miR-107 inhibits the function of hsa-miR 107 (SEQ ID NO: 11). In some embodiments, an anti-miR that inhibits the function of miR-155 inhibits the function of hsa-miR-155-5p (SEQ ID NO: 12).

In some embodiments, the method further comprises administering a therapeutic agent for the treatment of hypertension as disclosed herein.

Another aspect of the present invention relates to an isolated anti-miR oligonucleotide that is at least 90% complementary to the nucleobase sequence of any one of miR-103, miR-105, miR-107 or miR-155 (e.g., at least 90% complementary to any one of SEQ ID NO: 1, 7, 11 and 12) for increasing ANP levels in a subject. Another aspect of the present invention relates to an isolated oligonucleotide comprising a nucleotide sequence that is complementary to, and specifically binds to the seed sequence of any one or a combination of miR-103, miR-105, miR-107, or miR-155 wherein the seed sequence of miR-103 or miR-107 is SEQ ID NO: 4, the seed sequence of miR-105 is SEQ ID NO: 9, and the seed sequence of miR-155 is SEQ ID NO: 14. Another aspect of the present invention relates to an isolated oligonucleotide comprising a nucleotide sequence that is at least 90% complementary to, and specifically binds to the target sequence of miR-103, miR-105, miR-107 or miR-155 in the 3′UTR of the NPPA gene, wherein the target sequence of miR-103 or miR-107 is SEQ ID NO: 6, and the target sequence of miR-105 is SEQ ID NO: 10, SEQ ID NO: 207 or 208, and the target sequence of miR-155 in the 3′UTR is SEQ ID NO: 199 or 200. In some embodiments, an anti-miR-155 agent is an isolated oligonucleotide comprising a nucleotide sequence that is at least 90% complementary to, and specifically binds to the miR-155 target sequence in the 3′UTR of the NPPA gene, where the target sequence is UAAUGCAUGGGGUGGGAGAGG (SEQ ID NO: 199) where the subject has the major “A” allele for rs61764044, or the where the target sequence is UAAUGCGUGGGGUGGGAGAGG (SEQ ID NO: 200) where the subject has the minor “G” allele for rs61764044.

Another aspect of the present invention relates to a method for decreasing ANP levels in a subject, comprising administering to the subject a miR agent that increases the function of at least one of; miR-103, miR-105, miR-107 or miR-155, wherein the miR agent increases miR-103, miR-105, miR-107 or miR-155 mediated repression of the 3′UTR of the NPPA gene. In some embodiments, such a miR agent is at least 90% identical to the nucleobase sequence of any one of miR-103, miR-105, miR-107 or miR-155. In some embodiments, the subject has been identified to have hypotension and has been selected for administration with the miR agent. In some embodiments, the composition comprises at least two miR agents selected from the groups of; miR-103 and miR-107; miR-103 and miR-105, miR-105 and miR-107, miR-105 and miR-155, miR-103 and miR-155 or miR-103 and miR-155. In some embodiments the composition can comprise at least 2, or at least 3 or all four of miR-103, miR-105, miR-107 or miR-155. In some embodiments, the composition can further comprise a miR agent which is miR-425 (e.g., SEQ ID NO: 15) or a mimetic or variant of miR-425 of SEQ ID NO: 15. In some embodiments, an exogenously added miR agent would complement the function of that miR, for example a miR-103 agent would have an additive or synergistic effect to hsa-miR 103a-3p, a miR-105 agent would have an additive or synergistic effect to hsa-miR 105-5p, and a miR-107 agent would have an additive or synergistic effect to hsa-miR-107, and a miR-155 agent would have an additive or synergistic effect to hsa-mir-155-5p. In some embodiments, the method futher comprises administering a therapeutic agent for the treatment of hypotension.

Another aspect of the present invention relates to an assay comprising; (i) measuring the levels of any of miR-103, miR-105, miR-107 or miR-155 in a biological sample obtained from the subject; (ii) comparing the measured levels of miR-103, miR-105, miR-107 or miR-155 with a reference value for the miR measured and if the measured amount of miR-103, miR-105, miR-107 or miR-155 is increased relative to the reference value; (iii) identifying the subject as being at risk of having high blood pressure, hypertension or a cardiovascular disease or disorder. In some embodiments, the assay comprises administering to the subject identified in (ii) an anti-miR agent which inhibits any one of miR-103, miR-105, miR-107 or miR-155, where the anti-miR agent is administered alone or in combination with an anti-miR-425 inhibitor.

Another aspect of the present invention relates to a method of treating a subject with high blood pressure, hypertension or cardiovascular disease or disorder comprising administering to a subject an anti-miR agent that inhibits the function of at least one of; miR-103, miR-105, miR-107 or miR-155, wherein the anti-miR agent prevents miR-103, miR-105, miR-107 or miR-155 mediated repression of the 3′UTR of the NPPA gene, and wherein the subject has been identified to have the plasma expression of any one of; miR-103, miR-105, miR-107 or miR-155 above a pre-defined reference control level for that miRNA.

Another aspect of the present inventon relates to a kit that can contain at least two anti-miR agents from any one of; miR-103, miR-105, miR-107 or miR-155 or miR-425 for administration to the subject, e.g., an anti-miR-155 agent in combination with an anti-miR-425 agent, or an anti-miR-105 agent in combination with an anti-miR-245 agent. In some embodiments, the kit contains at least one anti-miR agents from any one of; miR-103, miR-105, miR-107 or miR-155 or miR-425 for administration formulated for intrathecal or intracranial injection or infusion.

### BRIEF DESCRIPTION OF DRAWINGS

FIGS. 1A-1C show that miR-103, miR-105 and miR-107 reduce luciferase activity of the NPPA 3′UTR luciferase construct. FIG. 1A shows the effect of miR-103 on deceasing luciferase activity. FIG. 1B shows the effect of miR-105 on deceasing luciferase activity. FIG. 1C shows the effect of miR-107 on deceasing luciferase activity. Data are expressed as mean±SEM (n=6).

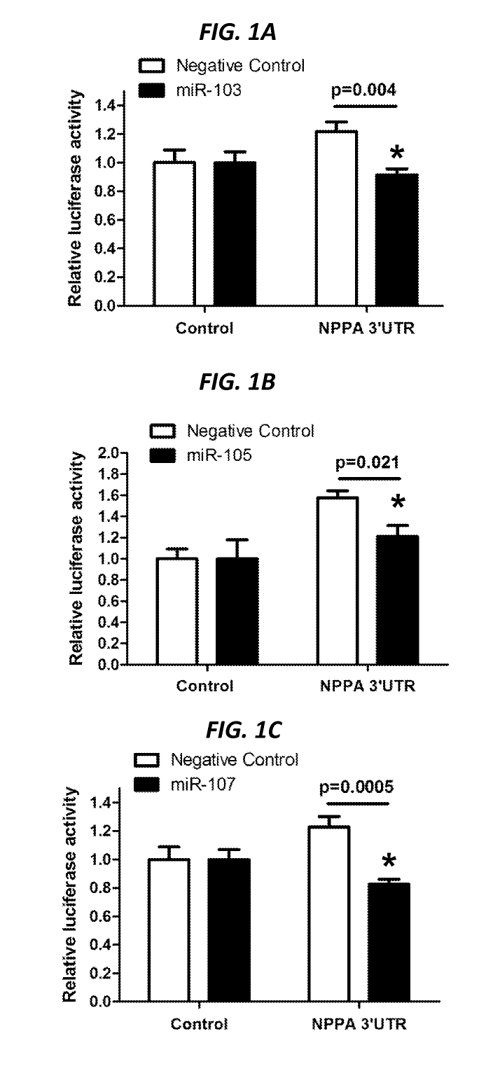

FIGS. 2A-2C show that the NPPA 3′UTR is the direct target of miRs-103, miR-105 and miR-107 and that mutation of the target sequence of NPPA 3′ UTR abolishes the negative regulatory effect of each miR. The effects of miR-103 (SEQ ID NO: 1), miR-105 (SEQ ID NO: 7) and miR-107 (SEQ ID NO: 11) on luciferase activity encoded by the NPPA 3′UTR-luciferase reporters; Luc-NPPA 3′UTR or Luc-Mut, where Luc-Mut comprises a mutation in the target binding site of for miR-103/miR-107 (Luc-Mut comprises TGTGC; SEQ ID NO: 5) in the NPPA 3′UTR. Stated another way Luc-Mut comprises one nucleotide in the region complementary to the miR-105 seed sequence mutated (Luc-Mut NPPA 3′UTR)). The ratio of firefly luciferase activity to renilla luciferase activity was calculated and normalized to that in cells transfected with a plasmid directing expression of firefly luciferase without the NPPA 3′UTR (Control). FIG. 2A shows that miR-103 decreases luciferase activity operatively linked to the wild-type 3′UTR NPPA gene. FIG. 2B shows that miR-105 decreases luciferase activity operatively linked to the wild-type 3′UTR NPPA gene. FIG. 2C shows that miR-107 decreases luciferase activity operatively linked to the wild-type 3′UTR NPPA gene. Data are expressed as mean±SEM (n=6).

FIG. 3 shows the effects of anti-miR-105 (e.g., of SEQ ID NO: 197) on luciferase activity encoded by the NPPA 3′UTR-luciferase reporters, Luc-NPPA 3′UTR and Luc-Mut NPPA 3′UTR. The ratio of firefly luciferase activity to renilla luciferase activity was calculated and normalized to that in cells transfected with a plasmid directing expression of firefly luciferase without the NPPA 3′UTR (Control). Data are expressed as mean±SEM (n=6).

FIGS. 4A-4C show the effect of anti-miR-103, anti-miR-105 and miR-107 on endogenous NPPA mRNA expression levels in human cardiomyocyte progenitor cells isolated from adult human heart auricle tissue. FIG. 4A shows increased NPPA mRNA levels on treatment with anti-miR-105 (of SEQ ID NO: 197). FIG. 4B shows increased NPPA mRNA levels on treatment with anti-miR-103 (SEQ ID NO: 196) or anti-miR-107 (SEQ ID NO: 198). FIG. 4C shows increased NPPA mRNA levels on treatment with anti-miR-425 (SEQ ID NO: 17).

FIGS. 5A-5B shows the effect of miR-105 and anti-miR-105 on endogenous NPPA mRNA expression levels in HepG2 cells. FIG. 5A shows a decrease in NPPA mRNA levels in the presence of miR-105 and FIG. 5B shows the increase in NPPA mRNA levels in the presence of anti-miR-105.

FIG. 6 shows that anti-miR-105 increases Nt-proANP production in COST cells transfected with a plasmid directing expression of a human NPPA cDNA.

FIG. 7 shows the effect of miR-103, miR-105, miR-107 and miR-425 on endogenous NPPA mRNA expression levels in human embryonic stem cell (ESC)-derived cardiomyocytes (hESC-CM), showing that each miR species decreases NPPA mRNA expression.

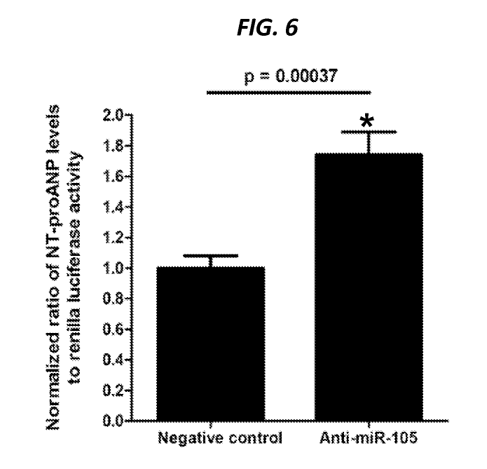

FIG. 8 shows NPPA mRNA levels in hESC-derived cardiomyocytes (hESC-CM) transfected with anti-miR-105, anti-miR-103 and anti-miR-107, showing that each anti-miR increases NPPA mRNA levels and increases Nt-proANP production in human ESC-derived cardiomyocytes. Data are expressed as mean±SEM (n=4). *P<0.001 vs. negative control.

FIG. 9 shows the expression of pri-miR-425, pri-miR-105-1, and pri-miR-105-2 in human left atrial and ventricular tissues.

FIG. 10 shows the location of the predicted miR binding sites of miR-103, miR-107, miR-105, miR-155 and miR-425 in the 3′UTR of NPPA gene.

FIG. 11 shows the predicted target binding sites of miR-103 (SEQ ID NO: 1), miR-107 (SEQ ID NO: 11) and miR-105 (SEQ ID NO: 7) in the 3′UTR of NPPA gene. (The sequences of the regions of the NPPA 3′UTR that shows the predicted miR binding sites are shown 5′ to 3′ and the miR sequences are shown 3′ to 5′). The miR-105 seed sequence is shown in CAPS as 5′-CAAAUGC-3′ (SEQ ID NO: 9); the miR-103 and miR-107 seed sequence is shown in CAPS as 5′-GCAGCAU-3′ (SEQ ID NO: 4). There is only one nucleotide difference between miR-103 (SEQ ID NO: 1) and miR-107 (SEQ ID NO: 11) indicated with the box at the 2^{nd} to last 3′ end. The predicted target binding site for miR-105 in the 3′UTR NPPA gene is underlined in bold and is 5′-UGCUGCAUUUG-3′ (SEQ ID NO: 10). The predicted target binding site for miR-103 and miR-107 in the 3′UTR NPPA gene is 5′-ctctGCtGC-3′ (SEQ ID NO: 6).

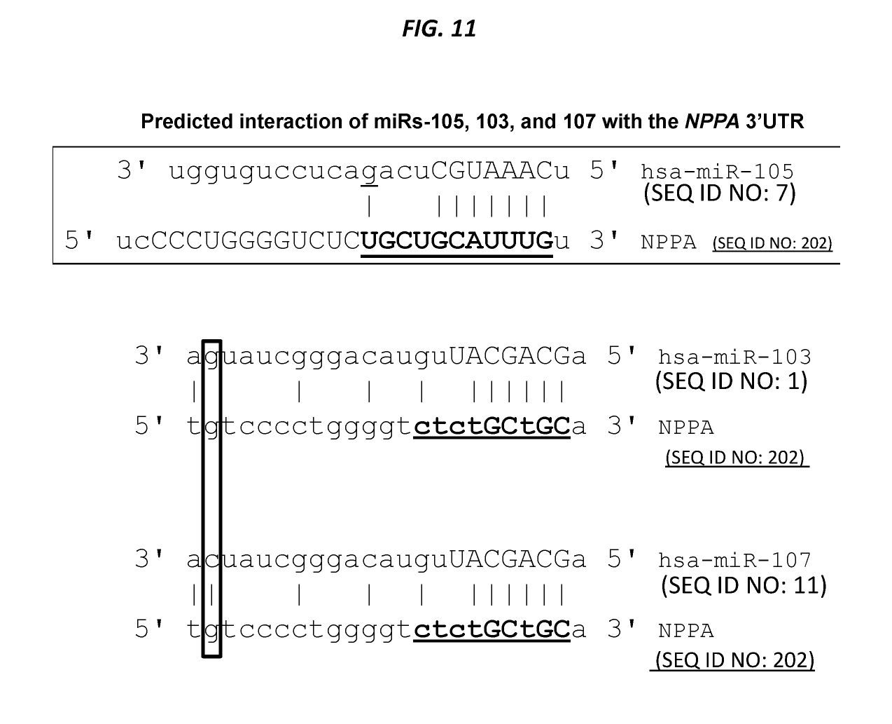

FIG. 12 shows the sequences and homologues of specific members of miR-105, miR-103 and miR-107 families which bind to the 3′UTR of NPPA gene. Shown is the human (hsa-), monkey (mml-) and mouse (mmu-) sequences of miR-105-5p, miR-103a-3p and miR-107.

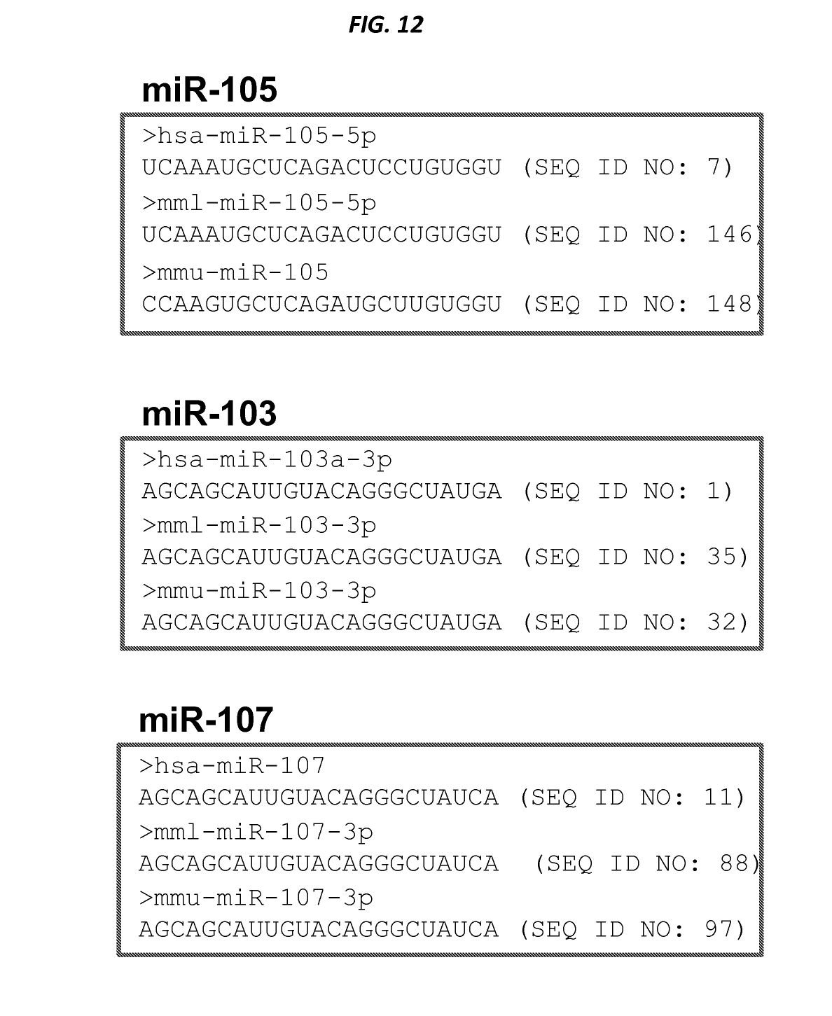

FIG. 13 shows a sequence alignment of a region of the 3′UTR of NPPA gene comprising the target binding sites for miR-103, miR-105, miR-107, miR-155 and miR-425 across multiple species, showing that the binding site is generallyconserved across species.

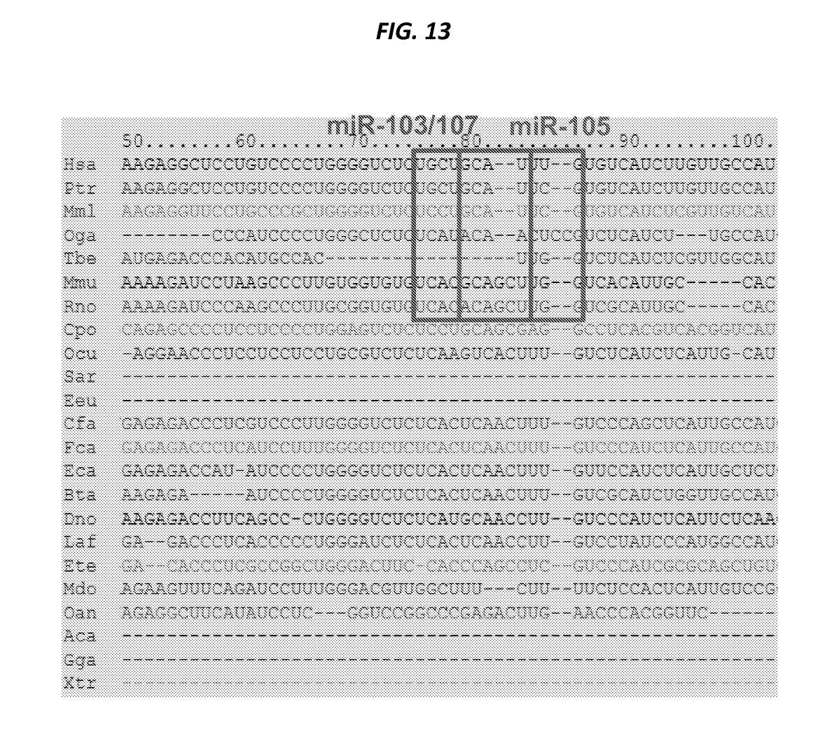

FIG. 14 is a schematic workflow for identification of NPPA-targeting miRNAs. In silico miRNA predictions were based on mirDIP. Evidence of expression in human atrial tissues were based on Hsu et al. (Circ Cardiovasc Genet 2012; 5:327-35).

FIGS. 15A-15D shows four miRNAs (miR-155, miR-103, miR-107 and miR-105) predicted in silico to target the NPPA 3′UTR reduce the luciferase activity produced by the firefly luciferase-NPPA 3′UTR construct. The ratio of firefly luciferase activity to renilla luciferase activity was normalized to the ratio in COS-7 cells transfected with constructs encoding luciferase without the NPPA 3′UTR and renilla. The effect of each miRNA was compared to that of a negative control miRNA (relative luciferase activity). FIG. 15A shows miR-155, FIG. 15B shows miR-103, FIG. 15C shows miR-107, and FIG. 15D shows miR-105 reduced the activity of the WT-Luc construct. Data are mean±SEM (n=6); 2-tailed independent t test.

FIG. 16A-16D shows miR-155 reduces and anti-miR-155 increases NPPA mRNA and secreted Nt-proANP protein levels in hESC-CMs. Cardiomyocytes (˜1×10⁵ per well) were transfected with miR-155, anti-miR-155, a negative control miRNA, or a negative control anti-miR. Twenty-four hours later, cells were washed and incubated in 1 mL of media. After an additional 24 hours, cells and media were harvested. FIG. 16A shows NPPA mRNA levels in hESC-CMs transfected with miR-155 relative to cells transfected with negative control. FIG. 16B shows Nt-proANP protein levels (nmol/L) in the media of hESC-CMs transfected with miR-155 relative to negative control. FIG. 16C shows NPPA mRNA levels in hESC-CMs transfected with anti-miR-155 relative to negative control. FIG. 16D shows Nt-proANP protein levels in the media of hESC-CMs transfected with anti-miR-155 relative to negative control. Data are mean±SEM (n=6), 2-tailed independent t test.

FIGS. 17A-17B show allele-specific effects of miR-155 and anti-miR-155 on the luciferase activity produced by the firefly luciferase-NPPA 3′UTR constructs. The rs61764044 Minor-Luc construct contains the minor alleles of both rs61764044 and rs5068. FIG. 17A shows miR-155 reduced the activity of the WT-Luc construct but not the rs61764044 Minor-Luc construct. FIG. 17B shows Anti-miR-155 increased the activity of the WT-Luc construct but not the rs61764044 Minor-Luc construct. Data are mean±SEM (n=6), 2-tailed independent t test.

FIGS. 18A-18D show the effects of co-transfection of miR-425 and miR-155 (and their corresponding anti-miRs) on the luciferase activity produced by the firefly luciferase-NPPA 3′UTR constructs and on NPPA mRNA and secreted Nt-proANP protein levels in hESC-CMs. The rs61764044 Minor-Luc construct contains the minor alleles of rs61764044 and rs5068. FIG. 18A shows the allele-specific effects of miR-425 in combianation with miR-155, and FIG. 18B shows the allele-specific effects of anti-miR-425 in combination with anti-miR-155 on the luciferase activity produced by the WT-Luc and rs61764044 Minor-Luc constructs. FIG. 18C shows NPPA mRNA levels in hESC-CMs transfected with the indicated miRNAs relative to negative control. FIG. 18D shows Nt-proANP protein levels in the media of hESC-CMs transfected with the indicated miRNAs relative to negative control. Data are mean±SEM (n=6); P values from 2-tailed independent t test are shown. *P<0.001 vs. miR-425 and ^{#}P<0.001 vs. miR-155 by one-way ANOVA with Bonferroni post-hoc testing.

FIGS. 19A-19B show the Allele-specific effects of miR-105 and anti-miR-105 on the luciferase activity produced by the firefly luciferase-NPPA 3′UTR constructs. FIG. 19A shows miR-105 reduced the activity of the WT-Luc construct but not the rs5067 Minor-Luc construct. FIG. 19B shows anti-miR-105 increased the activity of the WT-Luc construct but not rs5067 Minor-Luc construct. Data are mean±SEM (n=6), 2-tailed independent t test.

FIGS. 20A-20D shows the effects of miR-105 and anti-miR-105 on NPPA mRNA and secreted Nt-proANP protein levels in hESC-CMs. Cardiomyocytes (˜1×10⁵ per well) were transfected with miR-105, anti-miR-105, a negative control miRNA, or a negative control anti-miR. Twenty-four hours later, cells were washed and incubated in 1 mL of media. After an additional 24 hours, cells and media were harvested. FIG. 20A shows NPPA mRNA levels in hESC-CMs transfected with miR-105 relative to negative control. FIG. 20B shows Nt-proANP protein levels in the media of hESC-CMs transfected with miR-105 relative to negative control. FIG. 20C shows NPPA mRNA levels in hESC-CMs transfected with anti-miR-105 relative to negative control. FIG. 20D shows Nt-proANP protein levels in the media of hESC-CMs transfected with anti-miR-105 relative to negative control. Data are mean±SEM (n=6), 2-tailed independent t test.

FIG. 21 is a schematic illustration depicting the use of a multidimensional approach combining bioinformatics, next-generation sequencing data, human genetic association data, and cellular models to identify miR-155 and miR-105 as novel ANP-modulating miRNAs with functional/biological relevance in humans. Bioinformatics is used to identify miRNAs predicted to target the NPPA 3′UTR by at least 3 algorithms. Next-generation sequencing data is used to verify the expression of the miRNAs in human atrial tissues. Human genetic association data is used to highlight miRNAs (miR-155 and miR-105) that target genetic variants whose minor alleles are associated with higher circulating human ANP levels (rs61764044 and rs5067, respectively) and thus are likely to have biological relevance in regulating ANP levels in humans. Cellular models are used to validate the ability of the miRNAs to interact with NPPA mRNA in an allele-specific manner, with the minor allele of each respective variant conferring resistance to the miRNA, and modulate ANP production in human cardiomyocytes.

FIGS. 22A-22C are schematic representations of the NPPA 3′UTR and the predicted interaction between miR-155 or miR-105 and the NPPA mRNA. FIG. 22A shows a schematic diagram of the location of rs5067, rs5068, and rs61764044 in the NPPA 3′UTR. FIG. 22B shows the position of rs61764044 is shown in red. The sequence complementary to the NPPA mRNA is shown in the figure to highlight the binding of miR-155 to sequences around the rs61764044 DNA variant. The minor G allele of rs61764044 creates a G:U wobble base pairing, as indicated by the dashed line. FIG. 22C shows the position of rs5067 is shown in blue. The sequence complementary to the NPPA mRNA is shown in the figure to highlight the binding of miR-105 to sequences around the rs5067 DNA variant. The minor G allele of rs5067 disrupts the base pairing, as indicated by the X.

FIGS. 23A-23B show that NPPA 3′UTR is a direct target of miR-155. FIG. 23A shows miR-155 reduced the activity of the WT-Luc construct but not the 155 Mut-Luc construct with a 6-nucleotide mutation at the predicted miR-155 seed binding site. FIG. 23B shows anti-miR-155 increased the activity of the WT-Luc construct but not the 155 Mut-Luc construct. Data are mean±SEM (n=6), 2-tailed independent t test.

FIGS. 24A-24B show the effects of miR-103 and anti-miR-103 on the luciferase activity produced by the firefly luciferase-NPPA 3′UTR constructs. FIG. 24A shows miR-103 reduced the activity of the WT-Luc construct but not the 103/107 Mut-Luc construct with a 5-nucleotide mutation at the predicted miR-103/107 seed binding site. FIG. 24B shows that anti-miR-103 increased the activity of the WT-Luc construct but not the 103/107 Mut-Luc construct. Data are mean±SEM (n=6), 2-tailed independent t test.

FIGS. 25A-25B show the effects of miR-107 and anti-miR-107 on the luciferase activity produced by the firefly luciferase-NPPA 3′UTR constructs. FIG. 25A shows that miR-107 reduced the activity of the WT-Luc construct but not the 103/107 Mut-Luc construct with a 5-nucleotide mutation at the predicted miR-103/107 seed binding site. FIG. 25B shows that Anti-miR-107 increased the activity of the WT-Luc construct but not the 103/107 Mut-Luc construct. Data are mean±SEM (n=6), 2-tailed independent t test.

FIGS. 26A-26B show the effects of miR-105 and anti-miR-105 on secreted Nt-proANP protein levels by heterologous cells transfected with a plasmid directing expression of a human NPPA cDNA. NPPA cDNA expression plasmid containing the rs5067 major allele was transfected into COS-7 cells together with a plasmid directing expression of renilla luciferase (as a control for transfection efficiency). Culture media was collected for measurement of secreted Nt-proANP protein levels (nmol/L), and cells were collected for measurement of renilla luciferase activity. FIG. 26A shows that miR-105 reduced the secreted Nt-proANP protein levels relative to negative control. FIG. 26B shows that anti-miR-105 increased the secreted Nt-proANP protein levels relative to negative control. Data are expressed as the ratio of Nt-proANP protein levels measured in the culture medium normalized to the renilla luciferase activity (mean±SEM; n=6, 2-tailed independent t test).

### DESCRIPTION OF THE INVENTION

The inventors have previously reported that rs5068 (A/G) is one of the genetic variants associated with ANP and BNP levels, which is located in the 3′ untranslated region (3′UTR) of NPPA. The rs5068 minor allele (G) is associated with increased plasma ANP levels, lower blood pressure, and reduced risk of hypertension. The inventors previously identified a microRNA, miR-425, which is expressed in the human heart, that binds to a sequence spanning rs5068 for the major allele (A) (but not the minor allele (G)) and represses ANP expression, which is discussed in P Arora et al. Journal of Clinical Investigation 2013, 123:3378-3382, and International Application WO2013/188787, which is incorporated herein in its entirety. Accordingly, the inventors previously discovered that rs5068 single nucleotide polymorphism (SNP) located in the 3′ UTR of NPPA gene disrupts the binding of the miR-425. Thus the minor allele (G) is resistant to the negative regulatory effects of miR-425, leading to increased NPPA mRNA levels and increased ANP production. In the International Application WO2013/188787, the inventors previously reported use of an anti-miR to miR-425 (i.e., an anti-miR-425 agent) to decrease or inhibit miR-425 function and thus elevate ANP levels in subjects who are AA (major homozygotes) or AG (heterozygotes) for the rs5068 variant of the 3′ untranslated region (3′UTR) of the NPPA gene.

Herein, using bioinformatics methods, the inventors have discovered at least 4 additioanal miRs, miR-103, miR-105, miR-107 and miR-155 that bind to the 3′ untranslated region (3′UTR) of NPPA. While there are some SNPs at some of the miR binding sites on NPPA (e.g., rs5067 in the 3′UTR of NPPA gene is located in a region which is the binding site for miR-105, and rs5066 is located in the 3′UTR of NPPA gene in a region which is are the binding sites for miR-103 and miR-107), the majority of the population have a genotype with at least one allele that comprises the binding site for miR-103/miR-107 or miR-105. Furthermore, because these SNPs (i.e., rs5067, rs5066 and rs5068) are not associated or correlated with each other, the presence of a non-susceptible genotype at one miR binding site would still be associated with susceptible genotypes at other miR binding sites, supporting the value of combination therapy. In some embodiments, an anti-miR to at least one, or a combination of any of miR-103, miR-105, miR-107 and miR-155 can be used in combination with an anti-miR-425 agent, such as disclosed in WO2013/188787, thereby preventing the binding of these miRs to the 3′ untranslated region (3′UTR) of the NPPA gene, and thus elevate ANP levels in virtually all subjects, as well as, subject who are AA (major homozygotes) or AG (heterozygotes) for the rs5068 variant of the 3′ untranslated region (3′UTR) of the NPPA gene.

The inventors herein have also discovered that miR-155 seed sequence binds to the 3′UTR of NPPA gene that contains the major allele (A) of the rs61764044 variant. The inventors have discovered that this rs61764044 variant is perfectly assopciated (e.g., linked) with the rs5068 variant, which lies 123 nucleotides upstream in the 3′ UTR of the NPPA gene, indicating that the minor alleles of rs61764044 and rs5068 are inherted together (perfect linkage disequlibrium, r²=1). Although the inventors have previously discovered that the minor allele (G) of the rs5068 variant disrupts the binding of miR-425 and therefore avoids miR-425 repression of NPPA gene, resulting in increased NPPA mRNA and ANP production, (and increased plasma ANP levels, lower blood pressure (BP) and reduced risk of hypertension, the inventors herein have extended this discovery, and demonstrate that the minor allele (G) of the rs61764044 variant results in a G:U wobble base pairing of the miR-155 seed sequence to the 3′UTR of the NPPA gene, therefore disrupts the binding of miR-155 preventing miR-155-mediated repression of NPPA gene, resulting in increased NPPA mRNA and ANP production. As a result, human subjects with the minor allele (G) of the rs61764044 variant have increased plasma ANP levels, lower blood pressure (BP) and reduced risk of hypertension.

Accordingly, in some embodiments, an anti-miR-155 agent can be used in combination with an anti-miR-425 agent to inhibit miR-155-mediated-, and miR-425-mediated respectively, repression of the NPPA gene in subjects who have the major “A” allele for rs61764044, and the major “A” allele for rs5068, therefore increasing ANP prodiction. In some embodiments, the anti-miR-155 agent and the anti-miR-425 agent act synergistically or additively to increase ANP production, leading to increase plasma ANP levels, lower BP and reduced risk of hypertension.

In alternative embodiments, where a subject suffers from hypotension and/or low blood pressure, and/or a subject whom has the minor (G) allele for rs61764044, and the minor “G” allele for rs5068 can be administered a miR-155 agonist agent and/or miR-425 agonist, thereby increasing NPPA gene repression and decreasing ANP levels, increasing BP and reducing hypotension. In some embodiments, the miR-155 agent is modified to bind to the minor (G) allele for rs61764044, and/or the miR-425 agonist agent is modified to bind to the minor “G” allele for rs5068.

In some embodiments, anti-miRs to at least one or a combination of miR-103, miR-105, miR-107 and/or miR-155 can be used to decrease the function of their respective miRs (e.g., decrease the function of miR-103, miR-105, miR-107 and/or miR-155), preventing their binding to the 3′ untranslated region (3′UTR) of the NPPA gene, and thus elevate ANP levels. In some embodiments, an anti-miR to at least one or a combination of miR-103, miR-105, miR-107 and/or miR-155 can be used in combination with an anti-miR-425 agent as disclosed in WO2013/188787 preventing their binding to the 3′ untranslated region (3′UTR) of the NPPA gene, and thus elevate ANP levels in virtually all subjects, as well as, subjects who are AA (major homozygotes) or AG (heterozygotes) for the rs5068 variant, and/or subjects who are AA (major homozygotes) or AG (heterozygotes) for the rs61764044 variant in the 3′ untranslated region (3′UTR) of the NPPA gene. Even if individuals carry a genetic variant at the binding site of one miR, the use of multiple anti-miRs (e.g. anti-miR-103 and/or anti-miR-107 and anti-miR-425, or anti-miR-105 and anti-miR-425 or anti-miR-155 and miR-425) will ensure that release of inhibitory effects of other miRs occurs.

Accordingly, the present invention generally relates to a method to treat a cardiovascular disease or disorder, hypertension, or a volume overload state (e.g., congestive heart failure) comprising administering a pharmaceutical composition comprising at least one anti-miR agent which inhibits the function of at least one of, or a combination of miR-103, miR-105, miR-107 and/or miR-155 to increase ANP biosynthesis and ANP plasma levels in the subject. In some embodiments, such a pharmaceutical composition can comprise an additional agent, for example an anti-miR agent that inhibits miR-425 for the treatment of subjects who are AA (major homozygotes) or AG (heterozygotes) for the rs5068 variant. In some embodiments, such a pharmaceutical composition can comprise an anti-miR agent that inhibits miR-155 for the treatment of subjects who are AA (major homozygotes) or AG (heterozygotes) for the rs61764044 variant.

In some embodiments, a pharmaceutical composition comprising at least one anti-miR agent which inhibits the function of at least one of, or a combination of miR-103, miR-105, miR-107 and/or miR-155, alone or in combination with an anti-miR-425 agent can be administered to a subject to increase ANP biosynthesis to prevent adverse left ventricular remodeling, which occurs in some patients after myocardial infarction.

In alternative embodiments, co-administration of low doses of anti-miRs directed against miR-425 and either miR-103, miR-105, miR-107 and/or miR-155, or alternatively, low doses of anti-miRs directed to at least 2, or at least 3, or at least 4 or all 5 of the miRs, can be used in a method to augment ANP production for the treatment of hypertension and congestive heart failure, while minimizing adverse “off-target” effects.

miRs are small RNA molecules that contain sequences complementary to their target mRNAs. The biogenesis of a miR begins with the transcription of the miR gene into a long primary transcript (pri-miR), which is then processed into a hairpin-shaped precursor miR (pre-miR). The pre-miR is exported out of the nucleus into the cytoplasm, where it is processed into a double-stranded miR duplex. The two strands of the duplex are then separated and one of the strands (the passenger strand) is degraded, while the other strand (the guide strand) becomes the mature single-stranded miR. The mature miR regulates gene expression by binding to partially complementary sequences that are usually located in the 3′UTR of target mRNAs. Binding of miRs to mRNAs causes translational repression and/or target mRNA degradation, leading to reduced protein levels. The region in the miR that is thought to be important for target mRNA recognition is known as the seed sequence, comprising nucleotides 2 to 7 at the miR 5′-end.

The previous report by the inventors that miR-425 targets the NPPA mRNA rs5068 major allele revealed a novel mechanism regulating ANP biosynthesis, and demonstrated that miR-425 antagonists could be used as a strategy for increasing ANP biosynthesis (e.g., increasing gene expression from the NPPA gene) with the goal of reducing blood pressure and treating volume overload. The inventors have previously reported use of a miR-425 antagonist (e.g., an “anti-miR-425 agent”) for the treatment of hypertension and other disorders associated with salt and water accumulation, e.g., see WO2013/188787, which is incorporated herein in its entirety by reference.

mRNAs typically contain multiple miR-binding sites, and the function of any given mRNA is often coordinately regulated by binding of different miRs (Balaga et al. Nucleic Acids Research 2012. 40:9404-9416). Thus, herein, the inventors have discovered four other miRs, in addition to miR-425, which bind to and target the 3′UTR of the NPPA mRNA and identified these miRs as having important roles in the regulation of ANP biosynthesis, and demonstrated the development of anti-miR therapies to miR-103, miR-105, miR-107 and/or miR-155 to augment or increase ANP synthesis in a subject. Moreover, The inventors demonstrate that utilizing multiple anti-miRs (e.g., miR-155 and miR-425 in combination, and/or miR-105 and miR-425 in combination) permits the use of low doses of individual anti-miRs, thereby minimizing adverse “off-target” effects induced by interfering with miR interactions with other mRNAs.

### Definitions

For convenience, certain terms employed in the entire application (including the specification, examples, and appended claims) are collected here. Unless defined otherwise, all technical and scientific terms used herein have the same meaning as commonly understood by one of ordinary skill in the art to which this invention belongs.

As used herein, the term “ANP” refers to atrial natriuretic peptide (ANP), and is also known as atrial natriuretic factor (ANF), atrial natriuretic hormone (ANH), Cardionatrine, Cardiodilatine (CDD) or atriopeptin, and is a protein hormone secreted by heart muscle cells that functions as a vasodilator and a potent mediator of natriuresis. ANP is expressed from the NPPA (natriuretic peptide A) gene. ANP is involved in the homeostatic control of body water, sodium, potassium and fat (adipose tissue). ANP is released by muscle cells in the upper chambers (atria) of the heart (atrial myocytes) in response to high blood pressure. ANP acts to reduce the water, sodium and adipose loads on the circulatory system, thereby reducing blood pressure. ANP is produced, stored, and released mainly by cardiac myocytes of the atria of the heart. Synthesis of ANP also takes place in the ventricles, brain, suprarenal glands, and renal glands. ANP is released in response to atrial stretch and a variety of other signals induced by hypervolemia, exercise, or caloric restriction. ANP is constitutively expressed in the ventricles in response to stress induced by increased afterload (e.g. increased ventricular pressure from aortic stenosis) or injury (e.g. myocardial infarction). The ANP gene has 3 exons and 2 introns; it codes 151-amino acid preproANP protein. Cleaving the 25-amino acid N-terminal signal sequence results in pro-ANP. Pro-ANP is cleaved by the serin protease corin, into ANP (the 28-amino acid C-terminal fragment) and NT-proANP (the N-terminal fragment). Accordingly, ANP is a 28-amino acid peptide corresponding to amino acids 124-151 of accession number NP_006163.1. The accession number NP_006163.1 corresponds to the ANP pre-proprotein, which includes a signal peptide (amino acids 1-25) and the proprotein (amino acids 26-151). ANP is closely related to BNP (brain natriuretic peptide) and CNP (C-type natriuretic peptide).

As used herein, the term “NT-proANP” refers to the N-terminal atrial natriuretic peptide and is the N-terminal fragment (proANP₁₋₃₀ fragment) as a result of cleavage of the proANP protein (proANP₁₋₁₂₆) to NT-proANP and ANP. The 126-amino acid peptide atrial natriuretic peptide pro-hormone (proANP₁₋₁₂₆) is synthesized and stored in atrial myocyte. Upon distension of the cardiac atria, proANP₁₋₁₂₆ is cleaved and equimolar amounts of the 28-amino acid ANP protein (e.g., C-terminal fragment proANP₉₉₋₁₂₆) and an N-terminal fragment (proANP₁₋₉₈) which are secreted from the atria. Whereas ANP is rapidly removed from the circulation, the N-terminal fragments, such as the proANP₁₋₃₀ fragment (Nt-proANP), which is also termed long-acting natriuretic peptide and has strong salt-excreting properties itself, are stable and remain in the circulation at manifold higher concentrations than ANP. Nt-proANP measured in peripheral plasma is therefore less prone to fluctuation and may thus be a more reliable measure of atrial ANP secretion than peripheral plasma concentration of ANP itself.

As used herein, the term “NPPA gene” refers to the natriuretic peptide A gene which encodes the pre-proANP peptide. The human NPPA gene expresses the mRNA identified by accession number: NM_006172.3 which is 856 bp in length, and includes a signal peptide (nucleic acids 100-174 of NM_006172.3) and the ANP pro-protein (nucleic acids 175-552 of NM_006172.3), which includes the mature ANP peptide (nucleic acids 175-375 of NM_006172.3).

As used herein, the term “BNP” refers to B-type natriuretic peptide, also known as Basic natriuretic peptide, and is a 32 amino acid polypeptide secreted by the ventricles of the heart in response to excessive stretching of heart muscle cells (e.g., cardiomyocytes). The release of BNP is modulated by calcium ions. BNP is secreted along with a 76 amino acid N-terminal fragment (NT-proBNP) which is biologically inactive. BNP binds to and activates the atrial natriuretic factor receptors NPRA, and to a lesser extent NPRB, in a fashion similar to atrial natriuretic peptide (ANP) but with 10-fold lower affinity. The biological half-life of BNP, however, is twice as long as that of ANP, and that of NT-proBNP is even longer. BNP is encoded as a pre-proprotein by the NPPB gene, corresponding to accession number NM_002521.2.

The term “miR-103” means the mature miRNA having the nucleobase sequence set forth in SEQ ID NO: 1 (AGCAGCAUUGUACAGGGCUAUGA), where the underlined portion is the seed sequence GCAGCAU (SEQ ID NO: 4).

The term “miR-105” means the mature miRNA of miR-105-5p having the nucleobase sequence set forth in SEQ ID NO: 7 (UCAAAUGCUCAGACUCCUGUGGU), where the underlined portion is the seed sequence CAAAUGC (SEQ ID NO: 9).

The term “miR-107” means the mature miRNA having the nucleobase sequence set forth in SEQ ID NO: 11 (AGCAGCAUUGUACAGGGCUAUCA), where the underlined portion is the seed sequence GCAGCAU (SEQ ID NO: 4).

The term “miR-103/miR-107” means a microRNA having the nucleobase sequence of SEQ ID NO: 1 or SEQ ID NO: 11.

The term “miR-103-1 stem-loop sequence” means the miR-103 precursor (e.g., human pre-mir-103a-1 (stem loop) or hsa-mir-103a-1 stem loop) having the nucleobase sequence set forth in SEQ ID NO: 21 (UACUGCCCUCGGCUUCUUUACAGUGCUGCCUUGUUGCAUAUGGAUCAAGCAGCAUUGU ACA GGGCUAUGAAGGCAUUG).

The term “miR-103-2” means the miR-103 precursor (e.g., human pre-mir-103a-2 (stem loop) or hsa-mir-103a-2 stem loop) having the nucleobase sequence set forth in SEQ ID NO: 22 (UUGUGCUUUCAGCUUCUUUACAGUGCUGCCUUGUAGCAUUCAGGUCAAGCAGCAUUGU ACA GGGCUAUGAAAGAACCA).

The term “miR-107 stem loop sequence” means the miR-107 (e.g., human pre-mir-107 (stem loop) or hsa-mir-107 stem loop) precursor having the nucleobase sequence set forth in SEQ ID NO: 27 (CUCUCUGCUUUCAGCUUCUUUACAGUGUUGCCUUGUGGCAUGGAGUUCAAGCAGCAUU GUA CAGGGCUAUCAAAGCACAGA).

The term “miR-105-1 stem-loop sequence” means the miR-105 precursor (e.g., human pre-mir-105-1 (stem loop) or hsa-mir-105-1 stem loop) having the nucleobase sequence set forth in SEQ ID NO: 25 (UGUGCAUCGUGGUCAAAUGCUCAGACUCCUGUGGUGGCUGCUCAUGCACCACGGAUGU UUGAGCAUGUGCUACGGUGUCUA).

The term “miR-105-2 stem-loop sequence” means the miR-105 precursor (e.g., human pre-mir-105-2 (stem loop) or hsa-mir-105-2 stem loop) having the nucleobase sequence set forth in SEQ ID NO: 26 (UGUGCAUCGUGGUCAAAUGCUCAGACUCCUGUGGUGGCUGCUUAUGCACCACGGAUGU UUGAGCAUGUGCUAUGGUGUCUA).

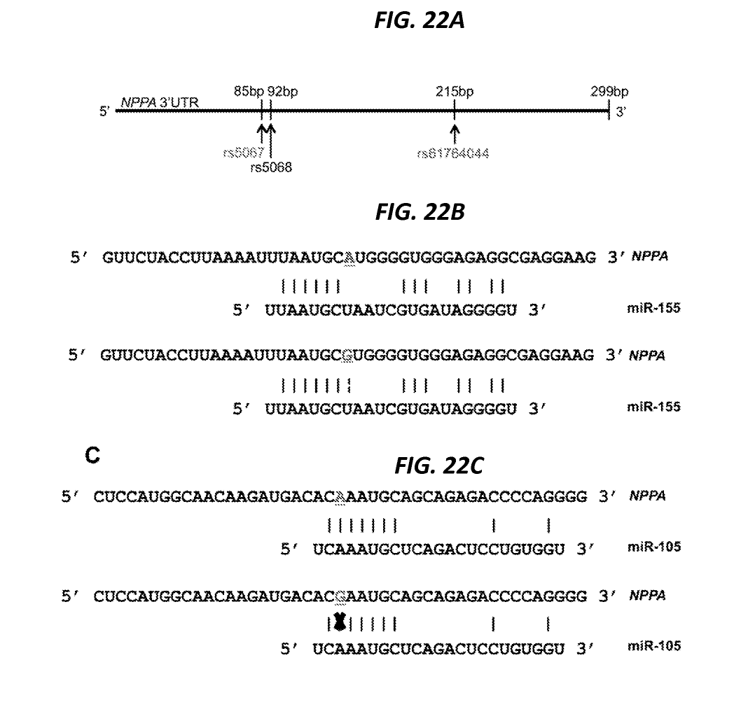

As used herein, the term “miR-155” refers to either mature (e.g., mature miR-155) or precursor forms of mi-R155 (e.g., pre-miR155 or pri-miR155). In a preferred embodiment, the miR-155 is a human miR 155 sequence, e.g., a human pri-miR155 (also known as the BIC transcript), human pre-miR155, or human mature miR155. Human pre-miR-155 (hsa miR-155; MirBase Accession No. MI0000681) was predicted based on homology to a cloned miR from mouse (mmu-miR-155; MiBase Accession No. MI0001777) [Lagos-Quintana M, et al., Curr Biol. 12:735-739 (2002)], and later experimentally validated in human HL-60 leukemia cells [Kasashima K, et al., Biochem Biophys Res Commun. 322:403-410 (2004)]. Like the mouse pri-miRNA155, human pri-miR-155 corresponds to a ^{˜}1421 b.p. non-coding BIC transcript (EMBL:AF402776), located on chromosome 21 [Weber M J, FEBS J. 272:59-73 (2005)]. The mature form of human miR-155 (MIMAT0000646) differs from that in mouse at a single position. In some embodiments, “miR-155” means the mature miRNA of miR-155-5p having the nucleobase sequence set forth in SEQ ID NO: 12 (UUAAUGCUAAUCGUGAUAGGGGU), where the underlined portion is the miR-155 seed sequence of AAUGCU (SEQ ID NO: 14). The seed sequence of mouse miR155 is identical to that of human. miR-155 refers to a nucleic acid or an agent which has a biological activity of binding to (or hybridizing to) the 3′ UTR of the NPPA gene which comprise the major A allele of the rs61764044 SNP, where the target sequence is a portion of the following sequence 5′-UAAUGCAUGGGGUGGGAGAGG-3′ (SEQ ID NO: 199), which is the A allele of rs61764044. miR-155 can also bind to some extent to the 3′UTR of the NPPA gene which comprises the minor G allele of the rs61764044 SNP, where the target sequence is a portion of the following sequence 5′-UAAUGCGUGGGGUGGGAGAGG-3′ (SEQ ID NO: 200), which is the G (minor) allele of rs61764044, resulting in G:U wobble base pairing with the miR-155 seed sequence AAUGCU (SEQ ID NO: 14), impairing the ability of miR-155 from repressing gene expression from the 3′UTR of NPPA gene (see FIG. 22B). In some embodiments, the target site for the seed sequence of miR-155 (SEQ ID NO: 14) binds to 5′-UAAUGCA-3′ (SEQ ID NO: 236) which is the A(major) allele of rs61764044 in the 3′UTR of the NPPA gene. In some embodiments, the seed sequence of miR-155 (SEQ ID NO: 14) binds to the target site of 5′-UAAUGCG-3′ (SEQ ID NO: 237) which is the G (minor) allele of rs61764044 in the 3′UTR of the NPPA gene.

The term “miR-155 stem-loop sequence” means the miR-155 precursor (e.g., human pre-mir-155 (stem loop) or hsa-mir-155 stem loop) having the nucleobase sequence set forth in SEQ ID NO: 203 (CUGUUAAUGCUAAUCGUGAUAGGGGUUUUUGCCUCCAACUGACUCCUACAUAUUAGCA UUAACAG) which can be arranged as follows:

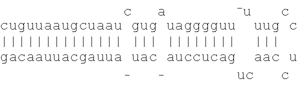

A miR-155 can be assessed for its ability to bind to and inhibit the target sequence SEQ ID NO: 200 using the luciferase assay as disclosed herein in the Examples, for example by using the WT-Luc construct MIR-REPORT™ luciferase reporter vector with a major (A) allele of rs61764044 in the 3′UTR of the NPPA exon 3 gene sequence. The term “anti-miR-155 agent” herein refers to any agent which binds to, at least part of the miR-155 sequence of SEQ ID NO: 12, or at least part of the miR-425 miRNA seed sequence SEQ ID NO: 14 (AAUGCU). The term anti-miR-155 also encompasses an “anti-miR-425 mimetic” which means an agent which binds to and inhibits the miR-155 nucleic acid of SEQ ID NO: 12. In this context, an anti-miR-155 agent can be any agent or RNA interference-inducing molecule, for example but not limited to unmodified and modified double stranded (ds) RNA molecules including, short-temporal RNA (stRNA), small interfering RNA (siRNA), short-hairpin RNA (shRNA), microRNA (miRNA), double-stranded RNA (dsRNA). Alternatively, an anti-miR-155 agent can be a small molecule, protein, aptamer, nucleic acid analogue, antibody etc., that binds to the miRNA seed sequence SEQ ID NO: 14 and/or inhibits miR-4155 activity (e.g., an anti-miR complementary to at least part of the miR-155 sequence of SEQ ID NO: 12, or at least part of the miR-155 miRNA seed sequence SEQ ID NO: 14). The terms “same activity” or “same function” as used in reference to the same activity or function of an anti-miR-155 agent means an anti-miR-155 agent molecule which can bind to at least part of the miRNA seed sequence of SEQ ID NO: 14 (AAUGCU), or at least part of the miR-155 sequence of SEQ ID NO: 12, and/or inhibit miR-155 with at least 80% of the efficiency, or greater efficiency, as the anti-miR of SEQ ID NO: 204 (CCCCTATCACGATTAGCATTAA) or a modified version thereof, e.g., SEQ ID NO: 205 (CCCCTATCACGATTAGCATTAA PS/MOE) or SEQ ID NO: 206 (CCCCTATCACGATTAGCATTAA PS/MOE 5-10-7) as assessed using, for example, the luciferase assay as disclosed herein in the Examples.

As used herein, the term “miR-425” refers to a nucleic acid or an agent which has a biological activity of binding to (or hybridizing to) the 3′ UTR of the NPPA gene which comprise the major A allele of the rs5068 SNP. In particular, the miR-425 corresponds to nucleic acid AAUGACACGAUCACUCCCGUUGA (SEQ ID NO: 15) and inhibiting the expression of a gene comprising the miR-425 target sequence, where the target sequence is 5′-ATGACAC-3′ (SEQ ID NO: 18), which is the A allele of rs5068. A miR-425 can be assessed for its ability to bind to and inhibit the target sequence SEQ ID NO: 18 using the luciferase assay as disclosed herein in the Examples, for example by using the pMIR-REPORT™ luciferase reporter vector with a major (A) allele of rs5068 in the 3′UTR of the NPPA exon 3 gene sequence. The term “anti-miR-425 agent” herein refers to any agent which binds to, at least part of the miR-425 sequence of SEQ ID NO: 15, or at least part of the miR-425 miRNA seed sequence SEQ ID NO: 16 (AUGACA) and also encompasses an “anti-miR-425 mimetic” and means an agent which binds to and inhibits the miR-425 nucleic acid of SEQ ID NO: 15. In this context, an anti-miR-425 agent can be any agent or RNA interference-inducing molecule, for example but not limited to unmodified and modified double stranded (ds) RNA molecules including, short-temporal RNA (stRNA), small interfering RNA (siRNA), short-hairpin RNA (shRNA), microRNA (miRNA), double-stranded RNA (dsRNA). Alternatively, an anti-miR-425 agent can be a small molecule, protein, aptamer, nucleic acid analogue, antibody etc. that binds to the miRNA seed sequence SEQ ID NO: 16 and/or inhibits miR-425 activity (e.g., an anti-miR complementary to at least part of the miR-425 sequence of SEQ ID NO: 15, or at least part of the miR-425 miRNA seed sequence SEQ ID NO: 16). The terms “same activity” or “same function” as used in reference to the same activity or function of an anti-miR-425 agent means an anti-miR-425 agent molecule which can bind to at least part of the miRNA seed sequence AUGACA (SEQ ID NO: 16) or at least part of the miR-425 sequence of SEQ ID NO: 15, and/or inhibit miR-425 with at least 80% of the efficiency, or greater efficiency, as the anti-miR of SEQ ID NO: 17, as assessed using, for example, the luciferase assay as disclosed herein in the Examples.

The terms “anti-miR”, “anti-miR agent,” “antimir” “microRNA inhibitor” or “miR inhibitor” are synonymous and refers to any agent which interferes with and inhibits the function of the target miR. In some embodiments, an anti-miR as disclosed herein is an antagomir that binds to and inhibits the function of the target miR. However, as the target miR may have different functions, in order to avoid undesired side effects of such an antagomir anti-miR agent, in alternative embodiments, an anti-miR is a blockmir, which is an anti-miR agent that has a sequence that is complementary to the mRNA sequence that is the target binding site for the miR, thereby inhibiting or interacting with the binding of the miR to the target mRNA sequence on the 3′ UTR of the NPPA gene. Accordingly, blockmirs bind to the target binding site of the miR-103, miR-105, miR-107 and/or miR-155 on the 3′ UTR of the NPPA gene, thereby blocking the binding of the respective miR (e.g., miR-103, miR-105, miR-107 and/or miR-155) and therefore specifically inhibiting the repressive function of miR-103, miR-105, miR-107 and/or miR-155 in repressing the NPPA gene transcription and their subsequent decrease in ANP expression. Such specific antagonism of the miR-NPPA interaction rather than generalized antagonism of miR-103, miR-105, miR-107 and/or miR-155 and all the mRNAs regulated by these miRs is encompassed to reduce off-target effects. In some embodiments, an anti-miR is an oligonucleotide having a nucleotide base sequence complementary to part, of the miRNA that it targets. In some embododiments, a anti-miR is a modified oligonucleotide. For instance, in some embodiments an anti-miR to miR-103/miR-107 is a blockmir that binds to the target sequence of SEQ ID NO: 6 in the 3′UTR of NPPA gene and inhibits/blocks miR-103 and/or miR-107 seed sequence of SEQ ID NO: 4 from binding. In some embodiments an anti-miR to miR-105 is a blockmir that binds to the target sequence of SEQ ID NO: 10 or SEQ ID NO: 207 (CAAAUGAAGCAGAGACCCCAG; the target site for miR-105 of the major allele of rs5067) or SEQ ID NO: 208 (CGAAUGAAGCAGAGACCCCAG; the target site for miR-105 of the minor allele of rs5067) in the 3′UTR of NPPA gene and inhibits/blocks miR-105 seed sequence of SEQ ID NO: 9 from binding. In some embodiments an anti-miR to miR-155 is a blockmir that binds to the target sequence of SEQ ID NO: 199 or 200 in the 3′UTR of NPPA gene and inhibits/blocks miR-105 seed sequence of SEQ ID NO: 14 from binding.

As used herein, “RNA” refers to a molecule comprising at least one or more ribonucleotide residues. A “ribonucleotide” is a nucleotide with a hydroxyl group at the 2′ position of a beta-D-ribofuranose moiety. The term RNA, as used herein, includes double-stranded RNA, single-stranded RNA, isolated RNA, such as partially purified RNA, essentially pure RNA, synthetic RNA, recombinantly-produced RNA, as well as altered RNA that differs from naturally-occurring RNA by the addition, deletion, substitution and/or alteration of one or more nucleotides. Nucleotides of the RNA molecules can also comprise non-standard nucleotides, such as non-naturally occurring nucleotides or chemically synthesized nucleotides or deoxynucleotides.

The terms “microRNA” or “miRNA” or “mir” or “miR” are used interchangeably herein, are endogenous RNAs, some of which are known to regulate the expression of protein-coding genes at the posttranscriptional level. As used herein, the term “microRNA” refers to any type of micro-interfering RNA, including but not limited to, endogenous microRNA and artificial microRNA. “MicroRNA” also means a non-coding RNA between 18 and 25 nucleobases in length, which is the product of cleavage of a pre-miRNA by the enzyme Dicer. Examples of mature miRNAs are found in the miRNA database known as miRBase (http://microma.sanger.ac.uk/). In certain embodiments, microRNA is abbreviated as “miRNA” or “miR.” Typically, endogenous microRNA are small RNAs encoded in the genome which are capable of modulating the productive utilization of mRNA. A mature miRNA is a single-stranded RNA molecule of about 21-23 nucleotides in length which is complementary to a target sequence, and hybridizes to the target RNA sequence to inhibit expression of a gene which encodes a miRNA target sequence. miRNAs themselves are encoded by genes that are transcribed from DNA but not translated into protein (non-coding RNA); instead they are processed from primary transcripts known as pri-miRNA to short stem-loop structures called pre-miRNA and finally to functional miRNA. Mature miRNA molecules are partially complementary to one or more messenger RNA (mRNA) molecules, and their main function is to downregulate gene expression. MicroRNA sequences have been described in publications such as, Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated by reference. Multiple microRNAs can also be incorporated into the precursor molecule.

A “mature microRNA” (mature miRNA) typically refers to a single-stranded RNA molecules of about 21-23 nucleotides in length, which regulates gene expression. miRNAs are encoded by genes from whose DNA they are transcribed, but miRNAs are not translated into protein; instead each primary transcript (pri-miRNA) is processed into a short stem-loop structure (precursor microRNA) before undergoing further processing into a functional mature miRNA. Mature miRNA molecules are partially complementary to one or more messenger RNA (mRNA) molecules, and their main function is to down-regulate gene expression. As used throughout, the term “microRNA” or “miRNA” includes both mature microRNA and precursor microRNA.

A mature miRNA is produced as a result of a series of miRNA maturation steps; first a gene encoding the miRNA is transcribed. The gene encoding the miRNA is typically much longer than the processed mature miRNA molecule; miRNAs are first transcribed as primary transcripts or “pri-miRNA” with a cap and poly-A tail, which is subsequently processed to short, about 70-nucleotide “stem-loop structures” known as “pre-miRNA” in the cell nucleus. This processing is performed in animals by a protein complex known as the Microprocessor complex, consisting of the nuclease Drosha and the double-stranded RNA binding protein Pasha. These pre-miRNAs are then processed to mature miRNAs in the cytoplasm by interaction with the endonuclease Dicer, which also initiates the formation of the RNA-induced silencing complex (RISC). This complex is responsible for the gene silencing observed due to miRNA expression and RNA interference. The pathway is different for miRNAs derived from intronic stem-loops; these are processed by Drosha but not by Dicer. In some instances, a given region of DNA and its complementary strand can both function as templates to give rise to at least two miRNAs. Mature miRNAs can direct the cleavage of mRNA or they can interfere with translation of the mRNA, either of which results in reduced protein accumulation, rendering miRNAs capable of modulating gene expression and related cellular activities.

“Pri-miRNA” or “pri-miR” means a non-coding RNA having a hairpin structure that is a substrate for the double-stranded RNA-specific ribonuclease Drosha. A “pri-miRNA” is a precursor to a mature miRNA molecule which comprises; (i) a microRNA sequence and (ii) stem-loop component which are both flanked (i.e. surrounded on each side) by “microRNA flanking sequences”, where each flanking sequence typically ends in either a cap or poly-A tail. Pri-microRNA, (also referred to as large RNA precursors), are composed of any type of nucleic acid based molecule capable of accommodating the microRNA flanking sequences and the microRNA sequence. Examples of pri-miRNAs and the individual components of such precursors (flanking sequences and microRNA sequence) are provided herein. The nucleotide sequence of the pri-miRNA precursor and its stem-loop components can vary widely. In one aspect a pre-miRNA molecule can be an isolated nucleic acid; including microRNA flanking sequences and comprising a stem-loop structure and a microRNA sequence incorporated therein. A pri-miRNA molecule can be processed in vivo or in vitro to an intermediate species caller “pre-miRNA”, which is further processed to produce a mature miRNA.

A “pre-miRNA” or “pre-miR” means a non-coding RNA having a hairpin structure, which is the product of cleavage of a pri-miR by the double-stranded RNA-specific ribonuclease known as DroshaA. The term “pre-miRNA” refers to the intermediate miRNA species in the processing of a pri-miRNA to mature miRNA, where pri-miRNA is processed to pre-miRNA in the nucleus, whereupon pre-miRNA translocates to the cytoplasm where it undergoes additional processing in the cytoplasm to form mature miRNA. Pre-miRNAs are generally about 70 nucleotides long, but can be less than 70 nucleotides or more than 70 nucleotides.

The term “miRNA precursor” means a transcript that originates from a genomic DNA and that comprises a non-coding, structured RNA comprising one or more miRNA sequences. For example, in certain embodiments a miRNA precursor is a pre-miRNA. In certain embodiments, a miRNA precursor is a pri-miRNA

The term “microRNA flanking sequence” as used herein refers to nucleotide sequences including microRNA processing elements. MicroRNA processing elements are the minimal nucleic acid sequences which contribute to the production of mature miRNA from precursor microRNA. Often these elements are located within a 40 nucleotide sequence that flanks a microRNA stem-loop structure. In some instances the microRNA processing elements are found within a stretch of nucleotide sequences of between 5 and 4,000 nucleotides in length that flank a microRNA stem-loop structure. Thus, in some embodiments the flanking sequences are 5-4,000 nucleotides in length. As a result, the length of the precursor molecule can be, in some instances at least about 150 nucleotides or 270 nucleotides in length. The total length of the precursor molecule, however, can be greater or less than these values. In other embodiments the minimal length of the microRNA flanking sequence is 10, 20, 30, 40, 50, 60, 70, 80, 90, 100, 150, 200 and any integer there between. In other embodiments the maximal length of the microRNA flanking sequence is 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900 4,000 and any integer there between.

MicroRNA flanking sequences can be native microRNA flanking sequences or artificial microRNA flanking sequences. A native microRNA flanking sequence is a nucleotide sequence that is ordinarily associated in naturally existing systems with microRNA sequences, i.e., these sequences are found within the genomic sequences surrounding the minimal microRNA hairpin in vivo. Artificial microRNA flanking sequences are nucleotides sequences that are not found to be flanking to microRNA sequences in naturally existing systems. microRNA flanking sequences within the pri-miRNA molecule can flank one or both sides of the stem-loop structure encompassing the microRNA sequence. Thus, one end (i.e., 5′) of the stem-loop structure can be adjacent to a single flanking sequence and the other end (i.e., 3′) of the stem-loop structure cannot be adjacent to a flanking sequence. Preferred structures have flanking sequences on both ends of the stem-loop structure. The flanking sequences can be directly adjacent to one or both ends of the stem-loop structure or can be connected to the stem-loop structure through a linker, additional nucleotides or other molecules.

A “stem-loop structure” refers to a nucleic acid having a secondary structure that includes a region of nucleotides which are known or predicted to form a double strand (stem portion) that is linked on one side by a region of predominantly single-stranded nucleotides (loop portion). “Stem-loop sequence” means an RNA having a hairpin structure and containing a mature miRNA sequence. Pre-miRNA sequences and stem-loop sequences may overlap. Examples of stem-loop sequences are found in the miRNA database known as miRBase (located at world-wide-web at: microma.sanger.ac.uk/). The terms “hairpin” and “fold-back” structures are also used herein to refer to stem-loop structures. Such structures are well known in the art and the term is used consistently with its known meaning in the art. The actual primary sequence of nucleotides within the stem-loop structure is not critical to the practice of the invention as long as the secondary structure is present. As is known in the art, the secondary structure does not require exact base-pairing. Thus, the stem can include one or more base mismatches. Alternatively, the base-pairing can be exact, i.e. not include any mismatches. In some instances the precursor microRNA molecule can include more than one stem-loop structure. The multiple stem-loop structures can be linked to one another through a linker, such as, for example, a nucleic acid linker or by a microRNA flanking sequence or other molecule or some combination thereof.

Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways. As used herein, the term “miRNA mimetic” refers to an artificial miRNA or RNAi (RNA interference molecule) which is flanked by the stem-loop like structures of a pri-miRNA.

The term “artificial microRNA” includes any type of RNA sequence, other than endogenous microRNA, which is capable of modulating the productive utilization of mRNA. For instance, the term artificial microRNA also encompasses a nucleic acid sequence which would be previously identified as siRNA, where the siRNA is incorporated into a vector and surrounded by miRNA flanking sequences as described herein.

The term “seed sequence” means nucleotides 2 to 6 or 2 to 7 from the 5′-end of a mature miRNA sequence.

As used herein, “double stranded RNA” or “dsRNA” refers to RNA molecules that are comprised of two substantially complementary strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 116:281-297), comprises a dsRNA molecule.

As used herein, “gene silencing” or “gene silenced” by a miRNA and/or RNA interference molecule “refers to a decrease in the mRNA level in a cell for a target gene by at least about 5%, at least about 10%, at least about 20%, at least about 30%, at least about 40%, at least about 50%, at least about 60%, at least about 70%, at least about 80%, at least about 90%, at least about 95%, at least about 99% up to and including 100%, and any integer in between of the mRNA level found in the cell without the presence of the miRNA or RNA interference molecule. In one preferred embodiment, the mRNA levels are decreased by at least about 70%, at least about 80%, at least about 90%, at least about 95%, at least about 99%, up to and including 100% and any integer in between 5% and 100%.”

The term “reduced” or “reduce” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The term “increased” or “increase” as used herein generally means an increase by a statically significant amount; for the avoidance of any doubt, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “tissue” refers to a group or layer of similarly specialized cells which together perform certain special functions. The term “tissue-specific” refers to a source of cells from a specific tissue.

By the term “inhibitory RNA” is meant a nucleic acid molecule that contains a sequence that is complementary to a target nucleic acid (e.g., a target miR as disclosed herein, e.g., miR-103, miR-105, miR-107, miR-155) that mediates a decrease in NPPA gene expression). Non-limiting examples of inhibitory RNAs include interfering RNA, shRNA, siRNA, ribozymes, antagomirs, and antisense oligonucleotides. Methods of making inhibitory RNAs are described herein. Additional methods of making inhibitory RNAs are known in the art.

As used herein, “an interfering RNA” refers to any double stranded or single stranded RNA sequence, capable—either directly or indirectly (i.e., upon conversion)—of inhibiting or down regulating gene expression by mediating RNA interference. Interfering RNA includes but is not limited to small interfering RNA (“siRNA”) and small hairpin RNA (“shRNA”). “RNA interference” refers to the selective degradation of a sequence-compatible messenger RNA transcript.

As used herein “an shRNA” (small hairpin RNA) refers to a type of siRNA, and is an RNA molecule comprising an antisense region, a loop portion and a sense region, wherein the sense region has complementary nucleotides that base pair with the antisense region to form a duplex stem. Following post-transcriptional processing, the small hairpin RNA is converted into a small interfering RNA (siRNA) by a cleavage event mediated by the enzyme Dicer, which is a member of the RNase III family. A “shRNA” or “small hairpin RNA” is also called “stem loop” and in some embodiments, a shRNA is composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

As used herein a “siRNA” refers to a nucleic acid that forms a double stranded RNA, which double stranded RNA has the ability to reduce or inhibit expression of a particular gene or target gene when the siRNA is expressed in the same cell as the gene or target gene. A “small interfering RNA” or “siRNA” as used herein refers to any small RNA molecule capable of inhibiting or down regulating gene expression by mediating RNA interference in a sequence specific manner. The small RNA can be, for example, about 18 to 21 nucleotides long. The double stranded RNA siRNA can be formed by the complementary strands. The complementary portions of the siRNA that hybridize to form the double stranded molecule typically have substantial or complete identity. In one embodiment, a siRNA refers to a nucleic acid that has substantial or complete identity to sequence of a target gene and forms a double stranded RNA. The sequence of the siRNA can correspond to the full length target gene, or to a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 18-30 base nucleotides, preferably about 18-25 or 18-21 nucleotides in length, e.g., 20, 21, 22, 23, 24, 25, 26, 27, 28, 29, or 30 nucleotides in length).

As used herein, an “antagomir” refers to a small synthetic RNA having complementarity to a specific microRNA target, optionally with either mispairing at the cleavage site or one or more base modifications to inhibit cleavage.

As used herein, the phrase “post-transcriptional processing” refers to mRNA processing that occurs after transcription and is mediated, for example, by the enzymes Dicer and/or Drosha.

The term “biological sample” as used herein means a sample of biological tissue or fluid that comprises nucleic acids. Such samples include, but are not limited to, tissue isolated from animals. Biological samples can also include sections of tissues such as biopsy and autopsy samples, frozen sections taken for histologic purposes, blood, plasma, serum, sputum, stool, tears, mucus, hair, and skin. Biological samples also include explants and primary and/or transformed cell cultures derived from patient tissues. A biological sample can be provided by removing a sample of cells from an animal, but can also be accomplished by using previously isolated cells (e.g., isolated by another person, at another time, and/or for another purpose), or by performing the methods as disclosed herein in vivo. Archival tissues, such as those having treatment or outcome history can also be used.

The term “tissue” is intended to include, blood, blood preparations such as plasma and serum, bones, joints, muscles, smooth muscles, and organs.

The terms “disease” or “disorder” are used interchangeably herein, and refer to any alteration in state of the body or of some of the organs, interrupting or disturbing the performance of the functions and/or causing symptoms such as discomfort, dysfunction, distress, or even death to the person afflicted or those in contact with a person. A disease or disorder can also be related to a distemper, ailing, ailment, malady, disorder, sickness, illness, complaint, indisposition or affliction.

By the phrase “risk of developing disease” is meant the relative probability that a subject will develop hypertension or cardiac disorder in the future as compared to a control subject or population (e.g., a healthy subject or population). Provided herein are methods for determining a subject's risk of developing hypertension or a cardiac disease in the future that include determining the level of one or more of the miR-103, miR-105, miR-107, miR-155.

The terms “subject” and “individual” are used interchangeably herein, and refer to an animal, for example a human, to whom treatment, including prophylactic treatment, with a pharmaceutical composition as disclosed herein, is provided. The term “subject” as used herein refers to human and non-human animals. The term “non-human animals” includes all vertebrates, e g, mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In one embodiment, the subject is human. In another embodiment, the subject is an experimental animal or animal substitute as a disease model.

The term ‘effective amount” as used herein refers to the amount of therapeutic agent of pharmaceutical composition to alleviate at least one of the symptoms of the disease or disorder.

The term “Administering” means providing a pharmaceutical agent or composition to a subject, and includes, but is not limited to, administering by a medical professional and self-administering.

The term “Parenteral administration,” means administration through injection or infusion. Parenteral administration includes, but is not limited to, subcutaneous administration, intravenous administration, or intramuscular administration.

The term “Subcutaneous administration” means administration just below the skin.

The term “Intravenous administration” means administration into a vein.

The term “Administered concomitantly” refers to the administration of at least two agents to a subject in any manner in which the pharmacological effects of both are manifest in the subject at the same time. Concomitant administration does not require that both agents be administered in a single pharmaceutical composition, in the same dosage form, or by the same route of administration. The time during which the effects of the agents need not be identical. The effects need only be overlapping for a period of time and need not be coextensive. “Duration” means the period of time during which an activity or event continues. In certain embodiments, the duration of treatment is the period of time during which doses of a pharmaceutical agent or pharmaceutical composition are administered.

The term “Therapy” means a disease treatment method. In certain embodiments, therapy includes, but is not limited to, chemotherapy, surgical resection, liver transplant, and/or chemoembolization.

The term “Treatment” means the application of one or more specific procedures used for the cure or amelioration of a disease. In certain embodiments, the specific procedure is the administration of one or more pharmaceutical agents.

The term “Amelioration” means a lessening of severity of at least one indicator of a condition or disease. In certain embodiments, amelioration includes a delay or slowing in the progression of one or more indicators of a condition or disease. The severity of indicators may be determined by subjective or objective measures which are known to those skilled in the art.

The term “At risk for developing” means a subject is predisposed to developing a condition or disease. In certain embodiments, a subject at risk for developing a condition or disease exhibits one or more symptoms of the condition or disease, but does not exhibit a sufficient number of symptoms to be diagnosed with the condition or disease. In certain embodiments, a subject at risk for developing a condition or disease exhibits one or more symptoms of the condition or disease, but to a lesser extent required to be diagnosed with the condition or disease.

The term “Prevent the onset of” means to prevent the development a condition or disease in a subject who is at risk for developing the disease or condition. In certain embodiments, a subject at risk for developing the disease or condition receives treatment similar to the treatment received by a subject who already has the disease or condition.

The term “Delay the onset of” means to delay the development of a condition or disease in a subject who is at risk for developing the disease or condition. In certain embodiments, a subject at risk for developing the disease or condition receives treatment similar to the treatment received by a subject who already has the disease or condition.

The term “Therapeutic agent” means a pharmaceutical agent used for the cure, amelioration or prevention of a disease.

The term “Dose” means a specified quantity of a pharmaceutical agent provided in a single administration. In certain embodiments, a dose may be administered in two or more boluses, tablets, or injections. For example, in certain embodiments, where subcutaneous administration is desired, the desired dose requires a volume not easily accommodated by a single injection. In such embodiments, two or more injections may be used to achieve the desired dose. In certain embodiments, a dose may be administered in two or more injections to minimize injection site reaction in an individual.

The term “Dosage unit” means a form in which a pharmaceutical agent is provided. In certain embodiments, a dosage unit is a vial containing lyophilized oligonucleotide. In certain embodiments, a dosage unit is a vial containing reconstituted oligonucleotide.

The term “Therapeutically effective amount” refers to an amount of a pharmaceutical agent that provides a therapeutic benefit to an animal.

The term “Pharmaceutical composition” means a mixture of substances suitable for administering to an individual that includes a pharmaceutical agent. For example, a pharmaceutical composition may comprise a sterile aqueous solution.

The term “Pharmaceutical agent” means a substance that provides a therapeutic effect when administered to a subject.

The term “Active pharmaceutical ingredient” means the substance in a pharmaceutical composition that provides a desired effect.

The term “Acceptable safety profile” means a pattern of side effects that is within clinically acceptable limits.

The term “Side effect” means a physiological response attributable to a treatment other than desired effects. In certain embodiments, side effects include, without limitation, injection site reactions, liver function test abnormalities, renal function abnormalities, liver toxicity, renal toxicity, central nervous system abnormalities, and myopathies. Such side effects may be detected directly or indirectly. For example, increased aminotransferase levels in serum may indicate liver toxicity or liver function abnormality. For example, increased bilirubin may indicate liver toxicity or liver function abnormality.

The term “target nucleic acid” means a nucleic acid sequence to which an oligomeric compound or miR hybridizes to, and includes the target/binding site of the seed sequence of a miR to the mRNA sequence it represses.

The term “targeting” means the process of design and selection of nucleobase sequence that will hybridize to a specific target nucleic acid.

The term “targeted to” means having a nucleobase sequence that will allow hybridization to a target nucleic acid.

The term “modulation” means to a perturbation of function or activity. In certain embodiments, modulation means an increase in gene expression. In certain embodiments, modulation means a decrease in gene expression.

The term “expression” means any functions and steps by which a gene's coded information is converted into structures present and operating in a cell.

The term “gene” as used herein refers to a genomic gene comprising transcriptional and/or translational regulatory sequences and/or a coding region and/or non-translated sequences (e.g., introns, 5′- and 3′-untranslated sequences). The coding region of a gene can be a nucleotide sequence coding for an amino acid sequence or a functional RNA, such as tRNA, rRNA, catalytic RNA, siRNA, miRNA and antisense RNA. A gene can also be an mRNA or cDNA corresponding to the coding regions (e.g. exons and miRNA) optionally comprising 5′- or 3′ untranslated sequences linked thereto. A gene can also be an amplified nucleic acid molecule produced in vitro comprising all or a part of the coding region and/or 5′- or 3′-untranslated sequences linked thereto.

The term “region” means a portion of linked nucleosides within a nucleic acid. In certain embodiments, an has a nucleobase sequence that is complementary to a region of a target nucleic acid. For example, in certain such embodiments an is complementary to a region of a miRNA stem-loop sequence. In certain such embodiments, an is fully complementary to a region of a miRNA stem-loop sequence.

The term “Segment” means a smaller or sub-portion of a region.

The term “Nucleobase sequence” means the order of contiguous nucleobases, in a 5′ to 3′ orientation, independent of any sugar, linkage, and/or nucleobase modification.

The term “Contiguous nucleobases” means nucleobases immediately adjacent to each other in a nucleic acid.

The term “Nucleobase complementarity” means the ability of two nucleobases to pair non-covalently via hydrogen bonding.

The term “Complementary” means that an oligomeric compound is capable of hybridizing to a target nucleic acid under stringent hybridization conditions. “Fully complementary” means each nucleobase of an oligomeric compound is capable of pairing a nucleobase at each corresponding position in a target nucleic acid. For example, in certain embodiments, an oligomeric compound wherein each nucleobase has complementarity to a nucleobase within a region of a miRNA stem-loop sequence is fully complementary to the miRNA stem-loop sequence.

The term “Percent complementarity” means the percentage of nucleobases of an oligomeric compound that are complementary to an equal-length portion of a target nucleic acid. Percent complementarity is calculated by dividing the number of nucleobases of the oligomeric compound that are complementary to nucleobases at corresponding positions in the target nucleic acid by the total length of the oligomeric compound. In certain embodiments, percent complementarity of an means the number of nucleobases that are complementary to the target nucleic acid, divided by the length of the modified oligonucleotide.

The term “Percent identity” means the number of nucleobases in first nucleic acid that are identical to nucleobases at corresponding positions in a second nucleic acid, divided by the total number of nucleobases in the first nucleic acid.

The term “Hybridize” means the annealing of complementary nucleic acids that occurs through nucleobase complementarity.

The term “Mismatch” means a nucleobase of a first nucleic acid that is not capable of pairing with a nucleobase at a corresponding position of a second nucleic acid.

The term “Identical” means having the same nucleobase sequence.

The term “nucleic acid” or “oligonucleotide” or “polynucleotide” used herein means at least two nucleotides covalently linked together. As will be appreciated by those in the art, the depiction of a single strand also defines the sequence of the complementary strand. Thus, a nucleic acid also encompasses the complementary strand of a depicted single strand. As will also be appreciated by those in the art, many variants of a nucleic acid can be used for the same purpose as a given nucleic acid. Thus, a nucleic acid also encompasses substantially identical nucleic acids and complements thereof. As will also be appreciated by those in the art, a single strand provides a probe that can hybridize to the target sequence under stringent hybridization conditions. Thus, a nucleic acid also encompasses a probe that hybridizes under stringent hybridization conditions.

Nucleic acids can be single stranded or double stranded, or can contain portions of both double stranded and single stranded sequence. The nucleic acid can be DNA, both genomic and cDNA, RNA, or a hybrid, where the nucleic acid can contain combinations of deoxyribo- and ribo-nucleotides, and combinations of bases including uracil, adenine, thymine, cytosine, guanine, inosine, xanthine hypoxanthine, isocytosine and isoguanine. Nucleic acids can be obtained by chemical synthesis methods or by recombinant methods.

A nucleic acid will generally contain phosphodiester bonds, although nucleic acid analogs can be included that can have at least one different linkage, e.g., phosphoramidate, phosphorothioate, phosphorodithioate, or O-methylphosphoroamidite linkages and peptide nucleic acid backbones and linkages. Other analog nucleic acids include those with positive backbones; non-ionic backbones, and non-ribose backbones, including those described in U.S. Pat. Nos. 5,235,033 and 5,034,506, which are incorporated by reference. Nucleic acids containing one or more non-naturally occurring or modified nucleotides are also included within one definition of nucleic acids. The modified nucleotide analog can be located for example at the 5′-end and/or the 3′-end of the nucleic acid molecule. Representative examples of nucleotide analogs can be selected from sugar- or backbone-modified ribonucleotides. It should be noted, however, that also nucleobase-modified ribonucleotides, i.e. ribonucleotides, containing a non-naturally occurring nucleobase instead of a naturally occurring nucleobase such as uridines or cytidines modified at the 5-position, e.g. 5-(2-amino)propyl uridine, 5-bromo uridine; adenines and guanosines modified at the 8-position, e.g. 8-bromo guanosine; deaza nucleotides, e. g. 7 deaza-adenine; O- and N-alkylated nucleotides, e.g. N6-methyl adenine are suitable. The 2′ OH— group can be replaced by a group selected from H. OR, R. halo, SH, SR, NH2, NHR, NR2 or CN, wherein R is C—C6 alkyl, alkenyl or alkynyl and halo is F. Cl, Br or I. Modifications of the ribose-phosphate backbone can be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs can be made.

The term “probe” as used herein refers to an oligonucleotide capable of binding to a target nucleic acid of complementary sequence through one or more types of chemical bonds, usually through complementary base pairing, usually through hydrogen bond formation. Probes can bind target sequences lacking complete complementarily with the probe sequence depending upon the stringency of the hybridization conditions. There can be any number of base pair mismatches which will interfere with hybridization between the target sequence and single stranded target nucleic acids, but a probe will bind a selected target specifically, i.e. to the substantial exclusion of non-target nucleic acids under at least one set of conditions. A probe can be single stranded or partially single and partially double stranded. A probe will generally be detectably labeled or carry a moiety that permits signal detection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, for example, by the local homology algorithm of Smith and Waterman (Adv. Appl. Math. 2:482 (1981), which is incorporated by reference herein), by the homology alignment algorithm of Needleman and Wunsch (J. Mol. Biol. 48:443-53 (1970), which is incorporated by reference herein), by the search for similarity method of Pearson and Lipman (Proc. Natl. Acad. Sci. USA 85:2444-48 (1988), which is incorporated by reference herein), by computerized implementations of these algorithms (e.g., GAP, BESTFIT, FASTA, and TFASTA in the Wisconsin Genetics Software Package, Genetics Computer Group, 575 Science Dr., Madison, Wis.), or by visual inspection. (See generally Ausubel et al. (eds.), Current Protocols in Molecular Biology, 4th ed., John Wiley and Sons, New York (1999)).

One example of a useful algorithm is PILEUP. PILEUP creates a multiple sequence alignment from a group of related sequences using progressive, pairwise alignments to show the percent sequence identity. It also plots a tree or dendogram showing the clustering relationships used to create the alignment. PILEUP uses a simplification of the progressive alignment method of Feng and Doolittle (J. Mol. Evol. 25:351-60 (1987), which is incorporated by reference herein). The method used is similar to the method described by Higgins and Sharp (Comput. Appl. Biosci. 5:151-53 (1989), which is incorporated by reference herein). The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of an algorithm that is suitable for determining percent sequence identity and sequence similarity is the BLAST algorithm, which is described by Altschul et al. (J. Mol. Biol. 215:403-410 (1990), which is incorporated by reference herein). (See also Zhang et al., Nucleic Acid Res. 26:3986-90 (1998); Altschul et al., Nucleic Acid Res. 25:3389-402 (1997), which are incorporated by reference herein). Software for performing BLAST analyses is publicly available through the National Center for Biotechnology Information internet web site. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990), supra). These initial neighborhood word hits act as seeds for initiating searches to find longer HSPs containing them. The word hits are then extended in both directions along each sequence for as far as the cumulative alignment score can be increased. Extension of the word hits in each direction is halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a word length (W) of 11, the BLOSUM62 scoring matrix (see Henikoff and Henikoff, Proc. Natl. Acad. Sci. USA 89:10915-9 (1992), which is incorporated by reference herein) alignments (B) of 50, expectation (E) of 10, M=5, N=−4, and a comparison of both strands.

In addition to calculating percent sequence identity, the BLAST algorithm also performs a statistical analysis of the similarity between two sequences (see, e.g., Karlin and Altschul, Proc. Natl. Acad. Sci. USA 90:5873-77 (1993), which is incorporated by reference herein). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more typically less than about 0.01, and most typically less than about 0.001.

The term “variant” as used in the context of anti-miR agent, e.g., an anti-miR of miR-103, miR-107, miR-105, miR-155 or miR-425 variant means a modified anti-miR with at least one of the following; altered nucleic acid sequence, such as insertions, deletions, substitutions, fragments of at least 5 nucleic acids, modification of the nucleic acids or nucleic acid analogues as compared to an anti-miR that inhibits the miRs of SEQ ID NO: 1, 7, 11 or 12 or a modified nucleic acid which is complementary to at least part of any of the miRs miR-103, miR-105, miR-107 or miR-155 sequences of SEQ ID NO: 1, 7, 11 or 12, respectively.

As used herein, the terms “homologous” or “homologues” are used interchangeably, and when used to describe a polynucleotide or polypeptide, indicates that two polynucleotides or polypeptides, or designated sequences thereof, when optimally aligned and compared, for example using BLAST, version 2.2.14 with default parameters for an alignment (see below) are identical, with appropriate nucleotide insertions or deletions or amino-acid insertions or deletions, in at least 70% of the nucleotides, usually from about 75% to 99%, and more preferably at least about 98 to 99% of the nucleotides. The term “homolog” or “homologous” as used herein also refers to homology with respect to structure and/or function. With respect to sequence homology, sequences are homologs if they are at least 50%, at least 60 at least 70%, at least 80%, at least 90%, at least 95% identical, at least 97% identical, or at least 99% identical. The term “substantially homologous” refers to sequences that are at least 90%, at least 95% identical, at least 97% identical or at least 99% identical. Homologous sequences can be the same functional gene in different species.

Determination of homologs of the genes or peptides of the present invention can be easily ascertained by the skilled artisan. The terms “homology”, “identity” and “similarity” refer to the degree of sequence similarity between two peptides or between two optimally aligned nucleic acid molecules. Homology and identity can each be determined by comparing a position in each sequence which can be aligned for purposes of comparison. When an equivalent position in the compared sequences is occupied by the same base or amino acid, then the molecules are identical at that position; when the equivalent site occupied by similar amino acid residues (e.g., similar in steric and/or electronic nature such as, for example conservative amino acid substitutions), then the molecules can be referred to as homologous (similar) at that position. Expression as a percentage of homology/similarity or identity refers to a function of the number of similar or identical amino acids at positions shared by the compared sequences, respectfully. A sequence which is “unrelated” or “non-homologous” shares less than 40% identity, though preferably less than 25% identity with a sequence of the present application.

As used herein, the term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T. C, G. U. or 1) or residue occurs in both sequences to yield the number of matched positions, dividing the number of matched positions by the total number of positions in the comparison window (i.e., the window size), and multiplying the result by 100 to yield the percentage of sequence identity.

The terms “substantial identity” as used herein denotes a characteristic of a polynucleotide or amino acid sequence, wherein the polynucleotide or amino acid comprises a sequence that has at least 85 percent sequence identity, preferably at least 90 to 95 percent sequence identity, more usually at least 99 percent sequence identity as compared to a reference sequence over a comparison window of at least 18 nucleotide (6 amino acid) positions, frequently over a window of at least 24-48 nucleotide (8-16 amino acid) positions, wherein the; percentage of sequence identity is calculated by comparing the reference sequence to the sequence which can include deletions or additions which total 20 percent or less of the reference sequence over the comparison window. The reference sequence can be a subset of a larger sequence. The term “similarity”, when used to describe a polypeptide, is determined by comparing the amino acid sequence and the conserved amino acid substitutes of one polypeptide to the sequence of a second polypeptide.

The term “synergy” or “synergistic” as used herein, refers to the interaction of two or more agents so that their combined effect is greater than the sum of each of their individual effects at the same dose alone. For example, administration of anti-miR-155 in combination with anti-miR-425 as disclosed herein is synergistic in that their effect in increasing NPPA gene expression and production of ANP is greater than their effects alone or the sum of their effects at the same dose. Therefore, the dose (or amount) of an anti-miR-425 agent can be decreased in the presence of an anti-miR-155 agent to achieve the same increase in NPPA gene expression and increase in ANP production. Similarily, the dose or amount of an anti-miR-155 agent can be decreased in the presence of an anti-miR-425 agent to achieve the same increase in NPPA gene expression and incrase in ANP production, as compared to the amount of anti-miR-155 agent used alone. By way of example only, in some embodiments, the effective amount (e.g., dose) of an anti-miR-155 agent to increase NPPA gene expression and to increase ANP production when used in combination with an anti-miR-425 agent is at least 10%, or at least 20% or at least 30% or at least 40%, or at least 50% less (e.g., a decreased dose) than the effective amount of the same an anti-miR-155 agent when it is used alone and/or in the absence of an anti-miR-425 agent. Similarily, in some embodiments, the effective amount (e.g., dose) of an anti-miR-425 agent is at least 10%, or at least 20% or at least 30% or at least 40%, or at least 50% less than it's effective amount (e.g., dose) when used alone and/or in the absence of an anti-miR-155 agent increase NPPA gene expression and to increase ANP production.

The term “vectors” refers to a nucleic acid molecule capable of transporting another nucleic acid to which it has been linked; a plasmid is a species of the genus encompassed by “vector”. The term “vector” typically refers to a nucleic acid sequence containing an origin of replication and other entities necessary for replication and/or maintenance in a host cell. Vectors capable of directing the expression of genes and/or nucleic acid sequence to which they are operatively linked are referred to herein as “expression vectors”. In general, expression vectors of utility are often in the form of “plasmids” which refer to circular double stranded DNA loops which, in their vector form are not bound to the chromosome, and typically comprise entities for stable or transient expression or the encoded DNA. Other expression vectors can be used in the methods as disclosed herein for example, but are not limited to, plasmids, episomes, bacterial artificial chromosomes, yeast artificial chromosomes, bacteriophages or viral vectors, and such vectors can integrate into the host's genome or replicate autonomously in the particular cell. A vector can be a DNA or RNA vector. Other forms of expression vectors known by those skilled in the art which serve the equivalent functions can also be used, for example self replicating extrachromosomal vectors or vectors which integrates into a host genome.

As used herein, the terms “treat” or “treatment” or “treating” as used herein in reference to use of a anti-miR as disclosed herein, (e.g., but not limited to, an anti-miR-155 alone or in combination with an anti-miR-425 agent; or an anti-miR-105 agent alone, or in combination with an anti-miR-425) for the treatment of a cardiovascular disease or disorder, refers to therapeutic treatment, wherein the object is to prevent or slow the development of the disease, such as slow down the development of a cardiac disorder, or reducing at least one adverse effect or symptom of a cardiovascular condition, disease or disorder, i.e., any disorder characterized by insufficient or undesired cardiac function. Adverse effects or symptoms of cardiac disorders are well-known in the art and include, but are not limited to, dyspnea, chest pain, palpitations, dizziness, syncope, edema, cyanosis, pallor, fatigue and death. Treatment is generally “effective” if one or more symptoms or clinical markers are reduced as that term is defined herein. Alternatively, a treatment is “effective” if the progression of a disease is reduced or halted. That is, “treatment” includes not just the improvement of symptoms or decrease of markers of the disease, but also a cessation or slowing of progress or worsening of a symptom that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already diagnosed with a cardiac condition, as well as those likely to develop a cardiac condition due to genetic susceptibility or other factors such as weight, diet and health. In some embodiments, the term to treat also encompasses preventative measures and/or prophylactic treatment, which includes administering a pharmaceutical composition as disclosed herein to prevent the onset of a disease or disorder.

In some embodiment, the term “treating” when used in reference to a treatment of a cardiovascular disease or disorder is used to refer to the reduction of a symptom and/or a biochemical marker of a cardiovascular disease or disorder, for example a reduction in at least one biochemical marker of a cardiovascular disease by at least about 10% would be considered an effective treatment. Examples of such biochemical markers of cardiovascular disease include a reduction of, for example, creatine phosphokinase (CPK), aspartate aminotransferase (AST), lactate dehydrogenase (LDH) in the blood, and/or a decrease in a symptom of cardiovascular disease and/or an improvement in blood flow and cardiac function as determined by someone of ordinary skill in the art as measured by electrocardiogram (ECG or EKG), or echocardiogram (heart ultrasound), Doppler ultrasound and nuclear medicine imaging. A reduction in a symptom of a cardiovascular disease by at least about 10% would also be considered effective treatment by the methods as disclosed herein. As alternative examples, a reduction in a symptom of cardiovascular disease, for example a reduction of at least one of the following; dyspnea, chest pain, palpitations, dizziness, syncope, edema, cyanosis etc. by at least about 10% or a cessation of such systems, or a reduction in the size one such symptom of a cardiovascular disease by at least about 10% would also be considered as affective treatments by the methods as disclosed herein. In some embodiments, it is preferred, but not required that the therapeutic agent actually eliminate the cardiovascular disease or disorder, rather just reduce a symptom to a manageable extent.

Subjects amenable to treatment with a anti-miR or combination thereof as disclosed herein, (e.g., but not limited to, an anti-miR-155 alone or in combination with an anti-miR-425 agent; or an anti-miR-105 agent alone, or in combination with an anti-miR-425) according to the methods as disclosed herein can be identified by any method to diagnose high blood pressure, e.g., using a blood pressure cuff). Methods of diagnosing these conditions are well known by persons of ordinary skill in the art. By way of non-limiting example, myocardial infarction can be diagnosed by (i) blood tests to detect levels of creatine phosphokinase (CPK), aspartate aminotransferase (AST), lactate dehydrogenase (LDH) and other enzymes released during myocardial infarction; (ii) electrocardiogram (ECG or EKG) which is a graphic recordation of cardiac activity, either on paper or a computer monitor. An ECG can be beneficial in detecting disease and/or damage; (iii) echocardiogram (heart ultrasound) used to investigate congenital heart disease and assessing abnormalities of the heart wall, including functional abnormalities of the heart wall, valves and blood vessels; (iv) Doppler ultrasound can be used to measure blood flow across a heart valve; (v) nuclear medicine imaging (also referred to as radionuclide scanning in the art) allows visualization of the anatomy and function of an organ, and can be used to detect coronary artery disease, myocardial infarction, valve disease, heart transplant rejection, check the effectiveness of bypass surgery, or to select patients for angioplasty or coronary bypass graft.

The term “effective amount” as used herein refers to the amount of therapeutic agent of pharmaceutical composition to alleviate at least one or more symptom of the disease or disorder, and relates to a sufficient amount of pharmacological composition to provide the desired effect. With reference to the treatment of, for example, a cardiovascular condition or disease in a subject, the term “effective amount” refers to the amount that is safe and sufficient to prevent or delay the development or a cardiovascular disease or disorder. The amount can thus cure or cause the cardiovascular disease or disorder to go into remission, slow the course of cardiovascular disease progression, slow or inhibit a symptom of a cardiovascular disease or disorder, slow or inhibit the establishment of secondary symptoms of a cardiovascular disease or disorder or inhibit the development of a secondary symptom of a cardiovascular disease or disorder. The effective amount for the treatment of the cardiovascular disease or disorder depends on the type of cardiovascular disease to be treated, the severity of the symptoms, the subject being treated, the age and general condition of the subject, the mode of administration and so forth. Thus, it is not possible to specify the exact “effective amount”. However, for any given case, an appropriate “effective amount” can be determined by one of ordinary skill in the art using only routine experimentation. The efficacy of treatment can be judged by an ordinarily skilled practitioner, for example, efficacy can be assessed in animal models of a cardiovascular disease or disorder as discussed herein, for example treatment of a rodent with acute myocardial infarction or ischemia-reperfusion injury, and any treatment or administration of the compositions or formulations that leads to a decrease of at least one symptom of the cardiovascular disease or disorder as disclosed herein, for example, increased heart ejection fraction, decreased rate of heart failure, decreased infarct size, decreased associated morbidity (pulmonary edema, renal failure, arrhythmias) improved exercise tolerance or other quality of life measures, and decreased mortality indicates effective treatment. In embodiments where the compositions are used for the treatment of a cardiovascular disease or disorder, the efficacy of the composition can be judged using an experimental animal model of cardiovascular disease, e.g., animal models of ischemia-reperfusion injury (Headrick J P, Am J Physiol Heart circ Physiol 285; H1797; 2003) and animal models acute myocardial infarction. (Yang Z, Am J Physiol Heart Circ. Physiol 282:H949:2002; Guo Y, J Mol Cell Cardiol 33; 825-830, 2001). When using an experimental animal model, efficacy of treatment is evidenced when a reduction in a symptom of the cardiovascular disease or disorder, for example, a reduction in one or more symptom of dyspnea, chest pain, palpitations, dizziness, syncope, edema, cyanosis, pallor, fatigue and high blood pressure which occurs earlier in treated, versus untreated animals. By “earlier” is meant that a decrease occurs at least 5% earlier, but preferably more, e.g., one day earlier, two days earlier, 3 days earlier, or more.

The phrase “therapeutically effective amount” as used herein, e.g., of an anti-miR or miR agent as disclosed herein means a sufficient amount of the composition to treat a disorder, at a reasonable benefit/risk ratio applicable to any medical treatment. The term “therapeutically effective amount” therefore refers to an amount of the composition as disclosed herein that is sufficient to effect a therapeutically significant reduction in a symptom or clinical marker associated with a cardiovascular disease or disorder, or hypertension or associated diseases.

A compound comprising an oligonucleotide consisting of a number of linked nucleosides means a compound that includes an oligonucleotide having the specified number of linked nucleosides. Thus, the compound may include additional substituents or conjugates. Unless otherwise indicated, the compound does not include any additional nucleosides beyond those of the oligonucleotide. “Oligomeric compound” means a compound comprising a polymer of linked monomeric subunits.

The term “oligonucleotide” means a polymer of linked nucleosides, each of which can be modified or unmodified, independent from one another.

A therapeutically significant reduction in a symptom is, e.g. at least about 10%, at least about 20%, at least about 30%, at least about 40%, at least about 50%, at least about 60%, at least about 70%, at least about 80%, at least about 90%, at least about 100%, at least about 125%, at least about 150% or more in a measured parameter as compared to a control or non-treated subject. Measured or measurable parameters include clinically detectable markers of disease, for example, elevated or depressed levels of a biological marker, as well as parameters related to a clinically accepted scale of symptoms or markers for a disease or disorder. It will be understood, that the total daily usage of the compositions and formulations as disclosed herein will be decided by the attending physician within the scope of sound medical judgment. The exact amount required will vary depending on factors such as the type of disease being treated.

As used herein, the phrase “cardiovascular condition, disease or disorder” is intended to include all disorders characterized by insufficient, undesired or abnormal cardiac function, e.g. hypertension, ischemic heart disease, hypertensive heart disease and pulmonary hypertensive heart disease, valvular disease, congenital heart disease and any condition which leads to congestive heart failure in a subject, particularly a human subject. Insufficient or abnormal cardiac function can be the result of disease, injury and/or aging. By way of background, a response to myocardial injury follows a well-defined path in which some cells die while others enter a state of hibernation where they are not yet dead but are dysfunctional. This is followed by infiltration of inflammatory cells, deposition of collagen as part of scarring, all of which happen in parallel with in-growth of new blood vessels and a degree of continued cell death. As used herein, the term “ischemia” refers to any localized tissue ischemia due to reduction of the inflow of blood. The term “myocardial ischemia” refers to circulatory disturbances caused by coronary atherosclerosis and/or inadequate oxygen supply to the myocardium. For example, an acute myocardial infarction represents an irreversible ischemic insult to myocardial tissue. This insult results in an occlusive (e.g., thrombotic or embolic) event in the coronary circulation and produces an environment in which the myocardial metabolic demands exceed the supply of oxygen to the myocardial tissue.

The terms “coronary artery disease” and “acute coronary syndrome” as used interchangeably herein, and refer to myocardial infarction refer to a cardiovascular condition, disease or disorder, include all disorders characterized by insufficient, undesired or abnormal cardiac function, e.g. ischemic heart disease, hypertensive heart disease and pulmonary hypertensive heart disease, valvular disease, congenital heart disease and any condition which leads to congestive heart failure in a subject, particularly a human subject. Insufficient or abnormal cardiac function can be the result of disease, injury and/or aging. By way of background, a response to myocardial injury follows a well-defined path in which some cells die while others enter a state of hibernation where they are not yet dead but are dysfunctional. This is followed by infiltration of inflammatory cells, deposition of collagen as part of scarring, all of which happen in parallel with in-growth of new blood vessels and a degree of continued cell death.

As used herein, the term “ischemia” refers to any localized tissue ischemia due to reduction of the inflow of blood. The term “myocardial ischemia” refers to circulatory disturbances caused by coronary atherosclerosis and/or inadequate oxygen supply to the myocardium. For example, an acute myocardial infarction represents an irreversible ischemic insult to myocardial tissue. This insult results in an occlusive (e.g., thrombotic or embolic) event in the coronary circulation and produces an environment in which the myocardial metabolic demands exceed the supply of oxygen to the myocardial tissue.

The term “hypertension” is also referred to as “HTN” or “high blood pressure” or “arterial hypertension” refers to a medical condition in which the blood pressure in the arteries is elevated. This requires the heart to work harder than normal to circulate blood through the blood vessels. Blood pressure is summarised by two measurements, systolic and diastolic, which depend on whether the heart muscle is contracting (systole) or relaxed between beats (diastole) and equate to a maximum and minimum pressure, respectively. Normal blood pressure at rest is within the range of 100-140 mmHg systolic (top reading) and 60-90 mmHg diastolic (bottom reading). High blood pressure is said to be present if it is persistently at or above 140/90 mmHg Without wishing to be bound by theory, hypertension is classified as either primary (essential) hypertension or secondary hypertension; about 90-95% of cases are categorized as “primary hypertension” which means high blood pressure with no obvious underlying medical cause. The remaining 5-10% of cases (secondary hypertension) are caused by other conditions that affect the kidneys, arteries, heart or endocrine system. Hypertension is a major risk factor for stroke, myocardial infarction (heart attacks), heart failure or chronic heart failure (CHF), aneurysms of the arteries (e.g. aortic aneurysm), peripheral arterial disease and is a cause of chronic kidney disease. Even moderate elevation of arterial blood pressure is associated with a shortened life expectancy. Dietary and lifestyle changes can improve blood pressure control and decrease the risk of associated health complications, although drug treatment is often necessary in people for whom lifestyle changes prove ineffective or insufficient.

The term “hypotension” as used herein refers to low blood pressure, especially in the arteries of the systemic circulation. Blood pressure is the force of blood pushing against the walls of the arteries as the heart pumps out blood. Hypotension is generally considered to be systolic blood pressure less than 90 millimeters of mercury (mm Hg) or diastolic less than 60 mm Hg. Hypotension is the opposite of hypertension, which is high blood pressure. For many people, low blood pressure can cause dizziness and fainting or indicate serious heart, endocrine or neurological disorders. Severely low blood pressure can deprive the brain and other vital organs of oxygen and nutrients, leading to a life-threatening condition called shock.

The term “orthostatic hypotension” as used herein is also known as postural hypotension, orthostasis, and refers to a form of hypotension in which a person's blood pressure suddenly falls when standing up or stretching. Medically it is defined as a fall in systolic blood pressure of at least 20 mm Hg and diastolic blood pressure of at least 10 mm Hg when a person assumes a standing position. The symptom is caused by blood pooling in the lower extremities upon a change in body position.

As used herein, the terms “administering,” and “introducing” are used interchangeably herein and refer to the placement of the therapeutic agents as disclosed herein into a subject by a method or route which results in delivering of such agent(s) at a desired site. The compounds can be administered by any appropriate route which results in an effective treatment in the subject, including topical administration.

The phrases “parenteral administration” and “administered parenterally” as used herein mean modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. The phrases “systemic administration,” “administered systemically”, “peripheral administration” and “administered peripherally” as used herein mean the administration therapeutic compositions other than directly into a tumor such that it enters the animal's system and, thus, is subject to metabolism and other like processes.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” as used herein means a pharmaceutically acceptable material, composition or vehicle, such as a liquid or solid filler, diluent, excipient, solvent or encapsulating material, involved in maintaining the activity of or carrying or transporting the subject agents from one organ, or portion of the body, to another organ, or portion of the body. In addition to being “pharmaceutically acceptable” as that term is defined herein, each carrier must also be “acceptable” in the sense of being compatible with the other ingredients of the formulation. The pharmaceutical formulation contains a compound of the invention in combination with one or more pharmaceutically acceptable ingredients. The carrier can be in the form of a solid, semi-solid or liquid diluent, cream or a capsule. These pharmaceutical preparations are a further object of the invention. Usually the amount of active compounds is between 0.1-95% by weight of the preparation, preferably between 0.2-20% by weight in preparations for parenteral use and preferably between 1 and 50% by weight in preparations for oral administration. For the clinical use of the methods of the present invention, targeted delivery composition of the invention is formulated into pharmaceutical compositions or pharmaceutical formulations for parenteral administration, e.g., intravenous; mucosal, e.g., intranasal; enteral, e.g., oral; topical, e.g., transdermal; ocular, e.g., via corneal scarification or other mode of administration. The pharmaceutical composition contains a compound of the invention in combination with one or more pharmaceutically acceptable ingredients. The carrier can be in the form of a solid, semi-solid or liquid diluent, cream or a capsule.

The terms “composition” or “pharmaceutical composition” used interchangeably herein refer to compositions or formulations that usually comprise an excipient, such as a pharmaceutically acceptable carrier that is conventional in the art and that is suitable for administration to mammals, and preferably humans or human cells. Such compositions can be specifically formulated for administration via one or more of a number of routes, including but not limited to, oral, ocular parenteral, intravenous, intraarterial, subcutaneous, intranasal, sublingual, intraspinal, intracerebroventricular, and the like. In addition, compositions for topical (e.g., oral mucosa, respiratory mucosa) and/or oral administration can form solutions, suspensions, tablets, pills, capsules, sustained-release formulations, oral rinses, or powders, as known in the art are described herein. The compositions also can include stabilizers and preservatives. For examples of carriers, stabilizers and adjuvants, University of the Sciences in Philadelphia (2005) Remington: The Science and Practice of Pharmacy with Facts and Comparisons, 21st Ed.

The term “agent” refers to any entity which is normally not present or not present at the levels being administered to a cell, tissue or subject. Agent can be selected from a group comprising: chemicals; small molecules; nucleic acid sequences; nucleic acid analogues; proteins; peptides; aptamers; antibodies; or functional fragments thereof. A nucleic acid sequence can be RNA or DNA, and can be single or double stranded, and can be selected from a group comprising: nucleic acid encoding a protein of interest; oligonucleotides; and nucleic acid analogues; for example peptide-nucleic acid (PNA), pseudo-complementary PNA (pc-PNA), locked nucleic acid (LNA), etc. Such nucleic acid sequences include, but are not limited to nucleic acid sequence encoding proteins, for example that act as transcriptional repressors, antisense molecules, ribozymes, small inhibitory nucleic acid sequences, for example but not limited to RNAi, shRNAi, siRNA, micro RNAi (mRNAi), antisense oligonucleotides etc. A protein and/or peptide or fragment thereof can be any protein of interest, for example, but not limited to; mutated proteins; therapeutic proteins; truncated proteins, wherein the protein is normally absent or expressed at lower levels in the cell. Proteins can also be selected from a group comprising; mutated proteins, genetically engineered proteins, peptides, synthetic peptides, recombinant proteins, chimeric proteins, antibodies, midibodies, tribodies, humanized proteins, humanized antibodies, chimeric antibodies, modified proteins and fragments thereof. An agent can be applied to the media, where it contacts the cell and induces its effects. Alternatively, an agent can be intracellular as a result of introduction of a nucleic acid sequence encoding the agent into the cell and its transcription resulting in the production of the nucleic acid and/or protein environmental stimuli within the cell. In some embodiments, the agent is any chemical, entity or moiety, including without limitation synthetic and naturally-occurring non-proteinaceous entities. In certain embodiments the agent is a small molecule having a chemical moiety. For example, chemical moieties included unsubstituted or substituted alkyl, aromatic, or heterocyclyl moieties including macrolides, leptomycins and related natural products or analogues thereof. Agents can be known to have a desired activity and/or property, or can be selected from a library of diverse compounds.

The articles “a” and “an” are used herein to refer to one or to more than one (i.e., to at least one) of the grammatical object of the article. By way of example, “an element” means one element or more than one element.

Other than in the operating examples, or where otherwise indicated, all numbers expressing quantities of ingredients or reaction conditions used herein should be understood as modified in all instances by the term “about.” The term “about” when used in connection with percentages can mean±1%. The present invention is further explained in detail by the following examples, but the scope of the invention should not be limited thereto.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

miRNAs

miRs are small RNA molecules that contain sequences complementary to their target mRNAs. The biogenesis of a miR begins with the transcription of the miR gene into a long primary transcript (pri-miR), which is then processed into a hairpin-shaped precursor miR (pre-miR). The pre-miR is exported out of the nucleus into the cytoplasm, where it is processed into a double-stranded miR duplex. The two strands of the duplex are then separated and one of the strands (the passenger strand) is degraded, while the other strand (the guide strand) becomes the mature single-stranded miR. The mature miR regulates gene expression by binding to partially complementary sequences that are usually located in the 3′UTR of target mRNAs. Binding of miRs to mRNAs causes translational repression and/or target mRNA degradation, leading to reduced protein levels. The region in the miR that is thought to be important for target mRNA recognition is known as the seed sequence, comprising nucleotides 2 to 7 at the miR 5′-end.

### Anti-miR Agents

The inventors herein demonstrate that miR-103, miR-105, miR-107 and miR-155 each bind to a region in the 3′UTR of the NPPA gene, thereby repressesing the expression of ANP mRNA and production of ANP protein.

Accordingly, the inventors demonstrate that inhibitors (e.g., antagonists or antimirs) to at least one of miR-103, miR-105, miR-107 and miR-155, or any combination thereof, can be used to increase ANP levels in subjects. An inhibitor (e.g., antagonist) of miR-103, miR-105, miR-107 and/or miR-155 is referred to herein as an “anti-miR agent”, and can be an anti-miR agent which inhibits the function of miR-103, or an anti-miR agent which inhibits the function of miR-105 or an anti-miR agent which inhibits the function of miR-107 or an anti-miR which inhibits the fuction of miR-155.

In an alternative and opposite embodiment, an miR agent, e.g., agonists of miR-103, miR-105, miR-107 and/or miR-155 are encompassed in the methods, compositions and kits as disclosed herein for decreasinh ANP levels (e.g., plasma ANP levels) in a subject in need thereof, e.g., a subject with low blood pressure, hypotension, or a subject suffering from shock and the like.

In some embodiments, an anti-miR is an oligonucleotide or modified oligonucleotide that binds to, and interferes with the activity of a specific target miRNA, e.g., miR-103, miR-105, miR-107 and/or miR-155. In some embodiments, an anti-miR agent can also be a small molecule which interferes with the binding of the miR to its target binding sequence, a small molecule which binds to the seed sequence of any of miR-103, miR-105, miR-107 and/or miR-155, a small molecule which decreases expression of any of miR-103, miR-105, miR-107 or miR-155. In some embodiments, where an anti-miR is an oligonucleotide, an anti-miR can adopt a variety of configurations including single stranded, double stranded (RNA/RNA or RNA/DNA duplexes), and hairpin designs, in general, microRNA inhibitors comprise one or more sequences or portions of sequences that are complementary or partially complementary with the mature strand (or strands) of the miRNA to be targeted, in addition, the miRNA inhibitor can also comprise additional sequences located 5′ and 3′ to the sequence that is the reverse complement of the mature miRNA. The additional sequences can be the reverse complements of the sequences that are adjacent to the mature miRNA in the pri-miRNA from which the mature miRNA is derived, or the additional sequences can be arbitrary sequences (having a mixture of A, G, C, U, or dT). In some embodiments, one or both of the additional sequences are arbitrary sequences capable of forming hairpins. Thus, in some embodiments, the sequence that is the reverse complement of the miRNA is flanked on the 5′ side and on the 3′ side by hairpin structures. MicroRNA inhibitors, when double stranded, can include mismatches between nucleotides on opposite strands. Furthermore, microRNA inhibitors can be linked to conjugate moieties in order to facilitate uptake of the inhibitor into a cell.

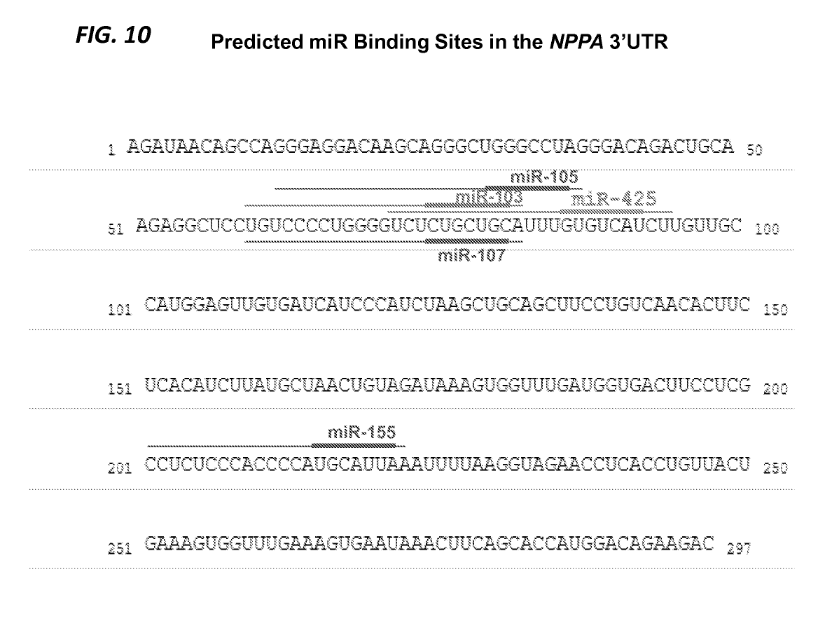

In some embodiments, an anti-miR is a blockmir, which is an anti-miR agent that has a sequence that is complementary to the mRNA sequence that is the target binding site for the miR, thereby inhibiting (i.e., blocking) the binding of the miR to the target mRNA sequence on the 3′ UTR of the NPPA gene. Accordingly, blockmirs bind to the target binding site of the miR-103, miR-105, miR-107 and/or miR-155 on the 3′ UTR of the NPPA gene, thereby blocking the binding of the respective miR (e.g., miR-103, miR-105, miR-107 and/or miR-155) and therefore specifically inhibiting the function of miR-103, miR-105, miR-107 and/or miR-155 in repressing the NPPA gene transcription and their subsequent decrease in ANP expression. The blockmirs as disclosed herein can be designed based on the secondary structure (e.g., the folding) of the region of the 3′UTR of the NPPA gene which contains target binding sites for miR-103/miR-107, miR-105, miR-155 and miR-425. In some embodiments, a blockmir binds to at least one or more of the following sequences; SEQ ID NO: 6 (the target site for miR-103/miR-107), SEQ ID NO: 10 (the target site for miR-105) or SEQ ID NO: 207 (the target site for miR-105 on the major allele (A) for rs5067) or SEQ ID NO: 208 (the target site for miR-105 on minor allele (G) for rs5067), or SEQ ID NO: 199 (the target site for miR-155 on AA or AG for rs61764044). In some embodiments, at least one of those blockmirs (that bind to SEQ ID NO: 6, 10, 207, 208 or 199 at the 3′UTR of NPPA gene) can be used in combination with blockmir for miR-435, e.g., a blockmir binds to SEQ ID NO: 18 (the target site for miR-425 on AA or AG for rs5068) In some embodiments, a bioinformatic tools can be used by one of ordinary skill in the art to predict secondary structure and folding of NPPA mRNA, including the 3′UTR, so that blockmirs can be designed to this region to inhibit binding of one or more of miR-103/miR-107, miR-105, miR-155 and miR-425 to this region. In some embodiments, a blockmir is an oligonucleotide, modified RNA (modRNA), or LNA-modified oligonucleotide which binds to a portion of nucleic acids 51-100, and/or nucleic acids 201-223 of the sequence shown in FIG. 10, thereby blocking at least one of miR-103/miR-107, miR-105, miR-155 and miR-425 to these regions. In some embodiments, a blockmir is an oligonucleotide or LNA-modified oligonucleotide that binds to the NPPA mRNA at a region distinct nucleic acids 51-100 of the sequence shown FIG. 10, but inhibits the binding of any one or more of miR-103, miR-105, miR-107, miR-155 or miR-425 to the 3′UTR of NPPA mRNA. In some embodiments, a blockmir that inhibits the binding of miR-155 is used alone, or in combination with a blockmir that inhibits the binding of miR-425 to the 3′UTR of NPPA mRNA. In some embodiments, a blockmir that inhibits the binding of miR-155 is used alone, or in combination with a blockmir that inhibits the binding of miR-425 to the 3′UTR of NPPA mRNA.

Anti-miR that Inhibits miR-103

Encompassed in the methods of the present invention is the use of any anti-miR agent which inhibits the function of miR-103 (herein referred to an “anti-miR-103 agent”). The miR-103 microRNA precursor (homologous to miR-107), is a short non-coding RNA gene involved in gene regulation. In some embodiments, such an anti-miR-103 agent binds to hsa-miR-103a-3p (SEQ ID NO: 1), or hsa-miR-103b (SEQ ID NO: 2) and/or hsa-miR-103a-2-5p (SEQ ID NO: 3) or to at least part of the human miR-103 seed sequence of SEQ ID NO: 4. In all embodiments, an anti-miR-103 agent inhibits miR-103 mediated suppression of NPPA gene expression by the inhibiting the binding of miR-103 (e.g., hsa-miR-103a-3p (SEQ ID NO: 1), or hsa-miR-103b (SEQ ID NO: 2) and/or hsa-miR-103a-2-5p (SEQ ID NO: 3)) to the 3′UTR coding region of the NPPA mRNA. In some embodiments, an anti-miR-103 agent inhibits the binding of hsa-miR-103a-3p (SEQ ID NO: 1) to the 3′UTR coding region of the NPPA mRNA.

In some embodiments, an anti-miR-103 agent can be a small molecule or oligonucleotide which inhibits binding of a miR-103 (e.g., hsa-miR-103a-3p (SEQ ID NO: 1), or hsa-miR-103b (SEQ ID NO: 2) and/or hsa-miR-103a-2-5p (SEQ ID NO: 3)) to the miR-103 binding sequence, such as SEQ ID NO: 6 in the 3′UTR of the NPPA gene, or alternatively, inhibits the expression of a miR-103 molecule, e.g., inhibits the expression of hsa-miR-103a-3p (SEQ ID NO: 1), or hsa-miR-103b (SEQ ID NO: 2) and/or hsa-miR-103a-2-5p (SEQ ID NO: 3). Such an anti-miR-103 agent which inhibits binding of a miR-103 (e.g., hsa-miR-103a-3p (SEQ ID NO: 1), or hsa-miR-103b (SEQ ID NO: 2) and/or hsa-miR-103a-2-5p (SEQ ID NO: 3)) to SEQ ID NO: 6 are useful to increase ANP levels in any subject in need of increased ANP, e.g., a subject with high blood pressure, in need of decreased blood pressure, in need of decrease hypertension or at risk of a cardiovascular disease or disorder.

An anti-miR-103 agent encompassed for use in the methods, compositions and kits as disclosed herein is a nucleic acid, or analogue or mimetic thereof which is complementary to at least part of any miR-103 sequences of hsa-miR-103a-3p (SEQ ID NO: 1), or hsa-miR-103b (SEQ ID NO: 2) and/or hsa-miR-103a-2-5p (SEQ ID NO: 3), or at least part of the miR-103 miRNA seed sequence SEQ ID NO: 4.

In some embodiments, an anti-miR-103 agent is an anti-miR which is complementary to, or in part, to the miRNA seed sequence GCAGCAU (SEQ ID NO: 4).

In some embodiments, an anti-miR agent to miR-103 is an anti-miR of UCGUCGUAACAUGUCCCGAUACU (SEQ ID NO: 196) or homologue or variant thereof. In some embodiments, an anti-miR-103 agent is an oligonucleotide of UCAUAGCCCUGUACAAUGCUGCU (SEQ ID NO: 13) or a homologue thereof. In some embodiments, where an anti-miR-103 is an oligonucleotide, such as an anti-miR which is complementary, at least in part, to the miRNA seed sequence is encoded SEQ ID NO: 4, the anti-miR-103 agent is encoded by a nucleic acid construct.

In some embodiments, an anti-miR-103 oligonucleotide can comprise a nucleic acid sequence that is complementary to at least a portion of any one of the pre-miRNA-103 nucleic acid sequences (e.g., hsa-mir-103) of hsa-mir-103a-1 stem loop (SEQ ID NO: 21); hsa-mir-103a-2 1 (SEQ ID NO: 22), hsa-mir-103b-1 (SEQ ID NO: 23) or has-mir-103b-2 (SEQ ID NO: 24). In some embodiments, an anti-miR-103 oligonucleotide comprises at least 10, or at least 12, or at least 14, or at least 15 or more contigious nucleotides of the sequence of: UCGUCGUAACAUGUCCCGAUACU (SEQ ID NO: 196), or an oligonucleotide anti-miR-103 to hsa-miR-103a-3p. An anti-miR 103 oligonucleotide can be at least 75% (e.g., 80%, 85%, 90%, 91%, 92%, 93%, 94%, 95%, 96%, 97%, 98%, 99%, or 100% (i.e. completely complementary)) complementary over a sequence comprising at least 6 (e.g., 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more) consecutive nucleotides of sequence of SEQ ID NOs: 1-3 or 21-24.

In some embodiments, the anti-miR oligonucleotide comprises a nucleotide sequence that is complementary to at least a portion of any one of the miR-103 nucleic acid sequences of hsa-miR-103a-3p (SEQ ID NO: 1), or hsa-miR-103b (SEQ ID NO: 2) and/or hsa-miR-103a-2-5p (SEQ ID NO: 3). In some embodiments, the anti-miR-103 oligonucleotide can be at least 75% (e.g., 80%, 85%, 90%, 91%, 92%, 93%, 94%, 95%, 96%, 97%, 98%, 99% complementary, or 100% (i.e. completely complementary)) over a sequence comprising at least 6 (e.g., 6, 7, 8, 9, 10, 11, 12, 13, 14, 15, 16, 17, 18, 19, 20, 21, 22, or 23) consecutive nucleotides of sequence of SEQ ID NO: 1, 2 or 3. In some embodiments, an anti-miR-103 comprises a nucleotide sequence that is complementary to nucleic acid sequence of SEQ ID NO: 4 or 5.

In some embodiments, an anti-miR-103 agent is an inhibitory nucleic acid sequence comprising a sequence that is complementary to a contiguous sequence of at least 5, or at least 6, or at least 7 or at least 8, or at least 9 or at least 10, nucleotides present in hsa-miR-103, e.g., .hsa-miR-103-3p.

Also encompassed for use in the methods, kits, compositions and assays as disclosed herein are anti-miR-103 agents which are well known in the art, including but not limited to those anti-miR-103 agents disclosed in US2014/0113953 and US2012/0122959, which are incorporated herein in their entirety by reference.

Anti-miR that Inhibits miR-105

Encompassed in the methods of the present invention is the use of any anti-miR agent which inhibits the function of miR-105 (herein referred to an “anti-miR-105 agent”). The miR-105 microRNA precursor (homologous to miR-105), is a short non-coding RNA gene involved in gene regulation. In some embodiments, such an anti-miR-105 agent binds to hsa-miR-105-5p (SEQ ID NO: 7), and/or hsa-miR-105-3p (SEQ ID NO: 8) or to at least part of the human miR-105 seed sequence of SEQ ID NO: 9 (5′-CAAAUGC-3′). In all embodiments, an anti-miR-105 agent inhibits miR-105 mediated suppression of NPPA gene expression by inhibiting at least 50% of the binding of miR-105 (e.g., hsa-miR-105-5p (SEQ ID NO: 7), and/or hsa-miR-105-3p (SEQ ID NO: 8)) to the 3′UTR coding region of the NPPA mRNA. In some embodiments, an anti-miR-105 agent inhibits the binding of hsa-miR-105-5p (SEQ ID NO: 7) to the 3′UTR coding region of the NPPA mRNA.

In some embodiments, an anti-miR-105 agent can be a small molecule or oligonucleotide which inhibits binding of a miR-105 (e.g., hsa-miR-105-5p (SEQ ID NO: 7), and/or hsa-miR-105-3p (SEQ ID NO: 8)) to the miR-105 binding sequence of SEQ ID NO: 10 in the 3′UTR of the NPPA gene, or alternatively, inhibits the expression of a miR-105 molecule, e.g., inhibits the expression of hsa-miR-105-5p (SEQ ID NO: 7), and/or hsa-miR-105-3p (SEQ ID NO: 8). Such an anti-miR-105 agent which inhibits binding of a miR-105 (e.g., hsa-miR-105-5p (SEQ ID NO: 7), and/or hsa-miR-105-3p (SEQ ID NO: 8)) to SEQ ID NO: 10 are useful to increase ANP levels in any subject in need of increased ANP, e.g., a subject with high blood pressure, in need of decreased blood pressure, in need of decreade hypertension or at risk of a cardiovascular disease or disorder. In some embodiments an anti-miR to miR-105 is a blockmir that binds to the target sequence of SEQ ID NO: 10 or SEQ ID NO: 207 (CAAAUGAAGCAGAGACCCCAG; the target site for miR-105 of the major allele of rs5067) or SEQ ID NO: 208 (CGAAUGAAGCAGAGACCCCAG; the target site for miR-105 of the minor allele of rs5067) in the 3′UTR of NPPA gene and inhibits/blocks miR-105 seed sequence of SEQ ID NO: 9 from binding.

An anti-miR-105 agent encompassed for use in the methods, compositions and kits as disclosed herein is a nucleic acid, or analogue or mimetic thereof which is complementary to at least part of any miR-105 sequences of hsa-miR-105-5p (SEQ ID NO: 7), and/or hsa-miR-105-3p (SEQ ID NO: 8), or at least part of the miR-105 miRNA seed sequence SEQ ID NO: 9.

In some embodiments, an anti-miR-105 agent is an anti-miR which is complementary to, or in part, to the miRNA seed sequence CAAAUGC (SEQ ID NO: 9).

In some embodiments, an anti-miR agent to miR-105 is an anti-miR of SEQ ID NO: 197 or a fragment or homologue or variant thereof. In some embodiments, an anti-miR-105 agent is an oligonucleotide comprising at least 10, or at least 12, or at least 14, or at leas 15 or more contigious nucleotides of the sequence of: AGUUUACGAGUCUGAGGACACCA (SEQ ID NO: 197) or a homologue thereof. Another exemplary antagomir of miR-105 includes ACCACAGGAGTCTGAGCATTTGA (SEQ ID NO:238), which is disclosed in US application US Patent Application, 20140274769, which is incorporated herein in its entirety by reference). In some embodiments, where an anti-miR-105 is an oligonucleotide, such as an anti-miR which is complementary, at least in part, to the miRNA seed sequence is encoded SEQ ID NO: 9, the anti-miR-105 agent is encoded by a nucleic acid construct.

In some embodiments, an anti-miR-105 oligonucleotide can comprise a nucleic acid sequence that is complementary to at least a portion of any one of the pre-miRNA-105 nucleic acid sequences (e.g., hsa-mir-105) of hsa-mir-105-1 stem loop (SEQ ID NO: 25) or hsa-mir-105-2 (SEQ ID NO: 26). In some embodiments, an anti-miR-105 oligonucleotide is an anti-miR-105-5p oligonucleotide of; AGUUUACGAGUCUGAGGACACCA (SEQ ID NO: 197). An anti-miR 105 oligonucleotide can be at least 75% (e.g., 80%, 85%, 90%, 91%, 92%, 93%, 94%, 95%, 96%, 97%, 98%, 99%, or 100% (i.e. completely complementary)) complementary over a sequence comprising at least 6 (e.g., 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more) consecutive nucleotides of sequence of SEQ ID NOs: 7, 8, 25 or 26.

In some embodiments, the anti-miR oligonucleotide comprises a nucleotide sequence that is complementary to at least a portion of any one of the miR-105 nucleic acid sequences of hsa-miR-105-5p (SEQ ID NO: 7), and/or hsa-miR-105-3p (SEQ ID NO: 8). In some embodiments, the anti-miR-105 oligonucleotide can be at least 75% (e.g., 80%, 85%, 90%, 91%, 92%, 93%, 94%, 95%, 96%, 97%, 98%, 99% complementary, or 100% (i.e. completely complementary)) over a sequence comprising at least 6 (e.g., 6, 7, 8, 9, 10, 11, 12, 13, 14, 15, 16, 17, 18, 19, 20, 21, 22, or 23) consecutive nucleotides of sequence of SEQ ID NO: 8 or 9. In some embodiments, an anti-miR-105 comprises a nucleotide sequence that is complementary to nucleic acid sequence of SEQ ID NO: 9 (CAAAUGC).

In some embodiments, an anti-miR-105 agent is an inhibitory nucleic acid sequence comprising a sequence that is complementary to a contiguous sequence of at least 5, or at least 6, or at least 7 or at least 8, or at least 9 or at least 10, nucleotides present in hsa-miR-105, e.g., .hsa-miR-105-5p.

Also encompassed for use in the methods, kits, compositions and assays as disclosed herein are anti-miR-105 agents which are well known in the art, including but not limited to those anti-miR-105 agents disclosed in US application 2014/0274769 and Zhou et al., Cancer cell, 2014, 25(4); 501-515, which are incorporated herein in their entirety by reference.

Anti-miR that Inhibits miR-107

Encompassed in the methods of the present invention is the use of any anti-miR agent which inhibits the function of miR-107 (herein referred to an “anti-miR-107 agent”). The miR-107 microRNA precursor (homologous to miR-107), is a short non-coding RNA gene involved in gene regulation. In some embodiments, such an anti-miR-107 agent binds to hsa-miR-107 (SEQ ID NO: 11) or to at least part of the human miR-107 seed sequence of SEQ ID NO:4. In all embodiments, an anti-miR-107 agent inhibits miR-107 mediated suppression of NPPA gene expression by inhibiting at least 50% of the binding of miR-107 (e.g., hsa-miR-107 (SEQ ID NO: 11)) to the 3′UTR coding region of the NPPA mRNA. In some embodiments, an anti-miR-107 agent inhibits the binding of hsa-miR-107 (SEQ ID NO: 11) to the 3′UTR coding region of the NPPA mRNA.

In some embodiments, an anti-miR-107 agent can be a small molecule or oligonucleotide which inhibits binding of a miR-107 (e.g., hsa-miR-107 (SEQ ID NO: 11)) to the miR-107 target/binding sequence of SEQ ID NO: 6 in the 3′UTR of the NPPA gene, or alternatively, inhibits the expression of a miR-107 molecule, e.g., inhibits the expression of hsa-miR-107 (SEQ ID NO: 11). Such an anti-miR-107 agent which inhibits binding of a miR-107 (e.g., hsa-miR-107 (SEQ ID NO: 11)) to SEQ ID NO: 6 are useful to increase ANP levels in any subject in need of increased ANP, e.g., a subject with high blood pressure, in need of decreased blood pressure, in need of decreade hypertension or at risk of a cardiovascular disease or disorder.

An anti-miR-107 agent encompassed for use in the methods, compositions and kits as disclosed herein is a nucleic acid, or analogue or mimetic thereof which is complementary to at least part of any miR-107 sequences of hsa-miR-107 (SEQ ID NO: 11), or at least part of the miR-107 miRNA seed sequence SEQ ID NO: 4.

In some embodiments, an anti-miR-107 agent is an anti-miR which is complementary to, or in part, to the miR-107 seed sequence GCAGCAU (SEQ ID NO: 4).

In some embodiments, an anti-miR agent to miR-107 is an anti-miR of UCGUCGUAACAUGUCCCGAUAGU (SEQ ID NO: 198) or UGAUAGCCCUGUACAAUGCUGCU (SEQ ID NO: 187) or homologue or variant thereof. In some embodiments, an anti-miR-107 agent is an oligonucleotide to hsa-miR-107 and comprises at least 10, or at least 12, or at least 14, or at leas 15 or more contigious nucleotides of the sequence of: UCGUCGUAACAUGUCCCGAUAGU (SEQ ID NO: 198) or a homologue thereof. In some embodiments, where an anti-miR-107 is an oligonucleotide, such as an anti-miR which is complementary, at least in part, to the miRNA seed sequence is encoded SEQ ID NO: 4, the anti-miR-107 agent is encoded by a nucleic acid construct.

In some embodiments, an anti-miR-107 oligonucleotide can comprise a nucleic acid sequence that is complementary to at least a portion of any one of the pre-miRNA-107 nucleic acid sequences (e.g., hsa-mir-107) of hsa-mir-107 stem loop (SEQ ID NO: 27). In some embodiments, an anti-miR-107 oligonucleotide is UCGUCGUAACAUGUCCCGAUAGU (SEQ ID NO: 198). An anti-miR 107 oligonucleotide can be at least 75% (e.g., 80%, 85%, 90%, 91%, 92%, 93%, 94%, 95%, 96%, 97%, 98%, 99%, or 100% (i.e. completely complementary)) complementary over a sequence comprising at least 6 (e.g., 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more) consecutive nucleotides of sequence of SEQ ID NOs: 11 or 27.

In some embodiments, the anti-miR oligonucleotide comprises a nucleotide sequence that is complementary to at least a portion of any one of the miR-107 nucleic acid sequences of hsa-miR-107 (SEQ ID NO: 11). In some embodiments, the anti-miR-107 oligonucleotide can be at least 75% (e.g., 80%, 85%, 90%, 91%, 92%, 93%, 94%, 95%, 96%, 97%, 98%, 99% complementary to, or 100% (i.e. completely complementary)) over a sequence comprising at least 6 (e.g., 6, 7, 8, 9, 10, 11, 12, 13, 14, 15, 16, 17, 18, 19, 20, 21, 22, or 23) consecutive nucleotides of sequence of SEQ ID NO: 11. In some embodiments, an anti-miR-107 comprises a nucleotide sequence of SEQ ID NO: 13 or 198.

In some embodiments, an anti-miR-107 agent is an inhibitory nucleic acid sequence comprising a sequence that is complementary to a contiguous sequence of at least 5, or at least 6, or at least 7 or at least 8, or at least 9 or at least 10, nucleotides present in hsa-miR-107.

Also encompassed for use in the methods, kits, compositions and assays as disclosed herein are anti-miR-107 agents which are well known in the art, including but not limited to those anti-miR-10 agents disclosed in US2014/0113953 and UA2012/0122959, which are incorporated herein in their entirety by reference.

Anti-miR that Inhibits miR-155

Encompassed in the methods of the present invention is the use of any anti-miR agent which inhibits the function of miR-155 (herein referred to an “anti-miR-155 agent”). An “anti-miR-155 agent” or “miR155 antagonist”, as used herein, is an agent that reduces or inhibits the expression, stability, or activity of miR155. A miR155 antagonist may function, for example, by blocking miR155 activity (e.g., by blocking repression of NPPA mRNA gene expression by the miR-155). Additionally or alternatively, the miR155 antagonist may inhibit the biogenesis of miR-155, for example, by blocking expression or processing of pre-miR155 or pri-miR155.

Exemplary miR155 antagonists are nucleic acid agents. These agents may include oligonucleotide antagonists, for example, antisense locked nucleic acid molecules (LNAs), antagomirs, or 2′O-methyl antisense RNAs targeting miR-155. Other exemplary miR-155 antagonists include anti-miR-155 RNAi agents. The effect of a miR-155 antagonist on the level of miR-155 in a cell can be determined by contacting the cell with a miR-155 antagonist, and comparing the level of miR-155 to a suitable control. In this embodiment, a anti-miR-155 agent, or a preferred quantity of an anti-miR-155 agent, is one which decreases (e.g., by 30%, preferably 50% or more, more preferably 70% or more, still more preferably, 90% or more) the level of miR-155, or increases the level of pro-nt-ANP or ANP in the plasma of a subject by (e.g., by 30%, preferably 50% or more, more preferably 70% or more, still more preferably, 90% or more), when compared to a suitable control, e.g., a comparable cell not contacted with an anti-miR-155 agent.

In certain embodiments, a miR-155 antagonist is a nucleic acid having a nucleotide sequence that is complementary to a miRNA-155 sequence, meaning that the nucleotide sequence is a least 60%, 65%, 70%, 75%, 80%, 85%, 90%, 95%, 97%, 98%, 99% or 100% identical to the complement of miR-155 precursor thereof over the entire length of the miRNA sequence or within a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleobases, or that the two sequences hybridize under stringent hybridization conditions. Accordingly, in certain embodiments the miR-155 antagonist may have one or more mismatched basepairs (e.g., 1, 2, 3, 4 or 5 mismatches) with respect to its target miR-155 sequence, and is capable of hybridizing to its target sequence. In certain embodiments, a miR-155 nucleic acid antagonist is perfectly complementary to miR-155 sequence. In certain embodiments, the nucleobase sequence of a modified oligonucleotide has full-length complementary to a miRNA.

The miR-155 microRNA precursor (homologous to miR-155), is a short non-coding RNA gene involved in gene regulation. In some embodiments, such an anti-miR-155 agent binds to hsa-miR-105-5p (SEQ ID NO: 12), and/or hsa-miR-105-3p (SEQ ID NO: 209) or to at least part of the human miR-105 seed sequence of SEQ ID NO: 14 (5′-AAUGCU-3′). In all embodiments, an anti-miR-155 agent inhibits miR-155 mediated suppression of NPPA gene expression by inhibiting at least 50% of the binding of miR-155 (e.g., hsa-miR-155-5p (SEQ ID NO: 12), and/or hsa-miR-155-3p (SEQ ID NO: 209)) to the 3′UTR coding region of the NPPA mRNA. In some embodiments, an anti-miR-155 agent inhibits the binding of hsa-miR-155-5p (SEQ ID NO: 12) to SEQ ID NO: 199 (e.g., the miR-155 target site comprising the major (A) allele of rs61764044) in the 3′UTR coding region of the NPPA mRNA. In some embodiments, an anti-miR-155 agent inhibits the binding of hsa-miR-155-5p (SEQ ID NO: 12) to SEQ ID NO: 200 (e.g., the miR-155 target site comprising the minor (G) allele of rs61764044) in the 3′UTR coding region of the NPPA mRNA.

In some embodiments, an anti-miR-155 agent can be a small molecule or oligonucleotide which inhibits binding of a miR-155 (e.g., hsa-miR-155-5p (SEQ ID NO: 12), and/or hsa-miR-155-3p (SEQ ID NO: 209)) to the miR-155 target/binding sequence of SEQ ID NO: 199 in the 3′UTR of the NPPA gene, or alternatively, inhibits the expression of a miR-155 molecule, e.g., inhibits the expression of hsa-miR-155-5p (SEQ ID NO: 12), and/or hsa-miR-155-3p (SEQ ID NO: 209). Such an anti-miR-155 agent which inhibits binding of miR-155 (e.g., hsa-miR-155-5p (SEQ ID NO: 12), and/or hsa-miR-155-3p (SEQ ID NO: 209)) to SEQ ID NO: 199 or 200 is useful in the methods, compositions and kits as disclosed herein to increase ANP levels in any subject in need of increased ANP, e.g., a subject with high blood pressure, in need of decreased blood pressure, in need of decreade hypertension or at risk of a cardiovascular disease or disorder. In some embodiments an anti-miR to miR-155 is a blockmir that binds to the target sequence of SEQ ID NO: 199 (UAAUGCAUGGGGUGGGAGAGG; the target site for miR-155 of the major (A) allele of rs61764044) or SEQ ID NO: 200 (UAAUGCGUGGGGUGGGAGAGG; the target site for miR-155 of the minor (G) allele of rs61764044) in the 3′UTR of NPPA gene and inhibits/blocks miR-155 seed sequence of SEQ ID NO: 14 from binding.

An anti-miR-105 agent encompassed for use in the methods, compositions and kits as disclosed herein is a nucleic acid, or analogue or mimetic thereof which is complementary to at least part of any miR-155 sequences of hsa-miR-155-5p (SEQ ID NO: 12), and/or hsa-miR-155-3p (SEQ ID NO: 209), or at least part of the miR-155 miRNA seed sequence AAUGCU (SEQ ID NO: 14).

In some embodiments, an anti-miR-155 agent is an anti-miR which is complementary to, or in part, to the miRNA seed sequence AAUGCU (SEQ ID NO: 14).

In some embodiments, an anti-miR agent to miR-155 is an anti-miR of CCCCTATCACGATTAGCATTAA (SEQ ID NO: 204) or a fragment or homologue or variant thereof. In some embodiments, an anti-miR-155 agent is an oligonucleotide comprising at least 10, or at least 12, or at least 14, or at leas 15 or more contigious nucleotides of the sequence of: CCCCTATCACGATTAGCATTAA (SEQ ID NO: 204) or a homologue thereof. In some embodiments, where an anti-miR-155 is an oligonucleotide, such as an anti-miR which is complementary, at least in part, to the miRNA seed sequence is encoded SEQ ID NO: 14, the anti-miR-155 agent is encoded by a nucleic acid construct.

In some embodiments, an anti-miR-155 oligonucleotide can comprise a nucleic acid sequence that is complementary to at least a portion of any one of the pre-miRNA-155 nucleic acid sequences (e.g., hsa-pre-mir-155) of SEQ ID NO: 203. In some embodiments, an anti-miR-155 oligonucleotide is an anti-miR-155-5p oligonucleotide of CCCCTATCACGATTAGCATTAA (SEQ ID NO: 204). In some embodiments, an anti-miR-155 agent is CCCCUAUCACAATUAGCAUUAA (SEQ ID NO: 239), as disclosed in Koval et al., Hum. Mol Genetics, 2013; 22(20); 41-4135, which is incorporated herein in its entirety by reference. An anti-miR 155 oligonucleotide can be at least 75% (e.g., 80%, 85%, 90%, 91%, 92%, 93%, 94%, 95%, 96%, 97%, 98%, 99% complementary to, or 100% (i.e. completely complementary)) to a sequence comprising at least 6 (e.g., 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more) consecutive nucleotides of sequence of SEQ ID NO 12, 14, 203, 199 or 200.

In some embodiments, the subject is administered at least one inhibitory nucleic acid comprising a sequence that is complementary to a contiguous sequence present in hsa-miR-155 (e.g., a contiguous sequence present in mature or precursor hsa-miR-155). In non-limiting embodiments, the inhibitory nucleic acid can be an antisense oligonucleotide, a ribozyme, an siRNA, or an antagomir. In some embodiments, the at least one inhibitory nucleic acid is injected into the cerebrospinal fluid of a subject. In some embodiments, the injection is intracranial injection or intrathecal injection. In some embodiments, the at least one inhibitory nucleic acid is complexed with one or more cationic polymers and/or cationic lipids (e.g., any of the cationic polymers described herein or known in the art). Antagomirs to decrease the expression and/or activity of a specific target miRNA (e.g., hsa-miR-155) can be designed using methods known in the art (see, e.g., Krutzfeld et al., Nature 438:685-689, 2005). Additional exemplary methods for designing and making antagomirs and other types of inhibitory nucleic acids are described herein.

In some embodiments, the inhibitory nucleic acid that decreases miR-155 levels is the antogmir-155 LNA sequence+TC+AC+A+A+TTA+G+C+AT+T+A (SEQ ID NO: 240) (wherein the + indicates the presence of an LNA moiety). Methods for designing antagomirs to target microRNA molecules are described in Obad et al., Nature Genetics 43:371-378, 2011. Additional inhibitory nucleic acids for decreasing the levels or expression of hsa-miR-155 are described in Worm et al., Nucleic Acids Res. 37:5784-5792, 2009, and Murugaiyan et al., J. Immunol. 187:2213-2221, 2011.

In some embodiments, the anti-miR oligonucleotide comprises a nucleotide sequence that is complementary to at least a portion of any one of the miR-155 nucleic acid sequences of hsa-miR-155-5p (SEQ ID NO: 12), and/or hsa-miR-105-3p (SEQ ID NO: 209). In some embodiments, the anti-miR-155 oligonucleotide can be at least 75% (e.g., 80%, 85%, 90%, 91%, 92%, 93%, 94%, 95%, 96%, 97%, 98%, 99% complementary to, or 100% (i.e. completely complementary)) to a sequence comprising at least 6 (e.g., 6, 7, 8, 9, 10, 11, 12, 13, 14, 15, 16, 17, 18, 19, 20, 21, 22, or 23) consecutive nucleotides of sequence of SEQ ID NO: 12 or 14. In some embodiments, an anti-miR-155 comprises a nucleotide sequence that is complementary to nucleic acid sequence of AAUGCU (SEQ ID NO: 14).

In some embodiments, an anti-miR-155 agent is an inhibitory nucleic acid sequence comprising a sequence that is complementary to a contiguous sequence of at least 5, or at least 6, or at least 7 or at least 8, or at least 9 or at least 10, nucleotides present in hsa-miR-155, e.g., .hsa-miR-155-5p.

Also encompassed for use in the methods, kits, compositions and assays as disclosed herein are anti-miR-155 agents which are well known in the art, including but not limited to those anti-miR-155 agents disclosed in U.S. Pat. No. 8,106,025 and US Patent Applications, 2014/0235697 and 2011/0293653 which are incorporated herein in their entirety by reference. In some embodiments, anti-miRs as disclosed herein, e.g., anti-miR-155 were synthesized with a full phosphorothioate backbone with alternating blocks of 2′-MOE and 2′fluoro sugar-modified nucleosides.

Inhibitors of miR-103, miR-105, miR-107 and miR-155 encompassed for use in the methods, compositions and kits as disclosed herein include inhibitory peptides or polypeptides. As used herein, the term peptide, polypeptide, protein or peptide portion is used broadly herein to mean two or more amino acids linked by a peptide bond. Protein, peptide and polypeptide are also used herein interchangeably to refer to amino acid sequences. The term fragment is used herein to refer to a portion of a full-length polypeptide or protein. It should be recognized that the term polypeptide is not used herein to suggest a particular size or number of amino acids comprising the molecule and that a peptide of the invention can contain up to several amino acid residues or more.

Optionally, the inhibitor of miR-103, miR-105, miR-107 and miR-155 is a functional nucleic acids. Such functional nucleic acids include, but are not limited to, antisense molecules and ribozymes. Thus, for example, an antisense oligonucleotide could be used to reduce or eliminate miR-103, miR-105, miR-107 and miR-155. Functional nucleic acids are generally designed using algorithms and a conventional nucleic acid synthesizer. As discussed in more detail below, such nucleic acids can, optionally, comprise one or more chemical modifications to improve in vitro and in vivo stability or delivery.

Functional nucleic acids are nucleic acid molecules that have a specific function, such as binding a target molecule or catalyzing a specific reaction. Functional nucleic acid molecules can interact with any macromolecule, such as DNA, RNA, polypeptides, or carbohydrate chains. Thus, functional nucleic acids can interact with any of miR-103, miR-105, miR-107 and miR-155 directly. Optionally, the inhibitor is an indirect inhibitor that interacts with the targets of miR-103, miR-105, miR-107 and miR-155 to inhibit the activity of miR-103, miR-105, miR-107 and miR-155 or reduce or eliminate the presence of miR-103, miR-105, miR-107 and miR-155. Often functional nucleic acids are designed to interact with other nucleic acids based on sequence homology between the target molecule and the functional nucleic acid molecule.

Antisense molecules or antisense oligonucleotides (ASOs) are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. See for example, Vermeulen et al., RNA 13: 723-730 (2007) and in WO2007/095387 and WO 2008/036825; Yue, et al., Curr. Genomics, 10(7):478-92 (2009) and Lennox Gene Ther. 18(12):1111-20 (2011), which are incorporated by reference herein in their entireties.

Thus, antisense molecules that inhibit any of miR-103, miR-105, miR-107 and miR-155 can be designed and made using standard nucleic acid synthesis techniques or obtained from a commercial entity, e.g., Regulus Therapeutics (San Diego, Calif.). Optionally, the antisense molecule is single-stranded and comprises RNA and/or DNA. Optionally, the backbone of the molecule is modified by various chemical modifications to improve the in vitro and in vivo stability and to improve the in vivo delivery of antisense molecules. Modifications of antisense molecules include, but are not limited to, 2′-O-methyl modifications, 2′-O-methyl modified ribose sugars with terminal phosphorothioates and a cholesterol group at the 3′ end, 2′-O-methoxyethyl (2′-MOE) modifications, 2′-fluoro modifications, and 2′,4′ methylene modifications (referred to as “locked nucleic acids” or LNAs). Thus, inhibitory nucleic acids include, for example, modified oligonucleotides (2′-O-methylated or 2′-O-methoxyethyl), locked nucleic acids (LNA; see, e.g, Valoczi et al., Nucleic Acids Res. 32(22):e175 (2004)), morpholino oligonucleotides (see, e.g, Kloosterman et al., PLoS Biol 5(8):e203 (2007)), peptide nucleic acids (PNAs), PNA-peptide conjugates, and LNA/2′-O-methylated oligonucleotide mixmers (see, e.g., Fabiani and Gait, RNA 14:336-46 (2008)). Optionally, the antisense molecule is an antagomir. Antagomirs are oligonucleotides comprising 2′-O-methyl modified ribose sugars with terminal phosphorothioates and a cholesterol group at the 3′ end.

miRs comprising LNA (typically identified in capitals, DNA in lower case, complete phosphorothioate backbone, where a capital C denotes LNA methylcytosine, are described in Lanford et al., Science 327(5962:198-201 (2010), which is incorporated by reference herein in its entirety. See also Elmen et al., Nature 452:896-9 (2008); and Elmen et al., Nucleic Acids Res. 36:1153-1162 (2008), which are incorporated by reference herein in their entireties. Optionally, the nucleic acid comprises a targeting sequence of miR-103, miR-105, miR-107 and miR-155. Such miRNA-binding nucleic acids are referred to as miRNA decoys or miRNA sponges. For example, mRNAs with multiple copies of the miRNA target can be engineered into the 3′ UTR of the mRNA creating an miRNA “sponge.” The miRNA inhibitors function by sequestering the cellular miRNAs away from the mRNAs that normally would be targeted by them. Such nucleic acid decoys can be delivered, e.g., by viral vectors, and expressed to inhibit the activity of any of miR-103, miR-105, miR-107 and miR-155.

Ribozymes are nucleic acid molecules that are capable of catalyzing a chemical reaction, either intramolecularly or intermolecularly. Typically, ribozymes cleave RNA or DNA substrates. There are a number of different types of ribozymes that catalyze chemical reactions which are based on ribozymes found in natural systems, such as hammerhead ribozymes, and hairpin ribozymes. There are also a number of ribozymes that are not found in natural systems, but which have been engineered to catalyze specific reactions. See, for example, U.S. Pat. Nos. 5,807,718, and 5,910,408. Representative examples of how to make and use ribozymes to catalyze a variety of different reactions can be found in, for example, U.S. Pat. Nos. 5,837,855, 5,877,022, 5,972,704, 5,989,906, and 6,017,756.

Thus, based on the sequences of miR-103, miR-105, miR-107 and miR-155, inhibitory nucleic acids can be designed to bind to any form of the miRNA, e.g., the pri-, pre- or mature form Inhibitory nucleic acids typically bind to at least a portion of the targeted sequence, in this case miR-103, miR-105, miR-107 and miR-155. The inhibitory nucleic acids are at least partially complementary to the pri-, pre-, or mature sequence of miR-103, miR-105, miR-107 and miR-155. A percent complementarity indicates the percentage of residues in a nucleic acid molecule that can form hydrogen bonds (e.g., Watson-Crick base pairing) with a second nucleic acid sequence (e.g., 5, 6, 7, 8, 9, or 10 nucleotides out of a total of 10 nucleotides in the first oligonucleotide being based paired to a second nucleic acid sequence having 10 nucleotides represents 50%, 60%, 70%, 80%, 90%, and 100% complementary respectively). 100% complementary means that all the residues of a nucleic acid sequence will hydrogen bond with the same number of residues in a second nucleic acid sequence. Thus, the microRNA inhibitor sequence has 100%, 95%, 90%, 85%, 80%, 75%, 70% complementarity, or any percent complementarity between 100% and 70%, to the pri-, pre-, or mature sequence of miR-103, miR-105, miR-107 and miR-155. Optionally, a first portion of the microRNA inhibitor sequence is identical (i.e., has 100% complementary) to the pri-, pre-, or mature sequence of miR-103, miR-105, miR-107 and miR-155, while a second portion of the microRNA inhibitor sequence has less than 100% complementarity, e.g. 50%, to the pri-, pre-, or mature sequence of miR-103, miR-105, miR-107 and miR-155.

Optionally, the inhibitory nucleic acids specifically hybridizes to one or more of the pre-, pri- or mature forms of miR-103, miR-105, miR-107 and miR-155. The phrase selectively (or specifically) hybridizes to refers to the binding, duplexing, or hybridizing of a molecule only to a particular nucleotide sequence with a higher affinity, e.g., under more stringent conditions, than to other nucleotide sequences (e.g., total cellular or library DNA or RNA). One of skill in the art will appreciate that specific hybridization between nucleotides usually relies on Watson-Crick pair bonding between complementary nucleotide sequences, which is discussed in more detail above. Optionally, the inhibitory nucleic acids bind to the pri-, pre-, or mature forms of miR-103, miR-105, miR-107 and miR-155 under stringent or highly stringent conditions. The degree of stringency can be controlled by temperature, ionic strength, pH and/or the presence of a partially denaturing solvent such as formamide. For example, the stringency of hybridization is conveniently varied by changing the concentration of formamide within the range up to and about 50%. The degree of complementarity (sequence identity) required for detectable binding will vary in accordance with the stringency of the hybridization medium and/or wash medium.

High stringency conditions for nucleic acid hybridization are well known in the art. For example, conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleotide content of the target sequence(s), the charge composition of the nucleic acid(s), and by the presence or concentration of formamide, tetramethylammonium chloride or other solvent(s) in a hybridization mixture. As discussed above, nucleic acids can be completely complementary to a target sequence or exhibit one or more mismatches.

In certain embodiments, an anti-miR agent as disclosed herein is an oligonucleotide that has a sequence that is complementary to a miR as disclosed herein (e.g., SEQ ID NOs 1, 7, 11 and 12), or a precursor (e.g., pre-miR or pri-miR, or stem loop miRs) thereof. Nucleobase sequences of mature miRNAs and their corresponding stem-loop sequences described herein are the sequences found in miRBase, an online searchable database of miRNA sequences and annotation, found at http://microma.sanger.ac.uk/. Entries in the miRBase Sequence database represent a predicted hairpin portion of a miRNA transcript (the stem-loop), with information on the location and sequence of the mature miRNA sequence. The miRNA stem-loop sequences in the database are not strictly precursor miRNAs (pre-miRNAs), and may in some instances include the pre-miRNA and some flanking sequence from the presumed primary transcript. The miRNA nucleobase sequences described herein encompass any version of the miRNA, including the sequences described in Release 10.0 of the miRBase sequence database and sequences described in any earlier Release of the miRBase sequence database. A sequence database release may result in the re-naming of certain miRNAs. The compositions of the present invention encompass modified oligonucleotides that are complementary to any nucleobase sequence version of the miRNAs described herein.

In certain embodiments, an oligonucleotide has a nucleobase sequence that is complementary to a miR or a precursor of SEQ ID NO: 1, 7, 11, or 12. Accordingly, in certain embodiments the nucleobase sequence of an oligonucleotide may have one or more mismatched basepairs with respect to its target miR or precursor sequence, and remains capable of hybridizing to its target sequence. In certain embodiments, an oligonucleotide has a nucleobase sequence that is fully complementary to a miR or precursor thereof. In certain embodiments, an oligonucleotide has a nucleobase sequence that is fully (i.e., 100%) complementary to a miR seed sequences of SEQ ID NO: 4, 9 or 14. In certain embodiments, an oligonucleotide has a nucleobase sequence that is at least 99%, or at least 98%, or at least 97%, or at least 96%, or at least 95%, or at least 94%, or at least 93% complementary to a miR seed sequences of SEQ ID NO: 4, 9 or 14. In some embodiments, an oligonucleotide is a blockmir that has a nucleobase sequence that is 100%, or at least 99%, or at least 98%, or at least 97%, or at least 96%, or at least 95%, or at least 94%, or at least 93% complementary t any of SEQ ID NO: 6, 10, 199 or 200, i.e., the miR target/binding sites for miR-103/107, miR-105 and miR-155 in the 3′UTR of the NPPA gene.

In certain embodiments, an oligonucleotide has a sequence that is complementary to a nucleobase sequence of a miRNA stem-loop sequence selected from the miR-103-1 stem-loop sequence, the miR-103-2 stem loop sequence, and the miR-107 stem loop sequence.

In certain embodiments, an oligonucleotide has a sequence that is complementary to a nucleobase sequence of a miRNA, where the nucleobase sequence of the miRNA is selected from SEQ ID NO: 1, 7, 11 or 12. In certain embodiments, an oligonucleotide has a nucleobase sequence that is complementary to a region of the miR-103-1 stem-loop sequence (SEQ ID NO: 21). In certain embodiments, an oligonucleotide has a nucleobase sequence that is complementary to the region of nucleobases 48-70 of SEQ ID NO: 21. In certain embodiments, an oligonucleotide has a nucleobase sequence that is complementary to a region of the miR-103-2 stem-loop sequence (SEQ ID NO: 22). In certain embodiments, an oligonucleotide has a nucleobase sequence that is complementary to the region of nucleobases 48-70 of SEQ ID NO:22.

In certain embodiments, an oligonucleotide has a nucleobase sequence that is complementary to a region of the miR-107 stem-loop sequence (SEQ ID NO: 27). In certain embodiments, an oligonucleotide has a nucleobase sequence that is complementary to the region of nucleobases 50-72 of SEQ ID NO: 27.

In certain embodiments, an oligonucleotide has a nucleobase sequence that is complementary to the nucleobase sequence of miR-103 (SEQ ID NO: 1). In certain embodiments, an oligonucleotide has a nucleobase sequence comprising the nucleobase sequence UCAUAGCCCUGUACAAUGCUGCU (SEQ ID NO: 13). In certain embodiments, an oligonucleotide has a nucleobase sequence consisting of the nucleobase sequence UCAUAGCCCUGUACAAUGCUGCU (SEQ ID NO: 13).

In certain embodiments, an oligonucleotide has a nucleobase sequence that is complementary to the nucleobase sequence of miR-107 (SEQ ID NO: 11). In certain embodiments, an oligonucleotide has a nucleobase sequence comprising the nucleobase sequence UGAUAGCCCUGUACAAUGCUGCU (SEQ ID NO: 187). In certain embodiments, an oligonucleotide has a nucleobase sequence consisting of the nucleobase sequence UGAUAGCCCUGUACAAUGCUGCU (SEQ ID NO: 187).

In certain embodiments, an oligonucleotide has a nucleobase sequence that is complementary to the nucleobase sequence of miR-103 or miR-107, and is capable of inhibiting the activity of both miR-103 and miR-107, as a result of the sequence similarity between miR-103 and miR-107. An oligonucleotide having a nucleobase sequence fully complementary to miR-103 will have only one mismatch relative to miR-107, thus such an oligonucleotide fully complementary to miR-103 may inhibit the activity of both miR-103 and miR-107. Likewise, an oligonucleotide having a nucleobase sequence fully complementary to miR-107 will have only one mismatch relative to miR-103, thus such an oligonucleotide fully complementary to miR-107 may inhibit the activity of both miR-103 and miR-107. As such, oligonucleotides complementary to one or both of miR-103 and miR-107 may be used in the methods provided herein. In certain embodiments, an oligonucleotide has a nucleobase sequence that is complementary to nucleobases 1-21 of SEQ ID NO: 21 (miR-103) or to nucleobases 1-21 of SEQ ID NO: 27 (miR-107). Such oligonucleotides are 100% complementary to both miR-103 and miR-107.

In certain embodiments, an oligonucleotide comprises a nucleobase sequence that is complementary to a seed sequence of SEQ ID NO: 4, which is the seed sequence for both miR-103 and miR-107. Oligonucleotides having any length described herein may comprise a seed-match sequence. In certain such embodiments, the modified oligonucleotide consists of 7 linked nucleosides. In certain embodiments, the modified oligonucleotide consists of 8 linked nucleosides. In certain embodiments, the modified oligonucleotide consists of 9 linked nucleosides. In certain embodiments, the modified oligonucleotide consists of 10 linked nucleosides. In certain embodiments, the modified oligonucleotide consists of 11 linked nucleosides. In certain embodiments, the modified oligonucleotide consists of 12 linked nucleosides.

In certain embodiments, the nucleobase sequence of the modified oligonucleotide comprises the nucleobase sequence AUGCUGCU (SEQ ID NO: 188), which is complementary to nucleotides 1-8 of miR-103 (SEQ ID NO: 1) and miR-107 (SEQ ID NO: 11). In certain embodiments, the nucleobase sequence of the modified oligonucleotide comprises the nucleobase sequence AUGCUGC (SEQ ID NO: 189) which is complementary to nucleotides 2-8 of miR-103 and miR-107. In certain embodiments, the nucleobase sequence of the modified oligonucleotide comprises the nucleobase sequence UGCUGCU (SEQ ID NO: 190) which is complementary to nucleotides 1-7 of miR-103 and miR-107. In certain embodiments, the nucleobase sequence of the modified oligonucleotide comprises the nucleobase sequence AUGCUGC (SEQ ID NO: 191) which is complementary to nucleotides 2-8 of miR-103 and miR-107. In certain embodiments, the nucleobase sequence of the modified oligonucleotide comprises the nucleobase sequence GCUGCU (SEQ ID NO: 192) which is complementary to nucleotides 1-6 of miR-103 or miR-107. In certain embodiments, the nucleobase sequence of the modified oligonucleotide comprises the nucleobase sequence UGCUGC (SEQ ID NO: 193) which is complementary to nucleotides 2-7 of miR-103 and miR-107. In certain embodiments, the nucleobase sequence of the modified oligonucleotide comprises the nucleobase sequence AUGCUG (SEQ ID NO: 194) which is complementary to nucleotides 3-8 of miR-103 and miR-107.

Modified oligonucleotides consisting of 7, 8, 9, 10, or 11 linked nucleosides and complementary to nucleotides 2 through 8 or 2 through 7 of a miRNA have been shown to inhibit activity of the miRNA. Modified oligonucleotides consisting of 8 linked nucleosides and complementary to nucleotides 2 through 9 of a miRNA have also been shown to inhibit activity of the miRNA. Certain of these modified oligonucleotides have an LNA sugar modification at each nucleoside. Such inhibitory activity is described in PCT Publication No. WO/2009/043353, which is herein incorporated by reference in its entirety for its description of modified oligonucleotides targeting miRNA seed sequences.

In certain embodiments, an oligonucleotide has a nucleobase sequence that is complementary to a nucleobase sequence having at least 80% identity to a nucleobase sequence of a miR stem-loop sequence selected from SEQ ID NOs: 21-27. In certain embodiments, an oligonucleotide has a nucleobase sequence that is complementary to a nucleobase sequence having at least 85%, at least 90%, at least 92%, at least 94%, at least 96%, at least 98% identity, or 100% identity to a nucleobase sequence of a miR stem-loop sequence selected from SEQ ID NOs 21-27.

In certain embodiments, an oligonucleotide has a nucleobase sequence that is complementary to a nucleobase sequence having at least 80% identity to a nucleobase sequence of a miR having a nucleobase sequence selected from SEQ ID NOs 1, 7, 11 or 12. In certain embodiments, an oligonucleotide has a nucleobase sequence that is complementary to a nucleobase sequence having at least 85%, at least 90%, at least 92%, at least 94%, at least 96%, at least 98% identity, or 100% identity to a nucleobase sequence of a miR nucleobase sequence selected from SEQ ID NOs 1, 7, 11 or 12.

Anti-miR-425 Agents

Encompassed in the methods of the present invention is the use of any anti-miR agent, or a combination thereof, as disclosed herein, e.g., an anti-miR-103 agent, anti-miR-105 agent, an anti-miR-107 agent or anti-miR-155 agent, used alone or in combination with an anti-miR agent which inhibits the function of miR-425 (herein referred to an “anti-miR-425 agent”). In some embodiments, such an anti-miR-425 agent binds to the miR-425 of SEQ ID NO: 15 or to at least part of the seed sequence SEQ ID NO: 16 and inhibits miR-425 mediated suppression of NPPA gene expression by inhibiting the binding of miR-425 to the 3′UTR of the NPPA mRNA. An anti-miR-425 agent can be a small molecule or oligonucleotide which inhibits binding of the miR-425 to SEQ ID NO: 18 in the 3′UTR of the NPPA gene, or alternatively, inhibits the expression of the miR-425 molecule. Such an anti-miR-425 agent which inhibits binding of the miR-425 to SEQ ID NO: 18 are useful to increase ANP levels in a subject whom is either AA or AG for the rs5068 SNP. In alternative embodiments, an anti-miR which inhibits a miRNA which binds to the miRNA target site of SEQ ID NO: 19, (e.g., the G variant of the rs5068 SNP) is also encompassed for use in the present invention, e.g., to increase ANP levels in a subject whom is either AG or GG for the rs5068 SNP.

An anti-miR agent encompassed for use in the methods, compositions and kits as disclosed herein is a nucleic acid, or analogue or mimetic thereof which is complementary to at least part of the miR-425 sequence of SEQ ID NO: 15, or at least part of the miR-425 miRNA seed sequence SEQ ID NO: 16. In some embodiments, an anti-miR agent to miRNA-425 is an anti-miR of SEQ ID NO: 17 or homologue or variant thereof.

In some embodiments, the target sequence is a nucleic acid encoding, expressing or comprising the nucleotide sequence of pre-miRNA-425, e.g., SEQ ID NO: 249 (GAAAGCGCUUUGGAAUGACACGAUCACUCCCGUUGAGUGGGCACCCGAGAAGCCAUCG GG AAUGUCGUGUCCGCCCAGUGCUCUUUC). In some embodiments, the target sequence is a nucleic acid encoding, expressing or comprising the nucleotide sequence of miR-425, e.g., SEQ ID NO: 15. In some embodiments, the target sequence is a nucleic acid encoding, expressing or comprising the nucleotide sequence of miR-425 seed sequence, e.g., SEQ ID NO: 16. In some embodiments, the anti-miR-425 inhibitor is an oligonucleotide, or an anti-miR, antagomir, ribozyme, or siRNA. In some embodiments, the anti-miR-425 inhibitor comprises a nucleotide sequence that is complementary to at least a portion of the pre-miRNA-425 nucleic acid sequence of: GAAAGCGCUUUGGAAUGACACGAUCACUCCCGUUGAGUGGGCACCCGAGAAGCCAUCG GG AAUGUCGUGUCCGCCCAGUGCUCUUUC (SEQ ID No: 249). In some embodiments, the anti-miR-425 inhibitor comprises a nucleotide sequence that is complementary to at least a portion nucleic acid sequence AAUGACACGAUCACUCCCGUUGA (SEQ ID NO: 15), or a nucleotide sequence that is complementary to nucleic acid sequence AUGACA (SEQ ID NO: 16).

In some embodiments, an anti-miR-425 agent is an anti-miR which is complementary to, or in part, to the miRNA seed sequence AUGACA (SEQ ID NO: 16). In some embodiments, an anti-miR-425 agent is an oligonucleotide of TTACTGTGCTAGTGAGGGCAACT (SEQ ID NO: 17) or a homologue thereof or a fragment of at least 10, or at least 12, or at least 14, or at least 16, or at least 18 or more concecutive nucleotides thereof. In some embodiments, where an anti-miR-425 is an oligonucleotide, such as an anti-miR which is complementary, at least in part, to the miRNA seed sequence is encoded SEQ ID NO: 16, the anti-miR-425 agent is encoded by a nucleic acid construct.

In some embodiments, an anti-miR-425 oligonucleotide can comprise a nucleic acid sequence that is complementary to at least a portion of a pre-miRNA-425 nucleic acid sequence (e.g. a portion of nucleic acid sequence GAAAGCGCUUUGGAAUGACACGAUCACUCCCGUUGAGUGGGCACCCGAGAAGCCAUCG GGAAUGUCGUGUCCGCCCAGUGCUCUUUC (SEQ ID No. 28). An anti-miR-425 oligonucleotide can be at least 75% (e.g., 80%, 85%, 90%, 91%, 92%, 93%, 94%, 95%, 96%, 97%, 98%, 99% conmplementary, or 100% (i.e. completely complementary)) complementary over a sequence comprising at least 6 (e.g., 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more) consecutive nucleotides of sequence of SEQ ID NO: 28.

In some embodiments, the anti-miR-425 oligonucleotide comprises a nucleotide sequence that is complementary to at least a portion of miRNA-425 nucleic acid sequence AAUGACACGAUCACUCCCGUUGA (SEQ ID NO: 15. The anti-miR-425 oligonucleotide can be at least 75% (e.g., 80%, 85%, 90%, 91%, 92%, 93%, 94%, 95%, 96%, 97%, 98%, 99% complementary or 100% (i.e. completely complementary)) over a sequence comprising at least 6 (e.g., 6, 7, 8, 9, 10, 11, 12, 13, 14, 15, 16, 17, 18, 19, 20, 21, 22, or 23) consecutive nucleotides of sequence of SEQ ID NO: 15. In some embodiments, an anti-miR-425 agent comprises a nucleotide sequence that is complementary to nucleic acid sequence AUGACA (SEQ ID NO: 16).

TABLE 2
discloses sequences of miR-425 and its seed sequence, pre-mir sequence, target
binding sequence on the 3′UTR of NPPA and homologues thereof:

| | | SEQ ID |
|---|---|---|
| Name | Sequence | NO: |
| miR-425 | AAUGACACGAUCACUCCCGUUGA | 15 |
| miR-425 seed | AUGACA | 16 |
| sequence | | |
| anti miR-425 | TTACTGTGCTAGTGAGGGCAACT | 17 |
| miR-425 “A” | 5′-ATGACAC′-3′ | 18 |
| allele miRNA | | |
| TARGET | | |
| sequence: | | |
| miR-425 “G” | 5′-GTGACAC′-3′ | 19 |
| allele miRNA | | |
| TARGET | | |
| sequence: | | |
| sequence of | 1 gagacaggga cagacgtagg ccaagagagg ggaaccagag aggaaccaga ggggagagac | 152 |
| NPPA mRNA | 61 agagcagcaa gcagtggatt gctccttgac gacgccagca tgagctcctt ctccaccacc | |
| with 3′UTR | 121 accgtgagct tcctcctttt actggcattc cagctcctag gtcagaccag agctaatccc | |
| | 181 atgtacaatg ccgtgtccaa cgcagacctg atggatttca agaatttgct ggaccatttg | |
| | 241 gaagaaaaga tgcctttaga agatgaggtc gtgcccccac aagtgctcag tgagccgaat | |
| | 301 gaagaagcgg gggctgctct cagccccctc cctgaggtgc ctccctggac cggggaagtc | |
| | 361 agcccagccc agagagatgg aggtgccctc gggcggggcc cctgggactc ctctgatcga | |
| | 421 tctgccctcc taaaaagcaa gctgagggcg ctgctcactg cccctcggag cctgcggaga | |
| | 481 tccagctgct tcgggggcag gatggacagg attggagccc agagcggact gggctgtaac | |
| | 541 agcttccggt actgaagata acagccaggg aggacaagca gggctgggcc tagggacaga | |
| | 601 ctgcaagagg ctcctgtccc ctggggtctc tgctgcattt gtgtcatctt gttgccatgg | |
| | 661 agttgtgatc atcccatcta agctgcagct tcctgtcaac acttctcaca tcttatgcta | |
| | 721 actgtagata aagtggtttg atggtgactt cctcgcctct cccaccccat gcattaaatt | |
| | 781 ttaaggtaga acctcacctg ttactgaaag tggtttgaaa gtgaataaac ttcagcacca | |
| | 841 tggacagaag acaaaaaa | |
| pre-miRNA-425 | GAAAGCGCUUUGGAAUGACACGAUCACUCCCGUUGAGUGGGCA | 28 |
| | CCCGAGAAGCCAUCGGGAAUGUCGUGUCCGCCCAGUGCUCUUU | |
| | C | |

### Anti-miR Agents in General

As discussed herein, encompassed for use in the methods, compositions and kits herein are anti-miR agents that inhibit at least one of miR-103, miR-105, miR-107 and/or miR-155, and can be any or a combination of RNA interference molecules, for example anti-miRs, oligonucleotides, LNA, miRNA, siRNA, shRNA, or proteins, small molecules, nucleic acids, nucleic acid analogues, aptamers, antibodies, peptides and variants and analogues thereof. In some embodiments, where the anti-miR agents that inhibits at least one of miR-103, miR-105, miR-107 and/or miR-155, is an antibody, the antibody can be a recombinant antibody, humanized antibody, chimeric antibody, modified antibody, monoclonal antibody, polyclonal antibody, miniantibody, dimeric miniantibody, minibody, diabody or tribody or antigen-binding variants, analogues or modified versions thereof.

### Inhibitory Nucleic Acids

In some embodiments, an anti-miR agent (e.g., an anti-miR agent that inhibit at least one of miR-103, miR-105, miR-107 and/or miR-155) can be RNA-interference or RNA interference molecules, including, but not limited to double-stranded RNA, such as siRNA, double-stranded DNA or single-stranded DNA. In some embodiments, an anti-miR agent is a single-stranded RNA (ssRNA), a form of RNA endogenously found in eukaryotic cells as the product of DNA transcription. Cellular ssRNA molecules include messenger RNAs (and the progenitor pre-messenger RNAs), small nuclear RNAs, small nucleolar RNAs, transfer RNAs and ribosomal RNAs. Double-stranded RNA (dsRNA) induces a size-dependent immune response such that dsRNA larger than 30 bp activates the interferon response, while shorter dsRNAs feed into the cell's endogenous RNA interference machinery downstream of the Dicer enzyme.

Inhibitory agents useful in the methods of treatment described herein include inhibitory nucleic acid molecules that decrease the expression or activity of any of the microRNAs (e.g., mature microRNA or precursor microRNA) to miR-103, miR-105, miR-107 and miR-155). Inhibitory nucleic acids useful in the present methods and compositions include antisense oligonucleotides, ribozymes, external guide sequence (EGS) oligonucleotides, siRNA compounds, single- or double-stranded RNA interference (RNAi) compounds, such as siRNA compounds, modified bases/locked nucleic acids (LNAs), antagomirs, peptide nucleic acids (PNAs), and other oligomeric compounds, or oligonucleotide mimetics which hybridize to at least a portion of the target nucleic acid and modulate its function. In some embodiments, the inhibitory nucleic acids include antisense RNA, antisense DNA, chimeric antisense oligonucleotides, antisense oligonucleotides comprising modified linkages, interference RNA (RNAi), short interfering RNA (siRNA); a micro, interfering RNA (miRNA); a small, temporal RNA (stRNA); or a short, hairpin RNA (shRNA); small RNA-induced gene activation (RNAa); small activating RNAs (saRNAs), or combinations thereof. See, e.g., WO 2010/040112.

In some embodiments, the inhibitory nucleic acids are 10 to 50, 13 to 50, or 13 to 30 nucleotides in length. One having ordinary skill in the art will appreciate that this embodies oligonucleotides having antisense portions of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or any range therewithin. In some embodiments, the oligonucleotides are 15 nucleotides in length. In some embodiments, the antisense or oligonucleotide compounds of the invention are 12 or 13 to 30 nucleotides in length. One having ordinary skill in the art will appreciate that this embodies inhibitory nucleic acids having antisense portions of 12, 13, 14, 15, 16, 17, 18, 19, 20, 21, 22, 23, 24, 25, 26, 27, 28, 29, or 30 nucleotides in length, or any range therewithin.

In some embodiments, the inhibitory nucleic acids are chimeric oligonucleotides that contain two or more chemically distinct regions, each made up of at least one nucleotide. These oligonucleotides typically contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids. Chimeric inhibitory nucleic acids of the invention may be formed as composite structures of two or more oligonucleotides, modified oligonucleotides, oligonucleosides, and/or oligonucleotide mimetics as described above. Such compounds have also been referred to in the art as hybrids or gapmers. Representative United States patents that teach the preparation of such hybrid structures comprise, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference.

In some embodiments, the inhibitory nucleic acid comprises at least one nucleotide modified at the 2′ position of the sugar, most preferably a 2′-O-alkyl, 2′-O-alkyl-O-alkyl or 2′-fluoro-modified nucleotide. In other preferred embodiments, RNA modifications include 2′-fluoro, 2′-amino, and 2′ O-methyl modifications on the ribose of pyrimidines, abasic residues, or an inverted base at the 3′ end of the RNA. Such modifications are routinely incorporated into oligonucleotides and these oligonucleotides have been shown to have a higher Tm (i.e., higher target binding affinity) than 2′-deoxyoligonucleotides against a given target.

A number of nucleotide and nucleoside modifications have been shown to make the oligonucleotide into which they are incorporated more resistant to nuclease digestion than the native oligodeoxynucleotide—the modified oligos survive intact for a longer time than unmodified oligonucleotides. Specific examples of modified oligonucleotides include those comprising modified backbones, for example, phosphorothioates, phosphotriesters, methyl phosphonates, short-chain alkyl or cycloalkyl intersugar linkages, or short-chain heteroatomic or heterocyclic intersugar linkages. Most preferred are oligonucleotides with phosphorothioate backbones and those with heteroatom backbones, particularly CH2-NH—O—CH2, CH, ^{˜}N(CH3)^{˜}O^{˜}CH2 (known as a methylene(methylimino) or MMI backbone], CH2-O—N(CH3)-CH2, CH2-N(CH3)-N(CH3)-CH2 and O—N(CH3)-CH2-CH2 backbones, wherein the native phosphodiester backbone is represented as O—P—O—CH1); amide backbones (see De Mesmaeker et al., Ace. Chem. Res. 28:366-374, 1995); morpholino backbone structures (see U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone, see Nielsen et al., Science 254: 1497, 1991). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′ alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050 (each of which is incorporated by reference).

Morpholino-based oligomeric compounds are described in Braasch et al., Biochemistry 41(14):4503-4510, 2002; Genesis, volume 30, issue 3, 2001; Heasman, J., Dev. Biol., 243:209-214, 2002; Nasevicius et al., Nat. Genet. 26: 216-220, 2000; Lacerra et al., Proc. Natl. Acad. Sci. U.S.A. 97:9591-9596, 2000; and U.S. Pat. No. 5,034,506. Cyclohexenyl nucleic acid oligonucleotide mimetics are described in Wang et al., J. Am. Chem. Soc. 122, 8595-8602, 2000.

Modified oligonucleotide backbones that do not include a phosphorus atom therein have backbones that are formed by short-chain alkyl or cycloalkyl internucleoside linkages, mixed heteroatom and alkyl or cycloalkyl internucleoside linkages, or one or more short-chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts; see U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439 (each of which is herein incorporated by reference).

One or more substituted sugar moieties can also be included, e.g., one of the following at the 2′ position: OH, SH, SCH3, F, OCN, OCH3 OCH3, OCH3-O—(CH2)n CH3, O(CH2) n NH2 or O(CH2) n CH3, where n is from 1 to about 10; C1 to C10 lower alkyl, alkoxyalkoxy, substituted lower alkyl, alkaryl or aralkyl; Cl; Br; CN; CF3; OCF3; O—, S—, or N-alkyl; O—, S—, or N-alkenyl; SOCH3; SO2 CH3; ONO2; NO2; N3; NH2; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. A preferred modification includes 2′-methoxyethoxy[2′-0-CH2CH2OCH3, also known as 2′-O-(2-methoxyethyl)] (Martin et al., Helv. Chim. Acta 78:486, 1995). Other preferred modifications include 2′-methoxy(2′-0-CH3), 2′-propoxy(2′-OCH2 CH2CH3) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics, such as cyclobutyls in place of the pentofuranosyl group.

Inhibitory nucleic acids can also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC, and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl)adenine, and 2,6-diaminopurine. See Kornberg, A., DNA Replication, W. H. Freeman & Co., San Francisco, 1980, pp 75-77; and Gebeyehu et al., Nucl. Acids Res. 15:4513, 1987. A “universal” base known in the art, e.g., inosine, can also be included. 5-Me-C substitutions have been shown to increase nucleic acid duplex stability by 0.6-1.2<0>C (Sanghvi, Y. S., in Crooke, S. T. and Lebleu, B., Eds., Antisense Research and Applications, CRC Press, Boca Raton, 1993, pp. 276-278) and are presently preferred base substitutions.

It is not necessary for all positions in a given oligonucleotide to be uniformly modified, and in fact more than one of the aforementioned modifications may be incorporated in a single oligonucleotide or even at within a single nucleoside within an oligonucleotide.

In some embodiments, both a sugar and an internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative United States patents that teach the preparation of PNA compounds comprise, but are not limited to, U.S. Pat. Nos. 5,539,082; 5,714,331; and 5,719,262, each of which is herein incorporated by reference. Further teaching of PNA compounds can be found in Nielsen et al, Science 254:1497-1500, 1991.

Inhibitory nucleic acids can also include one or more nucleobase (often referred to in the art simply as “base”) modifications or substitutions. As used herein, “unmodified” or “natural” nucleobases comprise the purine bases adenine (A) and guanine (G), and the pyrimidine bases thymine (T), cytosine (C), and uracil (U). Modified nucleobases comprise other synthetic and natural nucleobases, such as 5-methylcytosine (5-me-C), 5-hydroxymethyl cytosine, xanthine, hypoxanthine, 2-aminoadenine, 6-methyl, and other alkyl derivatives of adenine and guanine, 2-propyl and other alkyl derivatives of adenine and guanine, 2-thiouracil, 2-thiothymine, and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine, and 7-deazaadenine, and 3-deazaguanine and 3-deazaadenine.

Further, nucleobases comprise those disclosed in U.S. Pat. No. 3,687,808, those disclosed in ‘The Concise Encyclopedia of Polymer Science And Engineering’, pages 858-859, Kroschwitz, J. I., Ed. John Wiley & Sons, 1990, those disclosed by Englisch et al., Angewandle Chemie, International Edition’, 1991, 30, page 613, and those disclosed by Sanghvi, Y. S., Chapter 15, Antisense Research and Applications’, pages 289-302, Crooke, S. T. and Lebleu, B. ea., CRC Press, 1993. Certain of these nucleobases are particularly useful for increasing the binding affinity of the oligomeric compounds of the invention. These include 5-substituted pyrimidines, 6-azapyrimidines, and N2, N-6 and 0-6 substituted purines, comprising 2-aminopropyladenine, 5-propynyluracil, and 5-propynylcytosine. 5-methylcytosine substitutions have been shown to increase nucleic acid duplex stability by 0.6-1.2<0>C (Sanghvi, Y. S., Crooke, S. T. and Lebleu, B., Eds, ‘Antisense Research and Applications’, CRC Press, Boca Raton, 1993, pp. 276-278) and are presently preferred base substitutions, even more particularly when combined with 2′-O-methoxyethyl sugar modifications. Modified nucleobases are described in U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,596,091; 5,614,617; 5,750,692, and U.S. Pat. No. 5,681,941 (each of which is herein incorporated by reference).

In some embodiments, the inhibitory nucleic acids are chemically linked to one or more moieties or conjugates that enhance the activity, cellular distribution, or cellular uptake of the oligonucleotide. Such moieties comprise but are not limited to, lipid moieties such as a cholesterol moiety (Letsinger et al., Proc. Natl. Acad. Sci. U.S.A. 86:6553-6556, 1989), cholic acid (Manoharan et al., Bioorg. Med. Chem. Left. 4:1053-1060, 1994), a thioether, e.g., hexyl-S-tritylthiol (Manoharan et al, Ann. N.Y. Acad. Sci. 660:306-309, 1992; Manoharan et al., Bioorg. Med. Chem. Lett. 3:2765-2770, 1993), a thiocholesterol (Oberhauser et al., Nucl. Acids Res. 20, 533-538, 1992), an aliphatic chain, e.g., dodecandiol or undecyl residues (Kabanov et al., FEBS Lett. 259:327-330, 1990; Svinarchuk et al., Biochimie 75:49-54, 1993), a phospholipid, e.g., di-hexadecyl-rac-glycerol or triethylammonium 1,2-di-O-hexadecyl-rac-glycero-3-H-phosphonate (Manoharan et al., Tetrahedron Lett. 36:3651-3654, 1995; Shea et al., Nucl. Acids Res. 18:3777-3783, 1990), a polyamine or a polyethylene glycol chain (Mancharan et al., Nucleosides & Nucleotides 14:969-973, 1995), or adamantane acetic acid (Manoharan et al., Tetrahedron Lett. 36:3651-3654, 1995), a palmityl moiety (Mishra et al., Biochim. Biophys. Acta 1264: 229-237, 1995), or an octadecylamine or hexylamino-carbonyl-t oxycholesterol moiety (Crooke et al., J. Pharmacol. Exp. Ther. 277:923-937, 1996). See also U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941 (each of which is herein incorporated by reference).

These moieties or conjugates can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve uptake, enhance resistance to degradation, and/or strengthen sequence-specific hybridization with the target nucleic acid. Groups that enhance the pharmacokinetic properties, in the context of this invention, include groups that improve uptake, distribution, metabolism, or excretion of the compounds of the present invention. Representative conjugate groups are disclosed in International Patent Application No. PCT/US92/09196, filed Oct. 23, 1992, and U.S. Pat. No. 6,287,860, which are incorporated herein by reference. Conjugate moieties include, but are not limited to, lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxy cholesterol moiety. See, e.g., U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941 (each of which is incorporated by reference).

The inhibitory nucleic acids useful in the present methods are sufficiently complementary to the target miRNA, i.e., hybridize sufficiently well and with sufficient specificity, to give the desired effect. “Complementary” refers to the capacity for pairing, through hydrogen bonding, between two sequences comprising naturally or non-naturally occurring bases or analogs thereof. For example, if a base at one position of an inhibitory nucleic acid is capable of hydrogen bonding with a base at the corresponding position of a miRNA, then the bases are considered to be complementary to each other at that position. In some embodiments, 100% complementarity is not required. In some embodiments, 100% complementarity is required. Routine methods can be used to design an inhibitory nucleic acid that binds to the target sequence with sufficient specificity.

While the specific sequences of certain exemplary target segments are set forth herein, one of skill in the art will recognize that these serve to illustrate and describe particular embodiments within the scope of the present invention. Additional target segments are readily identifiable by one having ordinary skill in the art in view of this disclosure. Target segments of 5, 6, 7, 8, 9, 10 or more nucleotides in length comprising a stretch of at least five (5) consecutive nucleotides within the seed sequence, or immediately adjacent thereto, are considered to be suitable for targeting as well. In some embodiments, target segments can include sequences that comprise at least the 5 consecutive nucleotides from the 5′-terminus of one of the seed sequence (the remaining nucleotides being a consecutive stretch of the same RNA beginning immediately upstream of the 5′-terminus of the seed sequence and continuing until the inhibitory nucleic acid contains about 5 to about 30 nucleotides). In some embodiments, target segments are represented by RNA sequences that comprise at least the 5 consecutive nucleotides from the 3′-terminus of one of the seed sequence (the remaining nucleotides being a consecutive stretch of the same miRNA beginning immediately downstream of the 3′-terminus of the target segment and continuing until the inhibitory nucleic acid contains about 5 to about 30 nucleotides). One having skill in the art armed with the sequences provided herein will be able, without undue experimentation, to identify further preferred regions to target. In some embodiments, an inhibitory nucleic acid contain a sequence that is complementary to at least 5, 6, 7, 8, 9, 10, 11, 12, 13, 14, 15, 16, 17, 18, 19, 20, 21, 22, 23, 24, or 25 continguous nucleotides present in the target (e.g., the target miRNA, e.g., mature or precursor miR-103, miR-105, miR-107 and miR-155, or the target mRNA).

Once one or more target regions, segments or sites have been identified, inhibitory nucleic acid compounds are chosen that are sufficiently complementary to the target, i.e., that hybridize sufficiently well and with sufficient specificity (i.e., do not substantially bind to other non-target RNAs), to give the desired effect.

In the context of this invention, hybridization means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. Complementary, as used herein, refers to the capacity for precise pairing between two nucleotides. For example, if a nucleotide at a certain position of an oligonucleotide is capable of hydrogen bonding with a nucleotide at the same position of a miRNA molecule or an mRNA molecule, then the inhibitory nucleic acid and the miRNA or mRNA are considered to be complementary to each other at that position. The inhibitory nucleic acids and the miRNA or mRNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the inhibitory nucleic acid and the miRNA target. For example, if a base at one position of an inhibitory nucleic acid is capable of hydrogen bonding with a base at the corresponding position of a miRNA or a mRNA, then the bases are considered to be complementary to each other at that position. 100% complementarity is not required.

It is understood in the art that a complementary nucleic acid sequence need not be 100% complementary to that of its target nucleic acid to be specifically hybridizable. A complementary nucleic acid sequence for purposes of the present methods is specifically hybridisable when binding of the sequence to the target miRNA or mRNA molecule interferes with the normal function of the target miRNA or mRNA to cause a loss of expression or activity, and there is a sufficient degree of complementarity to avoid non-specific binding of the sequence to non-target RNA sequences under conditions in which specific binding is desired, e.g., under physiological conditions in the case of in vivo assays or therapeutic treatment, and in the case of in vitro assays, under conditions in which the assays are performed under suitable conditions of stringency. For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci. U.S.A. 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

In general, the inhibitory nucleic acids useful in the methods described herein have at least 80% sequence complementarity to a target region within the target nucleic acid, e.g., 90%, 95%, or 100% sequence complementarity to the target region within an miRNA. For example, an antisense compound in which 18 of 20 nucleobases of the antisense oligonucleotide are complementary, and would therefore specifically hybridize, to a target region would represent 90 percent complementarity. Percent complementarity of an inhibitory nucleic acid with a region of a target nucleic acid can be determined routinely using basic local alignment search tools (BLAST programs) (Altschul et al., J. Mol. Biol. 215:403-410, 1990; Zhang and Madden, Genome Res. 7:649-656, 1997). Antisense and other compounds of the invention that hybridize to an miRNA or a mRNA are identified through routine experimentation. In general the inhibitory nucleic acids must retain specificity for their target, i.e., must not directly bind to, or directly significantly affect expression levels of, transcripts other than the intended target.

For further disclosure regarding inhibitory nucleic acids, please see US2010/0317718 (antisense oligos); US2010/0249052 (double-stranded ribonucleic acid (dsRNA)); US2009/0181914 and US2010/0234451 (LNAs); US2007/0191294 (siRNA analogues); US2008/0249039 (modified siRNA); and WO2010/129746 and WO2010/040112 (inhibitory nucleic acids).

Numerous specific siRNA molecules have been designed that have been shown to inhibit gene expression (Ratcliff et al. Science 276:1558-1560, 1997; Waterhouse et al. Nature 411:834-842, 2001). In addition, specific siRNA molecules have been shown to inhibit, for example, HIV-1 entry to a cell by targeting the host CD4 protein expression in target cells thereby reducing the entry sites for HIV-1 which targets cells expressing CD4 (Novina et al. Nature Medicine, 8:681-686, 2002). Short interfering RNA have further been designed and successfully used to silence expression of Fas to reduce Fas-mediated apoptosis in vivo (Song et al. Nature Medicine 9:347-351, 2003).

It has been shown in plants that longer, about 24-26 nt siRNA, correlates with systemic silencing and methylation of homologous DNA. Conversely, the about 21-22 nt short siRNA class correlates with mRNA degradation but not with systemic signaling or methylation (Hamilton et al. EMBO J. 2002 Sep. 2; 21(17):4671-9). These findings reveal an unexpected level of complexity in the RNA silencing pathway in plants that may also apply in animals. In higher order eukaryotes, DNA is methylated at cytosines located 5′ to guanosine in the CpG dinucleotide. This modification has important regulatory effects on gene expression, especially when involving CpG-rich areas known as CpG islands, located in the promoter regions of many genes. While almost all gene-associated islands are protected from methylation on autosomal chromosomes, extensive methylation of CpG islands has been associated with transcriptional inactivation of selected imprinted genes and genes on the inactive X-chromosomes of females. Aberrant methylation of normally unmethylated CpG islands has been documented as a relatively frequent event in immortalized and transformed cells and has been associated with transcriptional inactivation of defined tumor suppressor genes in human cancers. In this last situation, promoter region hypermethylation stands as an alternative to coding region mutations in eliminating tumor suppression gene function (Herman, et al.). The use of siRNA molecules for directing methylation of a target gene is described in U.S. Provisional Application No. 60/447,013, filed Feb. 13, 2003, referred to in U.S. Patent Application Publication No. 2004/0091918, which is incorporated herein in its entirety by reference.

It is also known that the RNA interference does not have to match perfectly to its target sequence. In some embodiments, however, the 5′ and middle part of the antisense (guide) strand of the siRNA is perfectly complementary to the target nucleic acid sequence.

The RNA interference-inducing molecule functioning as an anti-miR mimetics according to the present invention includes RNA molecules that have natural or modified nucleotides, natural ribose sugars or modified sugars and natural or modified phosphate backbone.

Accordingly, the RNA interference-inducing molecules functioning as an anti-miR mimetics (e.g., anti-miR-103, miR-105, miR-107 and/or miR-155) includes, but are not limited to, unmodified and modified double stranded (ds) RNA molecules including short-temporal RNA (stRNA), small interfering RNA (siRNA), short-hairpin RNA (shRNA), microRNA (miRNA), and double-stranded RNA (dsRNA), (see, e.g. Baulcombe, Science 297:2002-2003, 2002). The dsRNA molecules, e.g. siRNA, also may contain 3′ overhangs, preferably 3′UU or 3′TT overhangs. In one embodiment, the siRNA molecules do not include RNA molecules that comprise ssRNA greater than about 30-40 bases, about 40-50 bases, about 50 bases or more. In one embodiment, the siRNA molecules have a double stranded structure. In one embodiment, the siRNA molecules are double stranded for more than about 25%, more than about 50%, more than about 60%, more than about 70%, more than about 80% or more than about 90% of their length.

In some embodiments, an anti-miR-103 agent is any agent which binds to the miR-103 seed sequence of SEQ ID NO: 4 or 5, and inhibits the activity of miR-103 (e.g., inhibits hsa-miR-103a-3p (SEQ ID NO: 1, or hsa-miR-103b (SEQ ID NO: 2) and/or hsa-miR-103a-2-5p (SEQ ID NO: 3). In some embodiments, an anti-miR-105 agent is any agent which binds to the miR-105 seed sequence of SEQ ID NO: 9, and inhibits the activity of miR-105 (e.g., inhibits hsa-miR-105-5p (SEQ ID NO: 7), or hsa-miR-105-3p (SEQ ID NO: 8). In some embodiments, an anti-miR-107 agent is any agent which binds to the miR-107 seed sequence of SEQ ID NO: 4, and inhibits the activity of miR-107 (e.g., inhibits hsa-miR-107 (SEQ ID NO: 11). In some embodiments, an anti-miR-155 agent is any agent which binds to the miR-155 seed sequence of SEQ ID NO: 14, and inhibits the activity of miR-155 (e.g., inhibits hsa-miR-155 (SEQ ID NO: 12).

In such embodiments, an anti-miR agent that inhibit at least one of miR-103, miR-105, miR-107 and/or miR-155 agent can be an RNA interference-inducing molecule, including, but not limited to unmodified and modified double stranded (ds) RNA molecules including, short-temporal RNA (stRNA), small interfering RNA (siRNA), short-hairpin RNA (shRNA), microRNA (miRNA), and double-stranded RNA (dsRNA). In other embodiments, the agents may be any small molecule, protein, aptamer, nucleic acid analogue, antibody etc. that binds to any of the following; at least one member of the miR-103 family comprising hsa-miR-103a-3p (SEQ ID NO: 1), or hsa-miR-103b (SEQ ID NO: 2) and/or hsa-miR-103a-2-5p (SEQ ID NO: 3), or the miR-103 seed sequence of SEQ ID NO: 4; at least one member of the miR-105 family of hsa-miR-105-5p (SEQ ID NO: 7), or hsa-miR-105-3p (SEQ ID NO: 8) or the miR-105 seed sequence of SEQ ID NO: 9, or to the miR-107 molecule of hsa-miR-107 of SEQ ID NO: 11 or the miR-107 seed sequence of SEQ ID NO: 4, or or the miR-155 has-miR-155 of SEQ ID NO: 12, or the miR-155 seed sequence of SEQ ID NO: 14, thereby inhibiting the interference activity of miR-103, miR-105, miR-107 or miR-155, respectively.

The miRNA and RNA interference molecules according to the present invention can be produced using any known techniques such as direct chemical synthesis, through processing of longer double stranded RNAs by exposure to recombinant Dicer protein or Drosophila embryo lysates, through an in vitro system derived from S2 cells, using phage RNA polymerase, RNA-dependent RNA polymerase, and DNA based vectors. Use of cell lysates or in vitro processing may further involve the subsequent isolation of the short, for example, about 21-23 nucleotide, siRNAs from the lysate, etc. Chemical synthesis usually proceeds by making two single stranded RNA-oligomers followed by the annealing of the two single stranded oligomers into a double stranded RNA. Other examples include methods disclosed in WO 99/32619 and WO 01/68836 that teach chemical and enzymatic synthesis of siRNA, which are incorporated herein in their entirety by reference. Moreover, numerous commercial services are available for designing and manufacturing specific siRNAs (see, e.g., QIAGEN Inc., Valencia, Calif. and AMBION Inc., Austin, Tex.).

Examples of methods of preparing such RNA interference are shown, for example in an International Patent application Nos. PCT/US03/34424 and PCT/US03/34686 the contents and references of which are herein incorporated by reference in their entirety.

Various specific siRNA and miRNA molecules have been described and additional molecules can be easily designed by one skilled in the art. For example, the miRNA Database at http://www.sanger.ac.uk/Software/Rfam/mirna/index.shtml provides a useful source to identify additional miRNAs useful according to the present invention (Griffiths-Jones S. NAR, 2004, 32, Database Issue, D109-D111; Ambros V, Bartel B, Bartel D P, Burge C B, Carrington J C, Chen X, Dreyfuss G, Eddy S R, Griffiths-Jones S, Marshall M, Matzke M, Ruvkun G, Tuschl T. RNA, 2003, 9(3), 277-279).

The miRNA and RNA interference as described herein also includes RNA molecules having one or more non-natural nucleotides, i.e. nucleotides other than adenine “A”, guanine “G”, uracil “U”, or cytosine “C”, a modified nucleotide residue or a derivative or analog of a natural nucleotide are also useful. Any modified residue, derivative or analog may be used to the extent that it does not eliminate or substantially reduce (by at least 50%) RNAi activity of the dsRNA. For example, the activity of a miRNA or RNAi molecule with the modified residue can be compared with the activity of a miRNA or RNAi molecule with the same nucleic acid sequence without the modified residue in an assay for gene silencing the target gene. If the miRNA or RNAi with the modified residue(s) has an efficiency of gene silencing which is the same, greater or a least half as efficient as the miRNA or RNAi without the modification, the modified mRNA or RNAi is useful in the methods and compositions as disclosed herein. Examples of modified residues, derivatives or analogues include, but are not limited to, aminoallyl UTP, pseudo-UTP, 5-I-UTP, 5-I-CTP, 5-Br-UTP, alpha-S ATP, alpha-S CTP, alpha-S GTP, alpha-S UTP, 4-thio UTP, 2-thio-CTP, 2′NH2 UTP, 2′NH₂ CTP, and 2′F UTP. Such modified nucleotides include, but are not limited to, aminoallyl uridine, pseudo-uridine, 5-I-uridine, 5-I-cytidine, 5-Br-uridine, alpha-S adenine, alpha-S cytidine, alpha-S guanosine, alpha-S uridine, 4-thio uridine, 2-thio-cytidine, 2′NH2 uridine, 2′NH2 cytidine, and 2′ F uridine, including the free pho (NTP) RNA molecules as well as all other useful forms of the nucleotides.

RNA interference as referred to herein additionally includes RNA molecules which contain modifications in the ribose sugars, as well as modifications in the “phosphate backbone” of the nucleotide chain. For example, siRNA or miRNA molecules containing D-arabinofuranosyl structures in place of the naturally-occurring D-ribonucleosides found in RNA can be used in RNA interference according to the present invention (U.S. Pat. No. 5,177,196). Other examples include RNA molecules containing the o-linkage between the sugar and the heterocyclic base of the nucleoside, which confers nuclease resistance and tight complementary strand binding to the oligonucleotides and molecules similar to the oligonucleotides containing 2′-O-methyl ribose, arabinose and particularly D-arabinose (U.S. Pat. No. 5,177,196). Also, phosphorothioate linkages can be used to stabilize the siRNA and miRNA molecules (U.S. Pat. No. 5,177,196). siRNA and miRNA molecules having various “tails” covalently attached to either their 3′- or to their 5′-ends, or to both, are also been known in the art and can be used to stabilize the siRNA and miRNA molecules delivered using the methods of the present invention. Generally speaking, intercalating groups, various kinds of reporter groups and lipophilic groups attached to the 3′ or 5′ ends of the RNA molecules are well known to one skilled in the art and are useful according to the methods of the present invention. Descriptions of syntheses of 3′-cholesterol or 3′-acridine modified oligonucleotides applicable to preparation of modified RNA molecules useful according to the present invention can be found, for example, in the articles: Gamper, H. B., Reed, M. W., Cox, T., Virosco, J. S., Adams, A. D., Gall, A., Scholler, J. K., and Meyer, R. B. (1993) Facile Preparation and Exonuclease Stability of 3′-Modified Oligodeoxynucleotides. Nucleic Acids Res. 21 145-150; and Reed, M. W., Adams, A. D., Nelson, J. S., and Meyer, R. B., Jr. (1991) Acridine and Cholesterol-Derivatized Solid Supports for Improved Synthesis of 3′-Modified Oligonucleotides. Bioconjugate Chem. 2 217-225 (1993).

MiRNA inhibitors, including hairpin miRNA inhibitors, are described in detail in Vermeulen et al., “Double-Stranded Regions Are Essential Design Components Of Potent Inhibitors of RISC Function,” RNA 13: 723-730 (2007) and in WO2007/095387 and WO 2008/036825 each of which is incorporated herein by reference in its entirety. A person of ordinary skill in the art can select a sequence from the database for a desired miRNA and design an inhibitor useful for the compositions and methods disclosed herein. Anti-miRs can be used to efficiently silence endogenous miRNAs by forming duplexes comprising the anti-miR and endogenous miRNA, thereby preventing miRNA-induced gene silencing. In some embodiments, the anti-miR is an antagomir. In some embodiment, the anti-miR is a ribozyme that can cleave the target microRNA.

### Antisense

Antisense oligonucleotides are typically designed to block expression of a DNA or RNA target by binding to the target and halting expression at the level of transcription, translation, or splicing. Antisense oligonucleotides of the present invention are complementary nucleic acid sequences designed to hybridize under stringent conditions to the target microRNA or the target inflammatory marker mRNA. Thus, oligonucleotides are chosen that are sufficiently complementary to the target, i.e., that hybridize sufficiently well and with sufficient specificity, to give the desired effect.

Modified Bases/Locked Nucleic Acids (LNAs)

In some embodiments, the inhibitory nucleic acids used in the methods described herein comprise one or more modified bonds or bases. Modified bases include phosphorothioate, methylphosphonate, peptide nucleic acids, or locked nucleic acid (LNA) molecules. Preferably, the modified nucleotides are locked nucleic acid molecules, including [alpha]-L-LNAs. LNAs comprise ribonucleic acid analogues wherein the ribose ring is “locked” by a methylene bridge between the 2′-oxgygen and the 4′-carbon—i.e., oligonucleotides containing at least one LNA monomer, that is, one 2′-O, 4′-C-methylene-β-D-ribofuranosyl nucleotide. LNA bases form standard Watson-Crick base pairs but the locked configuration increases the rate and stability of the base pairing reaction (Jepsen et al., Oligonucleotides 14:130-146, 2004). LNAs also have increased affinity to base pair with RNA as compared to DNA. These properties render LNAs especially useful as probes for fluorescence in situ hybridization (FISH) and comparative genomic hybridization, as knockdown tools for miRNAs, and as antisense oligonucleotides to target mRNAs or other RNAs, e.g., miRNAs and mRNAs as described herein.

The LNA molecules can include molecules comprising 10-30, e.g., 12-24, e.g., 12, 13, 14, 15, 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the miRNA or the mRNA. The LNA molecules can be chemically synthesized using methods known in the art.

The LNA molecules can be designed using any method known in the art; a number of algorithms are known, and are commercially available (e.g., on the internet, for example at exiqon.com). See, e.g., You et al., Nuc. Acids. Res. 34:e60, 2006; McTigue et al., Biochemistry 43:5388-405, 2004; and Levin et al., Nucl. Acids. Res. 34:e142, 2006. For example, “gene walk” methods, similar to those used to design antisense oligos, can be used to optimize the inhibitory activity of the LNA; for example, a series of oligonucleotides of 10-30 nucleotides spanning the length of a target miRNA or mRNA can be prepared, followed by testing for activity. Optionally, gaps, e.g., of 5-10 nucleotides or more, can be left between the LNAs to reduce the number of oligonucleotides synthesized and tested. GC content is preferably between about 30-60%. General guidelines for designing LNAs are known in the art; for example, LNA sequences will bind very tightly to other LNA sequences, so it is preferable to avoid significant complementarity within an LNA. Contiguous runs of three or more Gs or Cs, or more than four LNA residues, should be avoided where possible (for example, it may not be possible with very short (e.g., about 9-10 nt) oligonucleotides). In some embodiments, the LNAs are xylo-LNAs.

In some embodiments, the LNA molecules can be designed to target a specific region of the miRNA. For example, a specific functional region can be targeted, e.g., a region comprising a seed sequence. Alternatively or in addition, highly conserved regions can be targeted, e.g., regions identified by aligning sequences from disparate species such as primate (e.g., human) and rodent (e.g., mouse) and looking for regions with high degrees of identity. Percent identity can be determined routinely using basic local alignment search tools (BLAST programs) (Altschul et al., J. Mol. Biol. 215:403-410, 1990; Zhang and Madden, Genome Res. 7:649-656, 1997), e.g., using the default parameters.

For additional information regarding LNAs see U.S. Pat. Nos. 6,268,490; 6,734,291; 6,770,748; 6,794,499; 7,034,133; 7,053,207; 7,060,809; 7,084,125; and 7,572,582; and U.S. Pre-Grant Pub. Nos. 2010/0267018; 2010/0261175; and 2010/0035968; Koshkin et al., Tetrahedron 54:3607-3630, 1998; Obika et al., Tetrahedron Lett. 39:5401-5404, 1998; Jepsen et al., Oligonucleotides 14:130-146, 2004; Kauppinen et al., Drug Disc. Today 2(3):287-290, 2005; and Ponting et al., Cell 136(4):629-641, 2009, and references cited therein.

See also U.S. Ser. No. 61/412,862, which is incorporated by reference herein in its entirety.

### Antagomirs:

In some embodiments, an anti-miR agent that inhibits at least one of miR-103, miR-105, and/or miR-107 is an antagomir. Antagomirs are anti-miRs that harbor various modifications for RNAse protection and pharmacologic properties, such as enhanced tissue and cellular uptake. In some embodiments, an anti-miR agent for use in the methods, compositions, kits and systems as disclosed herein are modified as disclosed in US Application US2014/0066491, which is incorporated herein in its entirety by reference.

In some embodiments, the antisense is an antagomir. Antagomirs are chemically-modified antisense oligonucleotides that target a microRNA (e.g., target any one of miR-103, miR-105, miR-107 and/or miR-155). For example, an antagomir for use in the methods described herein can include a nucleotide sequence sufficiently complementary to hybridize to a miRNA target sequence of about 12 to 25 nucleotides, preferably about 15 to 23 nucleotides.

In general, antagomirs include a cholesterol moiety, e.g., at the 3′-end. In some embodiments, antagomirs have various modifications for RNase protection and pharmacologic properties such as enhanced tissue and cellular uptake. For example, in addition to the modifications discussed above for antisense oligos, an antagomir can have one or more of complete or partial 2′-O-methylation of sugar and/or a phosphorothioate backbone. Phosphorothioate modifications provide protection against RNase activity and their lipophilicity contributes to enhanced tissue uptake. In some embodiments, the antagomir can include six phosphorothioate backbone modifications; two phosphorothioates are located at the 5′-end and four at the 3′-end. See, e.g., Krutzfeldt et al., Nature 438:685-689, 2005; Czech, N. Engl. J. Med. 354:1194-1195, 2006: Robertson et al., Silence 1:10, 2010; Marquez and McCaffrey, Human Gene Ther. 19(1):27-38, 2008; van Rooij et al., Circ. Res. 103(9):919-928, 2008; and Liu et al., Int. J. Mol. Sci. 9:978-999, 2008.

Antagomirs useful in the present methods can also be modified with respect to their length or otherwise the number of nucleotides making up the antagomir. In general, the antagomirs are about 20-21 nucleotides in length for optimal function, as this size matches the size of most mature microRNAs. The antagomirs must retain specificity for their target, i.e., must not directly bind to, or directly significantly affect expression levels of, transcripts other than the intended target.

In some embodiments, the inhibitory nucleic acid is locked and includes a cholesterol moiety (e.g., a locked antagomir).

siRNA

In some embodiments, the nucleic acid sequence that is complementary to a target miRNA or a target mRNA can be an interfering RNA, including but not limited to a small interfering RNA (“siRNA”) or a small hairpin RNA (“shRNA”). Methods for constructing interfering RNAs are well known in the art. For example, the interfering RNA can be assembled from two separate oligonucleotides, where one strand is the sense strand and the other is the antisense strand, wherein the antisense and sense strands are self-complementary (i.e., each strand comprises nucleotide sequence that is complementary to nucleotide sequence in the other strand: such as where the antisense strand and sense strand form a duplex or double stranded structure); the antisense strand comprises nucleotide sequence that is complementary to a nucleotide sequence in a target nucleic acid molecule or a portion thereof (i.e., an undesired gene) and the sense strand comprises nucleotide sequence corresponding to the target nucleic acid sequence or a portion thereof. Alternatively, interfering RNA is assembled from a single oligonucleotide, where the self-complementary sense and antisense regions are linked by means of nucleic acid based or non-nucleic acid-based linker(s). The interfering RNA can be a polynucleotide with a duplex, asymmetric duplex, hairpin or asymmetric hairpin secondary structure, having self-complementary sense and antisense regions, wherein the antisense region comprises a nucleotide sequence that is complementary to nucleotide sequence in a separate target nucleic acid molecule or a portion thereof and the sense region having nucleotide sequence corresponding to the target nucleic acid sequence or a portion thereof. The interfering can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siRNA molecule capable of mediating RNA interference.

In some embodiments, the interfering RNA coding region encodes a self-complementary RNA molecule having a sense region, an antisense region and a loop region. Such an RNA molecule when expressed desirably forms a “hairpin” structure, and is referred to herein as an “shRNA.” The loop region is generally between about 2 and about 10 nucleotides in length. In some embodiments, the loop region is from about 6 to about 9 nucleotides in length. In some embodiments, the sense region and the antisense region are between about 15 and about 20 nucleotides in length. Following post-transcriptional processing, the small hairpin RNA is converted into a siRNA by a cleavage event mediated by the enzyme Dicer, which is a member of the RNase III family. The siRNA is then capable of inhibiting the expression of a gene with which it shares homology. For details, see Brummelkamp et al., Science 296:550-553, 2002; Lee et al., Nature Biotechnol., 20, 500-505, 2002; Miyagishi and Taira, Nature Biotechnol. 20:497-500, 2002; Paddison et al., Genes & Dev. 16:948-958, 2002; Paul, Nature Biotechnol. 20, 505-508, 2002; Sui, Proc. Natl. Acad Sci. U.S.A., 99(6):5515-5520. 2002; Yu et al., Proc. Natl. Acad. Sci. U.S.A. 99:6047-6052, 2002.

The target RNA cleavage reaction guided by siRNAs is highly sequence specific. In general, siRNA containing a nucleotide sequences identical to a portion of the target nucleic acid (i.e., a target region comprising the seed sequence of a target miRNA or mRNA) are preferred for inhibition. However, 100% sequence identity between the siRNA and the target gene is not required to practice the present invention. Thus the invention has the advantage of being able to tolerate sequence variations that might be expected due to genetic mutation, strain polymorphism, or evolutionary divergence. For example, siRNA sequences with insertions, deletions, and single point mutations relative to the target sequence have also been found to be effective for inhibition. Alternatively, siRNA sequences with nucleotide analog substitutions or insertions can be effective for inhibition. In general the siRNAs must retain specificity for their target, i.e., must not directly bind to, or directly significantly affect expression levels of, transcripts other than the intended target.

In some embodiments, an anti-miR agent for use in the composition, methods and kits as disclosed herein are LNA-anti-miR oligonucleotides as disclosed in Montgomery et al., Circulation, 2011; 124; 1537-1547; and Hullinger et al., Circulation, 2012; 110; 71-81, each of which are incorporated herein in their entirety by reference.

In some embodiments, anti-miR agents as disclosed herein differ from normal RNA by, for example, complete 2′-O-methylation of sugar, phosphorothioate intersugar linkage and, for example, a cholesterol-moiety at 3′-end. In some embodiments, antagomir comprises a 2′-O-methylmodification at all nucleotides, a cholesterol moiety at 3′-end, two phsophorothioate intersugar linkages at the first two positions at the 5′-end and four phosphorothioate linkages at the 3′-end of the molecule. Antagomirs can be used to efficiently silence endogenous miRNAs by forming duplexes comprising the antagomir and endogenous miRNA, thereby preventing miRNA-induced gene silencing. An example of antagomir-mediated miRNA silencing is the silencing of miR-122, described in Krutzfeldt et al, Nature, 2005, 438: 685-689, which is expressly incorporated by reference herein in its entirety.

Ribozymes:

In some embodiments, an anti-miR agent that inhibits at least one of miR-103, miR-105, and/or miR-107 is a ribozyme. Ribozymes are oligonucleotides having specific catalytic domains that possess endonuclease activity (Kim and Cech, Proc Natl Acad Sci USA. 1987 December; 84(24):8788-92; Forster and Symons, Cell. 1987 Apr. 24; 49(2):211-20). At least six basic varieties of naturally-occurring enzymatic RNAs are known presently. In general, enzymatic nucleic acids act by first binding to a target RNA. Such binding occurs through the target binding portion of an enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

Methods of producing a ribozyme targeted to any target sequence are known in the art. Ribozymes can be designed as described in Int. Pat. Appl. Publ. No. WO 93/23569 and Int. Pat. Appl. Publ. No. WO 94/02595, each specifically incorporated herein by reference, and synthesized to be tested in vitro and in vivo, as described therein.

In general, enzymatic nucleic acids with RNA cleaving activity act by first binding to a target RNA. Such binding occurs through the target binding portion of an enzymatic nucleic acid which is held in close proximity to an enzymatic portion of the molecule that acts to cleave the target RNA. Thus, the enzymatic nucleic acid first recognizes and then binds a target RNA through complementary base pairing, and once bound to the correct site, acts enzymatically to cut the target RNA. Strategic cleavage of such a target RNA will destroy its activity. After an enzymatic nucleic acid has bound and cleaved its RNA target, it is released from that RNA to search for another target and can repeatedly bind and cleave new targets.

Several approaches such as in vitro selection (evolution) strategies (Orgel. Proc. R. Soc. London, B 205:435, 1979) have been used to evolve new nucleic acid catalysts capable of catalyzing a variety of reactions, such as cleavage and ligation of phosphodiester linkages and amide linkages, (Joyce, Gene, 82, 83-87, 1989; Beaudry et al., Science 257, 635-641, 1992; Joyce, Scientific American 267, 90-97, 1992; Breaker et al., TIBTECH 12:268, 1994; Bartel et al., Science 261:1411-1418. 1993; Szostak, TIBS 17, 89-93, 1993; Kumar et al., FASEB J., 9:1183, 1995; Breaker, Curr. Op. Biotech., 1:442, 1996). The development of ribozymes that are optimal for catalytic activity would contribute significantly to any strategy that employs RNA-cleaving ribozymes for the purpose of regulating gene expression. The hammerhead ribozyme, for example, functions with a catalytic rate (kcat) of about 1 min⁻¹ in the presence of saturating (10 mM) concentrations of Mg²⁺ cofactor. An artificial “RNA ligase” ribozyme has been shown to catalyze the corresponding self-modification reaction with a rate of about 100 min⁻¹. In addition, it is known that certain modified hammerhead ribozymes that have substrate binding arms made of DNA catalyze RNA cleavage with multiple turn-over rates that approach 100 min⁻¹.

As used herein, the term “oligonucleotide” refers to a polymer or an oligomer of nucleotide or nucleoside monomers consisting of naturally occurring bases, sugars and intersugar linkages. The term “oligonucleotide” also includes polymers or oligomers comprising non-naturally occurring monomers, or portions thereof, which function similarly. Such modified or substituted oligonucleotides are often preferred over native forms because of properties such as, for example, enhanced cellular uptake and increased stability in the presence of nucleases.

The oligonucleotide can be single-stranded or double-stranded. A single-stranded oligonucleotide can have double-stranded regions and a double-stranded oligonucleotide can have single-stranded regions. The oligonucleotide can have a hairpin structure or have a dumbbell structure. The oligonucleotide can be, e.g., wherein the 5′ end of the oligonucleotide is linked to the 3′ end of the oligonucleotide.

The oligonucleotides described herein can comprise any oligonucleotide modification described herein and below. In some embodiments, the oligonucleotide comprises at least one modification. In some embodiments, the modification is selected from the group consisting of a sugar modification, a non-phosphodiester inter-sugar (or inter-nucleoside) linkage, nucleobase modification, and ligand conjugation.

In some embodiments, the oligonucleotide comprises at least two different modifications selected from the group consisting of a sugar modification, a non-phosphodiester inter-sugar linkage, nucleobase modification, and ligand conjugation. In some embodiments, at least two different modifications are present in the same subunit of the oligonucleotide, e.g. present in the same nucleotide.

As used herein, an oligonucleotide can be of any length. In some embodiments, oligonucleotides can range from about 6 to 100 nucleotides in length. In various related embodiments, the oligonucleotide can range in length from about 10 to about 50 nucleotides, from about 10 to about 35 nucleotides, from about 15 to about 30 nucleotides, from about 20 to about 30 nucleotides in length. In some embodiments, oligonucleotide is from about 8 to about 39 nucleotides in length. In some embodiments, the oligonucleotide is 10 to 25 nucleotides in length (e.g., 10, 11, 12, 13, 14, 15, 16, 18, 19, 20, 21, 22, 23, or 24 nucleotides in length). In some embodiments the oligonucleotide is 25-30 nucleotides. In some embodiments, the single-stranded oligonucleotide is 15 to 29 nucleotides in length. In some other embodiments, the oligonucleotide is from about 18 to about 25 nucleotides in length. In some embodiments, the oligonucleotide is about 23 nucleotides in length.

The oligonucleotide can be completely DNA, completely RNA, or comprise both RNA and DNA nucleotides. It is to be understood that when the oligonucleotide is completely DNA, RNA or a mix of both, the oligonucleotide can comprise one or more oligonucleotide modifications described herein.

An oligonucleotide can be a chimeric oligonucleotide. As used herein, a “chimeric” oligonucleotide” or “chimera” refers to an oligonucleotide which contains two or more chemically distinct regions, each made up of at least one monomer unit, i.e., a modified or unmodified nucleotide in the case of an oligonucleotide. Chimeric oligonucleotides can be described as having a particular motif. In some embodiments, the motifs include, but are not limited to, an alternating motif, a gapped motif, a hemi-mer motif, a uniformly fully modified motif and a positionally modified motif. As used herein, the phrase “chemically distinct region” refers to an oligonucleotide region which is different from other regions by having a modification that is not present elsewhere in the oligonucleotide or by not having a modification that is present elsewhere in the oligonucleotide. An oligonucleotide can comprise two or more chemically distinct regions. As used herein, a region that comprises no modifications is also considered chemically distinct.

A chemically distinct region can be repeated within an oligonucleotide. Thus, a pattern of chemically distinct regions in an oligonucleotide can be realized such that a first chemically distinct region is followed by one or more second chemically distinct regions. This sequence of chemically distinct regions can be repeated one or more times. Preferably, the sequence is repeated more than one time. Both strands of a double-stranded oligonucleotides can comprise these sequences. Each chemically distinct region can actually comprise as little as a single nucleotide. In some embodiments, each chemically distinct region comprises 1, 2, 3, 4, 5, 6, 7, 8, 9, 10, 11, 12, 13, 14, 15, 16, 17 or 18 nucleotides.

In some embodiments, alternating nucleotides comprise the same modification, e.g. all the odd number nucleotides in a strand have the same modification and/or all the even number nucleotides in a strand have the similar modification to the first strand. In some embodiments, all the odd number nucleotides in an oligonucleotide have the same modification and all the even numbered nucleotides have a modification that is not present in the odd number nucleotides and vice versa.

When the oligonucleotide is double-stranded and both strands of the double-stranded oligonucleotide comprise the alternating modification patterns, nucleotides of one strand can be complementary in position to nucleotides of the second strand which are similarly modified. In an alternative embodiment, there is a phase shift between the patterns of modifications of the first strand, respectively, relative to the pattern of similar modifications of the second strand. Preferably, the shift is such that the similarly modified nucleotides of the first strand and second strand are not in complementary position to each other. In some embodiments, the first strand has an alternating modification pattern wherein alternating nucleotides comprise a 2′-modification, e.g., 2′-O-Methyl modification. In some embodiments, the first strand comprises an alternating 2′-O-Methyl modification and the second strand comprises an alternating 2′-fluoro modification. In other embodiments, both strands of a double-stranded oligonucleotide comprise alternating 2′-O-methyl modifications. When both strands of a double-stranded oligonucleotide comprise alternating 2′-O-methyl modifications, such 2′-modified nucleotides can be in complementary position in the duplex region. Alternatively, such 2′-modified nucleotides may not be in complementary positions in the duplex region.

In some embodiments, the oligonucleotide comprises two chemically distinct regions, wherein each region is 1-10 nucleotides in length.

In other embodiments, the oligonucleotide comprises three chemically distinct regions. The middle region is about 5-15, (e.g., 5, 6, 7, 8, 9, 10, 11, 12, 13, 14 or 15) nucleotide in length and each flanking or wing region is independently 1-10 (e.g., 1, 2, 3, 4, 5, 6, 7, 8, 9 or 10) nucleotides in length. All three regions can have different modifications or the wing regions can be similarly modified to each other. In some embodiments, the wing regions are of equal length, e.g. 1, 2, 3, 4, 5, 6, 7, 8, 9 or 10 nucleotides long.

As used herein the term “alternating motif” refers to an oligonucleotide comprising at least two different chemically distinct regions that alternate for essentially the entire sequence of the oligonucleotide. In an alternating motif length of each region is independent of the length of other regions.

As used herein, the term “uniformly fully modified motif” refers to an oligonucleotide wherein all nucleotides in the oligonucleotide have at least one modification that is the same.

As used herein, the term “hemi-mer motif” refers to an oligonucleotide having two chemically distinct regions, wherein one region is at the 5′ end of the oligonucleotide and the other region is at the 3 end of the oligonucleotide. In some embodiments, length of each chemically distinct region is independently 1 nucleotide to 1 nucleotide less than the length of the oligonucleotide.

As used herein the term “gapped motif” refers to an oligonucleotide having three chemically distinct regions. In some embodiments, the gapped motif is a symmetric gapped motif, wherein the two outer chemically distinct regions (wing regions) are identically modified. In another embodiment, the gapped motif is an asymmetric gaped motif in that the three regions are chemically distinct from each other

As used herein the term “positionally modified motif” refers to an oligonucleotide having three or more chemically distinct regions. Positionally modified oligonucleotides are distinguished from gapped motifs, hemi-mer motifs, blockmer motifs and alternating motifs because the pattern of regional substitution defined by any positional motif does not fit into the definition provided herein for one of these other motifs. The term positionally modified oligomeric compound includes many different specific substitution patterns.

In some embodiments, oligonucleotide comprises two or more chemically distinct regions and has a structure as described in International Application No. PCT/US09/038433, filed Mar. 26, 2009, content of which is incorporated herein by reference in its entirety. In some embodiments, the single-stranded oligonucleotide has a ZXY structure, such as is described in International Application No. PCT/US2004/07070 filed on Mar. 8, 2004, content of which is incorporated herein by reference in its entirety.

In some embodiments, the anti-miR oligonucleotide comprises 2′-MOE modifications at all positions and phosphorothioate inter-sugar linkages at all positions.

In some embodiments, the anti-miR comprises a mix of 2′-F and 2′-MOE modified nucleotides.

In some embodiments, the anti-miR comprises at least 1 (e.g., 1, 2, 3, 4, or 5) 2′-F modified nucleotides at the 5′ end (i.e., the first 1, 2, 3, 4, or 5 nucleotides at the 5′ end are 2′-F modified nucleotides).

In some embodiments, the anti-miR comprises at least 1 (e.g., 1, 2, 3, 4, or 5) 2′-F modified nucleotides at the 3′ end (i.e., the first 1, 2, 3, 4, or 5 nucleotides at the 3′ end are 2′-F modified nucleotides).

In some embodiments, the anti-miR comprises, independently, at least 1 (e.g., 1, 2, 3, 4, or 5) 2′-F modified nucleotides at the 5′ end and at the 3′ end and 2′-MOE modified nucleotides at all other positions.

In some embodiments, the anti-miR comprises two 2′-F modified nucleotides at the 5′ end and at the 3′ end and 2′-MOE modified nucleotides at all other positions, e.g., a 2′-F/2′-MOE mixmer.

In some embodiments, the anti-miR comprises two 2′-F modified nucleotides at the 5′ end and at the 3′ end, 2′-MOE modified nucleotides at all other positions, and phosphorothioate inter-sugar linkages at all positions.

In some embodiments, the anti-miR comprises a mix of LNA and DNA monomers, e.g., a LNA/DNA mixmer. The LNA and DNA monomers can be arranged in any pattern. In some embodiments, the LNA and DNA monomers are arranged in an alternative pattern, e.g., a LNA monomer followed by a DNA monomer. This alternating pattern can be repeated for the full length of the anti-miR.

The oligonucleotide can hybridize to a complementary RNA, e.g., mRNA, pre-mRNA, microRNA, or pre-microRNA and reduce the activity, expression, or amount of the complementary RNA, e.g., target RNA. This can be by reducing access of the translation machinery to the target RNA transcript, thereby preventing protein synthesis. The oligonucleotide can induce cleavage of the complementary RNA by an enzyme, such RISC mediated cleavage or RNase H and thus reducing the amount of the target RNA. The oligonucleotide itself can cleave the complementary RNA, e.g., a ribozyme, RISC mediated cleavage or RNase H and thus reducing the amount of the target RNA. The oligonucleotide, by hybridizing to the target RNA, can inhibit binding of the target RNA to another complementary strand.

The term “target sequence” to which a RNAi “targets” means any nucleic acid sequence whose expression or activity is to be modulated. The target nucleic acid can be DNA or RNA, such as endogenous DNA or RNA, viral DNA or viral RNA, or other RNA encoded by a gene, virus, bacteria, fungus, mammal, or plant.

In some embodiments, the target sequence is a nucleic acid encoding, expressing or comprising the nucleotide sequence of pre-miRNA-103, pre-miRNA-105, pre-miRNA-107, pre-miRNA-155 or pre-miRNA-425. In some embodiments, the target sequence is a nucleic acid encoding, expressing or comprising the nucleotide sequence of miR1-03, miR-105, miR-107 and/or miR-155. In some embodiments, the target sequence is a nucleic acid encoding, expressing or comprising the nucleotide seed sequences of, miR1-03, miR-105, miR-107 and/or miR-155, e.g., SEQ ID NO: 4, 9, and 14.

By “specifically hybridizable” and “complementary” is meant that a first nucleic acid strand can form hydrogen bond(s) with a second nucleic acid strand by either traditional Watson-Crick or other non-traditional types. A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” or 100% complementarity means that all the contiguous residues of a nucleic acid sequence will hydrogen bond with the same number of contiguous residues in a second nucleic acid sequence. Less than perfect complementarity refers to the situation in which some, but not all, nucleoside units of two strands can hydrogen bond with each other. “Substantial complementarity” refers to polynucleotide strands exhibiting 90% or greater complementarity, excluding regions of the polynucleotide strands, such as overhangs, that are selected so as to be non-complementary. Specific binding requires a sufficient degree of complementarity to avoid non-specific binding of the oligomeric compound to non-target sequences under conditions in which specific binding is desired, i.e., under physiological conditions in the case of in vivo assays or therapeutic treatment, or in the case of in vitro assays, under conditions in which the assays are performed. The non-target sequences typically differ by at least 1, 2, 3, 4, or 5 nucleotides.

miR-103, miR-105, miR-107 or miR-155 Agent for Use to Decrease ANP Levels in Subjects

As disclosed herein, the present invention also encompasses miR agents which are miR agonists to miR-103, miR-105, miR-107 and miR-155 in compositions, kits and methods to treat a subject where it is desirable to increase blood pressure, e.g., a subject with low blood pressure, or a subject suffering from severe shock).

In some embodiments, where a miR agent (e.g., an agonist or activator of miR-103, miR-105, miR-107 and/or miR-155) as disclosed herein is administered to a subject, (e.g., to a subject with low blood pressure, or where it is desirable to increase blood pressure in a subject, e.g., a subject suffering from severe shock), a miR agent can be a miR-103, miR-105, miR-107 and/or miR-155 that is a pri-miRNA, pre-miRNA, mature miR or a fragment or variant thereof effective in gene silencing the 3′UTR of the ANP mRNA.

Agents useful in the methods of treatment described herein include sense nucleic acid molecules that increase the expression or activity of any of the miRs disclosed herein (e.g., mature microRNA or precursor microRNA) e.g., miR-103, miR-105, miR-107 and/or miR-155. A sense nucleic acid can be contain a sequence that is at least 80% (e.g., at least 90%, 95%, 96%, 97%, 98%, 99%, or 100%) identical to the sequence of any one of the miRs disclosed herein (e.g., mature microRNA or precursor microRNA) e.g., miR-103, miR-105, miR-107 and/or miR-155. Sense nucleic acids can contain one or more of any of the modifications (e.g., backbone modifications, nucleobase modifications, sugar modifications, or one or more conjugated molecules) described herein without limitation. Methods of making and administering sense nucleic acids are known in the art. Additional methods of making and using sense nucleic acids are described herein.

miR-103 Agonists

In some embodiments, a miR-103 agent comprises hsa-miR-103a-3p (SEQ ID NO: 1), or hsa-miR-103b (SEQ ID NO: 2) and/or hsa-miR-103a-2-5p (SEQ ID NO: 3) or a fragment or homologue thereof effective in gene silencing the 3′ UTR of NPPA gene. In alternative embodiments, a miR-103 agent is a homologue of at least 75% identity, least 80%, or at least 85%, or at least about 90% or more than 90% identity to hsa-miR-103a-3p (SEQ ID NO: 1), or hsa-miR-103b (SEQ ID NO: 2) and/or hsa-miR-103a-2-5p (SEQ ID NO: 3), or a fragment thereof effective in gene silencing the 3′UTR of NPPA

miR-107 Agonists

In some embodiments, a miR-107 agent comprises hsa-miR-107 (SEQ ID NO: 11) or a fragment or homologue thereof effective in gene silencing the 3′ UTR of NPPA gene. In alternative embodiments, a miR-107 agent is a homologue of at least 75% identity, least 80%, or at least 85%, or at least about 90% or more than 90% identity to hsa-miR-107 (SEQ ID NO: 11), or a fragment thereof effective in gene silencing the 3′UTR of NPPA mRNA. Homologues of miR-107 are well know in the art and are disclosed herein as SEQ ID NO: 29-130. In some embodiments, a miR-107 agent is a pre-miR-105, e.g., a pre-miRNA-107 nucleic acid sequence with at least 80%, or at least 85% or at least about 90% or more than 90% identity to one of the following hsa-miR-107 stem loop (SEQ ID NO: 27). A hsa-miR-107 that results in the mature miR-107 sequence of SEQ ID NO: 11 is commercially available, and can be purchaced from Pre-mir™ miRNA Precusor molecules. In other embodiments, a miR-107 agent is an RNA interference-inducing (RNAi) molecule including, but not limited to, a siRNA, dsRNA, stRNA, shRNA and gene silencing variants thereof. In alternative embodiments a miR-107 agent binds to the 3′ UTR of NPPA, e.g., binds at miR-107 target site of SEQ ID NO: 6 and inhibits the expression of the NPPA mRNA transcript from the NPPA gene. Examples of such miR-107 agents include, but are not limited to an anti-miR, small molecule, protein, antibody, aptamer, ribozyme, nucleic acid or nucleic acid analogue.

miR-155 Agonists

In some embodiments, a miR-155 agent comprises hsa-miR-155 (SEQ ID NO: 12) or a fragment or homologue thereof effective in gene silencing the 3′ UTR of NPPA gene. In alternative embodiments, a miR-155 agent is a homologue of at least 75% identity, least 80%, or at least 85%, or at least about 90% or more than 90% identity to hsa-miR-155 (SEQ ID NO: 12), or a fragment thereof effective in gene silencing the 3′UTR of NPPA mRNA. Homologues of miR-miR-155 are well know in the art and are disclosed herein as SEQ ID NO: 211-235, as shown in Table 8. Examples of such miR-155 agents include, but are not limited to an anti-miR, small molecule, protein, antibody, aptamer, ribozyme, nucleic acid or nucleic acid analogue.

TABLE 8
The human miR-155 family is shown as follows:

| SEQ | | |
|---|---|---|
| ID | | |
| NO | Species of pre-miR | SEQUENCE |
| 12 | human mature miR-155-5p | UUAAUGCUAAUCGUGAUAGGGGU |
| 203 | human pre-miR-155 (hsa- | CUGUUAAUGCUAAUCGUGAUAGGGGUUUUUGCCUCCAACUG |
| | mir-155) MI0000681 | ACUCCUACAUAUUAGCAUUAACAG |
| 211 | mouse pre-miR- 155 (mml- | CUGUUAAUGCUAAUCGUGAUAGGGGUUUUUACCUCCAACUG |
| | mir-155; MI0007645) | ACUCCUACAUGUUAGCAUUAACAG |
| 212 | mouse mature miR-155 | UUAAUGCUAAUUGUGAUAGGGGU |
| 213 | aca-mir-155 MI0018764 | CACACGUUGUUAAUGCUAAUCGUGAUAGGGGUUUUUAUCUC |
| | | UGACUGACUCCUACAUGUUAGCAUUAAGAAUGUGUAGUG |
| 214 | bta-mir-155 MI0009752 | UGUUAAUGCUAAUCGUGAUAGGGGUUUUUACCUCGGACUGA |
| | | CUCCUACAUGUUAGCAUUAACA |
| 215 | ccr-mir-155 MI0023 336 | UCAGGUUUAAUGCUAAUCGUGAUAGGGGUUUUGUACUACU |
| | | GAACACCUAUGCUGUUAGCAUUAGUCCUGCG |
| 216 | cfa-mir-155 MI0008078 | UUAAUGCUAAUCGUGAUAGGGGUUUUUACCUCCGACUGACU |
| | | CCUACAUGUUAGCAUUAACA |
| 217 | cgr-mir-155 MI0020415 | CUGUAUGCUGUUAAUGCUAAUCGUGAUAGGGGUUUUUGCG |
| | | UCUGACUGACUCCUACAUGUUAGCAUUAACAGGGUACGAUG |
| | | CCUGCCUGC |
| 218 | chi-mir-155 MI0030643 | UUGCUGUGGGCUGUGUGCUAUUAAUGCUAAUCGUGAUAGG |
| | | GGUUUUUACCUCGGACUGACUCCUACAUGUUAGCAUUAACA |
| | | GGGCAUGGCGCCUGUUAC |
| 219 | dre-mir-155 MI0002023 | UGGUGCAGGUUUAAUGCUAAUCGUGAUAGGGGUUUAGUGC |
| | | UGAUGAACACCUAUGCUGUUAGCAUUAAUCUUGCGCUA |
| 220 | eca-mir-155 MI0012927 | CUGUUAAUGCUAAUCGUGAUAGGGGUUUUUACCUCGGACUG |
| | | ACUCCUACAUGUUAGCAUUAACAG |
| 221 | efu-mir-155 MI0028748 | CUGAAAGCCGGAGGCCUGCUGGAGGCUGUAUGCUGUUAAUG |
| | | CUAAUCGUGAUAGGGGUUUUUACCUCCGACUGACUCCUACA |
| | | UGUUAGCAUUAACAGGGUACGGUGCCUGAUACUAGCAUUCA |
| | | CACGGAACAGAU |
| 222 | gga-mir-155 MI0001176 | UGUUAAUGCUAAUCGUGAUAGGGGUUUUUACCUCUGAAUG |
| | | ACUCCUACAUGUUAGCAUUAACA |
| 223 | ggo-mir-155 MI0020768 | UUGCUGUAGGCUGUAUGCUGUUAAUGCUAAUCGUGAUAGG |
| | | GGUUUUUGCCUCCAACUGACUCCUACAUAUUAGCAUUAACA |
| | | GGGUAUGAUGCCUGUUACUAGCAUUCACAU |
| 224 | ipu-mir-155 MI0024518 | UUAAUGCUAAUCGUGAUAGGGGUUUAGUGCUGAUGAACACC |
| | | UAUGCUGUUAGCAUUAAUC |
| 225 | mmu-mir-155 MI0000177 | CUGUUAAUGCUAAUUGUGAUAGGGGUUUUGGCCUCUGACUG |
| | | ACUCCUACCUGUUAGCAUUAACAG |
| 226 | oan-mir-155 MI0006775 | GGGACGCCGUGGGCCGCAGGUCGUUAAUGCUAAUCGUGAUA |
| | | GGGGUUUUCUCUGUCUGACUGACCCCUGCCCGUUAGCAUUA |
| | | GCAACGUGCGGCCCCUCGAACCC |
| 227 | ppy-mir-155 MI0014843 | CUGUUAAUGCUAAUCGUGAUAGGGGUUUUUACCUCCAACUG |
| | | ACUCCUACAUGUUAGCAUUAACAG |
| 228 | ptr-mir-155 MI0008554 | UGUUAAUGCUAAUCGUGAUAGGGGUUUUUGCCUCCAAAUGA |
| | | CUCCUACAUAUUAGCAUUAACAG |
| 229 | rno-mir-155 MI0025509 | CUGUUAAUGCUAAUUGUGAUAGGGGUUUUGGCCUCUGACUG |
| | | ACUCCUACCUGUUAGCAUUAACAG |
| 230 | ssa-mir-155-1 MI0026520 | UUAAUGCUAAUCGUGAUAGGGGUUCUGUUGUUUAAAAAAA |
| | | AGACACCUAGCAUGUAAGCAUUAGC |
| 231 | ssa-mir-155-2 MI0026521 | UUAAUGCUAAUCGUGAUAGGGGUUCUGUUGUUAAAAAAAA |
| | | AGACACCUAGCAUGUAAGCAUUAGC |
| 232 | ssc-mir-155 MI0015907 | CUAUAUGCUGUUAAUGCUAAUUGUGAUAGGGGUUUUUACC |
| | | UCCGUCUGACUCCUACAUGUUAGCAUUAACAGGGUAUGAUG |
| 233 | tch-mir-155 MI0031131 | UUAAUGCUAAUCGUGAUAGGGGUUUUUGCCUCCGACUGACU |
| | | CCUACAUGUUAGCAUUAACA |
| 234 | tgu-mir-155 MI0013842 | CUGUAUGUUGUUAAUGCUAAUCGUGAUAGGGGUUUUUACC |
| | | UAUGACUGACUCCUACACGUUAGCAUUAACA |
| 235 | xtr-mir- 155 MI0004848 | UAUUAAUGCUAAUCGUGAUAGGGGUUUUUUAAUACAUAUU |
| | | GACUCCUACAUGUUAGCAUUAUUAUCAUG |

In some embodiments, a miR-miR-155 agent is a pre-miR-155, e.g., a pre-miRNA-155 nucleic acid sequence with at least 80%, or at least 85% or at least about 90% or more than 90% identity to one of the following hsa-miR-155 stem loop (SEQ ID NO: 203). A hsa-miR-155 that results in the mature miR-155 sequence of SEQ ID NO: 12 is commercially available, and can be purchaced from Pre-mir™ miRNA Precusor molecules. In other embodiments, a miR-155 agent is an RNA interference-inducing (RNAi) molecule including, but not limited to, a siRNA, dsRNA, stRNA, shRNA and gene silencing variants thereof. In alternative embodiments a miR-155 agent is a RNAi agent that binds to, and targets the 3′ UTR of NPPA, e.g., is a RNAi agent which binds at miR-155 target site of SEQ ID NO: 199 or 200 in the 3′UTR of the NPPA gene and inhibits the expression of the NPPA mRNA transcript from the NPPA gene. Examples of such miR-155 agents include, but are not limited to an anti-miR, small molecule, protein, antibody, aptamer, ribozyme, nucleic acid or nucleic acid analogue.

In some embodiments, a miR-155 agonist is a mature miR-155-3p (CUCCUACAUAUUAGCAUUAACA; SEQ ID NO: 209). In some embodiments, a miR-155 agent is a siRNA which targets and is complementary to the miR-155 target/binding sequence on 3′UTR of NPPA gene which comprisss the major A allele of rs61764044 (e.g., is complementary to AAUGCAUGGGGUGGGAGAGG (SEQ ID NO: 199)), thereby preventing the expression of the NPPA mRNA transcript from the NPPA gene comprising the major A allele of rs61764044. In some embodiments, a miR-155 agent is a siRNA which targets and is complementary to the miR-155 target/binding sequence on 3′UTR of NPPA gene which comprisss the minor G allele of rs61764044 (e.g., is complementary to UAAUGCGUGGGGUGGGAGAGG (SEQ ID NO: 200)), thereby preventing the expression of the NPPA mRNA transcript from the NPPA gene comprising the minor G allele of rs61764044.

MicroRNAs (also referred to as miRNAs or miRs) are a highly conserved class of small RNA molecules that are transcribed from DNA in the genomes of plants and animals, but are not translated into protein. Pre-microRNAs are processed into miRNAs. Processed microRNAs are single stranded ˜17-25 nucleotide (nt) RNA molecules that become incorporated into the RNA-induced silencing complex (RISC) and have been identified as key regulators of development, cell proliferation, apoptosis and differentiation. They are believed to play a role in regulation of gene expression by binding to the 3′-untranslated region of specific mRNAs. RISC mediates down-regulation of gene expression through translational inhibition, transcript cleavage, or both. RISC is also implicated in transcriptional silencing in the nucleus of a wide range of eukaryotes.

MicroRNAs have also been implicated in modulation of pathogens in hosts. For example, see Jopling, C. L., et al., Science (2005) vol. 309, pp 1577-1581. Without wishing to be bound by theory, administration of a microRNA, microRNA mimic, and/or anti microRNA oligonucleotide, leads to modulation of pathogen viability, growth, development, and/or replication. In some embodiments, the oligonucleotide is a microRNA, microRNA mimic, and/or anti microRNA, wherein microRNA is a host microRNA (miRNA).

Methods of making and stabilizing miRNAs for therapeutic use are well known in the art, and are discussed in Broderick et al., Gene Therapy, 2011; 18; 1104-1110, which is incorporated herein in its entirety by reference. Broderick et al., also discusses methods of artificially inhibiting miRNAs, suitable for generating anti-miR-425 agents as disclosed herein.

The number of miRNA sequences identified to date is large and growing, illustrative examples of which can be found, for example, in: “miRBase: microRNA sequences, targets and gene nomenclature” Griffiths-Jones S, Grocock R J, van Dongen S, Bateman A, Enright A J. NAR, 2006, 34, Database Issue, D140-D144; “The microRNA Registry” Griffiths-Jones S. NAR, 2004, 32, Database Issue, D109-D111; and also on the worldwide web at microrna.dot.sanger.dot.ac.dot.uk/sequences/.

miR Mimics:

A miR mimetic (also known as a miRNA mimics) represent a class of molecules that can be used to imitate the gene modulating activity of one or more miRNAs. Thus, the term “microRNA mimic” refers to synthetic non-coding RNAs (i.e. the miRNA is not obtained by purification from a source of the endogenous miRNA) that are capable of entering the RNAi pathway and regulating gene expression. miRNA mimics can be designed as mature molecules (e.g. single stranded) or mimic precursors (e.g., pri- or pre-miRNAs).

In one design, miRNA mimics are double stranded molecules (e.g., with a duplex region of between about 16 and about 31 nucleotides in length) and contain one or more sequences that have identity with the mature strand of a given miRNA. Double-stranded miRNA mimics have designs similar to as described above for double-stranded oligonucleotides.

In some embodiments, a miRNA mimic comprises a duplex region of between 16 and 31 nucleotides and one or more of the following chemical modification patterns: the sense strand contains 2′-O-methyl modifications of nucleotides 1 and 2 (counting from the 5′ end of the sense oligonucleotide), and all of the Cs and Us; the antisense strand modifications can comprise 2′ F modification of all of the Cs and Us, phosphorylation of the 5′ end of the oligonucleotide, and stabilized internucleotide linkages associated with a 2 nucleotide 3′ overhang.

Supermirs:

A supermir refers to an oligonucleotide, e.g., single stranded, double-stranded or partially double-stranded, which has a nucleotide sequence that is substantially identical to an miRNA and that is antisense with respect to its target. This term includes oligonucleotides which comprise at least one non-naturally-occurring portion which functions similarly. In a preferred embodiment, the supermir does not include a sense strand, and in another preferred embodiment, the supermir does not self-hybridize to a significant extent. A supermir featured in the invention can have secondary structure, but it is substantially single-stranded under physiological conditions. A supermir that is substantially single-stranded is single-stranded to the extent that less than about 50% (e.g., less than about 40%, 30%, 20%, 10%, or 5%) of the supermir is duplexed with itself. The supermir can include a hairpin segment, e.g., sequence, preferably at the 3′ end can self hybridize and form a duplex region, e.g., a duplex region of at least 1, 2, 3, or 4 and preferably less than 8, 7, 6, or 5 nucleotides, e.g., 5 nucleotides. The duplexed region can be connected by a linker, e.g., a nucleotide linker, e.g., 3, 4, 5, or 6 dTs, e.g., modified dTs. In another embodiment the supermir is duplexed with a shorter oligo, e.g., of 5, 6, 7, 8, 9, or 10 nucleotides in length, e.g., at one or both of the 3′ and 5′ end or at one end and in the non-terminal or middle of the supermir.

### Oligonucleotide Modifications

In some embodiments, anti-miRs (e.g., anti-miR-103 agents, anti-miR-105 agents, anti-miR-107 agents, anti-miR-155 agents and, optionally, anti-425 agents) and miR agonists (e.g., agonists of miR-103, miR-105, miR-107, miR-155 and, optionally miR-425) for use in the aspects of the invention as disclosed herein can include oligonucleotide modifications. Unmodified oligonucleotides can be less than optimal in some applications, e.g., unmodified oligonucleotides can be prone to degradation by e.g., cellular nucleases. However, chemical modifications to one or more of the subunits of oligonucleotide can confer improved properties, e.g., can render oligonucleotides more stable to nucleases. Typical oligonucleotide modifications can include one or more of: (i) alteration, e.g., replacement, of one or both of the non-linking phosphate oxygens and/or of one or more of the linking phosphate oxygens in the phosphodiester intersugar linkage; (ii) alteration, e.g., replacement, of a constituent of the ribose sugar, e.g., of the 2′ hydroxyl on the ribose sugar; (iii) wholesale replacement of the phosphate moiety with “dephospho” linkers; (iv) modification or replacement of a naturally occurring base with a non-natural base; (v) replacement or modification of the ribose-phosphate backbone, e.g. peptide nucleic acid (PNA); (vi) modification of the 3′ end or 5′ end of the oligonucleotide, e.g., removal, modification or replacement of a terminal phosphate group or conjugation of a moiety, e.g., conjugation of a ligand, to either the 3′ or 5′ end of oligonucleotide; and (vii) modification of the sugar, e.g., six membered rings.

The terms replacement, modification, alteration, and the like, as used in this context, do not imply any process limitation, e.g., modification does not mean that one must start with a reference or naturally occurring ribonucleic acid and modify it to produce a modified ribonucleic acid bur rather modified simply indicates a difference from a naturally occurring molecule. As described below, modifications, e.g., those described herein, can be provided as asymmetrical modifications.

A modification described herein can be the sole modification, or the sole type of modification included on multiple nucleotides, or a modification can be combined with one or more other modifications described herein. The modifications described herein can also be combined onto an oligonucleotide, e.g. different nucleotides of an oligonucleotide have different modifications described herein.

Modifications of phosphate group: The phosphate group in the intersugar linkage can be modified by replacing one of the oxygens with a different substituent. One result of this modification to RNA phosphate intersugar linkages can be increased resistance of the oligonucleotide to nucleolytic breakdown. Examples of modified phosphate groups include phosphorothioate, phosphoroselenates, borano phosphates, borano phosphate esters, hydrogen phosphonates, phosphoroamidates, alkyl or aryl phosphonates and phosphotriesters. In some embodiments, one of the non-bridging phosphate oxygen atoms in the intersugar linkage can be replaced by any of the following: S, Se, BR₃ (R is hydrogen, alkyl, aryl), C (i.e. an alkyl group, an aryl group, etc. . . . ), H, NR₂ (R is hydrogen, optionally substituted alkyl, aryl), or OR (R is optionally substituted alkyl or aryl). The phosphorous atom in an unmodified phosphate group is achiral. However, replacement of one of the non-bridging oxygens with one of the above atoms or groups of atoms renders the phosphorous atom chiral; in other words a phosphorous atom in a phosphate group modified in this way is a stereogenic center. The stereogenic phosphorous atom can possess either the “R” configuration (herein Rp) or the “S” configuration (herein Sp).

Phosphorodithioates have both non-bridging oxygens replaced by sulfur. The phosphorus center in the phosphorodithioates is achiral which precludes the formation of oligonucleotides diastereomers. Thus, while not wishing to be bound by theory, modifications to both non-bridging oxygens, which eliminate the chiral center, e.g. phosphorodithioate formation, can be desirable in that they cannot produce diastereomer mixtures. Thus, the non-bridging oxygens can be independently any one of O, S, Se, B, C, H, N, or OR (R is alkyl or aryl).

The phosphate linker can also be modified by replacement of bridging oxygen, (i.e. oxygen that links the phosphate to the nucleoside), with nitrogen (bridged phosphoroamidates), sulfur (bridged phosphorothioates) and carbon (bridged methylenephosphonates). The replacement can occur at the either one of the linking oxygens or at both linking oxygens. When the bridging oxygen is the 3′-oxygen of a nucleoside, replacement with carbon is preferred. When the bridging oxygen is the 5′-oxygen of a nucleoside, replacement with nitrogen is preferred.

Modified phosphate linkages where at least one of the oxygen linked to the phosphate has been replaced or the phosphate group has been replaced by a non-phosphorous group, are also referred to as “non-phosphodiester intersugar linkage” or “non-phosphodiester linker.”

Replacement of the Phosphate Group:

The phosphate group can be replaced by non-phosphorus containing connectors, e.g. dephospho linkers. Dephospho linkers are also referred to as non-phosphodiester linkers herein. While not wishing to be bound by theory, it is believed that since the charged phosphodiester group is the reaction center in nucleolytic degradation, its replacement with neutral structural mimics should impart enhanced nuclease stability. Again, while not wishing to be bound by theory, it can be desirable, in some embodiment, to introduce alterations in which the charged phosphate group is replaced by a neutral moiety.

Examples of moieties which can replace the phosphate group include, but are not limited to, amides (for example amide-3 (3′-CH₂—C(═O)—N(H)-5′) and amide-4 (3′-CH₂—N(H)—C(═O)-5′)), hydroxylamino, siloxane (dialkylsiloxxane), carboxamide, carbonate, carboxymethyl, carbamate, carboxylate ester, thioether, ethylene oxide linker, sulfide, sulfonate, sulfonamide, sulfonate ester, thioformacetal (3′-S—CH₂—O-5′), formacetal (3′-O—CH₂—O-5′), oxime, methyleneimino, methykenecarbonylamino, methylenemethylimino (MMI, 3′-CH₂—N(CH₃)—O-5′), methylenehydrazo, methylenedimethylhydrazo, methyleneoxymethylimino, ethers (C3′-O—C5′), thioethers (C3′-S—C5′), thioacetamido (C3′-N(H)—C(═O)—CH₂—S—C5′, C3′-O—P(O)—O—SS—C5′, C3′-CH₂—NH—NH—C5′, 3′-NHP(O)(OCH₃)—O-5′ and 3′-NHP(O)(OCH₃)—O-5′ and nonionic linkages containing mixed N, O, S and CH₂ component parts. See for example, Carbohydrate Modifications in Antisense Research; Y. S. Sanghvi and P. D. Cook Eds. ACS Symposium Series 580; Chapters 3 and 4, (pp. 40-65). Preferred embodiments include methylenemethylimino (MMI), methylenecarbonylamino, amides, carbamate and ethylene oxide linker.

One skilled in the art is well aware that in certain instances replacement of a non-bridging oxygen can lead to enhanced cleavage of the intersugar linkage by the neighboring 2′-OH, thus in many instances, a modification of a non-bridging oxygen can necessitate modification of 2′-OH, e.g., a modification that does not participate in cleavage of the neighboring intersugar linkage, e.g., arabinose sugar, 2′-O-alkyl, 2′-F, LNA and ENA.

Preferred non-phosphodiester intersugar linkages include phosphorothioates, phosphorothioates with an at least 1%, 5%, 10%, 20%, 30%, 40%, 50%, 60%, 70%, 80%, 90% 95% or more enantiomeric excess of Sp isomer, phosphorothioates with an at least 1%, 5%, 10%, 20%, 30%, 40%, 50%, 60%, 70%, 80%, 90% 95% or more enantiomeric excess of Rp isomer, phosphorodithioates, phsophotriesters, aminoalkylphosphotrioesters, alkyl-phosphonaters (e.g., methylphosphonate), selenophosphates, phosphoramidates (e.g., N-alkylphosphoramidate), and boranophosphonates.

Replacement of Ribophosphate Backbone:

Oligonucleotide-mimicking scaffolds can also be constructed wherein the phosphate linker and ribose sugar are replaced by nuclease resistant nucleoside or nucleotide surrogates. While not wishing to be bound by theory, it is believed that the absence of a repetitively charged backbone diminishes binding to proteins that recognize polyanions (e.g. nucleases). Again, while not wishing to be bound by theory, it can be desirable in some embodiment, to introduce alterations in which the bases are tethered by a neutral surrogate backbone. Examples include the morpholino, cyclobutyl, pyrrolidine, peptide nucleic acid (PNA), aminoethylglycyl PNA (aegPNA) and backnone-extended pyrrolidine PNA (bepPNA) nucleoside surrogates. In some embodiments, the oligonucleotide is a peptide nucleic acid, e.g., the ribophosphate backbone of the oligonucleotide is completely replaced by peptide nucleic acid (PNA).

Sugar Modifications:

An oligonucleotide can include modification of all or some of the sugar groups of the nucleic acid. For example, the 2′ position (H, DNA; or OH, RNA) can be modified with a number of different “oxy” or “deoxy” substituents. While not being bound by theory, enhanced stability is expected since the 2′-hydroxyl can no longer be deprotonated to form a 2′-alkoxide ion. The 2′-alkoxide can catalyze degradation by intramolecular nucleophilic attack on the linker phosphorus atom. Again, while not wishing to be bound by theory, it can be desirable to some embodiments to introduce alterations in which alkoxide formation at the 2′ position is not possible

Examples of “oxy”-2′ hydroxyl group modifications include alkoxy or aryloxy (OR, e.g., R═H, alkyl, cycloalkyl, aryl, aralkyl, heteroaryl or sugar); polyethyleneglycols (PEG), O(CH₂CH₂O)ₙCH₂CH₂OR, n=1-50; “locked” nucleic acids (LNA) in which the oxygen at the 2′ position is connected by (CH₂)ₙ, wherein n=1-4, to the 4′ carbon of the same ribose sugar, preferably n is 1 (LNA) or 2 (ENA); O-AMINE or O—(CH₂)ₙAMINE (n=1-10, AMINE=NH₂; alkylamino, dialkylamino, heterocyclyl, arylamino, diaryl amino, heteroaryl amino, diheteroaryl amino, ethylene diamine or polyamino); and O—CH₂CH₂(NCH₂CH₂NMe₂)₂.

Examples of “deoxy” modifications include halo (e.g., fluoro); amino (e.g. NH₂; alkylamino, dialkylamino, heterocyclyl, arylamino, diaryl amino, heteroaryl amino, diheteroaryl amino, or amino acid); NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino); —NHC(O)R (R=alkyl, cycloalkyl, aryl, aralkyl, heteroaryl or sugar); cyano; mercapto; alkyl-thio-alkyl; thioalkoxy; thioalkyl; alkyl; cycloalkyl; aryl; alkenyl and alkynyl, which can be optionally substituted with e.g., an amino functionality.

The sugar group can also contain one or more carbons that possess the opposite stereochemical configuration than that of the corresponding carbon in the ribose sugar. Thus, an oligonucleotide can include nucleotides containing e.g., arabinose, as the sugar. Similarly, a modification at the 2′ position can be present in the arabinose configuration The term “arabinose configuration” refers to the placement of a substituent on the C2′ of ribose in the same configuration as the 2′-OH is in the arabinose.

A nucleotide can have an alpha linkage at the 1′ position on the sugar, e.g., alpha-nucleosides. The monomer can also have the opposite configuration at the 4′-position, e.g., C5′ and H4′ or substituents replacing them are interchanged with each other. When the C5′ and H4′ or substituents replacing them are interchanged with each other, the sugar is said to be modified at the 4′ position.

Oligonucleotides can also include abasic sugars, i.e., monomers which lack a nucleobase at C-1′ or has other chemical groups in place of a nucleobase at C1′. See for example U.S. Pat. No. 5,998,203, contents of which are herein incorporated in their entirety. These abasic sugars can also be further containing modifications at one or more of the constituent sugar atoms. Oligonucleotides can also contain one or more sugars that are the L isomer, e.g. L-nucleosides. Modification to the sugar group can also include replacement of the 4′-O with a sulfur, optionally substituted nitrogen or CH₂ group. In some embodiments, linkage between C1′ and nucleobase is in the a configuration.

Oligonucleotide modifications can also include acyclic nucleotides, wherein a C—C bonds between ribose carbons (e.g., C1′-C2′, C2′-C3′, C3′-C4′, C4′-O4′, C1′-O4′) is absent and/or at least one of ribose carbons or oxygen (e.g., C1′, C2′, C3′, C4′ or O4′) are independently or in combination absent from the nucleotide. In some embodiments, acyclic nucleotide is

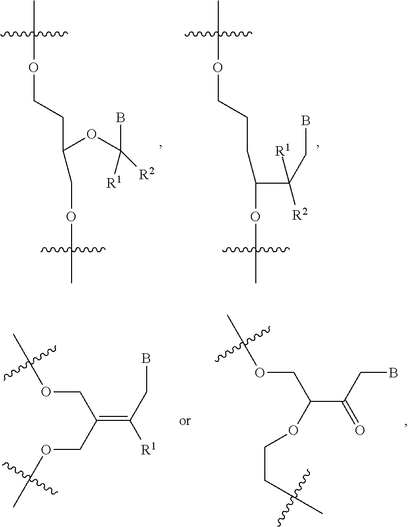

wherein B is a modified or unmodified nucleobase, R₁ and R₂ independently are H, halogen, OR₃, or alkyl; and R₃ is H, alkyl, cycloalkyl, aryl, aralkyl, heteroaryl or sugar).

Preferred sugar modifications are 2′-O-Me (2′-O-methyl), 2′-O-MOE (2′-O-methoxyethyl), 2′-F, 2′-O-[2-(methylamino)-2-oxoethyl] (2′-O-NMA), 2′-S-methyl, 2′-O—CH₂-(4′-C) (LNA), 2′-O—CH₂CH₂-(4′-C) (ENA), 2′-O-aminopropyl (2′-O-AP), 2′-O-dimethylaminoethyl (2′-O-DMAOE), 2′-O-dimethylaminopropyl (2′-O-DMAP), and 2′-O-dimethylaminoethyloxyethyl (2′-O-DMAEOE).

It is to be understood that when a particular nucleotide is linked through its 2′-position to the next nucleotide, the sugar modifications described herein can be placed at the 3′-position of the sugar for that particular nucleotide, e.g., the nucleotide that is linked through its 2′-position. A modification at the 3′ position can be present in the xylose configuration. The term “xylose configuration” refers to the placement of a substituent on the C3′ of ribose in the same configuration as the 3′-OH is in the xylose sugar.

The hydrogen attached to C4′ and/or C1′ can be replaced by a straight- or branched-optionally substituted alkyl, optionally substituted alkenyl, optionally substituted alkynyl, wherein backbone of the alkyl, alkenyl and alkynyl can contain one or more of O, S, S(O), SO₂, N(R′), C(O), N(R′)C(O)O, OC(O)N(R′), CH(R′), phosphorous containing linkage, optionally substituted aryl, optionally substituted heteroaryl, optionally substituted heterocyclic or optionally substituted cycloalkyl, where R′ is hydrogen, halogen, alkyl, alkenyl, alkynyl, alkoxy, aryl, heteroaryl, cyclyl, or heterocyclyl, each of which can be optionally substituted. In some embodiments, the hydrogen attached to the C4′ of the 5′ terminal nucleotide is replaced.

In some embodiments, C4′ and C5′ together form an optionally substituted heterocyclic, preferably comprising at least one —PX(Y)—, wherein X is H, OH, OM, SH, optionally substituted alkyl, optionally substituted alkoxy, optionally substituted alkylthio, optionally substituted alkylamino or optionally substituted dialkylamino, where M is independently for each occurrence an alki metal or transition metal with an overall charge of +1; and Y is O, S, or NR′, where R′ is hydrogen, optionally substituted aliphatic. Preferably this modification is at the 5 terminal of the oligonucleotide.

Nucleobase Modifications:

Adenine, cytosine, guanine, thymine and uracil are the most common bases (or nucleobases) found in nucleic acids. These bases can be modified or replaced to provide oligonucleotides having improved properties. For example, nuclease resistant oligonucleotides can be prepared with these bases or with synthetic and natural nucleobases (e.g., inosine, xanthine, hypoxanthine, nubularine, isoguanisine, or tubercidine) and any one of the above modifications. Alternatively, substituted or modified analogs of any of the above bases and “universal bases” can be employed. When a natural base is replaced by a non-natural and/or universal base, the nucleotide is said to comprise a modified nucleobase and/or a nucleobase modification herein. Modified nucleobase and/or nucleobase modifications also include natural, non-natural and universal bases, which comprise conjugated moieties, e.g. a ligand described herein. Preferred conjugate moieties for conjugation with nucleobases include cationic amino groups which can be conjugated to the nucleobase via an appropriate alkyl, alkenyl or a linker with an amide linkage.

An oligonucleotide can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as inosine, xanthine, hypoxanthine, nubularine, isoguanisine, tubercidine, 2-(halo)adenine, 2-(alkyl)adenine, 2-(propyl)adenine, 2-(amino)adenine, 2-(aminoalkyll)adenine, 2-(aminopropyl)adenine, 2-(methylthio)-N⁶-(isopentenyl)adenine, 6-(alkyl)adenine, 6-(methyl)adenine, 7-(deaza)adenine, 8-(alkenyl)adenine, 8-(alkyl)adenine, 8-(alkynyl)adenine, 8-(amino)adenine, 8-(halo)adenine, 8-(hydroxyl)adenine, 8-(thioalkyl)adenine, 8-(thiol)adenine, N⁶-(isopentyl)adenine, N⁶-(methyl)adenine, N⁶,N⁶-(dimethyl)adenine, 2-(alkyl)guanine, 2-(propyl)guanine, 6-(alkyl)guanine, 6-(methyl)guanine, 7-(alkyl)guanine, 7-(methyl)guanine, 7-(deaza)guanine, 8-(alkyl)guanine, 8-(alkenyl)guanine, 8-(alkynyl)guanine, 8-(amino)guanine, 8-(halo)guanine, 8-(hydroxyl)guanine, 8-(thioalkyl)guanine, 8-(thiol)guanine, N-(methyl)guanine, 2-(thio)cytosine, 3-(deaza)-5-(aza)cytosine, 3-(alkyl)cytosine, 3-(methyl)cytosine, 5-(alkyl)cytosine, 5-(alkynyl)cytosine, 5-(halo)cytosine, 5-(methyl)cytosine, 5-(propynyl)cytosine, 5-(propynyl)cytosine, 5-(trifluoromethyl)cytosine, 6-(azo)cytosine, N⁴-(acetyl)cytosine, 3-(3-amino-3-carboxypropyl)uracil, 2-(thio)uracil, 5-(methyl)-2-(thio)uracil, 5-(methylaminomethyl)-2-(thio)uracil, 4-(thio)uracil, 5-(methyl)-4-(thio)uracil, 5-(methylaminomethyl)-4-(thio)uracil, 5-(methyl)-2,4-(dithio)uracil, 5-(methylaminomethyl)-2,4-(dithio)uracil, 5-(2-aminopropyl)uracil, 5-(alkyl)uracil, 5-(alkynyl)uracil, 5-(allylamino)uracil, 5-(aminoallyl)uracil, 5-(aminoalkyl)uracil, 5-(guanidiniumalkyl)uracil, 5-(1,3-diazole-1-alkyl)uracil, 5-(cyanoalkyl)uracil, 5-(dialkylaminoalkyl)uracil, 5-(dimethylaminoalkyl)uracil, 5-(halo)uracil, 5-(methoxy)uracil, uracil-5-oxyacetic acid, 5-(methoxycarbonylmethyl)-2-(thio)uracil, 5-(methoxycarbonyl-methyl)uracil, 5-(propynyl)uracil, 5-(propynyl)uracil, 5-(trifluoromethyl)uracil, 6-(azo)uracil, dihydrouracil, N³-(methyl)uracil, 5-uracil (i.e., pseudouracil), 2-(thio)pseudouracil, 4-(thio)pseudouracil, 2,4-(dithio)psuedouracil, 5-(alkyl)pseudouracil, 5-(methyl)pseudouracil, 5-(alkyl)-2-(thio)pseudouracil, 5-(methyl)-2-(thio)pseudouracil, 5-(alkyl)-4-(thio)pseudouracil, 5-(methyl)-4-(thio)pseudouracil, 5-(alkyl)-2,4-(dithio)pseudouracil, 5-(methyl)-2,4-(dithio)pseudouracil, 1-substituted pseudouracil, 1-substituted 2(thio)-pseudouracil, 1-substituted 4-(thio)pseudouracil, 1-substituted 2,4-(dithio)pseudouracil, 1-(aminocarbonylethylenyl)-pseudouracil, 1-(aminocarbonylethylenyl)-2(thio)-pseudouracil, 1-(aminocarbonylethylenyl)-4-(thio)pseudouracil, 1-(aminocarbonylethylenyl)-2,4-(dithio)pseudouracil, 1-(aminoalkylaminocarbonylethylenyl)-pseudouracil, 1-(aminoalkylamino-carbonylethylenyl)-2(thio)-pseudouracil, 1-(aminoalkylaminocarbonylethylenyl)-4-(thio)pseudouracil, 1-(aminoalkylaminocarbonylethylenyl)-2,4-(dithio)pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-phenthiazin-1-yl, 7-substituted 1-(aza)-2-(thio)-3-(aza)-phenthiazin-1-yl, 7-(aminoalkylhydroxy)-1,3-(diaza)-2-(oxo)-phenoxazin-1-yl, 7-(aminoalkylhydroxy)-1-(aza)-2-(thio)-3-(aza)-phenoxazin-1-yl, 7-(aminoalkylhydroxy)-1,3-(diaza)-2-(oxo)-phenthiazin-1-yl, 7-(aminoalkylhydroxy)-1-(aza)-2-(thio)-3-(aza)-phenthiazin-1-yl, 7-(guanidiniumalkylhydroxy)-1,3-(diaza)-2-(oxo)-phenoxazin-1-yl, 7-(guanidiniumalkylhydroxy)-1-(aza)-2-(thio)-3-(aza)-phenoxazin-1-yl, 7-(guanidiniumalkyl-hydroxy)-1,3-(diaza)-2-(oxo)-phenthiazin-1-yl, 7-(guanidiniumalkylhydroxy)-1-(aza)-2-(thio)-3-(aza)-phenthiazin-1-yl, 1,3,5-(triaza)-2,6-(dioxa)-naphthalene, inosine, xanthine, hypoxanthine, nubularine, tubercidine, isoguanisine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl)isocarbostyrilyl, 5-(methyl)isocarbostyrilyl, 3-(methyl)-7-(propynyl)isocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenyl, tetracenyl, pentacenyl, difluorotolyl, 4-(fluoro)-6-(methyl)benzimidazole, 4-(methyl)benzimidazole, 6-(azo)thymine, 2-pyridinone, 5-nitroindole, 3-nitropyrrole, 6-(aza)pyrimidine, 2-(amino)purine, 2,6-(diamino)purine, 5-substituted pyrimidines, N²-substituted purines, N⁶-substituted purines, O⁶-substituted purines, substituted 1,2,4-triazoles, pyrrolo-pyrimidin-2-on-3-yl, 6-phenyl-pyrrolo-pyrimidin-2-on-3-yl, para-substituted-6-phenyl-pyrrolo-pyrimidin-2-on-3-yl, ortho-substituted-6-phenyl-pyrrolo-pyrimidin-2-on-3-yl, bis-ortho-substituted-6-phenyl-pyrrolo-pyrimidin-2-on-3-yl, para-(aminoalkylhydroxy)-6-phenyl-pyrrolo-pyrimidin-2-on-3-yl, ortho-(aminoalkylhydroxy)-6-phenyl-pyrrolo-pyrimidin-2-on-3-yl, bis-ortho-(aminoalkylhydroxy)-6-phenyl-pyrrolo-pyrimidin-2-on-3-yl, pyridopyrimidin-3-yl, 2-oxo-7-amino-pyridopyrimidin-3-yl, 2-oxo-pyridopyrimidine-3-yl, or any O-alkylated or N-alkylated derivatives thereof. Alternatively, substituted or modified analogs of any of the above bases and “universal bases” can be employed.

As used herein, a universal nucleobase is any modified or nucleobase that can base pair with all of the four naturally occurring nucleobases without substantially affecting the melting behavior, recognition by intracellular enzymes or activity of the oligonucleotide duplex. Some exemplary universal nucleobases include, but are not limited to, 2,4-difluorotoluene, nitropyrrolyl, nitroindolyl, 8-aza-7-deazaadenine, 4-fluoro-6-methylbenzimidazle, 4-methylbenzimidazle, 3-methyl isocarbostyrilyl, 5-methyl isocarbostyrilyl, 3-methyl-7-propynyl isocarbostyrilyl, 7-azaindolyl, 6-methyl-7-azaindolyl, imidizopyridinyl, 9-methyl-imidizopyridinyl, pyrrolopyrizinyl, isocarbostyrilyl, 7-propynyl isocarbostyrilyl, propynyl-7-azaindolyl, 2,4,5-trimethylphenyl, 4-methylinolyl, 4,6-dimethylindolyl, phenyl, napthalenyl, anthracenyl, phenanthracenyl, pyrenyl, stilbenyl, tetracenyl, pentacenyl, and structural derivatives thereof (see for example, Loakes, 2001, Nucleic Acids Research, 29, 2437-2447).

Further nucleobases include those disclosed in U.S. Pat. No. 3,687,808; those disclosed in International Application No. PCT/US09/038425, filed Mar. 26, 2009; those disclosed in the Concise Encyclopedia Of Polymer Science And Engineering, pages 858-859, Kroschwitz, J. I., ed. John Wiley & Sons, 1990; those disclosed by English et al., Angewandte Chemie, International Edition, 1991, 30, 613; those disclosed in Modified Nucleosides in Biochemistry, Biotechnology and Medicine, Herdewijin, P. Ed. Wiley-VCH, 2008; and those disclosed by Sanghvi, Y. S., Chapter 15, dsRNA Research and Applications, pages 289-302, Crooke, S. T. and Lebleu, B., Eds., CRC Press, 1993. Contents of all of the above are herein incorporated by reference.

Terminal Modifications:

In vivo applications of oligonucleotides can be limited due to presence of nucleases in the serum and/or blood. Thus in certain instances it is preferable to modify the 3′, 5′ or both ends of an oligonucleotide to make the oligonucleotide resistant against exonucleases. In some embodiments, the oligonucleotide comprises a cap structure at 3′ (3′-cap), 5′ (5′-cap) or both ends. In some embodiments, oligonucleotide comprises a 3′-cap. In another embodiment, oligonucleotide comprises a 5′-cap. In yet another embodiment, oligonucleotide comprises both a 3′ cap and a 5′ cap. It is to be understood that when an oligonucleotide comprises both a 3′ cap and a 5′ cap, such caps can be same or they can be different.

As used herein, “cap structure” refers to chemical modifications, which have been incorporated at either terminus of oligonucleotide. See for example U.S. Pat. No. 5,998,203 and International Patent Publication WO03/70918, contents of which are herein incorporated in their entireties.

Exemplary 5′-caps include, but are not limited to, ligands, 5′-5′-inverted nucleotide, 5′-5′-inverted abasic nucleotide residue, 2′-5′ linkage, 5′-amino, 5′-amino-alkyl phosphate, 5′-hexylphosphate, 5′-aminohexyl phosphate, bridging and/or non-bridging 5′-phosphoramidate, bridging and/or non-bridging 5′-phosphorothioate and/or 5′-phosphorodithioate, bridging or non bridging 5′-methylphosphonate, non-phosphodiester intersugar linkage between the end two nucleotides, 4′,5′-methylene nucleotide, I-(beta-D-erythrofuranosyl) nucleotide, 4′-thio nucleotide, carbocyclic nucleotide, 1,5-anhydrohexitol nucleotide, L-nucleotides, alpha-nucleotides, modified nucleobase nucleotide, phosphorodithioate linkage, threo-pentofuranosyl nucleotide, acyclic nucleotide, acyclic 3,4-dihydroxybutyl nucleotide, acyclic 3,5-dihydroxypentyl nucleotide, 5′-mercapto nucleotide and 5′-1,4-butanediol phosphate.

Exemplary 3′-caps include, but are not limited to, ligands, 3′-3′-inverted nucleotide, 3′-3′-inverted abasic nucleotide residue, 3′-2′-inverted nucleotide moiety, 3′-2′-inverted abasic moiety, 2′-5′-linkage, 3′-amino, 3′-amino-alkyl phosphate, 3′-hexylphosphate, 3′-aminohexyl phosphate, bridging and/or non-bridging 3′-phosphoramidate, bridging and/or non-bridging 3′-phosphorothioate and/or 3′-phosphorodithioate, bridging or non bridging 3′-methylphosphonate, non-phosphodiester intersugar linkage between the end two nucleotides, I-(beta-D-erythrofuranosyl) nucleotide, 4′-thio nucleotide, carbocyclic nucleotide, 1,5-anhydrohexitol nucleotide, L-nucleotides, alpha-nucleotides, modified nucleobase nucleotide, phosphorodithioate linkage, threo-pentofuranosyl nucleotide, acyclic nucleotide, acyclic 3,4-dihydroxybutyl nucleotide, acyclic 3,5-dihydroxypentyl nucleotide, and 3′-1,4-butanediol phosphate. For more details see Beaucage and Iyer, 1993, Tetrahedron 49, 1925, incorporated by reference herein.

Other 3′ and/or 5′ caps amenable to the invention are described in U.S. Provisional Application No. 61/223,665, filed Jul. 7, 2009, contents of which are herein incorporated in their entirety.

The 3′ and/or 5′ ends of an oligonucleotide can also be conjugated to other functional molecular entities such as labeling moieties, e.g., fluorophore (e.g., pyrene, TAMRA, fluorescein, Cy3 or Cy5 dyes) or protecting groups (based e.g., on sulfur, silicon, boron or ester). The functional molecular entities can be attached to the sugar through a phosphate group and/or a linker. The terminal atom of the linker can connect to or replace the linking atom of the phosphate group or the C-3′ or C-5′ O, N, S or C group of the sugar. Alternatively, the linker can connect to or replace the terminal atom of a nucleotide surrogate (e.g., PNAs).

Terminal modifications useful for modulating activity include modification of the 5′ end with phosphate or phosphate analogs. For example, in some embodiments the 5′ end of the oligonucleotide can be phosphorylated or includes a phosphoryl analog at the 5′ terminus. The 5′-phosphate modifications can include those which are compatible with RISC mediated gene silencing. Modifications at the 5′-terminal end can also be useful in stimulating or inhibiting the immune system of a subject. In some embodiments, the 5′-end of the oligonucleotide comprises the modification

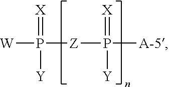

wherein W, X and Y are each independently selected from the group consisting of O, OR (R is hydrogen, alkyl, aryl), S, Se, BR₃ (R is hydrogen, alkyl, aryl), BH₃⁻, C (i.e. an alkyl group, an aryl group, etc. . . . ), H, NR₂ (R is hydrogen, alkyl, aryl), or OR (R is hydrogen, alkyl or aryl); A and Z are each independently for each occurrence absent, O, S, CH₂, NR (R is hydrogen, alkyl, aryl), or optionally substituted alkylene, wherein backbone of the alkylene can comprise one or more of O, S, SS and NR (R is hydrogen, alkyl, aryl) internally and/or at the end; and n is 0-2. In some embodiments n is 1 or 2. It is understood that A is replacing the oxygen linked to 5′ carbon of sugar.

In some embodiments, one or both hydrogen on C5′ of the 5′-terminal nucleotides can be replaced with a halogen, e.g., F.

Exemplary 5′-modificaitons include, but are not limited to, 5′-monophosphate ((HO)₂(O)P—O-5); 5′-diphosphate ((HO)₂(O)P—O—P(HO)(O)—O-5′); 5′-triphosphate ((HO)₂(O)P—O—(HO)(O)P—O—P(HO)(O)—O-5′); 5′-monothiophosphate (phosphorothioate; (HO)2(S)P—O-5′); 5′-monodithiophosphate (phosphorodithioate; (HO)(HS)(S)P—O-5′), 5′-phosphorothiolate ((HO)2(O)P—S-5′); 5′-alpha-thiotriphosphate; 5′-beta-thiotriphosphate; 5′-gamma-thiotriphosphate; 5′-phosphoramidates ((HO)₂(O)P—NH-5′, (HO)(NH₂)(O)P—O-5′). Other 5′-modification include 5′-alkylphosphonates (R(OH)(O)P—O-5′, R=alkyl, e.g., methyl, ethyl, isopropyl, propyl, etc. . . . ), 5′-alkyletherphosphonates (R(OH)(O)P—O-5′, R=alkylether, e.g., methoxymethyl (CH₂OMe), ethoxymethyl, etc. . . . ). Other exemplary 5′-modifications include where Z is optionally substituted alkyl at least once, e.g., ((HO)₂(X)P—O[—(CH₂)ₐ—O—P(X)(OH)—O]_{b}-5′, ((HO)2(X)P—O[—(CH₂)ₐ—O—P(X)(OH)—O]_{b}-5′, ((HO)₂(X)P—[—(CH₂)ₐ—O—P(X)(OH)—O]_{b}-5′; dialkyl terminal phosphates and phosphate mimics: HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein a and b are each independently 1-10. Other embodiments include replacement of oxygen and/or sulfur with BH₃, BH₃⁻ and/or Se.

Terminal modifications can also be useful for monitoring distribution, and in such cases the preferred groups to be added include fluorophores, e.g., fluorescein or an Alexa dye, e.g., Alexa 488. Terminal modifications can also be useful for enhancing uptake, useful modifications for this include targeting ligands. Terminal modifications can also be useful for cross-linking an oligonucleotide to another moiety; modifications useful for this include mitomycin C, psoralen, and derivatives thereof.

Ligands:

A wide variety of entities, e.g., ligands, can be coupled to the oligonucleotides described herein. Ligands can include naturally occurring molecules, or recombinant or synthetic molecules. Exemplary ligands include, but are not limited to, polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG, e.g., PEG-2K, PEG-5K, PEG-10K, PEG-12K, PEG-15K, PEG-20K, PEG-40K), MPEG, [MPEG]₂, polyvinyl alcohol (PVA), polyurethane, poly(2-ethylacryllic acid), N-isopropylacrylamide polymers, polyphosphazine, polyethylenimine, cationic groups, spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, thyrotropin, melanotropin, lectin, glycoprotein, surfactant protein A, mucin, glycosylated polyaminoacids, transferrin, bisphosphonate, polyglutamate, polyaspartate, aptamer, asialofetuin, hyaluronan, procollagen, immunoglobulins (e.g., antibodies), insulin, transferrin, albumin, sugar-albumin conjugates, intercalating agents (e.g., acridines), cross-linkers (e.g. psoralen, mitomycin C), porphyrins (e.g., TPPC4, texaphyrin, Sapphyrin), polycyclic aromatic hydrocarbons (e.g., phenazine, dihydrophenazine), artificial endonucleases (e.g., EDTA), lipophilic molecules (e.g, steroids, bile acids, cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine), peptides (e.g., an alpha helical peptide, amphipathic peptide, RGD peptide, cell permeation peptide, endosomolytic/fusogenic peptide), alkylating agents, phosphate, amino, mercapto, polyamino, alkyl, substituted alkyl, radiolabeled markers, enzymes, haptens (e.g. biotin), transport/absorption facilitators (e.g., naproxen, aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, AP, antibodies, hormones and hormone receptors, lectins, carbohydrates, multivalent carbohydrates, vitamins (e.g., vitamin A, vitamin E, vitamin K, vitamin B, e.g., folic acid, B12, riboflavin, biotin and pyridoxal), vitamin cofactors, lipopolysaccharide, an activator of p38 MAP kinase, an activator of NF-κB, taxon, vincristine, vinblastine, cytochalasin, nocodazole, japlakinolide, latrunculin A, phalloidin, swinholide A, indanocine, myoservin, tumor necrosis factor alpha (TNFalpha), interleukin-1 beta, gamma interferon, natural or recombinant low density lipoprotein (LDL), natural or recombinant high-density lipoprotein (HDL), and a cell-permeation agent (e.g., a.helical cell-permeation agent).

Peptide and peptidomimetic ligands include those having naturally occurring or modified peptides, e.g., D or L peptides; α, β, or γ peptides; N-methyl peptides; azapeptides; peptides having one or more amide, i.e., peptide, linkages replaced with one or more urea, thiourea, carbamate, or sulfonyl urea linkages; or cyclic peptides. A peptidomimetic (also referred to herein as an oligopeptidomimetic) is a molecule capable of folding into a defined three-dimensional structure similar to a natural peptide. The peptide or peptidomimetic ligand can be about 5-50 amino acids long, e.g., about 5, 10, 15, 20, 25, 30, 35, 40, 45, or 50 amino acids long.

Exemplary amphipathic peptides include, but are not limited to, cecropins, lycotoxins, paradaxins, buforin, CPF, bombinin-like peptide (BLP), cathelicidins, ceratotoxins, S. clava peptides, hagfish intestinal antimicrobial peptides (HFIAPs), magainines, brevinins-2, dermaseptins, melittins, pleurocidin, H₂A peptides, Xenopus peptides, esculentinis-1, and caerins.

As used herein, the term “endosomolytic ligand” refers to molecules having endosomolytic properties. Endosomolytic ligands promote the lysis of and/or transport of the composition of the invention, or its components, from the cellular compartments such as the endosome, lysosome, endoplasmic reticulum (ER), golgi apparatus, microtubule, peroxisome, or other vesicular bodies within the cell, to the cytoplasm of the cell. Some exemplary endosomolytic ligands include, but are not limited to, imidazoles, poly or oligoimidazoles, linear or branched polyethyleneimines (PEIs), linear and brached polyamines, e.g. spermine, cationic linear and branched polyamines, polycarboxylates, polycations, masked oligo or poly cations or anions, acetals, polyacetals, ketals/polyketals, orthoesters, linear or branched polymers with masked or unmasked cationic or anionic charges, dendrimers with masked or unmasked cationic or anionic charges, polyanionic peptides, polyanionic peptidomimetics, pH-sensitive peptides, natural and synthetic fusogenic lipids, natural and synthetic cationic lipids.

Exemplary endosomolytic/fusogenic peptides include, but are not limited to,

### SEQ ID NO: 195, AALEALAEALEALAEALEALAEAAAAGGC (GALA);

### SEQ ID NO: 153, AALAEALAEALAEALAEALAEALAAAAGGC (EALA);

### SEQ ID NO: 154, ALEALAEALEALAEA;

### SEQ ID NO: 155, GLFEAIEGFIENGWEGMIWDYG (INF-7);

### SEQ ID NO: 156, GLFGAIAGFIENGWEGMIDGWYG (Inf HA-2);

SEQ ID NO: 157, GLFEAIEGFIENGWEGMIDGWYGCGLFEAIEGFIENGWEGMID GWYGC (diINF-7);
SEQ ID NO: 158, GLFEAIEGFIENGWEGMIDGGCGLFEAIEGFIENGWEGMIDGGC (diINF-3);

### SEQ ID NO: 159, GLFGALAEALAEALAEHLAEALAEALEALAAGGSC (GLF);

### SEQ ID NO: 160, GLFEAIEGFIENGWEGLAEALAEALEALAAGGSC (GALA-INF3);

SEQ ID NO: 161, GLF EAI EGFI ENGW EGnI DG K GLF EAI EGFI ENGW EGnI DG (INF-5, n is norleucine);

### SEQ ID NO: 162, LFEALLELLESLWELLLEA (JTS-1);

SEQ ID NO: 163, GLFKALLKLLKSLWKLLLKA (ppTG1);
SEQ ID NO: 164, GLFRALLRLLRSLWRLLLRA (ppTG20);

### SEQ ID NO: 165, WEAKLAKALAKALAKHLAKALAKALKACEA (KALA);

### SEQ ID NO: 166, GLFFEAIAEFIEGGWEGLIEGC (HA);

### SEQ ID NO: 167, GIGAVLKVLTTGLPALISWIKRKRQQ (Melittin);

### SEQ ID NO: 168, H₅WYG; and

### SEQ ID NO: 169, CHK₆HC.

Without wishing to be bound by theory, fusogenic lipids fuse with and consequently destabilize a membrane. Fusogenic lipids usually have small head groups and unsaturated acyl chains. Exemplary fusogenic lipids include, but are not limited to, 1,2-dileoyl-sn-3-phosphoethanolamine (DOPE), phosphatidylethanolamine (POPE), palmitoyloleoylphosphatidylcholine (POPC), (6Z,9Z,28Z,31Z)-heptatriaconta-6,9,28,31-tetraen-19-ol (Di-Lin), N-methyl(2,2-di((9Z,12Z)-octadeca-9,12-dienyl)-1,3-dioxolan-4-yl)methanamine (DLin-k-DMA) and N-methyl-2-(2,2-di((9Z,12Z)-octadeca-9,12-dienyl)-1,3-dioxolan-4-yl)ethanamine.

Synthetic polymers with endosomolytic activity amenable to the present invention are described in U.S. Pat. App. Pub. Nos. 2009/0048410; 2009/0023890; 2008/0287630; 2008/0287628; 2008/0281044; 2008/0281041; 2008/0269450; 2007/0105804; 2007/0036865; and 2004/0198687, content of all of is incorporated herein by reference in its entirety.

Exemplary cell permeation peptides include, but are not limited to,

SEQ ID NO: 170, RQIKIWFQNRRMKWKK (penetratin);
SEQ ID NO: 171, GRKKRRQRRRPPQC (Tat fragment 48-60);
SEQ ID NO: 172, GALFLGWLGAAGSTMGAWSQPKKKRKV (signal sequence based peptide);

### SEQ ID NO: 173, LLIILRRRIRKQAHAHSK (PVEC);

SEQ ID NO: 174, GWTLNSAGYLLKINLKALAALAKKIL (transportan);
SEQ ID NO: 175, KLALKLALKALKAALKLA (amphiphilic model peptide);

### SEQ ID NO: 176, RRRRRRRRR (Arg9);

SEQ ID NO: 177, KFFKFFKFFK (Bacterial cell wall permeating peptide);

### SEQ ID NO: 178, LLGDFFRKSKEKIGKEFKRIVQRIKDFLRNLVPRTES (LL-37);

SEQ ID NO: 179, SWLSKTAKKLENSAKKRISEGIAIAIQGGPR (cecropin P1);
SEQ ID NO: 180, ACYCRIPACIAGERRYGTCIYQGRLWAFCC (α-defensin)
SEQ ID NO: 181, DHYNCVSSGGQCLYSACPIFTKIQGTCYRGKAKCCK (β-defensin);

### SEQ ID NO: 182, RRRPRPPYLPRPRPPPFFPPRLPPRIPPGFPPRFPPRFPGKR-NH2 (PR-39);

SEQ ID NO: 183, ILPWKWPWWPWRR-NH2 (indolicidin);

### SEQ ID NO: 184, AAVALLPAVLLALLAP (RFGF);

SEQ ID NO: 185, AALLPVLLAAP (RFGF analogue); and
SEQ ID NO: 186, RKCRIVVIRVCR (bactenecin).

Exemplary cationic groups include, but are not limited to, protonated amino groups, derived from e.g., O-AMINE (AMINE=NH₂; alkylamino, dialkylamino, heterocyclyl, arylamino, diaryl amino, heteroaryl amino, or diheteroaryl amino, ethylene diamine, polyamino); aminoalkoxy, e.g., O(CH₂)ₙAMINE, (e.g., AMINE=NH₂; alkylamino, dialkylamino, heterocyclyl, arylamino, diaryl amino, heteroaryl amino, or diheteroaryl amino, ethylene diamine, polyamino); amino (e.g. NH₂; alkylamino, dialkylamino, heterocyclyl, arylamino, diaryl amino, heteroaryl amino, diheteroaryl amino, or amino acid); and NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino).

As used herein the term “targeting ligand” refers to any molecule that provides an enhanced affinity for a selected target, e.g., a cell, cell type, tissue, organ, region of the body, or a compartment, e.g., a cellular, tissue or organ compartment. Some exemplary targeting ligands include, but are not limited to, antibodies, antigens, folates, receptor ligands, carbohydrates, aptamers, integrin receptor ligands, chemokine receptor ligands, transferrin, biotin, serotonin receptor ligands, PSMA, endothelin, GCPII, somatostatin, LDL and HDL ligands.

Carbohydrate based targeting ligands include, but are not limited to, D-galactose, multivalent galactose, N-acetyl-D-galactose (GalNAc), multivalent GalNAc, e.g. GalNAc2 and GalNAc3; D-mannose, multivalent mannose, multivalent lactose, N-acetyl-galactosamine, N-acetyl-gulucosamine, multivalent fucose, glycosylated polyaminoacids and lectins. The term multivalent indicates that more than one monosaccharide unit is present. Such monosaccharide subunits can be linked to each other through glycosidic linkages or linked to a scaffold molecule.

A number of folate and folate analogs amenable to the present invention as ligands are described in U.S. Pat. Nos. 2,816,110; 51,410,104; 5,552,545; 6,335,434 and 7,128,893, contents of which are herein incorporated in their entireties by reference.

As used herein, the terms “PK modulating ligand” and “PK modulator” refers to molecules which can modulate the pharmacokinetics of the oligoncucleotide. Some exemplary PK modulator include, but are not limited to, lipophilic molecules, bile acids, sterols, phospholipid analogues, peptides, protein binding agents, vitamins, fatty acids, phenoxazine, aspirin, naproxen, ibuprofen, suprofen, ketoprofen, (S)-(+)-pranoprofen, carprofen, PEGs, biotin, and transthyretia-binding ligands (e.g., tetraiidothyroacetic acid, 2,4,6-triiodophenol and flufenamic acid). Oligonucleotides that comprise a number of phosphorothioate intersugar linkages are also known to bind to serum protein, thus short oligonucleotides, e.g. oligonucleotides of comprising from about 5 to 30 nucleotides (e.g., 5 to 25 nulceotides, preferably 5 to 20 nucleotides, e.g., 5, 6, 7, 8, 9, 10, 11, 12, 13, 14, 15, 16, 17, 18, 19, or 20 nucleotides), and that comprise a plurality of phosphorothioate linkages in the backbone are also amenable to the present invention as ligands (e.g. as PK modulating ligands). The PK modulating oligonucleotide can comprise at least 3, 4, 5, 6, 7, 8, 9, 10, 11, 12, 13, 14, 15 or more phosphorothioate and/or phosphorodithioate linkages. In some embodiments, all internucleotide linkages in PK modulating oligonucleotide are phosphorothioate and/or phosphorodithioates linkages. In addition, aptamers that bind serum components (e.g. serum proteins) are also amenable to the present invention as PK modulating ligands. Binding to serum components (e.g. serum proteins) can be predicted from albumin binding assays, scuh as those described in Oravcova, et al., Journal of Chromatography B (1996), 677: 1-27.

When two or more ligands are present, the ligands can all have same properties, all have different properties or some ligands have the same properties while others have different properties. For example, a ligand can have targeting properties, have endosomolytic activity or have PK modulating properties. In a preferred embodiment, all the ligands have different properties.

Ligands can be coupled to the oligonucleotides at various places, for example, 3′-end, 5′-end, and/or at an internal position. When two or more ligands are present, the ligand can be on opposite ends of an oligonucleotide. In preferred embodiments, the ligand is attached to the oligonucleotides via an intervening tether/linker. The ligand or tethered ligand can be present on a monomer when said monomer is incorporated into the growing strand. In some embodiments, the ligand can be incorporated via coupling to a “precursor” monomer after said “precursor” monomer has been incorporated into the growing strand. For example, a monomer having, e.g., an amino-terminated tether (i.e., having no associated ligand), e.g., monomer-linker-NH₂ can be incorporated into a growing oligonucleotide strand. In a subsequent operation, i.e., after incorporation of the precursor monomer into the strand, a ligand having an electrophilic group, e.g., a pentafluorophenyl ester or aldehyde group, can subsequently be attached to the precursor monomer by coupling the electrophilic group of the ligand with the terminal nucleophilic group of the precursor monomer's tether.

In another example, a monomer having a chemical group suitable for taking part in Click Chemistry reaction can be incorporated e.g., an azide or alkyne terminated tether/linker. In a subsequent operation, i.e., after incorporation of the precursor monomer into the strand, a ligand having complementary chemical group, e.g. an alkyne or azide can be attached to the precursor monomer by coupling the alkyne and the azide together.

For double-stranded oligonucleotides, ligands can be attached to one or both strands.

In some embodiments, ligand can be conjugated to nucleobases, sugar moieties, or internucleosidic linkages of nucleic acid molecules. Conjugation to purine nucleobases or derivatives thereof can occur at any position including, endocyclic and exocyclic atoms. In some embodiments, the 2-, 6-, 7-, or 8-positions of a purine nucleobase are attached to a conjugate moiety. Conjugation to pyrimidine nucleobases or derivatives thereof can also occur at any position. In some embodiments, the 2-, 5-, and 6-positions of a pyrimidine nucleobase can be substituted with a conjugate moiety. When a ligand is conjugated to a nucleobase, the preferred position is one that does not interfere with hybridization, i.e., does not interfere with the hydrogen bonding interactions needed for base pairing.

Conjugation to sugar moieties of nucleosides can occur at any carbon atom. Example carbon atoms of a sugar moiety that can be attached to a conjugate moiety include the 2′, 3′, and 5′ carbon atoms. The 1′ position can also be attached to a conjugate moiety, such as in an abasic residue. Internucleosidic linkages can also bear conjugate moieties. For phosphorus-containing linkages (e.g., phosphodiester, phosphorothioate, phosphorodithiotate, phosphoroamidate, and the like), the conjugate moiety can be attached directly to the phosphorus atom or to an O, N, or S atom bound to the phosphorus atom. For amine- or amide-containing internucleosidic linkages (e.g., PNA), the conjugate moiety can be attached to the nitrogen atom of the amine or amide or to an adjacent carbon atom.

There are numerous methods for preparing conjugates of oligomeric compounds. Generally, an oligomeric compound is attached to a conjugate moiety by contacting a reactive group (e.g., OH, SH, amine, carboxyl, aldehyde, and the like) on the oligomeric compound with a reactive group on the conjugate moiety. In some embodiments, one reactive group is electrophilic and the other is nucleophilic.

For example, an electrophilic group can be a carbonyl-containing functionality and a nucleophilic group can be an amine or thiol. Methods for conjugation of nucleic acids and related oligomeric compounds with and without linking groups are well described in the literature such as, for example, in Manoharan in Antisense Research and Applications, Crooke and LeBleu, eds., CRC Press, Boca Raton, Fla., 1993, Chapter 17, which is incorporated herein by reference in its entirety.

Representative U.S. patents that teach the preparation of oligonucleotide conjugates include, but are not limited to, U.S. Pat. Nos. 4,828,979; 4,948,882; 5,218, 105; 5,525,465; 5,541,313; 5,545,730; 5,552,538; 5,578, 717, 5,580,731; 5,580,731; 5,591,584; 5,109,124; 5,118, 802; 5,138,045; 5,414,077; 5,486,603; 5,512,439; 5,578, 718; 5,608,046; 4,587,044; 4,605,735; 4,667,025; 4,762, 779; 4,789,737; 4,824,941; 4,835,263; 4,876,335; 4,904, 582; 4,958,013; 5,082,830; 5,112,963; 5,214,136; 5,082, 830; 5,112,963; 5,149,782; 5,214,136; 5,245,022; 5,254, 469; 5,258,506; 5,262,536; 5,272,250; 5,292,873; 5,317,098; 5,371,241, 5,391,723; 5,416,203, 5,451,463; 5,510, 475; 5,512,667; 5,514,785; 5,565,552; 5,567,810; 5,574, 142; 5,585,481; 5,587,371; 5,595,726; 5,597,696; 5,599, 923; 5,599,928; 5,672,662; 5,688,941; 5,714,166; 6,153, 737; 6,172,208; 6,300,319; 6,335,434; 6,335,437; 6,395, 437; 6,444,806; 6,486,308; 6,525,031; 6,528,631; 6,559, 279; content all of which is incorporated by reference in its entirety.

Ligand Carriers:

In some embodiments, the ligands, e.g. endosomolytic ligands, targeting ligands or other ligands, are linked to a monomer which is then incorporated into the growing oligonucleotide strand during chemical synthesis. Such monomers are also referred to as carrier monomers herein. The carrier monomer is a cyclic group or acyclic group; preferably, the cyclic group is selected from the group consisting of pyrrolidinyl, pyrazolinyl, pyrazolidinyl, imidazolinyl, imidazolidinyl, piperidinyl, piperazinyl, [1,3]-dioxolane, oxazolidinyl, isoxazolidinyl, morpholinyl, thiazolidinyl, isothiazolidinyl, quinoxalinyl, pyridazinonyl, tetrahydrofuryl and decalin; preferably, the acyclic group is selected from serinol backbone or diethanolamine backbone. In some embodiments, the cyclic carrier monomer is based on pyrrolidinyl such as 4-hydroxyproline or a derivative thereof.

Exemplary ligands and ligand conjugated monomers amenable to the invention are described in U.S. patent application Ser. No. 10/916,185, filed Aug. 10, 2004; Ser. No. 10/946,873, filed Sep. 21, 2004; Ser. No. 10/985,426, filed Nov. 9, 2004; Ser. No. 10/833,934, filed Aug. 3, 2007; Ser. No. 11/115,989 filed Apr. 27, 2005, Ser. No. 11/119,533, filed Apr. 29, 2005; Ser. No. 11/197,753, filed Aug. 4, 2005; Ser. No. 11/944,227, filed Nov. 21, 2007; Ser. No. 12/328,528, filed Dec. 4, 2008; and Ser. No. 12/328,537, filed Dec. 4, 2008, contents which are herein incorporated in their entireties by reference for all purposes. Ligands and ligand conjugated monomers amenable to the invention are also described in International Application Nos. PCT/US04/001461, filed Jan. 21, 2004; PCT/US04/010586, filed Apr. 5, 2004; PCT/US04/011255, filed Apr. 9, 2005; PCT/US05/014472, filed Apr. 27, 2005; PCT/US05/015305, filed Apr. 29, 2005; PCT/US05/027722, filed Aug. 4, 2005; PCT/US08/061289, filed Apr. 23, 2008; PCT/US08/071576, filed Jul. 30, 2008; PCT/US08/085574, filed Dec. 4, 2008 and PCT/US09/40274, filed Apr. 10, 2009, contents which are herein incorporated in their entireties by reference for all purposes.

Linkers:

In some embodiments, the covalent linkages between the oligonucleotide and other components, e.g. a ligand or a ligand carrying monomer can be mediated by a linker. This linker can be cleavable linker or non-cleavable linker, depending on the application. As used herein, a “cleavable linker” refers to linkers that are capable of cleavage under various conditions. Conditions suitable for cleavage can include, but are not limited to, pH, UV irradiation, enzymatic activity, temperature, hydrolysis, elimination and substitution reactions, redox reactions, and thermodynamic properties of the linkage. In some embodiments, a cleavable linker can be used to release the oligonucleotide after transport to the desired target. The intended nature of the conjugation or coupling interaction, or the desired biological effect, will determine the choice of linker group.

As used herein, the term “linker” means an organic moiety that connects two parts of a compound. Linkers typically comprise a direct bond or an atom such as oxygen or sulfur, a unit such as NR¹, C(O), C(O)NH, SO, SO₂, SO₂NH or a chain of atoms, such as substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, where one or more methylenes can be interrupted or terminated by O, S, S(O), SO₂, N(R¹)₂, C(O), cleavable linking group, substituted or unsubstituted aryl, substituted or unsubstituted heteroaryl, substituted or unsubstituted heterocyclic; where R¹ is hydrogen, acyl, aliphatic or substituted aliphatic.

In some embodiments, the linker is a branched linker. The branchpoint of the branched linker may be at least trivalent, but can be a tetravalent, pentavalent or hexavalent atom, or a group presenting such multiple valencies. In some embodiments, the branchpoint is —N, —N(Q)-C, —O—C, —S—C, —SS—C, —C(O)N(Q)-C, —OC(O)N(Q)-C, —N(Q)C(O)—C, or —N(Q)C(O)O—C; wherein Q is independently for each occurrence H or optionally substituted alkyl. In some embodiments, the branchpoint is glycerol or derivative thereof.

Cleavable Linking Groups:

A cleavable linking group is one which is sufficiently stable outside the cell, but which upon entry into a target cell is cleaved to release the two parts the linker is holding together. In a preferred embodiment, the cleavable linking group is cleaved at least 10 times or more, preferably at least 100 times faster in the target cell or under a first reference condition (which can, e.g., be selected to mimic or represent intracellular conditions) than in the blood or serum of a subject, or under a second reference condition (which can, e.g., be selected to mimic or represent conditions found in the blood or serum).

Cleavable linking groups are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; amidases; endosomes or agents that can create an acidic environment, e.g., those that result in a pH of five or lower; enzymes that can hydrolyze or degrade an acid cleavable linking group by acting as a general acid, peptidases (which can be substrate specific) and proteases, and phosphatases.

A linker can include a cleavable linking group that is cleavable by a particular enzyme. The type of cleavable linking group incorporated into a linker can depend on the cell to be targeted. For example, liver targeting ligands can be linked to the cationic lipids through a linker that includes an ester group. Liver cells are rich in esterases, and therefore the linker will be cleaved more efficiently in liver cells than in cell types that are not esterase-rich. Other cell-types rich in esterases include cells of the lung, renal cortex, and testis.

Linkers that contain peptide bonds can be used when targeting cell types rich in peptidases, such as liver cells and synoviocytes.

In some embodiments, cleavable linking group is cleaved at least 1.25, 1.5, 1.75, 2, 3, 4, 5, 10, 25, 50, or 100 times faster in the cell (or under in vitro conditions selected to mimic intracellular conditions) as compared to blood or serum (or under in vitro conditions selected to mimic extracellular conditions). In some embodiments, the cleavable linking group is cleaved by less than 90%, 80%, 70%, 60%, 50%, 40%, 30%, 20%, 10%, 5%, or 1% in the blood (or in vitro conditions selected to mimic extracellular conditions) as compared to in the cell (or under in vitro conditions selected to mimic intracellular conditions)

Exemplary cleavable linking groups include, but are not limited to, redox cleavable linking groups (e.g., —S—S— and —C(R)₂—S—S—, wherein R is H or C₁-C₆ alkyl and at least one R is C₁-C₆ alkyl such as CH₃ or CH₂CH₃); phosphate-based cleavable linking groups (e.g., —O—P(O)(OR)—O—, —O—P(S)(OR)—O—, —O—P(S)(SR)—O—, —S—P(O)(OR)—O—, —O—P(O)(OR)—S—, —S—P(O)(OR)—S—, —O—P(S)(ORk)-S—, —S—P(S)(OR)—O—, —O—P(O)(R)—O—, —O—P(S)(R)—O—, —S—P(O)(R)—O—, —S—P(S)(R)—O—, —S—P(O)(R)—S—, —O—P(S)(R)—S—, —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O—, —S—P(S)(H)—O—, —S—P(O)(H)—S—, and —O—P(S)(H)—S—, wherein R is optionally substituted linear or branched C₁-C₁₀ alkyl); acid celavable linking groups (e.g., hydrazones, esters, and esters of amino acids, —C═NN— and —OC(O)—); ester-based cleavable linking groups (e.g., —C(O)O—); peptide-based cleavable linking groups, (e.g., linking groups that are cleaved by enzymes such as peptidases and proteases in cells, e.g., —NHCHR^{A}C(O)NHCHR^{B}C(O)—, where R^{A} and R^{B} are the R groups of the two adjacent amino acids). A peptide based cleavable linking group comprises two or more amino acids. In some embodiments, the peptide-based cleavage linkage comprises the amino acid sequence that is the substrate for a peptidase or a protease found in cells.

In some embodiments, an acid cleavable linking group is cleaveable in an acidic environment with a pH of about 6.5 or lower (e.g., about 6.5, 6.0, 5.5, 5.0, or lower), or by agents such as enzymes that can act as a general acid.

General References:

General references for oligonucleotide modification are discussed below. The anti-miR-425 and miR-425 agents and oligonucleotides used in accordance with this invention can be synthesized with solid phase synthesis, see for example “Oligonucleotide synthesis, a practical approach”, Ed. M. J. Gait, IRL Press, 1984; “Oligonucleotides and Analogues, A Practical Approach”, Ed. F. Eckstein, IRL Press, 1991 (especially Chapter 1, Modern machine-aided methods of oligodeoxyribonucleotide synthesis, Chapter 2, Oligoribonucleotide synthesis, Chapter 3, 2′-O-Methyloligoribonucleotides: synthesis and applications, Chapter 4, Phosphorothioate oligonucleotides, Chapter 5, Synthesis of oligonucleotide phosphorodithioates, Chapter 6, Synthesis of oligo-2′-deoxyribonucleoside methylphosphonates, and. Chapter 7, Oligodeoxynucleotides containing modified bases. Other particularly useful synthetic procedures, reagents, blocking groups and reaction conditions are described in Martin, P., Helv. Chim. Acta, 1995, 78, 486-504; Beaucage, S. L. and Iyer, R. P., Tetrahedron, 1992, 48, 2223-2311 and Beaucage, S. L. and Iyer, R. P., Tetrahedron, 1993, 49, 6123-6194, or references referred to therein. Modification described in WO 00/44895, WO01/75164, or WO02/44321 can be used herein. The disclosure of all publications, patents, and published patent applications listed herein are hereby incorporated by reference.

Modification of the Phosphate Group References:

The preparation of phosphinate oligonucleotides is described in U.S. Pat. No. 5,508,270. The preparation of alkyl phosphonate oligonucleotides is described in U.S. Pat. No. 4,469,863. The preparation of phosphoramidite oligonucleotides is described in U.S. Pat. No. 5,256,775 or U.S. Pat. No. 5,366,878. The preparation of phosphotriester oligonucleotides is described in U.S. Pat. No. 5,023,243. The preparation of boranophosphate oligonucleotide is described in U.S. Pat. Nos. 5,130,302 and 5,177,198. The preparation of 3′-Deoxy-3′-amino phosphoramidate oligonucleotides is described in U.S. Pat. No. 5,476,925. 3′-Deoxy-3′-methylenephosphonate oligonucleotides is described in An, H, et al. J. Org. Chem. 2001, 66, 2789-2801. Preparation of sulfur bridged nucleotides is described in Sproat et al. Nucleosides Nucleotides 1988, 7,651 and Crosstick et al. Tetrahedron Lett. 1989, 30, 4693.

Replacement of the Phosphate Group References:

Methylenemethylimino linked oligonucleosides, also identified herein as MMI linked oligonucleosides, methylenedimethylhydrazo linked oligonucleosides, also identified herein as MDH linked oligonucleosides, and methylenecarbonylamino linked oligonucleosides, also identified herein as amide-3 linked oligonucleosides, and methyleneaminocarbonyl linked oligonucleosides, also identified herein as amide-4 linked oligonucleosides as well as mixed intersugar linkage compounds having, as for instance, alternating MMI and PO or PS linkages can be prepared as is described in U.S. Pat. Nos. 5,378,825, 5,386,023, 5,489,677 and in International Application Nos. PCT/US92/04294 and PCT/US92/04305 (published as WO 92/20822 WO and 92/20823, respectively). Formacetal and thioformacetal linked oligonucleosides can be prepared as is described in U.S. Pat. Nos. 5,264,562 and 5,264,564. Ethylene oxide linked oligonucleosides can be prepared as is described in U.S. Pat. No. 5,223,618. Siloxane replacements are described in Cormier, J. F. et al. Nucleic Acids Res. 1988, 16, 4583. Carbonate replacements are described in Tittensor, J. R. J. Chem. Soc. C 1971, 1933. Carboxymethyl replacements are described in Edge, M. D. et al. J. Chem. Soc. Perkin Trans. 1 1972, 1991. Carbamate replacements are described in Stirchak, E. P. Nucleic Acids Res. 1989, 17, 6129.

Replacement of the Phosphate-Ribose Backbone References:

Cyclobutyl sugar surrogate compounds can be prepared as is described in U.S. Pat. No. 5,359,044. Pyrrolidine sugar surrogate can be prepared as is described in U.S. Pat. No. 5,519,134. Morpholino sugar surrogates can be prepared as is described in U.S. Pat. Nos. 5,142,047 and 5,235,033, and other related patent disclosures. Peptide Nucleic Acids (PNAs) are known per se and can be prepared in accordance with any of the various procedures referred to in Peptide Nucleic Acids (PNA): Synthesis, Properties and Potential Applications, Bioorganic & Medicinal Chemistry, 1996, 4, 5-23. They can also be prepared in accordance with U.S. Pat. No. 5,539,083.

Sugar Modification References:

Modifications to the 2′ modifications can be found in Verma, S. et al. Annu. Rev. Biochem. 1998, 67, 99-134 and all references therein. Specific modifications to the ribose can be found in the following references: 2′-fluoro (Kawasaki et. al., J. Med. Chem., 1993, 36, 831-841), 2′-MOE (Martin, P. Helv. Chim. Acta 1996, 79, 1930-1938), “LNA” (Wengel, J. Acc. Chem. Res. 1999, 32, 301-310).

Modifications of Nucleobases References:

Representative U.S. patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,30; 5,134,066; 5,175,273; 5,367,066; 5,432,272; 5,457,187; 5,457,191; 5,459,255; 5,484,908; 5,502,177; 5,525,711; 5,552,540; 5,587,469; 5,594,121, 5,596,091; 5,614,617; 5,681,941; 5,750,692; 6,015,886; 6,147,200; 6,166,197; 6,222,025; 6,235,887; 6,380,368; 6,528,640; 6,639,062; 6,617,438; 7,045,610; 7,427,672; and 7,495,088, each of which is herein incorporated by reference in its entirety.

Terminal Modification References:

Terminal modifications are described in Manoharan, M. et al. Antisense and Nucleic Acid Drug Development 12, 103-128 (2002) and references therein.

Placement of Modifications within an Oligonucleotide (e.g., Placement within an Anti-miR or miR Agent)

As oligonucleotides, such as anti-miR agents (e.g., an agent that inhibits at least one of miR-103, miR-105, miR-107 and/or miR-155) or miR agents/agonists (e.g., miR-103, miR-105, miR-107 or miR-155 agent) can be are polymers of subunits or monomers, many of the modifications described herein can occur at a position which is repeated within an oligonucleotide, e.g., a modification of a nucleobase, a sugar, a phosphate moiety, or the non-bridging oxygen of a phosphate moiety. It is not necessary for all positions in a given oligonucleotide to be uniformly modified, and in fact more than one of the aforementioned modifications can be incorporated in a single oligonucleotide or even at a single nucleoside within an oligonucleotide.

In some cases the modification will occur at all of the subject positions in the oligonucleotide but in many, and in fact in most cases it will not. By way of example, a modification can occur at a 3′ or 5′ terminus position, can occur in the internal region, can occur in 3′, 5′ or both terminal regions, e.g. at a position on a termus nucleotide or in the last 2, 3, 4, 5, 6, 7, 8, 9, or 10 nucleotides of an oligonucleotide. In some embodiments, the terminus nucleotide does not comprise a modification.

In some embodiments, the terminus nucleotide or the last 2, 3, 4, 5, 6, 7, 8, 9, or 10 nucleotides of at least one end of the oligonucleotide all comprise at least one modification. In some embodiments, the modification is same. In some embodiments, the terminus nucleotide or the last 2, 3, 4, 5, 6, 7, 8, 9, or 10 nucleotides at both ends of the oligonucleotide all comprise at least one modification. It is to be understood that type of modification and number of modified nucleotides on one end of the oligonucleotide is independent of type of modification and number of modified nucleotides on the other end of the oligonucleotide.

When the oligonucleotide is double-stranded or partially double-stranded, a modification can occur in the double strand region, the single strand region, or in both the double- and single-stranded regions. In some embodiments, a modification described herein does not occur in the region corresponding to the target cleavage site region. For example, a phosphorothioate modification at a non-bridging oxygen position can occur at one or both termini, can occur in a terminal regions, e.g., at a position on a terminal nucleotide or in the last 2, 3, 4, 5, 6, 7, 8, 9, or 10 nucleotides of a strand, or can occur in double strand and single strand regions, particularly at termini.

Some modifications can preferably be included on an oligonucleotide at a particular location, e.g., at an internal position of a strand, or on the 5′ or 3′ end of an oligonucleotide. A preferred location of a modification on an oligonucleotide, can confer preferred properties on the oligonucleotide. For example, preferred locations of particular modifications can confer increased resistance to endonuclease or exonuclease activity.

### Production of Anti-miR Agents or miR Agents and Mimetics Thereof.

In some embodiments, the oligonucleotide compounds (e.g., an anti-miR agent that inhibits at least one of miR-103, miR-105, miR-107 and/or miR-155; or a miR agent (e.g., miR-103, miR-105, miR-107 and/or miR-155 agent/agonist)) as disclosed herein can be prepared using solution-phase or solid-phase organic synthesis, or enzymatically by methods known in the art. Organic synthesis offers the advantage that the oligonucleotide strands comprising non-natural or modified nucleotides can be easily prepared. Any other means for such synthesis known in the art can additionally or alternatively be employed. It is also known to use similar techniques to prepare other oligonucleotides, such as the phosphorothioates, phosphorodithioates and alkylated derivatives. The double-stranded oligonucleotide compounds of the invention can be prepared using a two-step procedure. First, the individual strands of the double-stranded molecule are prepared separately. Then, the component strands are annealed.

MicroRNAs have also been implicated in modulation of pathogens in hosts. For example, see Jopling, C. L., et al., Science (2005) vol. 309, pp 1577-1581.

Regardless of the method of synthesis, the oligonucleotide can be prepared in a solution (e.g., an aqueous and/or organic solution) that is appropriate for formulation. For example, the oligonucleotide preparation can be precipitated and redissolved in pure double-distilled water, and lyophilized. The dried oligonucleotide can then be resuspended in a solution appropriate for the intended formulation process.

Teachings regarding the synthesis of particular modified oligonucleotides can be found in the following U.S. patents or pending patent applications: U.S. Pat. Nos. 5,138,045 and 5,218,105, drawn to polyamine conjugated oligonucleotides; U.S. Pat. No. 5,212,295, drawn to monomers for the preparation of oligonucleotides having chiral phosphorus linkages; U.S. Pat. Nos. 5,378,825 and 5,541,307, drawn to oligonucleotides having modified backbones; U.S. Pat. No. 5,386,023, drawn to backbone-modified oligonucleotides and the preparation thereof through reductive coupling; U.S. Pat. No. 5,457,191, drawn to modified nucleobases based on the 3-deazapurine ring system and methods of synthesis thereof; U.S. Pat. No. 5,459,255, drawn to modified nucleobases based on N-2 substituted purines; U.S. Pat. No. 5,521,302, drawn to processes for preparing oligonucleotides having chiral phosphorus linkages; U.S. Pat. No. 5,539,082, drawn to peptide nucleic acids; U.S. Pat. No. 5,554,746, drawn to oligonucleotides having beta-lactam backbones; U.S. Pat. No. 5,571,902, drawn to methods and materials for the synthesis of oligonucleotides; U.S. Pat. No. 5,578,718, drawn to nucleosides having alkylthio groups, wherein such groups can be used as linkers to other moieties attached at any of a variety of positions of the nucleoside; U.S. Pat. Nos. 5,587,361 and 5,599,797, drawn to oligonucleotides having phosphorothioate linkages of high chiral purity; U.S. Pat. No. 5,506,351, drawn to processes for the preparation of 2′-O-alkyl guanosine and related compounds, including 2,6-diaminopurine compounds; U.S. Pat. No. 5,587,469, drawn to oligonucleotides having N-2 substituted purines; U.S. Pat. No. 5,587,470, drawn to oligonucleotides having 3-deazapurines; U.S. Pat. No. 5,223,168, and U.S. Pat. No. 5,608,046, both drawn to conjugated 4′-desmethyl nucleoside analogs; U.S. Pat. Nos. 5,602,240, and 5,610,289, drawn to backbone-modified oligonucleotide analogs; and U.S. Pat. Nos. 6,262,241, and 5,459,255, drawn to, inter alia, methods of synthesizing 2′-fluoro-oligonucleotides.

Additionally, in some embodiments, miRNA can be isolated from cells or tissues, recombinantly produced, or synthesized in vitro by a variety of techniques well known to one of ordinary skill in the art. In one approach, miRNA is isolated from cells or tissues.

Techniques for isolating miRNA from cells or tissues are well known to one of ordinary skill in the art. For example, miRNA can be isolated from total RNA using the miRNA isolation kit from Ambion, Inc. Another technique utilizes the flashPAGE Fractionator System (Ambion, Inc.) for PAGE purification of small nucleic acids.

The miRNA can be obtained by preparing a recombinant version thereof (i.e., by using the techniques of genetic engineering to produce a recombinant nucleic acid which can then be isolated or purified by techniques well known to one of ordinary skill in the art). This approach involves growing a culture of host cells in a suitable culture medium, and purifying the miRNA from the cells or the culture in which the cells are grown. For example, the methods include a process for producing a miRNA in which a host cell, containing a suitable expression vector that includes a nucleic acid encoding a miRNA, is cultured under conditions that allow expression of the encoded miRNA. The miRNA can be recovered from the culture, from the culture medium or from a lysate prepared from the host cells, and further purified. The host cell can be a higher eukaryotic host cell such as a mammalian cell, a lower eukaryotic host cell such as a yeast cell, or the host cell can be a prokaryotic cell such as a bacterial cell. Introduction of a vector containing the nucleic acid encoding the miRNA into the host cell can be effected by calcium phosphate transfection, DEAE-dextran mediated transfection, or electroporation (Davis, L. et al., Basic Methods in Molecular Biology (1986)).

Any host/vector system can be used to express one or more of the miRNAs. These include, but are not limited to, eukaryotic hosts such as HeLa cells and yeast, as well as prokaryotic host such as E. coli and B. subtilis. An anti-miR agent, e.g., anti-miR as disclosed herein can be expressed in mammalian cells, yeast, bacteria, or other cells where the anti-miR agent, e.g., anti-miR is under the control of an appropriate promoter. Appropriate cloning and expression vectors for use with prokaryotic and eukaryotic hosts are described by Sambrook, et al., in Molecular Cloning: A Laboratory Manual, Second Edition, Cold Spring Harbor, N.Y. (1989). In the preferred embodiment, the miRNA is expressed in mammalian cells. Examples of mammalian expression systems include C127, monkey COS cells, Chinese Hamster Ovary (CHO) cells, human kidney 293 cells, human epidermal A43 1 cells, human Colo205 cells, 3T3 cells, CV-1 cells, other transformed primate cell lines, normal diploid cells, cell strains derived from in vitro culture of primary tissue, primary explants, HeLa cells, mouse L cells, BILK, HL-60, U937, HaK or Jurkat cells.

Mammalian expression vectors will comprise an origin of replication, a suitable promoter, polyadenylation site, transcriptional termination sequences, and 5′ flanking non-transcribed sequences. DNA sequences derived from the SV40 viral genome, for example, SV40 origin, early promoter, enhancer, splice, and polyadenylation sites may be used to provide the required non-transcribed genetic elements. Potentially suitable yeast strains include Saccharomyces cerevsiae, Schizosaccharomyces pombe, Klayveromyces strains, Candida, or any yeast strain capable of expressing miRNA. Potentially suitable bacterial strains include Escherichia coli, Bacillus subtilis, Salmonella typhimurium, or any bacterial strain capable of expressing miRNA.

In another approach, genomic DNA encoding an anti-miR-425 agent, e.g., anti-miR is isolated, the genomic DNA is expressed in a mammalian expression system, and RNA is purified and modified as necessary for administration to a patient. In some embodiments, a miR-425 (e.g., to decrease ANP levels in a subject) is in the form of a pre-miRNA, which can be modified as desired (i.e. for increased stability or cellular uptake).

Knowledge of DNA sequences of miRNA allows for modification of cells to permit or increase expression of an endogenous miRNA. Cells can be modified (e.g., by homologous recombination) to provide increased miRNA expression by replacing, in whole or in part, the naturally occurring promoter with all or part of a heterologous promoter so that the cells express the miRNA at higher levels. The heterologous promoter is inserted in such a manner that it is operatively linked to the desired miRNA encoding sequences. See, for example, PCT International Publication No. WO 94/12650 by Transkaryotic Therapies, Inc., PCT International Publication No. WO 92/20808 by Cell Genesys, Inc., and PCT International Publication No. WO 91/09955 by Applied Research Systems. Cells also may be; engineered to express an endogenous gene comprising the miRNA under the control of inducible regulatory elements, in which case the regulatory sequences of the endogenous gene may be replaced by homologous recombination. Gene activation techniques are described in U.S. Pat. No. 5,272,071 to Chappel; U.S. Pat. No. 5,578,461 to Sherwin et al.; PCT/US92/09627 (WO93/09222) by Selden et al.; and PCT/US90/06436 (WO91/06667) by Skoultchi et al. The miRNA may be prepared by culturing transformed host cells under culture conditions suitable to express the miRNA. The resulting expressed miRNA may then be purified from such culture (i.e., from culture medium or cell extracts) using known purification processes, such as gel filtration and ion exchange chromatography. The purification of the miRNA may also include an affinity column containing agents which will bind to the protein; one or more column steps over such affinity resins as concanavalin A-agarose, heparin-Toyopearl™ or Cibacrom blue 3GA Sepharose™; one or more steps involving hydrophobic interaction chromatography using such resins as phenyl ether, butyl ether, or propyl ether; immunoaffnity chromatography, or complementary cDNA affinity chromatography.

The miRNA can also be expressed as a product of transgenic animals, which are characterized by somatic or germ cells containing a nucleotide sequence encoding the miRNA. A vector containing DNA encoding miRNA and appropriate regulatory elements can be inserted in the germ line of animals using homologous recombination (Capecchi, Science t 244:1288-1292 (1989)), such that they express the miRNA. Transgenic animals, preferably non-human mammals, are produced using methods as described in U.S. Pat. No. 5,489,743 to Robinson, et al., and PCT Publication No. WO 94/28122 by Ontario Cancer Institute. miRNA can be isolated from cells or tissue isolated from transgenic animals as discussed above.

In one approach, the miRNA can be obtained synthetically, for example, by chemically synthesizing a nucleic acid by any method of synthesis known to the skilled artisan. The synthesized miRNA can then be purified by any method known in the art. Methods for chemical synthesis of nucleic acids include, but are not limited to, in vitro chemical synthesis using phosphotriester, phosphate or phosphoramidite chemistry and solid phase techniques, or via deoxynucleoside H-phosphonate intermediates (see U.S. Pat. No. 5,705,629 to Bhongle).

In some circumstances, for example, where increased nuclease stability is desired, nucleic acids having nucleic acid analogs and/or modified internucleoside linkages may be preferred. Nucleic acids containing modified internucleoside linkages can also be synthesized using reagents and methods that are well known in the art. For example, methods of synthesizing nucleic acids containing phosphonate phosphorothioate, phosphorodithioate, phosphoramidate methoxyethyl phosphoramidate, formacetal, thioformacetal, diisopropylsilyl, acetamidate, carbamate, dimethylene-sulfide (—CH2-S-CH2), diinethylene-sulfoxide (—CH2-SO—CH2), dimethylene-sulfone (—CH2-SO2-CH2), 2′-O-alkyl, and 2′-deoxy-2′-fluoro′phosphorothioate internucleoside linkages are well known in the art (see Uhlmann et al., 1990, Chem. Rev. 90:543-584; Schneider et al., 1990, Tetrahedron Left. 31:335 and references cited therein). U.S. Pat. Nos. 5,614,617 and 5,223,618 to Cook, et al., U.S. Pat. No. 5,714,606 to Acevedo, et al, U.S. Pat. No. 5,378,825 to Cook, et al., U.S. Pat. Nos. 5,672,697 and 5,466,786 to Buhr, et al., U.S. Pat. No. 5,777,092 to Cook, et al., U.S. Pat. No. 5,602,240 to De Mesmacker, et al., U.S. Pat. No. 5,610,289 to Cook, et al. and U.S. Pat. No. 5,858,988 to Wang, also describe nucleic acid analogs for enhanced nuclease stability and cellular uptake.

In some embodiments, miR inhibitors, e.g., anti-miR agents as disclosed herein (e.g, anti-miR agents which inhibit miR-103, miR-105, miR-107, miR-155 or miR-425) can comprise locked nucleotides, e.g., as disclosed in U.S. Pat. No. 8,642,751, which is incorporated herein in its entirety by reference. Other chemical modifications of motifs for anti-miR agents as disclosed herein, (e.g, anti-miR agents which inhibit miR-103, miR-105, miR-107, miR-155 or miR-425) are disclosed in US application US 2012/0148664 and US 2014/0066491, which are incorporated herein in their entirety by reference. In alternative embodiments, miR inhibitors, e.g., anti-miR agents as disclosed herein (e.g, anti-miR agents which inhibit miR-103, miR-105, miR-107, miR-155 or miR-425) can comprise base modified oligonucleotides, e.g., as disclosed in International Applications WO 2012061810 and WO 2012061810, which are incorporated herein in their entirety by reference.

### Oligonucleotide Formulations:

A formulated oligonucleotide composition can assume a variety of states. In some examples, the composition can be at least partially crystalline, uniformly crystalline, and/or anhydrous (e.g., less than 80, 50, 30, 20, or 10% water). In another example, the oligonucleotide is in an aqueous phase, e.g., in a solution that includes water.

The aqueous phase or the crystalline compositions can, e.g., be incorporated into a delivery vehicle, e.g., a liposome (particularly for the aqueous phase) or a particle (e.g., a micro particle as can be appropriate for a crystalline composition). Generally, the oligonucleotide composition is formulated in a manner that is compatible with the intended method of administration.

In particular embodiments, the composition is prepared by at least one of the following methods: spray drying, lyophilization, vacuum drying, evaporation, fluid bed drying, or a combination of these techniques; or sonication with a lipid, freeze-drying, condensation and other self-assembly.

An oligonucleotide preparation can be formulated in combination with another agent, e.g., another therapeutic agent or an agent that stabilizes the oligonucleotide, e.g., a protein that complex with oligonucleotide to form an oligonucleotide-protein complex. Still other agents include chelators, e.g., EDTA (e.g., to remove divalent cations such as Mg²⁺), salts, DNAse inhibitors, RNAse inhibitors (e.g., a broad specificity RNAse inhibitor such as RNAsin) and so forth.

In some embodiments, the oligonucleotide preparation includes at least a second therapeutic agent (e.g., an agent other than RNA or DNA). Exemplary therapeutic agents that can formulated with an oligonucleotide preparation include, but are not limited to, those found in Harrison's Principles of Internal Medicine, 17^{th} Edition, 2008, McGraw-Hill N.Y., NY; Physicians Desk Reference, 63^{rd} Edition, 2008, Thomson Reuters, N.Y., N.Y.; Goodman & Gilman's The Pharmacological Basis of Therapeutics, 11^{th} Edition, 2005, McGraw-Hill N.Y., NY; United States Pharmacopeia, The National Formulary, USP-32 NF-27, 2008, U.S. Pharmacopeia, Rockville, Md., the complete contents of all of which are incorporated herein by reference.

In some embodiments, the second therapeutic agent is an anti-hypertension agent or anti-hypertensive.

### Exemplary Oligonucleotide Formulations

Liposomes:

The oligonucleotides of the invention (e.g., anti-miR-425 or miR-425 agent) can be formulated in liposomes. As used herein, a liposome is a structure having lipid-containing membranes enclosing an aqueous interior. Liposomes can have one or more lipid membranes. In some embodiments, liposomes have an average diameter of less than about 100 nm. More preferred embodiments provide liposomes having an average diameter from about 30-70 nm and most preferably about 40-60 nm. Oligolamellar large vesicles and multilamellar vesicles have multiple, usually concentric, membrane layers and are typically larger than 100 nm. Liposomes with several nonconcentric membranes, i.e., several smaller vesicles contained within a larger vesicle, are termed multivesicular vesicles.

Liposomes can further comprise one or more additional lipids and/or other components such as sterols, e.g., cholesterol. Additional lipids can be included in the liposome compositions for a variety of purposes, such as to prevent lipid oxidation, to stabilize the bilayer, to reduce aggregation during formation or to attach ligands onto the liposome surface. Any of a number of additional lipids and/or other components can be present, including amphipathic, neutral, cationic, anionic lipids, and programmable fusion lipids. Such lipids and/or components can be used alone or in combination. One or more components of the liposome can comprise a ligand, e.g., a targeting ligand.

Liposome compositions can be prepared by a variety of methods that are known in the art. See e.g., U.S. Pat. Nos. 4,235,871; 4,737,323; 4,897,355 and 5,171,678; published International Applications WO 96/14057 and WO 96/37194; Feigner, P. L. et al., Proc. Natl. Acad. Sci., USA (1987) 8:7413-7417, Bangham, et al. M. Mol. Biol. (1965) 23:238, Olson, et al. Biochim. Biophys. Acta (1979) 557:9, Szoka, et al. Proc. Natl. Acad. Sci. (1978) 75: 4194, Mayhew, et al. Biochim. Biophys. Acta (1984) 775:169, Kim, et al. Biochim. Biophys. Acta (1983) 728:339, and Fukunaga, et al. Endocrinol. (1984) 115:757.

Micelles and other Membranous Formulations: In some embodiments, the oligonucleotides (e.g., anti-miR-425 or miR-425 agent) as disclosed herein can be prepared and formulated as micelles. As used herein, “micelles” are a particular type of molecular assembly in which amphipathic molecules are arranged in a spherical structure such that all hydrophobic portions on the molecules are directed inward, leaving the hydrophilic portions in contact with the surrounding aqueous phase. The converse arrangement exists if the environment is hydrophobic.

In some embodiments, the formulations comprises micelles formed from an oligonucleotide of the invention and at least one amphiphilic carrier, in which the micelles have an average diameter of less than about 100 nm, preferably. More preferred embodiments provide micelles having an average diameter less than about 50 nm, and even more preferred embodiments provide micelles having an average diameter less than about 30 nm, or even less than about 20 nm.

Micelle formulations can be prepared by mixing an aqueous solution of the oligonucleotide composition, an alkali metal C₈ to C₂₂ alkyl sulphate, and an amphiphilic carrier. The amphiphilic carrier can be added at the same time or after addition of the alkali metal alkyl sulphate. Micelles will form with substantially any kind of mixing of the ingredients but vigorous mixing in order to provide smaller size micelles.

Emulsions:

In some embodiments, the oligonucleotides (e.g., anti-miR-425 or miR-425 agent) as disclosed herein can be prepared and formulated as emulsions. As used herein, “emulsion” is a heterogenous system of one liquid dispersed in another in the form of droplets. Emulsions are often biphasic systems comprising two immiscible liquid phases intimately mixed and dispersed with each other. Either of the phases of the emulsion can be a semisolid or a solid, as is the case of emulsion-style ointment bases and creams. The oligonucleotide can be present as a solution in either the aqueous phase, oily phase or itself as a separate phase.

In some embodiments, the compositions are formulated as microemulsions. As used herein, “microemulsion” refers to a system of water, oil and amphiphile which is a single optically isotropic and thermodynamically stable liquid solution. Microemuslions also include thermodynamically stable, isotropically clear dispersions of two immiscible liquids that are stabilized by interfacial films of surface-active molecules.

The application of emulsion formulations via dermatological, oral and parenteral routes and methods for their manufacture have been reviewed in the literature, for example see Idson, in Pharmaceutical Dosage Forms, Lieberman, Rieger and Banker (Eds.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., volume 1, p. 245; and Block, in Pharmaceutical Dosage Forms, Lieberman, Rieger and Banker (Eds.), 1988, Marcel Dekker, Inc., New York, N.Y., volume 1, p. 335, contents of which are herein incorporated by reference in their entirety.

Lipid Particles:

In some embodiments, the oligonucleotides (e.g., anti-miR-425 or miR-425 agent) as disclosed herein can be prepared and formulated as lipid particles, e.g., formulated lipid particles (FLiPs) comprising (a) an oligonucleotide of the invention, where said oligonucleotide has been conjugated to a lipophile and (b) at least one lipid component, for example an emulsion, liposome, isolated lipoprotein, reconstituted lipoprotein or phospholipid, to which the conjugated oligonucleotide has been aggregated, admixed or associated. The stoichiometry of oligonucleotide to the lipid component can be 1:1. Alternatively the stoichiometry can be 1:many, many:1 or many:many, where many is two or more.

The FLiP can comprise triacylglycerols, phospholipids, glycerol and one or several lipid-binding proteins aggregated, admixed or associated via a lipophilic linker molecule with an oligonucleotide. Surprisingly, it has been found that due to said one or several lipid-binding proteins in combination with the above mentioned lipids, the FLiPs show affinity to liver, gut, kidney, steroidogenic organs, heart, lung and/or muscle tissue. These FLiPs can therefore serve as carrier for oligonucleotides to these tissues. For example, lipid-conjugated oligonucleotides, e.g., cholesterol-conjugated oligonucleotides, bind to HDL and LDL lipoprotein particles which mediate cellular uptake upon binding to their respective receptors thus directing oligonucleotide delivery into liver, gut, kidney and steroidogenic organs, see Wolfrum et al. Nature Biotech. (2007), 25:1145-1157.

The FLiP can be a lipid particle comprising 15-25% triacylglycerol, about 0.5-2% phospholipids and 1-3% glycerol, and one or several lipid-binding proteins. FLiPs can be a lipid particle having about 15-25% triacylglycerol, about 1-2% phospholipids, about 2-3% glycerol, and one or several lipid-binding proteins. In some embodiments, the lipid particle comprises about 20% triacylglycerol, about 1.2% phospholipids and about 2.25% glycerol, and one or several lipid-binding proteins.

Another suitable lipid component for FLiPs is lipoproteins, for example isolated lipoproteins or more preferably reconstituted lipoprotieins. Exemplary lipoproteins include chylomicrons, VLDL (Very Low Density Lipoproteins), IDL (Intermediate Density Lipoproteins), LDL (Low Density Lipoproteins) and HDL (High Density Lipoproteins). Methods of producing reconstituted lipoproteins are known in the art, for example see A. Jones, Experimental Lung Res. 6, 255-270 (1984), U.S. Pat. Nos. 4,643,988 and 5,128,318, PCT publication WO87/02062, Canadian Pat. No. 2,138,925. Other methods of producing reconstituted lipoproteins, especially for apolipoproteins A-I, A-II, A-IV, apoC and apoE have been described in A. Jonas, Methods in Enzymology 128, 553-582 (1986) and G. Franceschini et al. J. Biol. Chem., 260(30), 16321-25 (1985).

One preferred lipid component for FLiP is Intralipid. Intralipid® is a brand name for the first safe fat emulsion for human use. Intralipid® 20% (a 20% intravenous fat emulsion) is made up of 20% soybean oil, 1.2% egg yolk phospholipids, 2.25% glycerin, and water for injection. It is further within the present invention that other suitable oils, such as saflower oil, can serve to produce the lipid component of the FLiP.

FLiP can range in size from about 20-50 nm or about 30-50 nm, e.g., about 35 nm or about 40 nm. In some embodiments, the FLiP has a particle size of at least about 100 nm. FLiPs can alternatively be between about 100-150 nm, e.g., about 110 nm, about 120 nm, about 130 nm, or about 140 nm, whether characterized as liposome- or emulsion-based. Multiple FLiPs can also be aggregated and delivered together, therefore the size can be larger than 100 nm.

The process for making the lipid particles comprises the steps of: (a) mixing a lipid components with one or several lipophile (e.g. cholesterol) conjugated oligonucleotides that can be chemically modified; and (b) fractionating this mixture. In some embodiments, the process comprises the additional step of selecting the fraction with particle size of 30-50 nm, preferably of about 40 nm in size.

Some exemplary lipid particle formulations amenable to the invention are described in U.S. patent application Ser. No. 12/412,206, filed Mar. 26, 2009, contents of which are herein incorporated by reference in their entirety.

Yeast Cell Wall Particles:

In some embodiments, the oligonucleotides (e.g., anti-miR-425 or miR-425 agent) as disclosed herein are formulated in yeast cell wall particles (“YCWP”). A yeast cell wall particle comprises an extracted yeast cell wall exterior and a core, the core comprising a payload (e.g., oligonucleotides). Exterior of the particle comprises yeast glucans (e.g. beta glucans, beta-1,3-glucans, beta-1,6-glucans), yeast mannans, or combinations thereof. Yeast cell wall particles are typically spherical particles about 1-4 μm in diameter.

Preparation of yeast cell wall particles is known in the art, and is described, for example in U.S. Pat. Nos. 4,992,540; 5,082,936; 5,028,703; 5,032,401; 5,322,841; 5,401,727; 5,504,079; 5,607,677; 5,741,495; 5,830,463; 5,968,811; 6,444,448; and 6,476,003, U.S. Pat. App. Pub. Nos. 2003/0216346 and 2004/0014715, and Int. App. Pub. No. WO 2002/12348, contents of which are herein incorporated by reference in their entirety. Applications of yeast cell like particles for drug delivery are described, for example in U.S. Pat. Nos. 5,032,401; 5,607,677; 5,741,495; and 5,830,463, and U.S. Pat. Pub Nos. 2005/0281781 and 2008/0044438, contents of which are herein incorporated by reference in their entirety. U.S. Pat. App. Pub. No. 2009/0226528, contents of which are herein incorporated by reference, describes formulation of nucleic acids with yeast cell wall particles for delivery of oligonucleotide to cells.

Additional exemplary formulations for oligonucleotides are described in U.S. Pat. Nos. 4,897,355; 4,394,448; 4,235,871; 4,231,877; 4,224,179; 4,753,788; 4,673,567; 4,247,411; 4,814,270; 5,567,434; 5,552,157; 5,565,213; 5,738,868; 5,795,587; 5,922,859; and 6,077,663, Int. App. Nos. PCT/US07/079203, filed Sep. 21, 2007; PCT/US07/080331, filed Oct. 3, 2007; U.S. patent application Ser. No. 12/123,922, filed May 28, 2008; U.S. Pat. Pub. Nos. 2006/0240093 and 2007/0135372 and U.S. Provisional App. Nos. 61/018,616, filed Jan. 2, 2008; 61/039,748, filed Mar. 26; 2008; 61/045,228, filed Apr. 15, 2008; 61/047,087, filed Apr. 22, 2008; 61/051,528, filed May 21, 2008; and 61/113,179 (filed Nov. 10, 2008), contents of which are herein incorporated by reference in their entirety. Behr (1994) Bioconjugate Chem. 5:382-389, and Lewis et al. (1996) PNAS 93:3176-3181), also describe formulations for oligonucleotides that are amenable to the invention, contents of which are herein incorporated by reference in their entirety.

### Pharmaceutical Compositions Comprising Oligonucleotide Anti-miR Agents or miR Agents:

In another aspect, provided herein are pharmaceutically acceptable compositions which comprise a therapeutically-effective amount of one or more of the anti-miR agent (e.g., that inhibits at least one of miR-103, miR-105, miR-107 and/or miR-155) or a miR agent (e.g., miR-103, miR-105, miR-107 and/or miR-155 agonist) as disclosed herein, formulated together with one or more pharmaceutically acceptable carriers (additives) and/or diluents.

Stratagies for delivery of miRNA's and anti-miRs are known by persons of ordinary skill in the art and are encompassed for use in the delivery of an anti-miR agent (e.g., that inhibits at least one of miR-103, miR-105, miR-107 and/or miR-155) or a miR agent (e.g., miR-103, miR-105, miR-107 and/or miR-155 agent) as disclosed herein. Delivery of miRNAs are discussed in, for example, Broderick et al., Gene Therapy, 2011, 18; 1104-1110, which is incorporated herein in its entirety by reference. In some embodiments, an anti-miR agent (e.g., that inhibits at least one of miR-103, miR-105, miR-107 and/or miR-155) or a miR agent (e.g., miR-103, miR-105, miR-107 and/or miR-155 agent) as disclosed herein can be modified to comprise 2′O-methyl, 2′O-methyloxyethyl or 2-fluoro modified nucleotides. In some embodiments, antagomirs are 2′-O-methyl oligonucleotides conjugated to cholesterol at their 3′ ends, and contain phosphorothioate linkages between nucleotides at both ends in place of natural phosphate linkages. In some embodiments, an anti-miR agent (e.g., that inhibits at least one of miR-103, miR-105, miR-107 and/or miR-155) or miR agent (e.g., miR-103, miR-105, miR-107 and/or miR-155 agent) as disclosed herein can be formulated in lipid nanoparticles, for example, lipid vesicles containing therapeutic RNA. The formulated lipid bilayer encapsulates the therapeutic RNA, delivering it to cells and promoting fusion with the phospholipid bilayer of cell membranes. Individual lipids within the vesicle bilayer can contain ionizable head groups that will disrupt the endosome at low pH to release the therapeutic RNA to the cytoplasm.

In some embodiments, an anti-miR agent (e.g., that inhibits at least one of miR-103, miR-105, miR-107 and/or miR-155) or miR agent (e.g., miR-103, miR-105, miR-107 and/or miR-155 agent) as disclosed herein can be formulated as golden lipid nanoparticles for therapeutic delivery, e.g., as disclosed in 2012/0128777, 2012/0244075 and 2013/0011339, and in Shi et al., Solid lipid nanoparticles loaded with anti-microRNA oligonucleotides (AMOs) for suppression of microRNA-21 functions in human lung cancer cells, Pharm Res, 2012; 29(1); 97-109, which are incorporated herein in its entirety by reference.

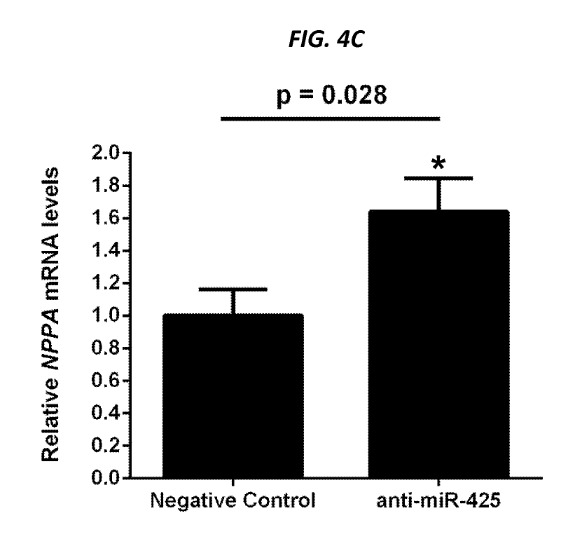

In some embodiments, chemical modifications that improve the stability, biodistribution and delivery of an anti-miR agent (e.g., that inhibits at least one of miR-103, miR-105, miR-107 and/or miR-155) or miR agent (e.g., miR-103, miR-105, miR-107 and/or miR-155 agent) is encompassed, for example as disclosed in FIG. 5 in Broderick et al., Gene Therapy, 2011, 18; 1104-1110.

As described in detail below, the pharmaceutical compositions comprising an anti-miR agent (e.g., that inhibits at least one of miR-103, miR-105, miR-107 and/or miR-155) or miR agent (e.g., miR-103, miR-105, miR-107 and/or miR-155 agent) can be specially formulated for administration in solid or liquid form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, compounds can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alchols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

A pharmaceutical composition is formulated to be compatible with its intended route of administration. Examples of routes of administration include parenteral, e.g., intravenous, intradermal, subcutaneous, intraperitoneal, intramuscular, oral (e.g., inhalation), transdermal (topical), transmucosal, and rectal administration.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Toxicity and therapeutic efficacy of an anti-miR agent (e.g., that inhibits at least one of miR-103, miR-105, miR-107 and/or miR-155 and/or miR-425) or miR agent (e.g., miR-103, miR-105, miR-107 and/or miR-155 agent and/or miR-425) can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects. The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the compound which achieves a half-maximal inhibition of symptoms) as determined in cell culture. Such information can be used to more accurately determine useful doses in humans. Levels in plasma may be measured, for example, by high performance liquid chromatography.

Toxicity and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions that exhibit large therapeutic indices, are preferred.

The phrase “therapeutically-effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment. Determination of a therapeutically effective amount is well within the capability of those skilled in the art. Generally, a therapeutically effective amount can vary with the subject's history, age, condition, sex, as well as the severity and type of the medical condition in the subject, and administration of other agents alleviate the disease or disorder to be treated.

The amount of oligonucleotide which can be combined with a carrier material to produce a single dosage form will generally be that amount of the compound which produces a therapeutic effect. Generally out of one hundred percent, this amount will range from about 0.1% to 99% of oligonucleotide anti-miR agent (e.g., that inhibits at least one of miR-103, miR-105, miR-107 and/or miR-155) or miR agent (e.g., miR-103, miR-105, miR-107 and/or miR-155 agent), preferably from about 5% to about 70%, most preferably from 10% to about 30%.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized.

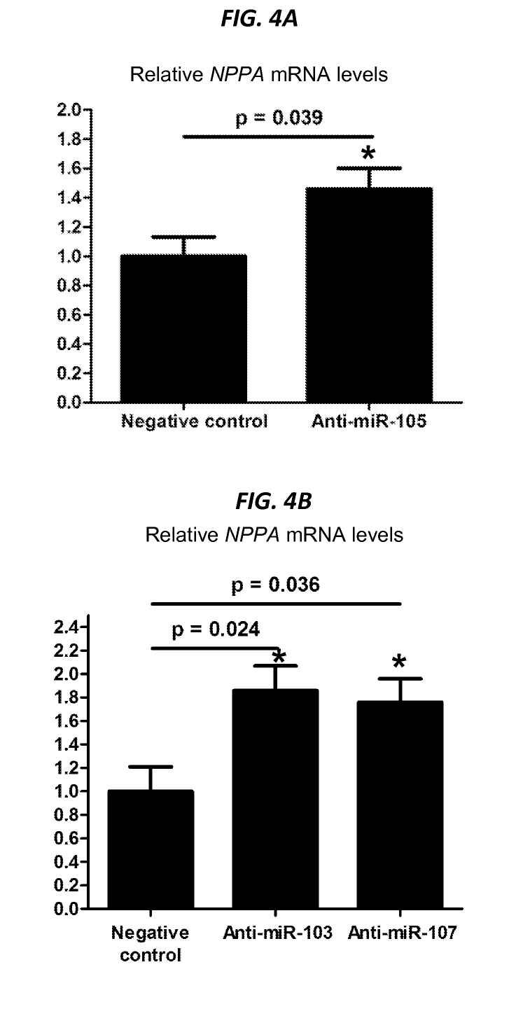

The therapeutically effective dose can be estimated initially from cell culture assays or as disclosed herein in the Examples, e.g., see FIGS. 4A, 4B, 4C, 5B, 6, 8, and 16C-16D, 20C-20D. A dose may be formulated in animal models to achieve a circulating plasma concentration range that includes the IC50 (i.e., the concentration of the therapeutic which achieves a half-maximal inhibition of symptoms) as determined in cell culture. Levels in plasma may be measured, for example, by high performance liquid chromatography. The effects of any particular dosage can be monitored by a suitable bioassay.

The dosage can be determined by a physician and adjusted, as necessary, to suit observed effects of the treatment. Generally, the compositions are administered so that oligonucleotide anti-miR agent ((e.g., that inhibits at least one of miR-103, miR-105, miR-107 and/or miR-155) or miR agent (e.g., miR-103, miR-105, miR-107 and/or miR-155 agent) is given at a dose from 1 μg/kg to 150 mg/kg, 1 μg/kg to 100 mg/kg, 1 μg/kg to 50 mg/kg, 1 μg/kg to 20 mg/kg, 1 μg/kg to 10 mg/kg, 1 μg/kg to 1 mg/kg, 100 μg/kg to 100 mg/kg, 100 μg/kg to 50 mg/kg, 100 μg/kg to 20 mg/kg, 100 μg/kg to 10 mg/kg, 100 μg/kg to 1 mg/kg, 1 mg/kg to 100 mg/kg, 1 mg/kg to 50 mg/kg, 1 mg/kg to 20 mg/kg, 1 mg/kg to 10 mg/kg, 10 mg/kg to 100 mg/kg, 10 mg/kg to 50 mg/kg, or 10 mg/kg to 20 mg/kg.

In some embodiments, a therapeutically effective amount of a pharmaceutical composition containing a miRNA antagonist (i.e., an anti-miR) of the invention (i.e., an effective dosage) is an amount that inhibits the repression of the NPPA gene expression by a miR disclosed herein (e.g., miR-103, miR-105, miR-107, miR-155 and/or miR-425) and/or inhibits the expression and/or activity of a miR disclosed herein (e.g., miR-103, miR-105, miR-107, miR-155 and/or miR-425) by at least 10 percent, more preferably at least 30%. Higher percentages of inhibition, e.g., 40, 50, 75, 85, 90 percent or higher may be preferred in certain embodiments. Exemplary doses include milligram or microgram amounts of the molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram.

In certain embodiments, pharmaceutical compositions comprise an anti-miR agent (e.g., that inhibits at least one of miR-103, miR-105, miR-107 and/or miR-155) or miR agent (e.g., miR-103, miR-105, miR-107 and/or miR-155 agent) at a dose selected from 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 270 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 670 mg, 675 mg, 680 mg, 685 mg, 690 mg, 695 mg, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, and 800 mg. In certain such embodiments, a pharmaceutical composition of the present invention comprises a dose of an anti-miR agent ((e.g., that inhibits at least one of miR-103, miR-105, miR-107 and/or miR-155) or miR agent (e.g., miR-103, miR-105, miR-107 and/or miR-155 agent) selected from 25 mg, 50 mg, 75 mg, 100 mg, 150 mg, 200 mg, 250 mg, 300 mg, 350 mg, 400 mg, 500 mg, 600 mg, 700 mg, and 800 mg.

The compositions can be administered one time per week for between about 1 to 10 weeks, e.g., between 2 to 8 weeks, or between about 3 to 7 weeks, or for about 4, 5, or 6 weeks. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a composition can include a single treatment or a series of treatments.

It is furthermore understood that appropriate doses of a composition depend upon the potency of composition with respect to the expression or activity to be modulated. When one or more of these molecules is to be administered to an animal (e.g., a human) to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment or make other alteration to treatment regimen. The dosing schedule can vary from once a week to daily depending on a number of clinical factors, such as the subject's sensitivity to the polypeptides. The desired dose can be administered at one time or divided into subdoses, e.g., 2-4 subdoses and administered over a period of time, e.g., at appropriate intervals through the day or other appropriate schedule. Such sub-doses can be administered as unit dosage forms. Examples of dosing schedules are administration once a week, twice a week, three times a week, daily, twice daily, three times daily or four or more times daily.

Combinations of Anti-miR-425 Agents or miR-425 with Additional Agents

In some embodiments, an oligonucleotide anti-miR agent (e.g., that inhibits at least one of miR-103, miR-105, miR-107 and/or miR-155) or miR agent (e.g., miR-103, miR-105, miR-107 and/or miR-155 agent) as disclosed herein, can be administrated to a subject to increase ANP levels and/or to decrease blood pressure and/or hypertension and/or for the treatment of a cardiovascular disease or disorder in combination with a pharmaceutically active agent, e.g., a second therapeutic agent. In some embodiments, an anti-miR agent (e.g., that inhibits at least one of miR-103, miR-105, miR-107 and/or miR-155) can be administered alone, or in combination with an anti-miR-425 agent, as disclosed in International Application WO2013/188787, which is incorporated herein in its entirety by reference.

In an alternative embodiment, a miR agent (e.g., agonist of miR-103, miR-105, miR-107 and/or miR-155) can be administered to a subject to increase blood pressure alone, or in combination with another agent, e.g., a miR-425 agent, as disclosed in International Application WO2013/188787.

In some embodiments, a subject can be administered an anti-miR-155 agent. In some embodiments, a subject is administered an anti-miR-155 agent as disclosed herein, in combination with an anti-miR-425 agent as disclosed in International Application WO2013/188787. In some embodiments, where an anti-miR-155 agent is administered in combination with an anti-miR-425 agent, the dose, or amount of each anti-miR is less as compared to when each of the anti-miR-155 and anti-miR-425 agent are used alone. In some embodiments, when an anti-miR-155 agent is used in combination with an anti-miR-425 agent, the amount of anti-miR-155 is about 5% less, or about 10% less, or about 15%, or about 20%, or about 25%, or about 30% less, or more than 30% less as compared to when an anti-miR-155 is used alone. Similarly, in some embodiments, when an anti-miR-425 agent is used in combination with an anti-miR-155 agent, the amount of anti-miR-425 is about 5% less, or about 10% less, or about 15%, or about 20%, or about 25%, or about 30% less, or more than 30% less as compared to when an anti-miR-425 is used alone.

In some embodiments, a subject can be administered an anti-miR-105 agent. In some embodiments, a subject is administered an anti-miR-105 agent as disclosed herein, in combination with an anti-miR-425 agent as disclosed in International Application WO2013/188787. In some embodiments, where an anti-miR-105 agent is administered in combination with an anti-miR-425 agent, the dose, or amount of each anti-miR is less as compared to when each of the anti-miR-105 and anti-miR-425 agent are used alone. In some embodiments, when an anti-miR-105 agent is used in combination with an anti-miR-425 agent, the amount of anti-miR-105 is about 5% less, or about 10% less, or about 15%, or about 20%, or about 25%, or about 30% less, or more than 30% less as compared to when an anti-miR-105 is used alone. Similarly, in some embodiments, when an anti-miR-425 agent is used in combination with an anti-miR-105 agent, the amount of anti-miR-425 is about 5% less, or about 10% less, or about 15%, or about 20%, or about 25%, or about 30% less, or more than 30% less as compared to when an anti-miR-425 is used alone.

In some embodiments, a subject is administered an anti-miR-103 or anti-miR-107 agent, or an anti-miR-103 and anti-miR-107 agent as disclosed herein, in combination with an anti-miR-425 agent as disclosed in International Application WO2013/188787.

In some embodiments, exemplary pharmaceutical compositions, comprising the anti-miRs agents alone, or combinations of the anti-miRs as disclosed herein, and in some embodiments, in combination with anti-miR-425 agents that can be administered to a subject to increase plasma ANP levels in a subject are disclosed in the Table 3:

Exemplary pharmaceutically active compound include, but are not limited to, those found in Harrison's Principles of Internal Medicine, 13^{th} Edition, Eds. T. R. Harrison et al. McGraw-Hill N.Y., NY; Physicians Desk Reference, 50^{th} Edition, 1997, Oradell N.J., Medical Economics Co.; Pharmacological Basis of Therapeutics, 8^{th} Edition, Goodman and Gilman, 1990; and United States Pharmacopeia, The National Formulary, USP XII NF XVII, 1990, the complete contents of all of which are incorporated herein by reference.

In some embodiments, where an anti-miR agent (e.g., that inhibits at least one of miR-103, miR-105, miR-107 and/or miR-155) is being administered, a second therapeutic agent is an anti-hypertension agent or anti-hypertensive. Anti-hypertensives are a class of drugs that are used to treat hypertension (high blood pressure). Antihypertensive therapy seeks to prevent the complications of high blood pressure, such as stroke and myocardial infarction. Exemplary types of anti-hypertension agents include, but are not limited to, statins, diuretics, adrenergic receptor antagonists, calcium channel blockers, renin inhibitors, ACE inhibitors, angiotensin II receptor antagonists, aldosterone antagonists, vasodilators; alpha-2-agonists, and any combination thereof.

Exemplary anti-hypertension agents include, but are not limited to, bumetanide; ethacrynic acid; furosemide; torsemide; epitizide; hydrochlorothiazide; chlorothiazide; bendroflumethiazide; indapamide; chlorthalidone; metolazone; amiloride; triamterene; spironolactone; atenolol; metoprolol; nadolol; oxprenolol; pindolol; propranolol; timolol; doxazosin; phentolamine; indoramin; phenoxybenzamine; prazosin; terazosin; tolazoline; bucindolol; carvedilol; labetalol; amlodipine; felodipine; isradipine; lercanidipine; nicardipine; nifedipine; nimodipine; nitrendipine; diltiazem; verapamil; Aliskiren; captopril; enalapril; fosinopril; lisinopril; perindopril; quinapril; ramipril; trandolapril; benazepril; candesartan; eprosartan; irbesartan; losartan; olmesartan; telmisartan; valsartan; eplerenone; spironolactone; sodium nitroprusside; hydralazine; hydralazine derivatives; Clonidine; Guanabenz; Methyldopa; Moxonidine; Guanethidine; Reserpine; atorvastatin; fluvastatin; lovastatin; pitavastatin; pravastatin; rosuvastatin; simvastatin; and any combinations thereof.

In some embodiments, an anti-miR agent (e.g., that inhibits at least one of miR-103, miR-105, miR-107 and/or miR-155) can be administered in combination with, or sequentially to, ANP and/or BNP, or ANP or BNP mimetics, for example, but not limited to nesiritide or ANP, or an agent which has substantially the same function as endogenous ANP, or mimic the effects of natriuretic peptides.

In some embodiments, an anti-miR agent (e.g., that inhibits at least one of miR-103, miR-105, miR-107 and/or miR-155) can be administered in combination, or sequentially to (either before or after) administration of the anti-miR with a PDE-5 inhibitor, or other agent which increases endogenous cGMP, which is a secondary messenger for ANP. In some embodiments, a PDE-5 inhibitor is selected from the group consisting of: drug tadalafil (CIALIS™, ADCIRCA™), sildenafil (VIAGRA™), sildenafil citrate, zaprinast, LASSBio596, E-4010, and vardenafil, or a combination thereof. In some embodiments, examples of PDE5 inhibitors which can be used include, without limitation, pyrimidine and pyrimidinone derivatives, such as the compounds described in U.S. Pat. Nos. 6,677,335, 6,458,951, 6,251,904, 6,787,548, 5,294, 612, 5,250,534, 6,469,012, WO 94/28902, WO96/16657, EP0702555, and Eddahibi, Br. J. Pharmacol., 125(4): 681688 (1988); griseolic acid derivatives, such as the compounds disclosed in U.S. Pat. No. 4,460,765; 1-arylnaphthalene ligands, such as those described in Ukita, J. Med. Chem. 42(7): 1293-1305 (1999); quinazoline derivatives, such as 4-[[3′,4′-(methylenedioxy)benzyl]amino]-6-methoxyquinazoline) and compounds described in U.S. Pat. Nos. 3,932,407, 4,146,718, and RE31,617; pyrroloquinolones and pyrrolopyridinones, such as those described in U.S. Pat. Nos. 6,686,349, 6,635,638, 6,818,646, US20050113402; carboline derivatives, such the compounds described in U.S. Pat. Nos. 6,492,358, 6,462,047, 6,821,975, 6,306,870, 6,117,881, 6,043,252, 3,819,631, US20030166641, WO 97/43287, Daugan et al, J Med. Chem, 46(21):4533-42 (2003), and Daugan et al, J Med. Chem, 9; 46(21):4525-32 (2003); imidazo derivatives, such as the compounds disclosed in U.S. Pat. Nos. 6,130,333, 6,566,360, 6,362,178, 6,582,351, US20050070541, and US20040067945; and compounds described in U.S. Pat. Nos. 6,825,197, 6,943,166, 5,981,527, 6,576,644, 5,859,009, 6,943,253, 6,864,253, 5,869,516, 5,488,055, 6,140,329, 5,859,006, 6,143,777, WO 96/16644, WO 01/19802, WO 96/26940, Dunn, Org. Proc. Res. Dev, 9: 88-97 (2005), and Bi et al, Bioorg Med Chem. Lett, 11(18):2461-4 (2001). Content of all of the above is incorporated herein by reference in its entirety.

Additional exemplary PDE5 inhibitors include, but are not limited to, zaprinast; MY-5445; dipyridamole; sulindac sulfone; vinpocetine; FR229934; 1-methyl-3-isobutyl-8-(methylamino)xanthine; furazlocillin; Sch-51866; E4021; GF-196960; IC-351; T-1032; sildenafil; tadalafil; vardenafil; DMPPO; RX-RA-69; KT-734; SKF-96231; ER-21355; BF/GP-385; NM-702; PLX650; PLX134; PLX369; PLX788; vesnarinone; sildenafil or a related compound disclosed in U.S. Pat. Nos. 5,346,901, 5,250,534, or 6,469,012; tadalafil or a related compound disclosed in U.S. Pat. Nos. 5,859,006, 6,140,329, 6,821,975, or 6,943,166; or vardenafil or a related compound disclosed in U.S. Pat. No. 6,362,178. Content of all of the above is incorporated herein by reference in its entirety.

In some embodiments, an anti-miR agent (e.g., that inhibits at least one of miR-103, miR-105, miR-107 and/or miR-155) and a pharmaceutically active agent can be administrated to the subject in the same pharmaceutical composition or in different pharmaceutical compositions (at the same time or at different times).

Delivery of Anti-miR Agents and miRs and miRNAs

In one embodiment, a vector encoding an anti-miR agent (e.g., that inhibits at least one of miR-103, miR-105, miR-107 and/or miR-155) or miR agent (e.g., miR-103, miR-105, miR-107 and/or miR-155 agent) is delivered into a specific target cell. Nucleic acid sequences necessary for expression in prokaryotes usually include a promoter, an operator (optional), often along with other sequences. Eukaryotic cells are known to utilize promoters, enhancers, and termination and polyadenylation signals.

One can also use localization sequences to deliver the released anti-miR agent (e.g., that inhibits at least one of miR-103, miR-105, miR-107 and/or miR-155) or miR agent (e.g., miR-103, miR-105, miR-107 and/or miR-155 agent) intracellularly to a cell compartment of interest. Typically, the delivery system first binds to a specific receptor on the cell. Thereafter, the targeted cell internalizes the delivery system, which is bound to the cell. For example, membrane proteins on the cell surface, including receptors and antigens can be internalized by receptor mediated endocytosis after interaction with the ligand to the receptor or antibodies. (Dautry-Varsat, A., et al., Sci. Am. 250:52-58 (1984)). This endocytic process is exploited by the present delivery system. Because this process may damage an anti-miR agent (e.g., that inhibits at least one of miR-103, miR-105, miR-107 and/or miR-155) or miR agent (e.g., miR-103, miR-105, miR-107 and/or miR-155 agent), for example a RNAi or siRNA agent, or anti-miR as it is being internalized, it may be desirable to use a segment containing multiple repeats of the RNA interference-inducing molecule of interest. One can also include sequences or moieties that disrupt endosomes and lysosomes. See, e.g., Cristiano, R. J., et al., Proc. Natl. Acad. Sci. USA 90:11548-11552 (1993); Wagner, E., et al., Proc. Natl. Acad. Sci. USA 89:6099-6103 (1992); Cotten, M., et al., Proc. Natl. Acad. Sci. USA 89:6094-6098 (1992).

In some embodiments, an anti-miR agent (e.g., that inhibits at least one of miR-103, miR-105, miR-107 and/or miR-155) or miR agent (e.g., miR-103, miR-105, miR-107 and/or miR-155 agent) can be complexed with desired targeting moieties by mixing an anti-miR agent, e.g., anti-miR RNA interference molecules with a targeting moiety in the presence of complexing agents. Examples of such complexing agents include, but are not limited to, poly-amino acids; polyimines; polyacrylates; polyalkylacrylates, polyoxethanes, polyalkylcyanoacrylates; cationized gelatins, albumins, starches, acrylates, polyethyleneglycols (PEG) and starches; polyalkylcyanoacrylates; DEAE-derivatized polyimines, pollulans, celluloses and starches. In some embodiments, the complexing agents include chitosan, N-trimethylchitosan, poly-L-lysine, polyhistidine, polyornithine, polyspermines, protamine, polyvinylpyridine, polythiodiethylaminomethylethylene P(TDAE), polyaminostyrene (e.g. p-amino), poly(methylcyanoacrylate), poly(ethylcyanoacrylate), poly(butylcyanoacrylate), poly(isobutylcyanoacrylate), poly(isohexylcynaoacrylate), DEAE-methacrylate, DEAE-hexylacrylate, DEAE-acrylamide, DE AE-albumin and DEAE-dextran, polymethylacrylate, polyhexylacrylate, poly(D,L-lactic acid), poly(DL-lactic-co-glycolic acid (PLGA), alginate, and polyethyleneglycol (PEG), and polyethylenimine.

In alternative embodiments, an anti-miR agent (e.g., that inhibits at least one of miR-103, miR-105, miR-107 and/or miR-155) or miR agent (e.g., miR-103, miR-105, miR-107 and/or miR-155 agent) is complexed to a complexing agent, e.g., anti-miR complexing agent such as a protamine or an RNA-binding domain, such as an siRNA-binding fragment or nucleic acid binding fragment of protamine. Protamine is a polycationic peptide with molecular weight about 4000-4500 Da. Protamine is a small basic nucleic acid binding protein, which serves to condense the animal's genomic DNA for packaging into the restrictive volume of a sperm head (Warrant, R. W., et al., Nature 271:130-135 (1978); Krawetz, S. A., et al., Genomics 5:639-645 (1989)). The positive charges of the protamine can strongly interact with negative charges of the phosphate backbone of nucleic acid, such as RNA, resulting in a neutral and stable interference RNA-protamine complex.

In one embodiment, the protamine fragment is encoded by a nucleic acid sequence disclosed in International Patent Application: PCT/US05/029111, which is incorporated herein in its entirety by reference. The methods, reagents and references that describe a preparation of a nucleic acid-protamine complex in detail are disclosed in the U.S. Patent Application Publication Nos. US2002/0132990 and US2004/0023902, and are herein incorporated by reference in their entirety.

### Targeting an Anti-miR Agent

In another embodiment of the invention an anti-miR agent (e.g., that inhibits at least one of miR-103, miR-105, miR-107 and/or miR-155) or miR agent (e.g., miR-103, miR-105, miR-107 and/or miR-155 agent) as disclosed herein are targeted to specific cells, for example cells expressing any one of miR-103, miR-105, and/or miR-107 and/or miR-155; and/or cells expressing the NPPA mRNA in order to avoid any potential undesired side effects of the anti-miR agent (e.g., that inhibits at least one of miR-103, miR-105, miR-107 and/or miR-155) or miR agent (e.g., miR-103, miR-105, miR-107 and/or miR-155 agent). In some embodiments, anti-miR agent (e.g., that inhibits at least one of miR-103, miR-105, miR-107 and/or miR-155) or miR agent (e.g., miR-103, miR-105, miR-107 and/or miR-155 agent) is targeted to a cardiac cell.

However, as miR-103, miR-105, miR-107 and/or miR-155 can be present in the plasma of subjects, it is not essential to have the anti-miR agent or miR agent targeted to any particular cell, as an anti-miR agent (e.g., antagonizing miRs) present in the plasma could have an effect even if the anti-miR does not reach the heart. For example, an anti-miR that inhibits at least one of miR-103, miR-105, miR-107 and/or miR-155) as disclosed herein in the plasma can have effect at inhibiting the effect of respective miR circulating in the blood. In some embodiments, an anti-miR agent (e.g., that inhibits at least one of miR-103, miR-105, miR-107, and/or miR-155) can be fused to a cell targeting moiety or protein, as disclosed in the International Patent Application PCT/US05/029111 which is incorporated herein in its entirety by reference.

In such embodiments, the target moiety specifically brings the delivery system to the target cell. The particular target moiety for delivering the interference RNAs, such as anti-miRs complementary to at least part of a miR-103 sequence of SEQ ID NO: 1-3, miR-105 sequence of SEQ ID NO: 7 or 8 or miR-107 sequence of SEQ ID NO: 11, or a miR-155 sequence of SEQ ID NO: 12, or at least part of the seed sequences or pre-mir thereof can be determined empirically based upon the present disclosure and depending upon the target cell.

In some embodiments of the present invention, an anti-miR agent as disclosed herein can be delivered to a limited number of cells thereby limiting, for example, potential side effects of therapies using the anti-miR agent. The particular cell surface targets that are chosen for the targeting moiety will depend upon the target cell. Cells can be specifically targeted, for example, by use of antibodies against unique proteins, lipids or carbohydrates that are present on the cell surface. A skilled artisan can readily determine such molecules based on the general knowledge in the art.

The strategy for choosing the targeting moiety is very adaptable. For example, any cell-specific antigen, including proteins, carbohydrates and lipids can be used to create an antibody that can be used to target the anti-miR agent, e.g., an agent that inhibits at least one of miR-103, miR-105, miR-107 and/or miR-155, to a specific cell type according to the methods described herein.

In some embodiments, a binding domain is used to complex the targeting moiety to an anti-miR agent (e.g., that inhibits at least one of miR-103, miR-105, miR-107 and/or miR-155) In some embodiments, the binding domain is selected from the nucleic acid binding domains present in proteins selected from the group consisting of GCN4, Fos, Jun, TFIIS, FMRI, yeast protein HX, Vigillin, Merl, bacterial polynucleotide phosphorylase, ribosomal protein S3, and heat shock protein.

Treating a Subject with an Anti-miR Agent

In some embodiments, an anti-miR agent (e.g., that inhibits at least one of miR-103, miR-105, miR-107 and/or miR-155) as disclosed herein is administered to a subject in a pharmaceutical composition where the subject has a high blood pressure, or a cardiovascular disease or disorder, or hypertension. The administration may be a treatment and/or prophylaxis for any one of high blood pressure, hypertension and the like.

In some embodiments, the methods described herein encompass administering a pharmaceutical composition to a subject comprising an anti-miR agent (e.g., that inhibits at least one of miR-103, miR-105, miR-107 and/or miR-155) to a subject, for example, where the subject has normal or high levels of a miR-103, miR-105, miR-107, and/or miR-155 above a predefined reference level. In some embodiments, an anti-miR agent as disclosed herein (e.g., an agent which inhibits at least one of miR-103, miR-105, miR-107 and/or miR-155) is administered to a subject who has circulating levels of miR-103, miR-105, miR-107 or miR-155 above a pre-defined level, where the anti-miR agent is administered in a location where miR-103, miR-105, miR-107, or miR-155 is expressed, e.g., but not limited to, the atrium and ventricle regions of the heart. In some embodiments, an anti-miR agent as disclosed herein is a small molecule which inhibits the function of at least one of miR-103, miR-105, miR-107 and/or miR-155, and/or inhibits the expression of at least one of miR-103, miR-105, miR-107 and/or miR-155. In some embodiments such an anti-miR agent is an oligonucleotide which inhibits the function and/or expression of at least one of miR-103, miR-105, miR-107 and/or miR-155. In some embodiments, an oligonucleotide anti-miR binds to any region of miR-103, miR-105, miR-107 and/or miR-155 as disclosed herein to inhibit its binding to the miRNA target region on the 3′UTR of the NPPA gene. In such embodiments, an oligonucleotide anti-miR agent as disclosed herein (e.g., an agent which inhibits at least one of miR-103, miR-105, miR-107 and/or miR-155) is a LNA or a Tiny LNA as disclosed herein. In some embodiments, an anti-miR agent for use in the methods, compositions, kits and systems as disclosed herein are modified as disclosed in US Application US2014/0066491, which is incorporated herein in its entirety by reference. In some embodiments, an anti-miR agent for use in the composition, methods and kits as disclosed herein are LNA-anti-miR oligonucleotides as disclosed in Montgomery et al., Circulation, 2011; 124; 1537-1547; and Hullinger et al., Circulation, 2012; 110; 71-81, each of which are incorporated herein in their entirety by reference. In some embodiments, a LNA-anti-miR oligonucleotides for use in the present invention are synthesized as fully phosphorothiolated oligonucleotides which are at least 90% complementary to the 5′ region of the mature miR-103, miR-107, miR-105, miR-155 or miR-425 sequence. In some embodiments, a LNA-anti-miR oligonucleotides for use in the present invention is synthesized as unconjugated and fully phosphorothiolated oligonucleotides which are at least 90% complementary to the 5′ region of the mature miR-103, miR-107, miR-105, miR-155 or miR-425 sequence. LNA-anti-miRs for use in the methods and compostion as disclosed herein allow robust reduction in miR levels in response to their anti-miR in vivo in cardiac tissue as demonstrated in Montgomery et al., Circulation, 2011; 124; 1537-1547; and Hullinger et al., Circulation, 2012; 110; 71-81.

In another aspect of the present invention, the methods described herein encompass administering a pharmaceutical composition comprising an anti-miR agent as disclosed herein (e.g., an agent which inhibits at least one of miR-103, miR-105, miR-107 and/or miR-155) to a subject, where the subject has high levels of miR-103, miR-105, miR-107 and/or miR-155 above a predefined reference level.

Effective, safe dosages can be experimentally determined in model organisms and in human trials by methods well known to one of ordinary skill in the art. An anti-miR agent as disclosed herein (e.g., an agent which inhibits at least one of miR-103, miR-105, miR-107 and/or miR-155) in a pharmaceutical composition can be administered alone or in combination with adjuvant therapy such as, but not limited to, decreased stress, increased exercise, healthier diet, as well as administration with other therapeutic agents well known in the art to treat hypertension and high-blood pressure, such as, but not limited to include thiazide diuretics, beta blockers, angiotensin converting enzyme (ACE) inhibitors, and calcium channel. In some embodiments, a dose of anti-miR agent as disclosed herein (e.g., an agent which inhibits at least one of miR-103, miR-105, miR-107 and/or miR-155) is determined by an ordinary physician or one of ordinary skill in the art when the subject is also administered an adjuvant therapy. In some embodiments, a dose of an anti-miR agent as disclosed herein (e.g., an agent which inhibits at least one of miR-103, miR-105, miR-107 and/or miR-155) is determined by an ordinary physician or one of ordinary skill in the art when the subject is not administered an adjuvant therapy, for example, a test dose with anti-miR agent as disclosed herein (e.g., an agent which inhibits at least one of miR-103, miR-105, miR-107 and/or miR-155) is administered to a subject and its efficacy can be determined by measuring the level of circulating miR-103, miR-105, miR-107 and/or miR-155 respectively (depending on the miR targeted by the anti-miR agent) and/or at least one symptom of hypertension or high blood pressure measured (e.g., blood pressure can be measured using a blood pressure cuff) and depending on the effect of the anti-miR agent, the dose of the anti-miR agent (e.g., an agent which inhibits at least one of miR-103, miR-105, miR-107 and/or miR-155) can be modified accordingly based on the efficacy and/or any adverse side effects.

Subjects Amenable to Treatment with an Anti-miR Agents

One aspect as disclosed herein relates to use of an anti-miR agent in methods for treating any type of disease or disorder in which it is desirable to increase ANP levels. These include, for example, diseases where ANP is reduced, or where ANP is not expressed at high levels and contributes to the disease pathology and/or progression of the disease, such as high blood pressure, hypertension and the like. These also include diseases in which ANP may be elevated but in which augmented ANP levels may have therapeutic benefit. For example, in some embodiments, subjects with heart failure where ANP levels are high, can benefit from increased ANP or BNP levels, and thus are amenable to administration of an anti-miR agent as disclosed herein (e.g., anti-miR-103, anti-miR-105, anti-miR-107 or anti-miR-155). For example, a recent PARADIGM-HF trial treated individuals with a neprilysin inhibitor which reduces the degradation of ANP and BNP and improved outcomes in patients with heart failure, confirming that enhanced natriuretic peptide activity even in states of elevated natriuretic peptides could be beneficial (McMurray et al NEJM 2014).

The invention provides methods for the treatment of any disease or disorder characterized by low levels of ANP. In some embodiments, the disease is a cardiovascular disease or disorder, such as but not limited to any one or a combination of; high blood, hypertension, heart failure, congestive heart failure, left ventriclar hypertrophy, metabolic syndrome, stroke and renal failure. Hypertension is a leading cause of human cardiovascular morbidity and mortality, with a prevalence rate of 25-30% of the adult Caucasian population of the United States (JNC Report, 1985). Pulmonary hypertension is a pathological condition in which the pulmonary arterial pressure rises above normal levels and may cause sequelae of haemodynamic changes that can become life threatening. Symptoms of pulmonary hypertension include shortness of breath with minimal exertion, fatigue, dizzy spells and fainting. When pulmonary hypertension occurs in the absence of a known cause, it is referred to as idiopathic pulmonary arterial hypertension (previously referred to as primary pulmonary hypertension). Idiopathic pulmonary arterial hypertension is rare, occurring in about two per million people worldwide. Secondary hypertension is much more common occurring as a result of other medical conditions, including congestive heart failure, chronic hypoxic lung disorder, including chronic obstructive pulmonary disease, inflammatory or collagen vascular diseases such as scleroderma and systemic lupus erythematosus, congenital heart diseases associated with left to right shunting and pulmonary thromboembolism.

In some embodiments, the present invention relates to use of an anti-miR agent (e.g., an agent which inhibits at least one of miR-103, miR-105, miR-107 and/or miR-155) as disclosed herein, for the treatment of cardiovascular disorders and/or congenital heart disease in a subject. In some embodiments, an anti-miR agent (e.g., an agent which inhibits at least one of miR-103, miR-105, miR-107 and/or miR-155) as disclosed herein can be used to treat any one or a combination of disorders selected from the group consisting of congestive heart failure, hypertension, systemic hypertension (thereby preventing the development of myocardial infarction, chronic heart failure (CHF), renal failure, and stroke), pulmonary hypertension, acute kidney injury, modulating inflammatory responses (either positively or negatively), lipolysis, obesity, diabetes mellitus, reducing intraocular pressure (preventing blindness in glaucoma, reducing ischemia-reperfusion (I/R) injury, (including spinal cord I/R injury and cardiac I/R injury), preventing atrial fibrillation, liver failure, liver fibrosis, cirrhosis, cancer, promote angiogenesis, weight loss.

In some embodiments, circulating miR-103, miR-105, miR-107 and/or miR-155 levels can be used to assess a subject with a specific heart failure phenotype referred to as preserved ejection fraction (HpEF aka Diastolic Heart failure) which is reported to be a “heterogenous subtype” of heart failure. Previous researchers have concerns of using elevated plasma concentrations of natriuretic peptides as a patient selection criterion in HFpEF trials (I-PRESERVE Trial, Anand et al Circulation: Heart failure 2011). Accordingly, in some embodiments the methods and assays as disclosed herein to measure circulating miR-103, miR-105, miR-107 and/or miR-155 can be useful for providing clinical care in these subset of patients with a heart failure phenotype having preserved ejection fraction (e.g., subjects with symptomatic HFpEF). Accordingly, circulating miR-103, and/or miR-105 and/or miR-107 levels can be used to determine a “low” natriuretic peptide concentration in a patient with symptomatic HFpEF.

In one embodiment of the invention relates to a method of treating a circulatory disorder comprising administering an effective amount of a composition comprising an anti-miR agent (e.g., an agent which inhibits at least one of miR-103, miR-105, miR-107 and/or miR-155) as disclosed herein to a subject with a circulatory disorder. In a further embodiment, the invention provides a method for treating hypertension (e.g., high blood pressure), comprising administering a composition comprising anti-miR agent (e.g., an agent which inhibits at least one of miR-103, miR-105, miR-107 and/or miR-155) as dislosed herein.

In one embodiment of the above methods, the subject is a human and the composition comprising anti-miR agent (e.g., an agent which inhibits at least one of miR-103, miR-105, miR-107 and/or miR-155) is useful to treat circulatory disorder is selected from the group consisting of cardiomyopathy, myocardial infarction, and congenital heart disease. In some embodiments, the circulatory disorder is a myocardial infarction. The present invention is also directed to a method of treating circulatory damage in the heart or peripheral vasculature which occurs as a consequence of genetic defect, physical injury, environmental insult or damage from a stroke, heart attack or cardiovascular disease (most often due to ischemia) in a subject, the method comprising administering anti-miR agent (e.g., an agent which inhibits at least one of miR-103, miR-105, miR-107 and/or miR-155) to a subject. Medical indications for such treatment include treatment of acute and chronic heart conditions of various kinds, such as coronary heart disease, cardiomyopathy, endocarditis, congenital cardiovascular defects, and congestive heart failure. Efficacy of treatment can be monitored by clinically accepted criteria, such as an improvement in developed pressure, systolic pressure, end diastolic pressure, patient mobility, and quality of life.

The methods as disclosed herein comprising administering anti-miR agent (e.g., an agent which inhibits at least one of miR-103, miR-105, miR-107 and/or miR-155) can be used to treat a wide variety of diseases including, but not limited to essential hypertension, hypertension associated with end stage renal failure, hypertension associated with pregnancy (preeclampsia), salt sensitivity hypertension, type II diabetes hypertension, hypertension associated with alcohol abuse, obesity associated hypertension, systolic hypertension in elderly, asthma, allergies, migraine headache, gastrointestinal motility disorders, Alzheimer's disease, senile dementia, angina pectoris, premature labor, cerebrovascular diseases, and convulsive epilepsy. The methods are also suited for treatment of essential hypertension and intra-ocular hypertension.

The pharmaceutical compositions and dosage forms of anti-miR agent (e.g., an agent which inhibits at least one of miR-103, miR-105, miR-107 and/or miR-155) may be used for treating a wide variety of disease states which involve one or more forms of cardiovascular, cerebrovascular, and intraocular dysfunction. The anti-miR agent (e.g., an agent which inhibits at least one of miR-103, miR-105, miR-107 and/or miR-155) as disclosed herein generally possess a broad spectrum of cardiovascular and cerebrovascular activities including increasing ANP levels. The invention compositions can therefore be beneficially used in treating cardiovascular disorders, cerebrovascular disorders.

In one embodiment, administration of multiple anti-miR agents (e.g., at least one or a combination of agents which inhibits at least one of miR-103, miR-105, miR-107 and/or miR-155) provides a safe, highly effective method for treating severe hypertension and may offer an alternative to side effects associated with other antihypertensive drugs (e.g., nitrendipine). The present invention relies on inhibiting the repression of the NPPA mRNA by an anti-miR agent (e.g., an agent which inhibits at least one of miR-103, miR-105, miR-107 and/or miR-155), and is useful for treatment or prevention of hypertension, while simultaneously reducing many of the undesirable side effects (e.g., headache, nausea) associated with other known anti-hypertensive drugs.

In particular, anti-miR agent (e.g., an agent which inhibits at least one of miR-103, miR-105, miR-107 and/or miR-155) as disclosed herein are useful in the treatment of essential hypertension and/or various secondary hypertensive conditions (e.g., end stage renal hypertension, pregnancy associated hypertension such as preeclampsia, hypertension associated with type II diabetes, salt sensitivity hypertension, hypertension associated with alcohol abuse, hypertension associated with obesity, and systolic hypertension in the elderly).

Treatment of hypertension using anti-miR agent (e.g., an agent which inhibits at least one of miR-103, miR-105, miR-107 and/or miR-155) as disclosed herein typically involves first diagnosing the hypertensive condition and whether treatment with an anti-miR agent (e.g., an agent which inhibits at least one of miR-103, miR-105, miR-107 and/or miR-155) is appropriate; administration of an anti-miR agent (e.g., an agent which inhibits at least one of miR-103, miR-105, miR-107 and/or miR-155) alone or in combination with one or more other drug(s) in a therapeutic regimen, monitoring response of the subject, and, if necessary, altering/optimizing dosage/treatment regimen.

Methods of diagnosing essential or secondary hypertension are well known to those of skill in the art (see, e.g., Isselbacher et al. (1994) Harrison's Principles of Internal Medicine, 13.sup.th Ed., McGraw-Hill, Inc., New York). Physical examination and laboratory tests are directed at (1) uncovering correctable secondary forms of hypertension; (2) establishing a pretreament baseline, (3) assessing factors which may influence the type or therapy or which may be adversely modified by therapy, (4) determining if target organ damage is present and (5) determining whether other risk factors for the development or arteriosclerotic cardiovascular diseases are present.

In some embodiments, a subject amenable to treatment with anti-miR agent (e.g., an agent which inhibits at least one of miR-103, miR-105, miR-107 and/or miR-155) as disclosed herein has normal or high levels of any one of miR-103, miR-105, miR-107 and/or miR-155. In some embodiments, a subject is screened for the presence or amount of miR-103, miR-105, miR-107 and/or miR-155 in a biological sample from the subject, e.g., a blood or plasma sample from the subject according to the methods and assays as disclosed herein. In some embodiments, a level of any one of miR-103, miR-105, miR-107 and/or miR-155 above a pre-defined standard level (e.g., reference level) indicates a subject is amenable to treatment with an anti-miR agent (e.g., an agent which inhibits at least one of miR-103, miR-105, miR-107 and/or miR-155) as disclosed herein.

In the event treatment with anti-miR agent is indicated, anti-miR agent (e.g., an agent which inhibits at least one of miR-103, miR-105, miR-107 and/or miR-155) as disclosed herein can be administered to the subject organism (e.g., patient) alone or in combination with other medications or medicaments (e.g., other anti-hypertensives such as diuretics, antiadrenergic agents, angiotensin-converting enzyme (ACE) inhibitors, other calcium channel antagonists (e.g., nifedipine, amlodipine, verapamil, diltiazem, etc.), or other pharmacological agents) as described below. The subject will be monitored and evaluated according to standard methods in the art and dosages adjusted accordingly.

In one method anti-miR agent (e.g., an agent which inhibits at least one of miR-103, miR-105, miR-107 and/or miR-155) can be administered to an individual suffering from hypertension. For example, a composition comprising an anti-miR agent (e.g., an agent which inhibits at least one of miR-103, miR-105, miR-107 and/or miR-155) and a pharmaceutically acceptable excipient is administered therapeutically to an individual to reduce or ameliorate hypertension. In another embodiment, anti-miR agent (e.g., an agent which inhibits at least one of miR-103, miR-105, miR-107 and/or miR-155) as disclosed herein-can be administered prophylactically to reduce the probability of occurrence of hypertension or to mitigate and/or prevent the onset of hypertension associated pathologies (e.g., stroke, kidney failure, etc.).

In some embodiments, compositions comprising anti-miR agent (e.g., an agent which inhibits at least one of miR-103, miR-105, miR-107, and/or miR-155) be used for the treatment of angina pectoris. Angina Pectoris results from the narrowing of the coronary arteries and subsequent reduction in blood supply to the myocardium. Total obstruction of these arteries lead to myocardial infarction. Accordingly, in one embodiment, anti-miR agent (e.g., an agent which inhibits at least one of miR-103, miR-105, miR-107, and/or miR-155) as disclosed herein can be used in the treatment of angina pectoris and myocardial infarction. For example, ANP can prevent adverse LV remodeling and thereby preventing the development of congestive heart failure.

As discussed herein, in some embodiments, anti-miR agent (e.g., an agent which inhibits at least one of miR-103, miR-105, miR-107 and/or miR-155) as disclosed herein can be used to treat any one or a combination of disorders selected from the group consisting of congestive heart failure, hypertension, systemic hypertension (thereby preventing the development of myocardial infarction, chronic heart failure (CHF), renal failure, and stroke), pulmonary hypertension, acute kidney injury, modulating inflammatory responses (either positively or negatively), lipolysis, obesity, diabetes mellitus, reducing intraocular pressure (preventing blindness in glaucoma, reducing ischemia-reperfusion (I/R) injury, (including spinal cord I/R injury and cardiac I/R injury), preventing atrial fibrillation, liver failure, liver fibrosis, cirrhosis, cancer, promote angiogenesis, weight loss.

In some embodiments, an anti-miR agent (e.g., that inhibits at least one of miR-103, miR-105, and/or miR-107) as disclosed herein can be used in a therapeutic treatment for any one or more of the following disorders; congestive heart failure (CHF) (Kobayashi D, et al., Human atrial natriuretic peptide treatment for acute heart failure: a systematic review of efficacy and mortality. Can J Cardiol; 28:102-9.), systemic hypertension—thereby preventing the development of myocardial infarction, CHF, renal failure, and stroke, pulmonary hypertension, acute kidney injury (Nigwekar et al., Atrial natriuretic peptide for preventing and treating acute kidney injury. Cochrane Database Syst Rev 2009:CD006028.), modulating inflammatory responses (either positively or negatively) (Casserly et al. The role of natriuretic peptides in inflammation and immunity. Recent Pat Inflamm Allergy Drug Discov; 4:90-104), lipolysis (enhancing breakdown of adipose tissue), obesity (Birkenfeld et al., Atrial natriuretic peptide induces postprandial lipid oxidation in humans. Diabetes 2008; 57:3199-204; Lafontan et al., Control of lipolysis by natriuretic peptides and cyclic GMP. Trends Endocrinol Metab 2008; 19:130-7), diabetes mellitus by enhancing insulin secretion in response to glucose (Ropero et al., The atrial natriuretic peptide and guanylyl cyclase-A system modulates pancreatic beta-cell function. Endocrinology; 151:3665-74), reducing intraocular pressure (and thereby preventing blindness in glaucoma, where topical administration of anti-miR-425 would be beneficial), (Wolfensberger et al., Evidence for a new role of natriuretic peptides: control of intraocular pressure. Br J Ophthalmol 1994; 78:446-8), reducing ischemia-reprefusion (I/R) injury, including spinal cord I/R injury 8 and cardiac I/R injury (Gerczuk P Z, Kloner R A. An update on cardioprotection: a review of the latest adjunctive therapies to limit myocardial infarction size in clinical trials. J Am Coll Cardiol; 59:969-78); preventing atrial fibrillation—NPPA gene mutations are a cause of familial atrial fibrillation (Perrin et al., The role of atrial natriuretic peptide in modulating cardiac electrophysiology. Heart Rhythm; 9:610-5), liver fibrosis (Ishigaki et al., Continuous intravenous infusion of atrial natriuretic peptide (ANP) prevented liver fibrosis in rat. Biochem Biophys Res Commun 2009; 378:354-9), cirrhosis, disorders associated with cirrhosis, treatment of ascites associated with cirrhosis, cancer (e.g., inhibit cancer cell growth (Vesely D L. Metabolic targets of cardiac hormones' therapeutic anti-cancer effects. Curr Pharm Des; 16:1159-66.); promote angiogenesis; weight loss (e.g., by enhancing brown adipocyte phenotype and energy expenditure (decreasing weight) (Bordicchia et al., Cardiac natriuretic peptides act via p38 MAPK to induce the brown fat thermogenic program in mouse and human adipocytes. J Clin Invest; 122: 1022-36)).

In alternative embodiments, an anti-miR agent (e.g., that inhibits at least one of miR-103, miR-105, miR-107, and/or miR-155) as disclosed herein can be used in a prophylactic treatment any one of the following diseases and disorders; congestive heart failure (CHF) (Kobayashi D, et al., Human atrial natriuretic peptide treatment for acute heart failure: a systematic review of efficacy and mortality. Can J Cardiol; 28:102-9.), systemic hypertension—thereby preventing the development of myocardial infarction, CHF, renal failure, and stroke, pulmonary hypertension, acute kidney injury (Nigwekar et al., Atrial natriuretic peptide for preventing and treating acute kidney injury. Cochrane Database Syst Rev 2009:CD006028.), modulating inflammatory responses (either positively or negatively) (Casserly et al., The role of natriuretic peptides in inflammation and immunity Recent Pat Inflamm Allergy Drug Discov; 4:90-104), lipolysis (enhancing breakdown of adipose tissue), obesity (Birkenfeld et al., Atrial natriuretic peptide induces postprandial lipid oxidation in humans. Diabetes 2008; 57:3199-204; Lafontan et al., Control of lipolysis by natriuretic peptides and cyclic GMP. Trends Endocrinol Metab 2008; 19:130-7), diabetes mellitus by enhancing insulin secretion in response to glucose (Ropero et al., The atrial natriuretic peptide and guanylyl cyclase-A system modulates pancreatic beta-cell function. Endocrinology; 151:3665-74), reducing intraocular pressure (and thereby preventing blindness in glaucoma, where topical administration of anti-miR-425 would be beneficial), (Wolfensberger et al., Evidence for a new role of natriuretic peptides: control of intraocular pressure. Br J Ophthalmol 1994; 78:446-8), reducing ischemia-reprefusion (I/R) injury, including spinal cord I/R injury 8 and cardiac I/R injury (Gerczuk P Z, Kloner R A. An update on cardioprotection: a review of the latest adjunctive therapies to limit myocardial infarction size in clinical trials. J Am Coll Cardiol; 59:969-78); preventing atrial fibrillation—NPPA gene mutations are a cause of familial atrial fibrillation (Perrin et al., The role of atrial natriuretic peptide in modulating cardiac electrophysiology. Heart Rhythm; 9:610-5), liver fibrosis (Ishigaki et al., Continuous intravenous infusion of atrial natriuretic peptide (ANP) prevented liver fibrosis in rat. Biochem Biophys Res Commun 2009; 378:354-9), cirrhosis, disorders associated with cirrhosis, treatment of ascites associated with cirrhosis, cancer (e.g., inhibit cancer cell growth (Vesely D L. Metabolic targets of cardiac hormones' therapeutic anti-cancer effects. Curr Pharm Des; 16:1159-66.); promote angiogenesis; weight loss (e.g., by enhancing brown adipocyte phenotype and energy expenditure (decreasing weight) (Bordicchia et al., Cardiac natriuretic peptides act via p38 MAPK to induce the brown fat thermogenic program in mouse and human adipocytes. J Clin Invest; 122: 1022-36))

In some embodiments, addition, an anti-miR agent (e.g., that inhibits at least one of miR-103, miR-105, miR-107, and/or miR-155) can be used for prophylactic treatment of high blood pressure. There are hereditary conditions and/or environmental situations (e.g. stress, diet and high Body Mass index (BMI) and obesity) known in the art that predispose an individual to developing high blood pressure and/or heart failure. Under these circumstances, it may be beneficial to treat these individuals with therapeutically effective doses an anti-miR as disclosed herein, e.g., an amount of a anti-miR to reduce the risk of developing high blood pressure, heart failure or other cardiovascular diseases and conditions as disclosed herein.

In some embodiments, an anti-miR agent (e.g., that inhibits at least one of miR-103, miR-105, miR-107, and/or miR-155) as disclosed herein is administered to a subject identified to be at risk of a cardiovascular disease or disorder identified by cardiovascular biomarkers, such as but not limited to CRP, creatine phosphokinase (CPK), aspartate aminotransferase (AST), lactate dehydrogenase (LDH) in the blood, or a symptom of cardiovascular disease as determined by someone of ordinary skill in the art as measured by electrocardiogram (ECG or EKG), or echocardiogram (heart ultrasound). In some embodiments, a subject at risk of cardiovascular disease is identified by the presence of one or more biomarkers such as, but not limited to cardiac troponins, C-reactive protein (CRP), ANP, BNP, adrenomedullin, copeptin NT-proBNP, NT-proANP, mid-regional-proANP, Galectin-3, ST-2, GDF-15 and others, which are well known by persons of ordinary skill in the art.

In one embodiment, an anti-miR agent (e.g., that inhibits at least one of miR-103, miR-105, miR-107, and/or miR-155) as disclosed herein present in a suitable formulation may be administered to a subject who has a family history of high blood pressure, or to a subject who has a genetic predisposition for high blood pressure. In other embodiments, an anti-miR agent (e.g., that inhibits at least one of miR-103, miR-105, miR-107, and/or miR-155) is administered to a subject who has reached a particular age, or to a subject more likely to get high blood pressure. In yet other embodiments, an anti-miR agent (e.g., that inhibits at least one of miR-103, miR-105, miR-107, and/or miR-155) alone or in combination with an anti-miR-425 as disclosed herein is administered to subjects who exhibit symptoms of high blood pressure. In still other embodiments, an anti-miR agent (e.g., that inhibits at least one of miR-103, miR-105, miR-107, and/or miR-155) alone or in combination with an anti-miR-425 as disclosed herein can be administered to a subject as a preventive measure. In some embodiments, an anti-miR agent (e.g., that inhibits at least one of miR-103, miR-105, miR-107, and/or miR-155) alone or in combination with an anti-miR-425 as disclosed herein can be administered to a subject based on demographics or epidemiological studies, or to a subject in a particular field or career, e.g., high-stress working environments, or fields and careers where it is desirable subject maintain a low blood pressure.

In addition, subjects being identified as being homozygous or heterozygous for the major allele (e.g., A/A or A/G) for the rs5068 SNP and/or identified as being homozygous or heterozygous for the major allele (e.g., A/A or A/G) for the rs61764044 SNP can be administered an anti-miR agent (e.g., that inhibits at least one of miR-103, miR-105, and/or miR-107) in combination with an anti-miR-425 as disclosed herein.

In some embodiments, subject amenable to treatment with an anti-miR agent (e.g., that inhibits at least one of miR-103, miR-105, miR-107, and/or miR-155) as disclosed herein have a blood pressure of above 120/80 mm Hg, above 115/75 mm Hg, where the risk of cardiovascular disease begins to increase. In some embodiments, subject amenable to treatment with an anti-miR agent (e.g., that inhibits at least one of miR-103, miR-105, miR-107, and/or miR-155) as disclosed herein have prehypertension, where their Prehypertension. Prehypertension is a systolic pressure ranges from 120 to 139 mm Hg or a diastolic pressure ranges from 80 to 89 mm Hg. In some embodiments, subject amenable to treatment with an anti-miR agent (e.g., that inhibits at least one of miR-103, miR-105, miR-107, and/or miR-155) as disclosed herein have stage 1 or Stage 2 hypertension, where subjects with stage 1 hypertension have a systolic pressure ranging from about 90-100 mm Hg to about 159 mm Hg or a diastolic pressure ranging from 90 to 99 mm Hg, and subjects with stage 2 hypertension have a systolic pressure of 160 mm Hg or higher or a diastolic pressure of 100 mm Hg or higher.

Subjects Amenable to Treatment with miR-103, miR-105 or miR-107 or a Mimetic Thereof.

In some embodiments, the present invention relates to a method of decreasing atrial natriuric peptide (ANP) in a subject in need thereof, comprising administering to the subject a composition comprising a miR agent as disclosed herein (e.g., at least one of, or a combination of miR-103, miR-105, miR-107, and/or miR-155), alone or in combination with a miR-425 (e.g., SEQ ID NO: 15) or a mimetic or homologue thereof to a subject. In some embodiments, a subject in need thereof is a subject who needs an increase in blood pressure. In such embodiments, the method of decreasing ANP in a subject who has high levels of ANP is useful for treating low blood pressure and associated disorders, e.g., shock, or treat any one or a combination of inhibiting angiogenesis, increased endothelial permeability, or orthostatic hypotension. In some embodiments, administration of a miR agent (e.g., miR-103, miR-105, miR-107, and/or miR-155) as disclosed herein, or mimetic thereof is administered to a subject to treat any one or a combination of inhibiting angiogenesis, low blood pressure, increased endothelial permeability, orthostatic hypotension.

In some embodiments, a miR agent as disclosed herein (e.g., miR-103, miR-105, miR-107, and/or miR-155 agent) is useful in a method of decreasing ANP in a subject for treating low blood pressure and associated disorders as disclosed herein.

In some embodiments, a miR-155 agent which binds to the miR-155 target sequence SEQ ID NO: 199 is administered to a subject whom has been identified as having at least one A allele of rs61764044 SNP, e.g., a AA or AG subject. In alternative embodiments, a modified miR-155 agent which binds to the “G” allele miR-155 target sequence of SEQ ID NO: 200 in the 3′UTR of the NPPA gene is administered to a subject who has been identified to have at least one G allele or rs61764044 SNP (e.g., GG or AG subjects). Stated another way, where a subject is being administered a miR-155 agent to a subject to treat any one or a combination of inhibiting angiogenesis, low blood pressure, increased endothelial permeability, or orthostatic hypotension, and the subject has at least one G (minor) allele of rs61764044 SNP, e.g., a GG or AG subject (i.e., miR-155 of SEQ ID NO: 12 does not typically or efficiently bind to the 3′UTR of the NPPA gene due to G:U wobble base pairing, therefore does not effectively repress NPPA gene expression), the subject the subject is administered a modified miR-155 agent which binds to the “G” allele miR-155 target sequence of SEQ ID NO: 200 (5′-UAAUGCGUGGGGUGGGAGAGG-3′) in the 3′UTR of the NPPA gene. In alternative embodiments, where a subject is being administered a miR-155 agent to a subject to treat any one or a combination of inhibiting angiogenesis, low blood pressure, increased endothelial permeability, or orthostatic hypotension, and the subject has at least one A (major) allele of rs61764044 SNP, e.g., a AA or AG subject (i.e., miR-155 of SEQ ID NO: 12 does repress NPPA gene expression), the subject the subject is administered a miR-155 agent which binds to the “A” allele miR-155 target sequence of SEQ ID NO: 199 (5′-UAAUGCAUGGGGUGGGAGAGG-3′). In some embodiments, where a subject is heterozygous (AG) for the rs61764044 SNP, the subject is administered a combination of miR-155 agents, where at least one miR-155 agent that targets/binds to the A rs61764044 allele miR-155 target sequence of SEQ ID NO: 199 (5′-UAAUGCAUGGGGUGGGAGAGG-3′), and at least one miR-155 agent is a modified miR-agent, targets/binds to the G rs61764044 allele miR-155 target sequence of SEQ ID SEQ ID NO: 200 (5′-UAAUGCGUGGGGUGGGAGAGG-3′).

In some embodiments, a subject, e.g., a AG heterozygous for rs61764044 variant subject is administered a miR-155 agent as disclosed herein in combination with an miR-425 agent of SEQ ID NO: 15 and/or a variant miRNA which binds miR-425 target site of to SEQ ID NO: 18, in combination with a modified miR-425 agent which binds to the miR-425 target sequence of SEQ ID NO: 19. In some embodiments, administration of a composition comprising a miR agent as disclosed herein (e.g., miR-103, miR-105, miR-107, and/or miR-155 agent) is administered to a subject to treat any one or a combination of inhibiting angiogenesis, low blood pressure, increased endothelial permeability, or orthostatic hypotension.

In some embodiments, a miR agent as disclosed herein is administered in combination with an miR-425 agent (e.g., SEQ ID NO: 15) or variant thereof which binds to the miRNA target sequence SEQ ID NO: 18 is administered to a subject whom has been identified as having at least one A allele of rs5068 SNP, e.g., a AA or AG subject. In alternative embodiments, a modified miR-425 which binds to the “G” allele miRNA target sequence of SEQ ID NO: 19 in the 3′UTR of the NPPA gene is administered to a subject who has been identified to have at least one G allele or rs5068 SNP (e.g., GG or AG subjects). In some embodiments, a subject, e.g., a AG heterozygous subject is administered a miR agent as disclosed herein (e.g., a miR-103, miR-105, miR-107 and/or miR-155 agent) in combination with an miR-425 agent of SEQ ID NO: 15 and/or a variant miRNA which binds to SEQ ID NO: 18, in combination with a modified miR-425 which binds to the miR target sequence SEQ ID NO: 19. In some embodiments, administration of a composition comprising a miR agent as disclosed herein (e.g., miR-103, miR-105, miR-107, and/or miR-155 agent) is administered to a subject to treat any one or a combination of inhibiting angiogenesis, low blood pressure, increased endothelial permeability, or orthostatic hypotension.

In some embodiments, a miR agent (e.g., miR-103, miR-105, miR-107, and/or miR-155 agent) as disclosed herein or mimetic thereof can be administered to a subject to treat a subject where there is leakage of the plasma into the extravascular space (increased endothelial permeability). (Kuhn M. Endothelial actions of atrial and B-type natriuretic peptides. Br J Pharmacol; 166:522-31.) In alternative embodiments, a miR agent (e.g., miR-103, miR-105, miR-107, and/or miR-155 agent) as disclosed herein or mimetic or homologue thereof can be administered to a subject in need of decreasing ANP levels in a subject, for example a subject where it is desirable to inhibit angiogenesis (Kong X, Wang X, Xu W, et al. Natriuretic peptide receptor a as a novel anticancer target. Cancer Res 2008; 68:249-56.), or a subject with orthostatic hypotension. In particular, there has been an association of the rs5068 variant with orthostatic hypotension 17 (Tunny et al., Inappropriately elevated levels of atrial natriuretic peptide may contribute to the pathophysiology of orthostatic hypotension. Clin Exp Pharmacol Physiol 1992; 19:283-6; Fedorowski A, et al., Orthostatic hypotension and novel blood pressure-associated gene variants: Genetics of Postural Hemodynamics (GPH) Consortium. Eur Heart J.)

### Administration

In one aspect, the invention provides methods of administering any, or a combination of an anti-miR agent (e.g., that inhibits at least one of miR-103, miR-105, miR-107 and/or miR-155) or miR agent (e.g., miR-103, miR-105, miR-107 or miR-155 agent) as disclosed herein to a subject. When administered, an anti-miR agent (e.g., that inhibits at least one of miR-103, miR-105, miR-107 and/or miR-155) or miR agent (e.g., miR-103, miR-105, miR-107 or miR-155 agent) as disclosed herein is administered to the subject in a therapeutically effective, pharmaceutically acceptable amount as a pharmaceutically acceptable formulation.

A therapeutically effective amount can be determined on an individual basis and will be based, at least in part, on consideration of the species of mammal, the mammal's age, sex, size, and health; the compound and/or composition used, the type of delivery system used; the time of administration relative to the severity of the disease; and whether a single, multiple, or controlled-release dose regimen is employed. A therapeutically effective amount can be determined by one of ordinary skill in the art employing such factors and using no more than routine experimentation.

In administering an anti-miR agent (e.g., that inhibits at least one of miR-103, miR-105, miR-107 and/or miR-155) or miR agent (e.g., miR-103, miR-105, miR-107 or miR-155 agent) as disclosed herein according to the systems and methods of the invention to a subject, dosing amounts, dosing schedules, routes of administration, and the like may be selected so as to affect known activities of these systems and methods. Dosage may be adjusted appropriately to achieve desired drug levels, local or systemic, depending upon the mode of administration. The doses may be given in one or several administrations per day. As one example, if daily doses are required, daily doses may be from about 0.01 mg/kg/day to about 1000 mg/kg/day, and in some embodiments, from about 0.1 to about 100 mg/kg/day or from about 1 mg/kg/day to about 10 mg/kg/day. Parenteral administration, in some cases, may be from one to several orders of magnitude lower dose per day, as compared to oral doses. For example, the dosage of an active compound of an anti-miR agent (e.g., that inhibits at least one of miR-103, miR-105, miR-107 and/or miR-155) or miR agent (e.g., miR-103, miR-105, miR-107 or miR-155 agent) as disclosed herein when parenterally administered may be between about 0.1 micrograms/kg/day to about 10 mg/kg/day, and in some embodiments, from about 1 microgram/kg/day to about 1 mg/kg/day or from about 0.01 mg/kg/day to about 0.1 mg/kg/day. In some embodiments, the concentration of the active compound(s), if administered systemically, is at a dose of about 1.0 mg to about 2000 mg for an adult of kg body weight, per day. In other embodiments, the dose is about 10 mg to about 1000 mg/70 kg/day. In yet other embodiments, the dose is about 100 mg to about 500 mg/70 kg/day. Preferably, the concentration, if applied topically, is about 0.1 mg to about 500 mg/gm of ointment or other base, more preferably about 1.0 mg to about 100 mg/gm of base, and most preferably, about 30 mg to about 70 mg/gm of base. The specific concentration partially depends upon the particular composition used, as some are more effective than others. The dosage concentration of a composition comprising an anti-miR agent (e.g., that inhibits at least one of miR-103, miR-105, miR-107 and/or miR-155) or miR agent (e.g., miR-103, miR-105, miR-107 or miR-155 agent) as disclosed herein actually administered is dependent at least in part upon the particular physiological response being treated, the final concentration of composition that is desired at the site of action, the method of administration, the efficacy of the particular composition, the longevity of the particular composition, and the timing of administration relative to the severity of the disease. Preferably, the dosage form is such that it does not substantially deleteriously affect the mammal. The dosage can be determined by one of ordinary skill in the art employing such factors and using no more than routine experimentation. In the event that the response of a particular subject is insufficient at such doses, even higher doses (or effectively higher doses by a different, more localized delivery route) may be employed to the extent that subject tolerance permits. Multiple doses per day are also contemplated in some cases to achieve appropriate systemic levels within the subject or within the active site of the subject. In some cases, dosing amounts, dosing schedules, routes of administration, and the like may be selected as described herein, whereby therapeutically effective levels for the treatment of cancer are provided.

In certain embodiments where high blood pressure, heart failure or other cardiovascular disorder is being treated, an anti-miR agent (e.g., that inhibits at least one of miR-103, miR-105, miR-107 and/or miR-155) as disclosed herein can be administered to a subject who has a family history cardiovascular disorder, e.g., a family history of any one or a combination of high blood pressure, heart failure, hypotrophy (e.g., left ventricle hypotrophy), or to a subject who has a genetic predisposition for cardiovascular disease, e.g., heart failure, high blood pressure and the like. In other embodiments, an anti-miR agent (e.g., that inhibits at least one of miR-103, miR-105, miR-107 and/or miR-155) can be administered to a subject who has reached a particular age, or to a subject more likely to get high blood pressure or heart failure. In yet other embodiments, an anti-miR agent (e.g., that inhibits at least one of miR-103, miR-105, miR-107 and/or miR-155) as disclosed herein can be administered to subjects who exhibit symptoms of high blood pressure or heart failure. In still other embodiments, the composition may be administered to a subject as a preventive measure.

In some embodiments, an anti-miR agent (e.g., that inhibits at least one of miR-103, miR-105, miR-107 and/or miR-155) or miR agent (e.g., miR-103, miR-105, miR-107 or miR-155 agent) as disclosed herein may be administered to a subject based on demographics or epidemiological studies, or to a subject in a particular field or career (e.g., a high stress career or career where low blood pressure is important). In some embodiments, an anti-miR agent (e.g., that inhibits at least one of miR-103, miR-105, miR-107 and/or miR-155) or miR agent (e.g., miR-103, miR-105, miR-107 or miR-155 agent) as disclosed herein can be administered to a subject that has had a prior therapy for a cardiovascular disease.

In some embodiments an anti-miR agent (e.g., that inhibits at least one of miR-103, miR-105, miR-107 and/or miR-155) as disclosed herein as disclosed herein can be administered to a subject who has renal failure, obesity or a risk of renal failure and/or obesity.

In some embodiments, an anti-miR agent (e.g., that inhibits at least one of miR-103, miR-105, miR-107 and/or miR-155) is administered to a subject who has low blood pressure, or has had a prior history of low blood pressure, or is in need of elevating their blood pressure (e.g., after a surgery etc.).

Administration of an anti-miR agent (e.g., that inhibits at least one of miR-103, miR-105, miR-107 and/or miR-155) or miR agent (e.g., miR-103, miR-105, miR-107 or miR-155 agent) as disclosed herein as disclosed herein to a subject may be accomplished by any medically acceptable method which allows the composition to reach its target. The particular mode selected will depend of course, upon factors such as those previously described, for example, the particular composition, the severity of the state of the subject being treated, the dosage required for therapeutic efficacy, etc. As used herein, a “medically acceptable” mode of treatment is a mode able to produce effective levels of the active compound(s) of the composition within the subject without causing clinically unacceptable adverse effects.

The methods to deliver an anti-miR agent (e.g., that inhibits at least one of miR-103, miR-105, miR-107 and/or miR-155) or miR agent (e.g., miR-103, miR-105, miR-107 or miR-155 agent) as disclosed herein to the cell or subject useful in the present invention are well known in the art, and include chemical transfection using lipid-based, amine based and polymer based techniques, viral vectors and combinations thereof (see, for example, products from Ambion Inc., Austin, Tex.; and Novagen, EMD Biosciences, Inc, an Affiliate of Merck KGaA, Darmstadt, Germany).

Other described ways to deliver an anti-miR agent (e.g., that inhibits at least one of miR-103, miR-105, miR-107 and/or miR-155) or miR agent (e.g., miR-103, miR-105, miR-107 or miR-155 agent) as disclosed herein is from vectors, such as lentiviral constructs, and introducing siRNA molecules into cells using electroporation. However, feline FIV lentivirus vectors which are based on the feline immunodeficiency virus (FIV) retrovirus and the HIV lentivirus vector system, which is based on the human immunodeficiency virus (HIV), carry with them problems related to permanent integration. Electroporation is also useful in the present invention, although it is generally only used to deliver siRNAs into cells in vitro.

The target cell types to which an anti-miR agent (e.g., that inhibits at least one of miR-103, miR-105, miR-107 and/or miR-155) or miR agent (e.g., miR-103, miR-105, miR-107 or miR-155 agent) as disclosed herein can be delivered using the methods of the invention include heart cells, such as cardiomyocytes and cardiac fibroblasts, neuronal tissues, prostate cells and other cells which express at least one of miR-103, miR-105, miR-107 or miR-155 and/or NPPA gene.

In one embodiment, the nucleic acid encoding an anti-miR agent (e.g., that inhibits at least one of miR-103, miR-105, miR-107 and/or miR-155) or miR agent (e.g., miR-103, miR-105, miR-107 or miR-155 agent) as disclosed herein is present on a vector. These vectors include a sequence encoding an anti-miR agent (e.g., that inhibits at least one of miR-103, miR-105, miR-107 and/or miR-155) or miR agent (e.g., miR-103, miR-105, miR-107 or miR-155 agent) as disclosed herein and an in vivo expression elements. In some embodiments, these vectors include a sequence encoding an anti-miR agent (e.g., that inhibits at least one of miR-103, miR-105, miR-107 and/or miR-155) or miR agent (e.g., miR-103, miR-105, miR-107 or miR-155 agent) as disclosed herein and in vivo expression elements such that the anti-miR agent or miR agent as disclosed herein is expressed and processed in vivo. In some embodiments, vectors that express miRs include a sequence encoding a miR agent (e.g., miR-103, miR-105, miR-107 or miR-155 agent) and in vivo expression elements, where the miR agent is first processed to produce the stem-loop precursor miRNA molecule, which can be processed to produce miR.

In some embodiments, an anti-miR agent (e.g., that inhibits at least one of miR-103, miR-105, miR-107 and/or miR-155) or miR agent (e.g., miR-103, miR-105, miR-107 or miR-155 agent) as disclosed herein can be delivered in vivo and in vitro. The in vivo delivery as used herein means delivery an anti-miR agent or miR agent into a living subject, including human. The in vitro delivery as used herein means delivery of an anti-miR or miR into cells and organs outside a living subject.

Vectors include, but are not limited to, plasmids, cosmids, phagemids, viruses, other vehicles derived from viral or bacterial sources that have been manipulated by the insertion or incorporation of the nucleic acid sequences for producing the microRNA, and free nucleic acid fragments which can be attached to these nucleic acid sequences. Viral and retroviral vectors are a preferred type of vector and include, but are not limited to, nucleic acid sequences from the following viruses: retroviruses, such as: Moloney murine leukemia virus; Murine stem cell virus, Harvey murine sarcoma virus; marine mammary tumor virus; Rous sarcoma virus; adenovirus; adeno-associated virus; SV40-type viruses; polyoma viruses; Epstein-Barr viruses; papilloma viruses; herpes viruses; vaccinia viruses; polio viruses; and RNA viruses such as any retrovirus. One of skill in the art can readily employ other vectors known in the art.

Viral vectors are generally based on non-cytopathic eukaryotic viruses in which non-essential genes have been replaced with the nucleic acid sequence of interest. Non-cytopathic viruses include retroviruses, the life cycle of which involves reverse transcription of genomic viral RNA into DNA with subsequent proviral integration into host cellular DNA.

Retroviruses have been approved for human gene therapy trials. Genetically altered retroviral expression vectors have general utility for the high efficiency transduction of nucleic acids in viva. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, M., “Gene Transfer and Expression, A Laboratory Manual,” W.H. Freeman Co., New York (1990) and Murry, E. J. Ed. “Methods in Molecular L Biology,” vol. 7, Humana Press, Inc., Cliffton, N.J. (1991).

In some embodiments the “in vivo expression elements” are any regulatory nucleotide sequence, such as a promoter sequence or promoter-enhancer combination, which facilitates the efficient expression of the nucleic acid to produce the microRNA. The in vivo expression element may, for example, be a mammalian or viral promoter, such as a constitutive or inducible promoter and/or a tissue specific promoter. Examples of which are well known to one of ordinary skill in the art. Constitutive mammalian promoters include, but are not limited to, polymerase promoters as well as the promoters for the following genes: hypoxanthine phosphoribosyl transferase (HPTR), adenine deaminase, pyruvate kinase, and beta.-actin. Exemplary viral promoters which function constitutively in eukaryotic cells include, but are not limited to, promoters from the simian virus, papilloma virus, adenovirus, human immunodeficiency virus (HIV), Rous sarcoma virus, cytomegalovirus, the long terminal repeats (LTR) of moloney leukemia virus and other retroviruses, and the thymidine kinase promoter of herpes simplex virus. Other constitutive promoters are known to those of ordinary skill in the art. Inducible promoters are expressed in the presence of an inducing agent and include, but are not limited to, metal-inducible promoters and steroid-regulated promoters. For example, the metallothionein promoter is induced to promote transcription in the presence of certain metal ions. Other inducible promoters are known to those of ordinary skill in the art.

Examples of tissue-specific promoters include, but are not limited to, the promoter for creatine kinase, which has been used to direct expression in muscle and cardiac tissue and immunoglobulin heavy or light chain promoters for expression in B cells. Other tissue specific promoters include the human smooth muscle alpha-actin promoter. Exemplary tissue-specific expression elements for the liver include but are not limited to HMG-COA reductase promoter, sterol regulatory element 1, phosphoenol pyruvate carboxy kinase (PEPCK) promoter, human C-reactive protein (CRP) promoter, human glucokinase promoter, cholesterol L 7-alpha hydroylase (CYP-7) promoter, beta-galactosidase alpha-2,6 sialylkansferase promoter, insulin-like growth factor binding protein (IGFBP-1) promoter, aldolase B promoter, human transferrin promoter, and collagen type I promoter. Exemplary tissue-specific expression elements for the prostate include but are not limited to the prostatic acid phosphatase (PAP) promoter, prostatic secretory protein of 94 (PSP 94) promoter, prostate specific antigen complex promoter, and human glandular kallikrein gene promoter (hgt-1). Exemplary tissue-specific expression elements for gastric tissue include but are not limited to the human H⁺/K⁺-ATPase alpha subunit promoter. Exemplary tissue-specific expression elements for the pancreas include but are not limited to pancreatitis associated protein promoter (PAP), elastase 1 transcriptional enhancer, pancreas specific amylase and elastase enhancer promoter, and pancreatic cholesterol esterase gene promoter. Exemplary tissue-specific expression elements for the endometrium include, but are not limited to, the uteroglobin promoter. Exemplary tissue-specific expression elements for adrenal cells include, but are not limited to, cholesterol side-chain cleavage (SCC) promoter. Exemplary tissue-specific expression elements for the general nervous system include, but are not limited to, gamma-gamma enolase (neuron-specific enolase, NSE) promoter. Exemplary tissue-specific expression elements for the brain include, but are not limited to, the neurofilament heavy chain (NF-H) promoter. Exemplary tissue-specific expression elements for lymphocytes include, but are not limited to, the human CGL-1/granzyme B promoter, the terminal deoxy transferase (TdT), lambda 5, VpreB, and lck (lymphocyte specific tyrosine protein kinase p561ck) promoter, the humans CD2 promoter and its 3′ transcriptional enhancer, and the human NK and T cell specific activation (NKG5) promoter. Exemplary tissue-specific expression elements for the colon include, but are not limited to, pp60c-src tyrosine kinase promoter, organ-specific neoantigens (OSNs) promoter, and colon specific antigen-P promoter.

Other elements aiding specificity of expression in a tissue of interest can include secretion leader sequences, enhancers, nuclear localization signals, endosmolytic peptides, etc. Preferably, these elements are derived from the tissue of interest to aid specificity. In general, the in vivo expression element shall include, as necessary, 5′ non-transcribing and 5′ non-translating sequences involved with the initiation of transcription. They optionally include enhancer sequences or upstream activator sequences.

An anti-miR agent (e.g., that inhibits at least one of miR-103, miR-105, miR-107 and/or miR-155) or miR agent (e.g., miR-103, miR-105, miR-107 or miR-155 agent) as disclosed herein either alone or expressed as a viral vector, or complexed to targeting moieties can be delivered using any delivery system such as topical administration, subcutaneous, intramuscular, intraperitoneal, intrathecal and intravenous injections, catheters for delivering an anti-miR agent (e.g., that inhibits at least one of miR-103, miR-105, miR-107 and/or miR-155) or miR agent (e.g., miR-103, miR-105, miR-107 or miR-155 agent) as disclosed herein into, for example, a specific organ, such as heart.

A pharmaceutically acceptable carrier as used herein means any pharmaceutically acceptable means to mix and/or deliver an anti-miR agent (e.g., that inhibits at least one of miR-103, miR-105, miR-107 and/or miR-155) or miR agent (e.g., miR-103, miR-105, miR-107 or miR-155 agent) as disclosed herein either alone or complexed to targeting moieties to a subject, or in combination with one or more pharmaceutically acceptable ingredients.

In the preparation of pharmaceutical formulations containing an anti-miR agent (e.g., that inhibits at least one of miR-103, miR-105, miR-107 and/or miR-155) or miR agent (e.g., miR-103, miR-105, miR-107 or miR-155 agent) as disclosed herein, either alone or complexed to targeting moieties of the present invention in the form of dosage units for oral administration the compound selected may be mixed with solid, powdered ingredients, such as lactose, saccharose, sorbitol, mannitol, starch, arnylopectin, cellulose derivatives, gelatin, or another suitable ingredient, as well as with disintegrating agents and lubricating agents such as magnesium stearate, calcium stearate, sodium stearyl fumarate and polyethylene glycol waxes. The mixture is then processed into granules or pressed into tablets.

Soft gelatin capsules may be prepared with capsules containing a mixture of the active compound or compounds of the invention in vegetable oil, fat, or other suitable vehicle for soft gelatin capsules. Hard gelatin capsules may contain granules of the active compound. Hard gelatin capsules may also contain an anti-miR agent (e.g., that inhibits at least one of miR-103, miR-105, miR-107 and/or miR-155) or miR agent (e.g., miR-103, miR-105, miR-107 or miR-155 agent) as disclosed herein either alone or complexed to targeting moieties in combination with solid powdered ingredients such as lactose, saccharose, sorbitol, mannitol, potato starch, corn starch, arnylopectin, cellulose derivatives or gelatin.

Dosage units for rectal or vaginal administration may be prepared (i) in the form of suppositories which contain the active substance, i.e. an anti-miR agent (e.g., that inhibits at least one of miR-103, miR-105, miR-107 and/or miR-155) or miR agent (e.g., miR-103, miR-105, miR-107 or miR-155 agent) as disclosed herein, either alone or complexed to targeting moieties, mixed with a neutral fat base; (ii) in the form of a gelatin rectal capsule which contains the active substance in a mixture with a vegetable oil, paraffin oil or other suitable vehicle for gelatin rectal capsules; (iii) in the form of a ready-made micro enema; or (iv) in the form of a dry micro enema formulation to be reconstituted in a suitable solvent just prior to administration.

Liquid preparations for oral administration may be prepared in the form of syrups or suspensions, e.g. solutions or suspensions containing from 0.2% to 20% by weight of the active ingredient and the remainder consisting of sugar or sugar alcohols and a mixture of ethanol, water, glycerol, propylene glycol and polyethylene glycol. If desired, such liquid preparations may contain coloring agents, flavoring agents, saccharin and carboxymethyl cellulose or other thickening agents. Liquid preparations for oral administration may also be prepared in the form of a dry powder to be reconstituted with a suitable solvent prior to use.

Solutions for parenteral administration may be prepared as a solution of a compound of the invention in a pharmaceutically acceptable solvent, preferably in a concentration from 0.1% to 10% by weight. These solutions may also contain stabilizing ingredients and/or buffering ingredients and are dispensed into unit doses in the form of ampoules or vials. Solutions for parenteral administration may also be prepared as a dry preparation to be reconstituted with a suitable solvent extemporaneously before use.

The methods of the present invention to also encompass delivery an anti-miR agent (e.g., that inhibits at least one of miR-103, miR-105, miR-107 and/or miR-155) or miR agent (e.g., miR-103, miR-105, miR-107 or miR-155 agent) as disclosed herein, either alone or complexed to targeting moieties orally in granular form including sprayed dried particles, or complexed to form micro or nanoparticles.

The subject or individual as referred to herein and throughout the specification includes mammals, such as murine, specifically mice and rats, bovine, and primates, such as human.

Other formuations for oral administration of an anti-miR agent (e.g., that inhibits at least one of miR-103, miR-105, miR-107 and/or miR-155) or miR agent (e.g., miR-103, miR-105, miR-107 or miR-155 agent) as disclosed herein for use with the invention include solutions or suspensions in aqueous or non-aqueous liquids such as a syrup, an elixir, or an emulsion. In another set of embodiments, an anti-miR agent (e.g., that inhibits at least one of miR-103, miR-105, miR-107 and/or miR-155) or miR agent (e.g., miR-103, miR-105, miR-107 or miR-155 agent) as disclosed herein may be used to fortify a food or a beverage.

Injections can be e.g., intravenous, intratumoral, intradermal, subcutaneous, intramuscular, or interperitoneal. The composition can be injected interdermally for treatment or prevention of infectious disease, for example. In some embodiments, the injections can be given at multiple locations. Implantation includes inserting implantable drug delivery systems, e.g., microspheres, hydrogels, polymeric reservoirs, cholesterol matrixes, polymeric systems, e.g., matrix erosion and/or diffusion systems and non-polymeric systems, e.g., compressed, fused, or partially-fused pellets. Inhalation includes administering the composition with an aerosol in an inhaler, either alone or attached to a carrier that can be absorbed. For systemic administration, it may be preferred that the composition is encapsulated in liposomes.

In some embodiments, an anti-miR agent (e.g., that inhibits at least one of miR-103, miR-105, miR-107 and/or miR-155) or miR agent (e.g., miR-103, miR-105, miR-107 or miR-155 agent) as disclosed herein, and/or nucleic acid encoding such, for example vectors are provided in a manner which enables tissue-specific uptake of the agent and/or nucleic acid delivery system. Techniques include using tissue or organ localizing devices, such as wound dressings or transdermal delivery systems, using invasive devices such as vascular or urinary catheters, and using interventional devices such as stents having drug delivery capability and configured as expansive devices or stent grafts.

An anti-miR agent (e.g., that inhibits at least one of miR-103, miR-105, miR-107 and/or miR-155) or miR agent (e.g., miR-103, miR-105, miR-107 or miR-155 agent) as disclosed herein may also be delivered using a bioerodible or bioresorbable implant by way of diffusion, or more preferably, by degradation of the polymeric matrix. Exemplary synthetic polymers which can be used to form the biodegradable delivery system include: polyamides, polycarbonates, polyalkylenes, s polyalkylene glycols, polyalkylene oxides, polyalkylene terepthalates, polyvinyl alcohols, polyvinyl ethers, polyvinyl esters, poly-vinyl halides, polyvinylpyrrolidone, polyglycolides, polysiloxanes, polyurethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, lo hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), i poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene, polyvinylpyrrolidone, and polymers of lactic; acid and glycolic acid, polyanhydrides, poly(ortho)esters, poly(butic acid), poly(valeric acid), and poly(lactide-cocaprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifcations routinely made by those skilled in the art), albumin and other hydrophilic proteins, zein and other prolamines and 2s hydrophobic proteins, copolymers and mixtures thereof. In general, these materials degrade either by enzymatic hydrolysis or exposure to water in vivo, by surface or bulk erosion. Examples of non-biodegradable polymers include ethylene vinyl acetate, poly(meth)acrylic acid, polyamides, copolymers and mixtures thereof.

Bioadhesive polymers of particular interest include bioerodible hydrogels described by H. S. Sawhney, C. P. Pathak and J. A. Hubell in Macromolecules, (1993) i 26:581-587, the teachings of which are incorporated herein, polyhyaluronic acids, casein, gelatin, glutin, polyanhydrides, polyacrylic acid, alginate, chitosan, poly(methyl I methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

In certain embodiments of the invention, the administration of an anti-miR agent (e.g., that inhibits at least one of miR-103, miR-105, miR-107 and/or miR-155) or miR agent (e.g., miR-103, miR-105, miR-107 or miR-155 agent) as disclosed herein may be designed so as to result in sequential exposures to the composition over a certain time period, for example, hours, days, weeks, months or years. This may be accomplished, for example, by repeated administrations of an anti-miR agent (e.g., that inhibits at least one of miR-103, miR-105, miR-107 and/or miR-155) or miR agent (e.g., miR-103, miR-105, miR-107 or miR-155 agent) as disclosed herein by one of the methods described above, or by a sustained or controlled release delivery system in which an anti-miR agent (e.g., that inhibits at least one of miR-103, miR-105, miR-107 and/or miR-155) or miR agent (e.g., miR-103, miR-105, miR-107 or miR-155 agent) as disclosed herein are delivered over a prolonged period without repeated administrations. Administration of an anti-miR agent (e.g., that inhibits at least one of miR-103, miR-105, miR-107 and/or miR-155) or miR agent (e.g., miR-103, miR-105, miR-107 or miR-155 agent) as disclosed herein using such a delivery system may be, for example, by oral dosage forms, bolus injections, transdermal patches or subcutaneous implants. Maintaining a substantially constant concentration of the composition may be preferred in some cases.

Other delivery systems suitable for use with the present invention include time release, delayed release, sustained release, or controlled release delivery systems. Such systems may avoid repeated administrations in many cases, increasing convenience to; the subject and the physician. Many types of release delivery systems are available and known to those of ordinary skill in the art. They include, for example, polymer-based systems such as polylactic and/or polyglycolic acids, polyanhydrides, polycaprolactones, copolyoxalates, polyesteramides, polyorthoesters, polyhydroxybutyric acid, and/or combinations of these. Microcapsules of the foregoing polymers containing drugs are described in, for example, U.S. Pat. No. 5,075,109. Other examples include nonpolymer systems that are lipid-based including sterols such as cholesterol, cholesterol esters, and fatty acids or neukal fats such as mono-, di- and triglycerides; hydrogel release systems; liposome-based systems; phospholipid based-systems; silastic systems; peptide based systems; wax coatings; compressed tablets using conventional binders and excipients; or partially fused implants. Specific examples include, but are not limited to, erosional systems in which the composition is contained in a form within a matrix (for example, as described in U.S. Pat. Nos. 4,452,775, 4,675,189, 5,736,152, 4,667,014, 4,748,034 and—29 5,239,660), or diffusional systems in which an active component controls the release rate I (for example, as described in U.S. Pat. Nos. 3,832,253, 3,854,480, 5,133,974 and 5,407,686). The formulation may be as, for example, microspheres, hydrogels, polymeric reservoirs, cholesterol matrices, or polymeric systems. In some embodiments, s the system may allow sustained or controlled release of the composition to occur, for example, through control of the diffusion or erosion/degradation rate of the formulation containing the composition. In addition, a pump-based hardware delivery system may be used to deliver one or more embodiments of the invention.

Examples of systems in which release occurs in bursts includes, e. g., systems in which the composition is entrapped in liposomes which are encapsulated in a polymer matrix, the liposomes being sensitive to specific stimuli, e.g., temperature, pH, light or a degrading enzyme and systems in which the composition is encapsulated by a tonically-coated microcapsule with a microcapsule core degrading enzyme. Examples of systems in which release of the inhibitor is gradual and continuous include, e.g., erosional Is systems in which the composition is contained in a forth within a matrix and effusional systems in which the composition permeates at a controlled rate, e.g., through a polymer. Such sustained release systems can be e.g., in the form of pellets, or capsules.

Examples of systems in which release occurs in bursts includes, e. g., systems in which the composition is entrapped in liposomes which are encapsulated in a polymer matrix, the liposomes being sensitive to specific stimuli, e.g., temperature, pH, light or a degrading enzyme and systems in which the composition is encapsulated by an tonically-coated microcapsule with a microcapsule core degrading enzyme. Examples of systems in which release of the inhibitor is gradual and continuous include, e.g., erosional systems in which the composition is contained in a form within a matrix and effusional systems in which the composition permeates at a controlled rate, e.g., through a polymer. Such sustained release systems can be e.g., in the form of pellets, or capsules.

Use of a long-term release implant may be particularly suitable in some; embodiments of the invention. “Long-term release,” as used herein, means that the implant containing an anti-miR-425 agent, e.g., anti-miR are constructed and arranged to deliver therapeutically effective levels of the composition for at least 30 or 45 days, and preferably at least 60 or days, or even longer in some cases. Long-term release implants are well known to those of ordinary skill in the art, and include some of the release systems described above.

In some embodiments, an anti-miR agent (e.g., that inhibits at least one of miR-103, miR-105, miR-107 and/or miR-155) or miR agent (e.g., miR-103, miR-105, miR-107 or miR-155 agent) as disclosed herein may include pharmaceutically acceptable carriers with formulation ingredients such as salts, carriers, buffering agents, emulsifiers, diluents, excipients, chelating agents, fillers, drying agents, antioxidants, antimicrobials, preservatives, binding agents, bulking agents, silicas, solubilizers, or stabilizers that may be used with the active compound. For example, if the formulation is a liquid, the carrier may be a solvent, partial solvent, or non-solvent, and may be aqueous or organically based. Examples of suitable formulation ingredients include, but are not limited to, diluents such as calcium carbonate, sodium carbonate, lactose, kaolin, calcium I phosphate, or sodium phosphate; granulating and disintegrating agents such as corn starch or algenic acid; binding agents such as starch, gelatin or acacia; lubricating agents such as magnesium stearate, stearic acid, or talc; time-delay materials such as glycerol monostearate or glycerol distearate; suspending agents such as sodium carboxymethylcellulose, methylcellulose, hydroxypropylmethylcellulose, sodium alginate, polyvinylpyrrolidone; dispersing or wetting agents such as lecithin or other naturally-occurring phosphatides; thickening agents such as cetyl alcohol or beeswax; buffering agents such as acetic acid and salts thereof, citric acid and salts thereof, boric lo acid and salts thereof, or phosphoric acid and salts thereof; or preservatives such as benzalkonium chloride, chlorobutanol, parabens, or thimerosal. Suitable carrier concentrations can be determined by those of ordinary skill in the art, using no more than routine experimentation. The compositions of the invention may be formulated into preparations in solid, semi-solid, liquid or gaseous forms such as tablets, capsules, elixirs, powders, granules, ointments, solutions, depositories, inhalants or injectables. Those of ordinary skill in the art will know of other suitable formulation ingredients, or will be able to ascertain such, using only routine experimentation.

Preparations include sterile aqueous or nonaqueous solutions, suspensions and; emulsions, which can be isotonic with the blood of the subject in certain embodiments. Examples of nonaqueous solvents are polypropylene glycol, polyethylene glycol, vegetable oil such as olive oil, sesame oil, coconut oil, arachis oil, peanut oil, mineral oil, injectable organic esters such as ethyl oleate, or fixed oils including synthetic mono or all-glycerides. Aqueous carriers include water, alcoholic/aqueous solutions, emulsions or suspensions, including saline and buffered media. Parenteral vehicles include sodium chloride solution, 1,3-butandiol, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, antioxidants, chelating agents and inert gases and the like. In addition, so sterile, fixed oils are conventionally employed as a solvent or suspending medium. For i this purpose any bland fixed oil may be employed including synthetic mono- or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa. Those of skill in the art can readily determine the various parameters for preparing and formulating the compositions of the invention without resort to undue experimentation.

In some embodiments, the present invention encompasses an anti-miR agent (e.g., that inhibits at least one of miR-103, miR-105, miR-107 and/or miR-155) or miR agent (e.g., miR-103, miR-105, miR-107 or miR-155 agent) as disclosed herein in association or contact with a suitable carrier, which may constitute one or more accessory ingredients. The final compositions may be prepared by any suitable technique, for example, by uniformly and intimately bringing the composition into association with a liquid carrier, a finely divided solid carrier or both, optionally with one or more formulation ingredients as previously described, and then, if necessary, shaping the product. In some embodiments, the compositions of the present invention may be present as pharmaceutically acceptable salts. The term “pharmaceutically acceptable salts” includes salts of the composition, prepared in combination with, for example, acids or bases, depending on the particular compounds found within the composition and the treatment modality desired. Pharmaceutically acceptable salts can be prepared as; alkaline metal salts, such as lithium, sodium, or potassium salts, or as alkaline earth salts, such as beryllium, magnesium or calcium salts. Examples of suitable bases that may be used to form salts include ammonium, or mineral bases such as sodium hydroxide, lithium hydroxide, potassium hydroxide, calcium hydroxide, magnesium hydroxide, and the like. Examples of suitable acids that may be used to form salts include inorganic or mineral acids such as hydrochloric, hydrobromic, hydroiodic, hydrofluoric, nitric, carbonic, monohydrogencarbonic, phosphoric, monohydrogenphosphoric, dihydrogenphosphoric, sulfuric, monohydrogensulfuric, phosphorous acids and the like.

Other suitable acids include organic acids, for example, acetic, propionic, isobutyric, maleic, malonic, benzoic, succinic, suberic, fumaric, mandelic, phthalic, benzenesulfonic, p-tolylsulfonic, citric, tartaric, methanesulfonic, glucuronic, galacturonic, salicylic, formic, naphthalene-2-sulfonic, and the like. Still other suitable acids include amino acids such as arginate, aspartate, glutamate, and the like.

For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, an anti-miR agent (e.g., that inhibits at least one of miR-103, miR-105, miR-107 and/or miR-155) or miR agent (e.g., miR-103, miR-105, miR-107 and/or miR-155 agent) can be incorporated with excipients and used in the form of tablets, troches, or capsules, e.g., gelatin capsules. Pharmaceutically compatible binding agents, and/or adjuvant materials can be included as part of the composition.

For administration by inhalation, an anti-miR agent (e.g., that inhibits at least one of miR-103, miR-105, miR-107 and/or miR-155) or miR agent (e.g., miR-103, miR-105, miR-107 and/or miR-155 agent) may be delivered in the form of an aerosol spray from pressured container or dispenser which contains a suitable propellant, e.g., a gas such as carbon dioxide, or a nebulizer. Such methods include those described in U.S. Pat. No. 6,468,798.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the miRNA antagonist may be formulated into ointments, salves, gels, or creams as generally known in the art.

An anti-miR agent (e.g., that inhibits at least one of miR-103, miR-105, miR-107 and/or miR-155) or miR agent (e.g., miR-103, miR-105, miR-107 and/or miR-155 agent) can also be administered by transfection or infection using methods known in the art, including but not limited to the methods described in McCaffrey et al. (2002), Nature, 418(6893), 38-9 (hydrodynamic transfection); Xia et al. (2002), Nature Biotechnol., 20(10), 1006-10 (viral-mediated delivery); or Putnam (1996), Am. J. Health Syst. Pharm. 53(2), 151-160, erratum at Am. J. Health Syst. Pharm. 53(3), 325 (1996).

In some embodiments, an anti-miR agent (e.g., that inhibits at least one of miR-103, miR-105, miR-107 and/or miR-155) or miR agent (e.g., miR-103, miR-105, miR-107 and/or miR-155 agent) (and other optional pharmacological agents, including anti-miR-425 agents) can be delivered directly via a pump device. For example, in some embodiments, an anti-miR agent (e.g., that inhibits at least one of miR-103, miR-105, miR-107 and/or miR-155 and/or miR-425) or miR agent (e.g., miR-103, miR-105, miR-107 and/or miR-155 agent and/or miR-425) of the invention are delivered directly by infusion into the diseased tissue, e.g. a tissue or cells of the heart.

In some embodiments, an anti-miR agent (e.g., that inhibits at least one of miR-103, miR-105, miR-107 and/or miR-155) or miR agent (e.g., miR-103, miR-105, miR-107 and/or miR-155 agent) can also be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al. (1998), Clin. Immunol Immunopathol., 88(2), 205-10. Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

In one embodiment, an anti-miR agent (e.g., that inhibits at least one of miR-103, miR-105, miR-107 and/or miR-155 and/or miR-425) or miR agent (e.g., miR-103, miR-105, miR-107 and/or miR-155 agent and/or miR-425) are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

In one embodiment, an anti-miR agent (e.g., that inhibits at least one of miR-103, miR-105, miR-107 and/or miR-155 and/or miR-425) or miR agent (e.g., miR-103, miR-105, miR-107 and/or miR-155 agent and/or miR-425) are formulated in the form of a yeast cell wall particle (YCWP, e.g., as described in US Patent Publication No. 20090226528, which is incorporated herein by reference in its entirety). YCWP are hollow and porous 2-4 micron microspheres prepared from yeast, for example, Baker's yeast, composed primarily of beta 1,3-D-glucan and, optionally, chitin and/or mannoprotein. Briefly, the process for producing the YCWPs involves the extraction and purification of the alkali-insoluble glucan particles from the yeast or fungal cell walls. Preparation of glucan particles involves treating the yeast with an aqueous alkaline solution at a suitable concentration to solubilize a portion of the yeast and form an alkali-hydroxide insoluble glucan particles having primarily .beta.(1,6) and .beta.(1,3) linkages. The alkali generally employed is an alkali-metal hydroxide, such as sodium or potassium hydroxide or an equivalent. The intracellular components and, optionally, the mannan portion, of the cell are solubilized in the aqueous hydroxide solution, leaving insoluble cell wall material which is substantially devoid of protein and having substantially unaltered .beta.(1,6) and .beta.(1,3) linked glucan. The intracellular constituents are hydrolyzed and released into the soluble phase. The conditions of digestion are such that at least in a major portion of the cells, the three dimensional matrix structure of the cell walls is not destroyed. In particular circumstances, substantially all the cell wall glucan remains unaltered and intact.

YCWPs can be used to deliver a payload of encapsulated anti-miR agent (e.g., that inhibits at least one of miR-103, miR-105, miR-107 and/or miR-155 and/or miR-425) or miR agent (e.g., miR-103, miR-105, miR-107 and/or miR-155 agent and/or miR-425) to a cell. In some embodiments, an anti-miR agent (e.g., that inhibits at least one of miR-103, miR-105, miR-107 and/or miR-155 and/or miR-425) or miR agent (e.g., miR-103, miR-105, miR-107 and/or miR-155 agent and/or miR-425) is complexed with polyelectrolyte trapping agent to form nanoparticle that is caged within the YCWP. Formation of the YCWP encapsulated polyelectrolyte nanoparticles follows a layer-by-layer (LbL) approach, with the different components assembled through electrostatic interactions. In some embodiments, the nanoparticle is formed around a core comprising an inert nucleic acid, such as tRNA or scrambled RNA, and a trapping agent. Other exemplary core components include, but are not limited to, anionic polysaccharides, proteins, synthetic polymers and inorganic matrices. Exemplary trapping agents are cationic polyeletrolytes and can include, but are not limited to, cationic polysaccharides, proteins and synthetic polymers. Exemplary YCWPs feature layers comprising a trapping molecule for the payload, which can be a cationic agent, such as an agent used to prepare nucleic acids for transfection into cells (e.g., polyethylenimine (PEI)); an inert nucleic acid, such as tRNA; and an anti-miR agent (e.g., that inhibits at least one of miR-103, miR-105, miR-107 and/or miR-155 and/or miR-425) or miR agent (e.g., miR-103, miR-105, miR-107 and/or miR-155 agent and/or miR-425) as a payload molecule. In certain exemplary embodiments, up to 100 mu.g of payload an anti-miR agent (e.g., that inhibits at least one of miR-103, miR-105, miR-107 and/or miR-155 and/or miR-425) or miR agent (e.g., miR-103, miR-105, miR-107 and/or miR-155 agent and/or miR-425) is added per 1.times.10.sup.9 YCWP with tRNA/PEI cores. A trapping molecule (e.g., PEI) is then added at a trapping molecule/nucleic acid ratio of 2.5 to coat the nucleic acid/tRNA-PEI core. Alternative embodiments include nanocomplexes and nanoparticles wherein a trapping molecule/layer is not applied to the YCWP/tRNA-PEI core/payload nucleic acid complex. Other embodiments include nanocomplexes and nanoparticles wherein a payload molecule is incorporate directly into the core, with or without tRNA.

Dosages for a particular patient can be determined by one of ordinary skill in the art using conventional considerations, (e.g. by means of an appropriate, conventional pharmacological protocol). A physician may, for example, prescribe a relatively low dose at first, subsequently increasing the dose until an appropriate response is obtained. The dose administered to a patient is sufficient to effect a beneficial therapeutic response in the patient over time, or, e.g., to reduce symptoms, or other appropriate activity, depending on the application. The dose is determined by the efficacy of the particular formulation, and the activity, stability or serum half-life of the anti-miR agent (e.g., that inhibits at least one of miR-103, miR-105, miR-107 and/or miR-155) or miR agent (e.g., miR-103, miR-105, miR-107 or miR-155 agent) employed and the condition of the patient, as well as the body weight or surface area of the patient to be treated. The size of the dose is also determined by the existence, nature, and extent of any adverse side-effects that accompany the administration of a particular vector, formulation, or the like in a particular subject. Therapeutic compositions comprising one or more nucleic acids are optionally tested in one or more appropriate in vitro and/or in vivo animal models of disease, to confirm efficacy, tissue metabolism, and to estimate dosages, according to methods well known in the art. In particular, dosages can be initially determined by activity, stability or other suitable measures of treatment vs. non-treatment (e.g., comparison of treated vs. untreated cells or animal models), in a relevant assay. Formulations are administered at a rate determined by the LD50 of the relevant formulation, and/or observation of any side-effects of the nucleic acids at various concentrations, e.g., as applied to the mass and overall health of the patient. Administration can be accomplished via single or divided doses.

In vitro models can be used to determine the effective doses of the nucleic acids as a potential hypertension treatment. In vivo models are the preferred models to determine the effective doses of an anti-miR agent (e.g., that inhibits at least one of miR-103, miR-105, miR-107 and/or miR-155) as disclosed herein as potential cardiovascular disease therapies, e.g., for the treatment of high blood pressure, chronic heart failure and the like. Suitable in vivo models include, but are not limited to, mice, rat, non-human primates, rabbit, guinea pig, pig. In some embodiments, an anti-miR agent (e.g., that inhibits at least one of miR-103, miR-105, miR-107 and/or miR-155) as disclosed herein can be assessed in a human cellular model expressing ANP as disclosed in Ma et al., 2011; AJP, 301; H2006-H2017 “High-purity human-induced pluripotent stem cell-derived cardiomyocytes; electrophysiological properties of action potentials and ionic currents”, which is incorporated herein in its entirety by reference. Alternatively, an animal model (e.g., rodent, porcrine, or monkey) expressing the human NPPA gene including the 3′ UTR is encompassed for use in assessing the effective dose of an anti-miR agent (e.g., that inhibits at least one of miR-103, miR-105, miR-107 and/or miR-155) as disclosed herein to knockdown miR-103, miR-105, miR-107 or miR-155 respectively and increase ANP respectively.

In some embodiments, a human cell line expressing ANP can be used, as well as cardiomyocytes differentiated from human iPS cells or ES cells as disclosed herein in the Examples. In some embodiments, the iPS cells are obtained from subjects who are AA homozygous or AG heterozygous for the rs5068 SNP or who are AA homozygous or AG heterozygous for the rs61764044 SNP. Such cells are commercially available from suppliers such as Cellular Dynamics or iCell.

An anti-miR agent (e.g., that inhibits at least one of miR-103, miR-105, miR-107 and/or miR-155) or miR agent (e.g., miR-103, miR-105, miR-107 or miR-155 agent) as disclosed herein can be assessed in other animal models, such as transgenic mouse models. In some embodiments, dogs can be used to assess the effect of an anti-miR agent (e.g., that inhibits at least one of miR-103, miR-105, miR-107 and/or miR-155) or miR agent (e.g., miR-103, miR-105, miR-107 or miR-155 agent)) as disclosed herein, which have a mature miR sequences similar to human miR sequences. Primate models can also be used. For example, different species express miR-103, miR-10, miR-107 and/or miR-155 (see SEQ ID NO: 29-151, or SEQ ID NO: 213-235), including chimpanzees and monkeys haver miR-103, miR-105, miR-107 and miR-155 sequences that are homologues of human miR-103, miR-105, miR-107 and/or miR-155, respectively, and thus are suitable to be used in animal models to assess anti-miR agents (e.g., that inhibits at least one of miR-103, miR-105, miR-107 and/or miR-155) to decrease ANP levels, or alternatively to assess anti-miR agents as disclosed herein. Similarily, mice and rats also express miR-103, miR-105, miR-107 and/or miR-155 homologues, and accordingly transgenic mouse and rat models where these bases have been substituted to resemble the human miR-103/miR-107 or human miR-105 target sequences of SEQ ID NO: 6 or 10, respectively, are also encompassed herein for assessing anti-miR agents as disclosed herein.

In determining the effective amount of an anti-miR agent (e.g., that inhibits at least one of miR-103, miR-105, miR-155 and/or miR-155) as disclosed herein to be administered in the treatment of a cardiovascular disease, the physician can evaluate circulating plasma levels of miR-103, miR-105, miR-107 or miR-155, by the methods as disclosed herein, as well as formulation toxicities, and progression of the disease.

The dose of an anti-miR agent (e.g., that inhibits at least one of miR-103, miR-105, miR-107 and/or miR-155) or miR agent (e.g., miR-103, miR-105, miR-107 or miR-155 agent) as disclosed herein administered to a 70 kilogram subject is typically in the range equivalent to dosages of currently-used therapeutic antisense oligonucleotides such as Vikavene (fomivirsen sodium injection) which is approved by the FDA for treatment of cytomegaloviral RNA, adjusted for the altered activity or serum half-life of the relevant composition.

In some embodiments, an anti-miR agent (e.g., that inhibits at least one of miR-103, miR-105, miR-107 and/or miR-155) or miR agent (e.g., miR-103, miR-105, miR-107 or miR-155 agent) as disclosed herein can supplement the treatment of any known additional therapy, including, but not limited to, antibody administration, vaccine administration, administration of cytotoxic agents, natural amino acid polypeptides, nucleic acids, nucleotide analogues, and biologic response modifiers. In some embodiments, an additional therapy for concurrent administration with an anti-miR agent (e.g., that inhibits at least one of miR-103, miR-105, miR-107 and/or miR-155) as disclosed herein, for example, surgery, chemotherapy, radiotherapy, thermotherapy, immunotherapy, hormone therapy and laser therapy. Two or more combined compounds may be used together or sequentially with an anti-miR agent or miR agent as disclosed herein. In some embodiments, an anti-miR agent (e.g., that inhibits at least one of miR-103, miR-105, miR-107 and/or miR-155) or miR agent (e.g., miR-103, miR-105, miR-107 or miR-155 agent) as disclosed herein can be administered before the additional therapy, after the additional therapy or at the same time as the additional therapy. In some embodiments, an anti-miR agent (e.g., that inhibits at least one of miR-103, miR-105, miR-107 and/or miR-155) or miR agent (e.g., miR-103, miR-105, miR-107 or miR-155 agent) as disclosed herein are administered a plurality of times, and in other embodiments, the additional therapies are also administered a plurality of times.

In some embodiments, an anti-miR agent (e.g., that inhibits at least one of miR-103, miR-105, miR-107 and/or miR-155) as disclosed herein can also be administered in therapeutically effective amounts as a portion of an anti-high blood pressure cocktail. An anti-high blood pressure cocktail is a mixture, for example of a least one anti-miR agent (e.g., anti-miR that inhibits at least one of miR-103, miR-105, miR-107 and/or miR-155) as disclosed herein with one or more additional agents to treat high-blood pressure, in addition to a pharmaceutically acceptable carrier for delivery. High blood pressure agents that are well known in the art and can be used as a treatment in combination with one or more anti-miR agents (e.g., that inhibits at least one of miR-103, miR-105, miR-107 and/or miR-155) as disclosed herein n include, but are not limited to: thiazide diuretics, beta blockers, angiotensin converting enzyme (ACE) inhibitors, angiotension II receptor blockers, calcium channel blockers and renin inhibitors (Aliskiren (Tekturna), alpha blockers, alpha-beta blockers, central-acting agents and vasodilators.

In some embodiments, an anti-miR agent (e.g., that inhibits at least one of miR-103, miR-105, miR-107 and/or miR-155) as disclosed herein can be administered alone or in combination with an additional therapeutic agent, such as a therapeutic agent is selected from the group consisting of bumetanide; ethacrynic acid; furosemide; torsemide; epitizide; hydrochlorothiazide; chlorothiazide; bendroflumethiazide; indapamide; chlorthalidone; metolazone; amiloride; triamterene; spironolactone; atenolol; metoprolol; nadolol; oxprenolol; pindolol; propranolol; timolol; doxazosin; phentolamine; indoramin; phenoxybenzamine; prazosin; terazosin; tolazoline; bucindolol; carvedilol; labetalol; amlodipine; felodipine; isradipine; lercanidipine; nicardipine; nifedipine; nimodipine; nitrendipine; diltiazem; verapamil; Aliskiren; captopril; enalapril; fosinopril; lisinopril; perindopril; quinapril; ramipril; trandolapril; benazepril; candesartan; eprosartan; irbesartan; losartan; olmesartan; telmisartan; valsartan; eplerenone; spironolactone; sodium nitroprusside; hydralazine; hydralazine derivatives; Clonidine; Guanabenz; Methyldopa; Moxonidine; Guanethidine; Reserpine; atorvastatin; fluvastatin; lovastatin; pitavastatin; pravastatin; rosuvastatin; simvastatin; and any combinations thereof

In some embodiments, a pharmaceutical composition comprising an anti-miR agent (e.g., that inhibits at least one of miR-103, miR-105, miR-107 and/or miR-155) as disclosed herein can be administered to a subject alone or in combination with agents which inhibit the degredation of ANP, for example, but not limited to inhibitors of neutral endopeptidases including OMAPATRILAT™, or ANP clearance by the clearance receptor. As discussed herein, a composition comprising an anti-miR agent (e.g., that inhibits at least one of miR-103, miR-105, miR-107 and/or miR-155) as disclosed herein can be administered to a subject alone or in combination with a PDE-5 inhibitor (e.g., but not limited to sildenafil or tadalafil) which increase cGMP, which is the secondary messenger for ANP.

In certain embodiments, the pharmaceutical compositions comprising an anti-miR agent (e.g., that inhibits at least one of miR-103, miR-105, miR-107 and/or miR-155) can optionally further comprise one or more additional therapies or agents. In certain embodiments, the additional agent or agents are agents for treatment of heart failure or hypertension. In some embodiments, the therapeutic agents include, but are not limited to thiazide diuretics, beta blockers, angiotensin converting enzyme (ACE) inhibitors, angiotension II receptor blockers, calcium channel blockers and renin inhibitors (Aliskiren (Tekturna), alpha blockers, alpha-beta blockers, central-acting agents and vasodilators.

Other therapeutic agents which can be administered in combination with an anti-miR agent (e.g., that inhibits at least one of miR-103, miR-105, miR-107 and/or miR-155) as disclosed herein is a mecamylamine agent, its stereoisomers together as the racemic mixture and as purified separate enantiomers, analogs, the free base, and/or salts thereof. Mecamylamine can be obtained according to the methods and processes described in U.S. Pat. No. 5,986,142, incorporated herein by reference for its teaching regarding method of producing mecamylamine. Purified exo-S-mecamylamine and exo-R-mecamylamine can be obtained according to methods discussed in U.S. Pat. No. 7,101,916, and references cited therein, also incorporated herein by reference for their teaching regarding the production of purified mecamylamine enantiomers.

ACE inhibitors which can be administered in combination with an anti-miR agent as disclosed herein include, but are not limited to, benazepril, captopril, ceronapril, enalapril, fosinopril, imidapril, lisinopril, moexipril, quinapril, ramipril, trandolapril, perindopril, and zofenopril. This list is not intended to be limiting, and other compounds known in the art as ACE inhibitors may also be used.

AT2 Inhibitors which can be administered in combination with an anti-miR agent (e.g., that inhibits at least one of miR-103, miR-105, miR-107 and/or miR-155) as disclosed herein include, but are not limited to, azilsartan, candesartan, losartan, irbesartan, eprosartan, olmesartan, tasosartan, telmisartan, valsartan, and zolarsartan. Below are representative estimated starting dosages and dosing ranges for selected AT2 inhibitors: andesartan: starting dose 16 mg once daily; dosing range 8 to 32 mg once daily rbesartan: starting dose 150 mg once daily; dosing range 150 to 300 mg once daily losartan: starting dose 50 mg once daily; dosing range 25 to 100 mg once daily telmisartan: starting dose 40 mg once daily; dosing range 20 to 80 mg once daily valsartan: starting dose 80 mg once daily; dosing range 80 to 320 mg once daily Source: Kaplan, N. M., Am Fam Physician 60:1185-90 (1999), herein incorporated by reference with regard to such dosages and ranges.

Renin antagonists/inhibitors which can be administered in combination with an anti-miR agent (e.g., that inhibits at least one of miR-103, miR-105, miR-107 and/or miR-155) as disclosed herein, include those taught in US Published Application, Publication No. 2008/0119557 A1, incorporated herein by reference for its teaching of renin inhibitors and method of obtaining such compounds. In some embodiments, a renin antagonist is aliskiren. In addition to the aforementioned compounds, renin inhibitors useful according to the present invention include, but are not limited to, ditekiren, enalkiren, remikiren, terlakiren, and zankiren.

One example of a further therapeutic agent which can be administered in combination with an anti-miR agent (e.g., that inhibits at least one of miR-103, miR-105, miR-107 and/or miR-155) as disclosed herein includes calcium channel blockers, namely compounds which work by blocking voltage-sensitive calcium channels in the heart and in the blood vessels. Calcium levels do not increase as much in the cells when stimulated, leading to less contraction. This decreases total peripheral resistance by dilating the blood vessels, and decreases cardiac output by lowering the force of contraction. Because resistance and output drop, so does blood pressure. With lowered blood pressure, the heart does not have to work as hard; this can ease problems with cardiomyopathy and coronary disease. Unlike with beta-blockers, the heart is still responsive to sympathetic nervous system stimulation, so blood pressure can be maintained more effectively. Calcium channel blockers useful in the present invention include dihydropyridine calcium channel blockers including amlodipine, felodipine, nicardipine, nifedipine, nimodipine, nisoldipine, nitrendipine, lacidipine, and lercanidipine; phenylalkylamine calcium channel blockers including verapamil and gallopamil; benzothiazepine calcium channel blockers including diltiazem; and other calcium channel blockers such as menthol.

Additionally, an anti-miR agent (e.g., that inhibits at least one of miR-103, miR-105, miR-107 and/or miR-155) as disclosed herein can be used in combination with any one or a combination of one or more of the following agents; anti-hyperlipidemic agents; anti-dyslipidemic agents; plasma HDL-raising agents; anti-hypercholesterolemic agents, including, but not limited to, cholesterol-uptake inhibitors; cholesterol biosynthesis inhibitors, e.g., HMG-CoA reductase inhibitors (also referred to as statins, such as lovastatin, simvastatin, pravastatin, fluvastatin, rosuvastatin, pitavastatin, and atorvastatin); HMG-CoA synthase inhibitors; squalene epoxidase inhibitors or squalene synthetase inhibitors (also known as squalene synthase inhibitors); acyl-coenzyme A cholesterol acyltransferase (ACAT) inhibitors, including, but not limited to, melinamide; probucol; nicotinic acid and the salts thereof; niacinamide; cholesterol absorption inhibitors, including, but not limited to, P-sitosterol or ezetimibe; bile acid sequestrant anion exchange resins, including, but not limited to cholestyramine, colestipol, colesevelam or dialkylaminoalkyl derivatives of a cross-linked dextran; LDL receptor inducers; fibrates, including, but not limited to, clofibrate, bezafibrate, fenofibrate and gemfibrozil; vitamin B6 (also known as pyridoxine) and the pharmaceutically acceptable salts thereof, such as the HCl salt; vitamin B12 (also known as cyanocobalamin); vitamin B3 (also known as nicotinic acid and niacinamide, supra); anti-oxidant vitamins, including, but not limited to, vitamin C and E and betacarotene; platelet aggregation inhibitors, including, but not limited to, fibrinogen receptor antagonists, i.e., glycoprotein IIb/IIIa fibrinogen receptor antagonists; hormones, including but not limited to, estrogen; insulin; ion exchange resins; omega-3 oils; benfluorex; ethyl icosapentate; and amlodipine; appetite-suppressing agents or anti-obesity agents including, but not limited to, insulin sensitizers, protein tyrosine phosphatase-1B (PTP-1 B) inhibitors, dipeptidyl peptidase IV (DPP-IV) inhibitors, insulin or insulin mimetics, sequestrants, nicotinyl alcohol, nicotinic acid, PPARα agonists, PPARγ agonists including, but not limited to thiazolidinediones, including but not limited to rosiglitazone, troglitazone and pioglitazone; PPARα/γ dual agonists, inhibitors of cholesterol absorption, acyl CoA:cholesterol acyltransferase inhibitors, anti-oxidants, anti-obesity compounds, neuropeptide Y5 inhibitors, β3 adrenergic receptor agonists, ileal bile acid transporter inhibitors, anti-inflammatories, including NSAIDs and COX-2 selective inhibitors; insulin; sulfonylureas, including but not limited to chlorpropamide, glipizide, glyburide, and glimepiride; biguanides, including but not limited to metformin; alpha-glucosidase inhibitors, including, but not limited to, acarbose and meglitol; cannabinoid antagonists, including, but not limited to rimonabant; camptothecin and camptothecin derivatives, D-phenylalanine derivatives; meglitinides; diuretics including, but not limited to, methyclothiazide, hydroflumethiazide, metolazone, chlorothiazide, methyclothiazide, hydrochlorothiazide, quinethazone, chlorthalidone, trichlormethiazide, bendroflumethiazide, polythiazide, hydroflumethiazide, spironolactone, triamterene, amiloride, bumetanide, torsemide, ethacrynic acid, furosemide; beta-blockers including, but not limited to acebutolol, atenolol, betaxolol, bisoprolol, carteolol, metoprolol, nadolol, pindolol, propranolol, and timolol; vasodilators including, but not limited to, nitric oxide, hydralazine, and prostacyclin; alpha blockers including, but not limited to, doxazosin, prazosin and terazosin; alpha 2 agonists including, but not limited to clonidine and guanfacine; curcumin, gugulipid, garlic, vitamin E, soy, soluble fiber, fish oil, green tea, carnitine, chromium, coenzyme Q10, anti-oxidant vitamins, grape seed extract, pantothine, red yeast rice, and royal jelly; NNR ligands (such as varenicline), antioxidants (such as free radical scavenging agents), antibacterial agents (such as penicillin antibiotics), antiviral agents (such as nucleoside analogs, like zidovudine and acyclovir), anticoagulants (such as warfarin), anti-inflammatory agents (such as NSAIDs), anti-pyretics, analgesics, anesthetics (such as used in surgery), acetylcholinesterase inhibitors (such as donepezil and galantamine), antipsychotics (such as haloperidol, clozapine, olanzapine, and quetiapine), immuno-suppressants (such as cyclosporin and methotrexate), neuroprotective agents, steroids (such as steroid hormones), corticosteroids (such as dexamethasone, predisone, and hydrocortisone), vitamins, minerals, nutraceuticals, anti-depressants (such as imipramine, fluoxetine, paroxetine, escitalopram, sertraline, venlafaxine, and duloxetine), anxiolytics (such as alprazolam and buspirone), anticonvulsants (such as phenytoin and gabapentin), vasodilators (such as prazosin and sildenafil), mood stabilizers (such as valproate and aripiprazole), anti-cancer drugs (such as anti-proliferatives), antihypertensive agents (such as atenolol, clonidine, amlopidine, verapamil, and olmesartan), laxatives, stool softeners, diuretics (such as furosemide), anti-spasmotics (such as dicyclomine), anti-dyskinetic agents, and anti-ulcer medications (such as esomeprazole), PDE5 inhibitor (e.g., sildenafil, preferably sildenafil citrate).

Systems to Identify Subjects Amenable to Treatment with an Anti-miR Agent

Another aspect of the present invention relates to a method, system and assay to identify subjects amenable to treatment with an anti-miR agent (e.g., that inhibits at least one of miR-103, miR-105, miR-107 and/or miR-155) as disclosed herein. In one embodiment, the present invention provides for an assay which enables one to measure, or quantify, the amount of at least one of; miR-103, miR-105, miR-107 and/or miR-155 in a biological sample, e.g., a blood or plasma sample, obtained from a subject; and compare the measured, or quantified amount of miR-103, miR-105, miR-107 and/or miR-155 with a reference value, and if the amount of miR-103, miR-105, miR-107 and/or miR-155 is increased relative to the reference value, the subject is identified as having an increased probability of having, or at risk of having high blood pressure, hypertension or cardiovascular disease. In such embodiments, the subject can be administered an anti-miR agent (e.g., that inhibits at least one of miR-103, miR-105, miR-107 and/or miR-155) as disclosed herein. In some embodiments, subjects with normal or high levels (e.g., above a predefined reference value) of any one or a combination of miR-103, miR-105, miR-107 and/or miR-155 are amenable to treatment with an anti-miR agent (e.g., that inhibits at least one of miR-103, miR-105, and/or miR-107) as disclosed herein, or where it is desirable to increase ANP levels in the subject.

Accordingly, one aspect of the present invention relates to an assay comprising: (a) contacting a biological sample obtained from a subject with a detectable antibody specific for miR-103, miR-105, miR-107 and/or miR-155 or at least one detectable nucleic acid complementary to at least part of one of the miRs selected from miR-103, miR-105, miR-107 and/or miR-155; (b) washing the sample to remove unbound antibody or unbound nucleic acid; (c) measuring the intensity of the signal from the bound, detectable antibody or bound detectable nucleic acid; (d) comparing the measured intensity of the signal with a reference value for the levels of miR-103, miR-105, miR-107 and/or miR-155 and if the measured intensity is normal and/or increased relative to the reference value; the subject is identified as having an increased probability of having high blood pressure or cardiovascular disease. In some embodiments, the reference value is the signal of the bound antibody or bound nucleic acid in a sample from a subject with low or normal blood pressure.

In some embodiments, subject amenable to treatment with an anti-miR agent (e.g., that inhibits at least one of miR-103, miR-105, miR-107 and/or miR-155) as disclosed herein have a blood pressure of above 120/80 mm Hg, above 115/75 mm Hg, where the risk of cardiovascular disease begins to increase. In some embodiments, subject amenable to treatment with an anti-miR agent (e.g., that inhibits at least one of miR-103, miR-105, miR-107 and/or miR-155) as disclosed herein have prehypertension, where their Prehypertension. Prehypertension is a systolic pressure ranges from 120 to 139 mm Hg or a diastolic pressure ranges from 80 to 89 mm Hg. In some embodiments, subject amenable to treatment with an anti-miR agent (e.g., that inhibits at least one of miR-103, miR-105, miR-107 and/or miR-155) as disclosed herein have stage 1 or Stage 2 hypertension, where subjects with stage 1 hypertension have a systolic pressure ranging from about 90-100 mm Hg to about 159 mm Hg or a diastolic pressure ranging from 90 to 99 mm Hg, and subjects with stage 2 hypertension have a systolic pressure of 160 mm Hg or higher or a diastolic pressure of 100 mm Hg or higher.

Another aspect of the present invention relates to a system for obtaining data from at least one test sample obtained from at least one subject, the system comprising: (a) a determination module configured to receive said at least one test sample and perform at least one analysis on said at least one test sample to determine the presence or absence of at least one of the following conditions: (i) the level of expression of NT-proANP or ANP mRNA is smaller than a pre-determined level; (ii) expression of any one of miR-103, miR-105, miR-107 and/or miR-155 is greater than a pre-determined standard; (b) a storage device configured to store data output from said determination module; and (c) a display module for displaying a content based in part on the data output from said determination module, wherein the content comprises a signal indicative of the presence of at least one of these conditions determined by the determination module, or a signal indicative of the absence of at least one of these conditions determined by the determination module. In some embodiments, the level of mRNA is determined with the levels of one or more other biomarkers, such as, but not limited to NT-proANP, NT-proBNP, cardiac troponins, C-reactive protein (CRP), ANP, BNP, adrenomedullin, copeptin, mid-regional-proANP, Galectin-3, ST-2, GDF-15 and other biomarkers, which are well known by persons of ordinary skill in the art.

In some embodiments, the nucleic acid sequence encoding NPPA is SEQ ID NO: 152 (NM_006172.3) which is as follows:

(SEQ ID NO: 152)

| | |
|---|---|
| 1 | gagacaggga cagacgtagg ccaagagagg ggaaccagag |
| | aggaaccaga ggggagagac |
| 61 | agagcagcaa gcagtggatt gctccttgac gacgccagca |
| | tgagctcctt ctccaccacc |
| 121 | accgtgagct tcctcctttt actggcattc cagctcctag |
| | gtcagaccag agctaatccc |
| 181 | atgtacaatg ccgtgtccaa cgcagacctg atggatttca |
| | agaatttgct ggaccatttg |
| 241 | gaagaaaaga tgcctttaga agatgaggtc gtgcccccac |
| | aagtgctcag tgagccgaat |
| 301 | gaagaagcgg gggctgctct cagccccctc cctgaggtgc |
| | ctccctggac cggggaagtc |
| 361 | agcccagccc agagagatgg aggtgccctc gggcggggcc |
| | cctgggactc ctctgatcga |
| 421 | tctgccctcc taaaaagcaa gctgagggcg ctgctcactg |
| | cccctcggag cctgcggaga |
| 481 | tccagctgct tcgggggcag gatggacagg attggagccc |
| | agagcggact gggctgtaac |
| 541 | agcttccggt actgaagata acagccaggg aggacaagca |
| | gggctgggcc tagggacaga |
| 601 | ctgcaagagg ctcctgtccc ctggggtctc tgctgcattt |
| | gtgtcatctt gttgccatgg |
| 661 | agttgtgatc atcccatcta agctgcagct tcctgtcaac |
| | acttctcaca tcttatgcta |
| 721 | actgtagata aagtggtttg atggtgactt cctcgcctct |
| | cccaccccat gcattaaatt |
| 781 | ttaaggtaga acctcacctg ttactgaaag tggtttgaaa |
| | gtgaataaac ttcagcacca |
| 841 | tggacagaag acaaaaaa |

Accordingly, as this is the nucleic acid encoding for NPPA mRNA, position 647(T) of SEQ ID NO: 152 (shown in bold, underline) shows the major allele for the rs5068 SNP (the corresponding mRNA shows the complementary “U” allele). The complementary genomic DNA sequence of SEQ ID NO: 152 encoding for the NPPA gene will show the adenosine (A) major allele rs5068 SNP and accordingly, will also have the adenosine (A) allele for rs61764044.

In some embodiments, the content displayed from the display module of the system as disclosed herein can further comprise a signal indicative of the subject being recommended to receive a particular treatment regimen, for example, if the subject has one or more of the above conditions, a signal is produced to recommend the subject be administered a composition comprising an anti-miR agent (e.g., that inhibits at least one of miR-103, miR-105, miR-107 and/or miR-155) as disclosed herein.

In some embodiments, the subject is recommended a treatment with a composition comprising an an anti-miR agent (e.g., that inhibits at least one of miR-103, miR-105, miR-107 and/or miR-155) as disclosed herein where the content from the display module produces a signal indicative of at least one of: (a) the expression of NT-proANP or ANP mRNA is smaller than a pre-determined level; and/or (b) the expression of any one or a combination of miR-103, miR-105, miR-107 and/or miR-155 is greater than a pre-determined standard.

In some embodiments, a subject is not recommended for treatment with a composition comprising an anti-miR agent (e.g., that inhibits at least one of miR-103, miR-105, miR-107 and/or miR-155) as disclosed herein where the content from the display module produces a signal indicative of at least one of: (a) the expression of NT-proANP or ANP mRNA is less than a pre-determined level; (b) the expression of any one of miR-103, miR-105, miR-107 and/or miR-155 is lower than a pre-determined standard.

In some embodiments, a predetermined level of expression of NT-proANP or ANP is the level of NT-proANP or ANP expression from a subject, or a representative pool of subjects who have normal levels of ANP.

Another embodiment of the present invention relates to a method of treating a subject having at least one or a combination of high blood pressure, hypertension, obesity, renal failure or a cardiovascular disease, comprising administering a pharmaceutically effective amount of one or a combination of an anti-miR agent as disclosed herein (e.g., an anti-miR agent that inhibits at least one, or at least two or more of miR-103, miR-105, miR-107 and/or miR-155) and in some embodiments, in combination with an anti-miR-425 agent. In some embodiments, subjects having high blood pressure, hypertension, obesity, renal failure, stress, or a cardiovascular disease administered the compositions as disclosed herein where the subject is selected as they have been identified to have a increased levels of miR-103, miR-105, miR-107 and/or miR-155 in the plasma relative to a control (e.g., pre-determined standard miR-103, miR-105, miR-107 and/or miR-155 expression level from a normal or healthy subject with normal blood pressure), and wherein the subject is not selected to be administered an anti-miR agent as disclosed herein if the subject is identified to have normal miR (e.g., miR-103, miR-105, miR-107 and/or miR-155) expression levels in the plasma, or where the subject is identified to have plasma miR-103, miR-105, miR-107 and/or miR-155 levels less than, or equal to that of a subject or a representative pool of normal or healthy subject with normal blood pressure.

Another embodiment of the present invention, a subject is admininstered a miR agent as disclosed herein, e.g., an anti-miR agent that inhibits at least one, or at least two, or more of miR-103, miR-105, miR-107 and/or miR-155, in combination with an anti-miR-425 agent, where the subject has been selected to have at least one copy of the A allele of the rs5068 SNP (e.g., AA homozygotes, or AG heterozygotes for rs5068) and/or selected to have at least one copy of the A allele of the rs61764044 SNP (e.g., AA homozygotes, or AG heterozygotes for rs61764044 SNP). Such a selection can be performed by one of ordinary skill in the art, as disclosed in International Application WO/2013/188787, including but not limited to assaying a blood sample for at least one copy of the A allele of the rs5068 SNP (e.g., AA homozygotes, or AG heteozygotes), or at least one copy of the A allele of the rs61764044 SNP (e.g., AA homozygotes, or AG heterozygotes for rs61764044 SNP). In some embodiments, a subject is administered a miR-agent as disclosed herein in combination with an anti-miR-425 agent if the subject is determined to have two copies of the A allele of the rs5068 SNP and/or 2 copies of the A allele for the rs61764044 SNP are detected (e.g., AA homozygotes) or if the subject is determined to have one copy of the A allele of the rs5068 SNP and/or one copy of A allele for the rs61764044 SNP (e.g., AG heterozygotes).

In some embodiments, the efficacy of an anti-miR agent and/or miR agent as disclosed herein can be assessed in a subject by measuring the levels of circulating ANP or NT-proANP in the plasma, were the efficacy of an anti-miR agent, or combination of anti-miR agents can be assessed by detecting an increase in ANP and/or NT-proANP in the plasma, and the efficacy of an miR agent can be assessed by a decrease in ANP and/or NT-pro-ANP levels in the plasma.

### Computer Systems

In some embodiments, the methods and assay can be carried out in an automated and/or high-throughput system. One aspect of the present invention relates to a computerized system for processing the assays as disclosed herein and identifying any one of the following: (i) the level of expression of NT-proANP or ANP mRNA is smaller than a pre-determined level; (ii) expression of any one or a combination of: miR-103, or miR-105, or miR-107 or miR-155 and/or miR-425 is greater than a pre-determined standard; and/or (iii) the level of expression of NPPA mRNA in circulating cells, (e.g., lymphocytes) is smaller than a pre-determined level.

In some embodiments, a computer system can include: (a) at least one memory containing at least one computer program adapted to control the operation of the computer system to implement a method that includes: (i) receiving data of the level of expression or intensity of signal of measured NT-proANP or ANP mRNA (ii) generating a report of intensity of expression or intensity of signal of measured NT-proANP or ANP mRNA in a biological sample and optionally a reference level for NT-proANP or ANP mRNA signal intensity; and (b) at least one processor for executing the computer program.

In some embodiments, a computer system can include: (a) at least one memory containing at least one computer program adapted to control the operation of the computer system to implement a method that includes: (i) receiving data of the level of expression or intensity of signal of measured miR-103, or miR-105, or miR-107 and/or miR-425 levels; and (ii) generating a report of intensity of expression or intensity of signal of measured miR-103, or miR-105, or miR-107, or miR-155 and/or miR-425 levels in a biological sample; and optionally a reference level for miR-103, or miR-105, or miR-107, or miR-155 and/or miR-425 level signal intensity; and (b) at least one processor for executing the computer program.

Another aspect of the present invention relates to a computer readable medium comprising instructions, such as computer programs and software, for controlling a computer system to process the data from signal intensity of one or more of (i) the level of expression of NT-proANP or ANP mRNA; (ii) miR-103, or miR-105, or miR-107, or miR-155 and/or miR-425 expression level, and generate a report of the presence or absence, or amount of (i) the expression of NT-proANP or ANP mRNA and/or (ii) miR-103, or miR-105, or miR-107, or miR-155 and/or miR-425 expression.

The computer system can include one or more general or special purpose processors and associated memory, including volatile and non-volatile memory devices. The computer system memory can store software or computer programs for controlling the operation of the computer system to make a special purpose computer system according to the invention or to implement a system to perform the methods and analysis according to the invention.

In some embodiments, a computer system can include, for example, an Intel or AMD x86 based single or multi-core central processing unit (CPU), an ARM processor or similar computer processor for processing the data. The CPU or microprocessor can be any conventional general purpose single-or multi-chip microprocessor such as an Intel and AMD processor, a SPARC processor, or an ARM processor. In addition, the microprocessor may be any conventional or special purpose microprocessor such as a digital signal processor or a graphics processor. The microprocessor typically has conventional address lines, conventional data lines, and one or more conventional control lines. As described below, the software according to the invention can be executed on dedicated system or on a general purpose computer having a DOS, CPM, Windows, Unix, Linix or other operating system. The system can include non-volatile memory, such as disk memory and solid state memory for storing computer programs, software and data and volatile memory, such as high speed ram for executing programs and software.

Computer-readable physical storage media useful in various embodiments of the invention can include any physical computer-readable storage medium, e.g., solid state memory (such as flash memory), magnetic and optical computer-readable storage media and devices, and memory that uses other persistent storage technologies. In some embodiments, a computer readable media can be any tangible media that allows computer programs and data to be accessed by a computer. Computer readable media can include volatile and nonvolatile, removable and non-removable tangible media implemented in any method or technology capable of storing information such as computer readable instructions, program modules, programs, data, data structures, and database information. In some embodiments of the invention, computer readable media includes, but is not limited to, RAM (random access memory), ROM (read only memory), EPROM (erasable programmable read only memory), EEPROM (electrically erasable programmable read only memory), flash memory or other memory technology, CD-ROM (compact disc read only memory), DVDs (digital versatile disks), Blue-ray, USB drives, micro-SD drives, or other optical storage media, magnetic cassettes, magnetic tape, magnetic disk storage or other magnetic storage media, other types of volatile and non-volatile memory, and any other tangible medium which can be used to store information and which can read by a computer including and any suitable combination of the foregoing.

The present invention can be implemented on a stand-alone computer or as part of a networked computer system. In a stand-alone computer, all the software and data can reside on local memory devices, for example an optical disk or flash memory device can be used to store the computer software for implementing the invention as well as the data. In alternative embodiments, the software or the data or both can be accessed through a network connection to remote devices. In one embodiment, the invention can use a client-server environment over a network, e.g., a public network such as the internet or a private network to connect to data and resources stored in remote and/or centrally located locations. In this embodiment, a server such as a web server can provide access, either open access, pay as you go or subscription based access to the information provided according to the invention. In a client server environment, a client computer executing a client software or program, such as a web browser, connects to the server over the network. The client software provides a user interface for a user of the invention to input data and information and receive access to data and information. The client software can be viewed on a local computer display or other output device and can allow the user to input information, such as by using a computer keyboard, mouse or other input device. The server executes one or more computer programs that receives data input through the client software, processes data according to the invention and outputs data to the user, as well as provide access to local and remote computer resources. For example, the user interface can include a graphical user interface comprising an access element, such as a text box, that permits entry of data from the assay, e.g., the data from a positive reference cancer cell, as well as a display element that can provide a graphical read out of the results of a comparison with a cancer cell with a known metastatic potential or invasive capacity, or data sets transmitted to or made available by a processor following execution of the instructions encoded on a computer-readable medium.

Embodiments of the invention also provide for systems (and computer readable medium providing instructions for causing computer systems) to perform a method for determining quality assurance of a pluripotent stem cell population according to the methods as disclosed herein.

In some embodiments of the invention, the computer system software can include one or more functional modules, which can be defined by computer executable instructions recorded on computer readable media and which cause a computer to perform, when executed, a method according to one or more embodiments of the invention. The modules can be segregated by function for the sake of clarity, however, it should be understood that the modules need not correspond to discreet blocks of code and the described functions can be carried out by the execution of various software code portions stored on various media and executed at various times. Furthermore, it should be appreciated that the modules can perform other functions, thus the modules are not limited to having any particular function or set of functions. In some embodiments, functional modules are, for example, but are not limited to, an array module, a determination module, a storage module, a reference comparison module, a normalization module, and a display module to display the results (e.g., the invasive potential of the test cancer cell population). The functional modules can be executed using one or multiple computers, and by using one or multiple computer networks.

The information embodied on one or more computer-readable media can include data, computer software or programs, and program instructions, that, as a result of being executed by a computer, transform the computer to special purpose machine and can cause the computer to perform one or more of the functions described herein. Such instructions can be originally written in any of a plurality of programming languages, for example, Java, J#, Visual Basic, C, C#, C++, Fortran, Pascal, Eiffel, Basic, COBOL assembly language, and the like, or any of a variety of combinations thereof. The computer-readable media on which such instructions are embodied can reside on one or more of the components of a computer system or a network of computer systems according to the invention.

In some embodiments, a computer-readable media can be transportable such that the instructions stored thereon can be loaded onto any computer resource to implement the aspects of the present invention discussed herein. In addition, it should be appreciated that the instructions stored on computer readable media are not limited to instructions embodied as part of an application program running on a host computer. Rather, the instructions may be embodied as any type of computer code (e.g., object code, software or microcode) that can be employed to program a computer to implement aspects of the present invention. The computer executable instructions may be written in a suitable computer language or combination of several languages. Basic computational biology methods are known to those of ordinary skill in the art and are described in, for example, Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001).

In some embodiments, a system as disclosed herein, can receive data of intensity of expression of NT pro-ANP or ANP mRNA, or miR-103, or miR-105, or miR-107 and/or miR-425 expression levels from any method of determining the level of expression. Where the quantity to be measured is protein expression, the system as disclosed herein can be configured to receive data from an automated protein analysis systems, for example, using immunoassay, for example western blot analysis or ELISA, or a high through-put protein detection method, for example but are not limited to automated immunohistochemistry apparatus, for example, robotically automated immunodetection apparatus which in an automated system can perform immunohistochemistry procedure and detect intensity of immunostaining, such as intensity of an antibody staining of the substrates and produce output data. Examples of such automated immunohistochemistry apparatus are commercially available, and can be readily adapted to automatically detect the level of protein expression in the assay as disclosed herein, and include, for example but not limited to such Autostainers 360, 480, 720 and Labvision PT module machines from LabVision Corporation, which are disclosed in U.S. Pat. Nos. 7,435,383; 6,998,270; 6,746,851, 6,735,531; 6,349,264; and 5,839; 091 which are incorporated herein in their entirety by reference. Other commercially available automated immunohistochemistry instruments are also encompassed for use in the present invention, for example, but not are limited BOND™ Automated Immunohistochemistry & In Situ Hybridization System, Automate slide loader from GTI vision. Automated analysis of immunohistochemistry can be performed by commercially available systems such as, for example, IHC Scorer and Path EX, which can be combined with the Applied spectral Images (ASI) CytoLab view, also available from GTI vision or Applied Spectral Imaging (ASI) which can all be integrated into data sharing systems such as, for example, Laboratory Information System (LIS), which incorporates Picture Archive Communication System (PACS), also available from Applied Spectral Imaging (ASI) (see world-wide-web: spectral-imaging.com). Other a determination module can be an automated immunohistochemistry systems such as NexES® automated immunohistochemistry (IHC) slide staining system or BenchMark® LT automated IHC instrument from Ventana Discovery SA, which can be combined with VIAS™ image analysis system also available Ventana Discovery. BioGenex Super Sensitive MultiLink® Detection Systems, in either manual or automated protocols can also be used as the detection module, preferably using the BioGenex Automated Staining Systems. Such systems can be combined with a BioGenex automated staining systems, the i6000™ (and its predecessor, the OptiMax® Plus), which is geared for the Clinical Diagnostics lab, and the GenoMx 6000™, for Drug Discovery labs. Both systems BioGenex systems perform “All-in-One, All-at-Once” functions for cell and tissue testing, such as Immunohistochemistry (IHC) and In Situ Hybridization (ISH).

In some embodiments, a system as disclosed herein, can receive data of intensity of protein expression of NT-proANP from an automated ELISA system (e.g. DSX® or DK® form Dynax, Chantilly, Va. or the ENEASYSTEM III®, Triturus®, The Mago® Plus); Densitometers (e.g. X-Rite-508-Spectro Densitometer®, The HYRYS™ 2 densitometer); automated Fluorescence in situ hybridization systems (see for example, U.S. Pat. No. 6,136,540); 2D gel imaging systems coupled with 2-D imaging software; microplate readers; Fluorescence activated cell sorters (FACS) (e.g. Flow Cytometer FACSVantage SE, Becton Dickinson); radio isotope analyzers (e.g. scintillation counters), or adapted systems thereof for detecting cells on the separated substrates as disclosed herein.

In some embodiments, a system as disclosed herein, can receive data can receive data of intensity of mRNA expression of ANP or any one or a combination of miR-103, or miR-105, or miR-107, or miR-155 and/or miR-425 levels using any method of determining gene or nucleic acid expression. In some embodiments, the system as disclosed herein can be configured to receive data from an automated gene expression analysis system, e.g., an automated protein expression analysis including but not limited Mass Spectrometry systems including MALDI-TOF, or Matrix Assisted Laser Desorption Ionization-Time of Flight systems; SELDI-TOF-MS ProteinChip array profiling systems, e.g. Machines with Ciphergen Protein Biology System II™ software; systems for analyzing gene expression data (see for example U.S. 2003/0194711); systems for array based expression analysis, for example HT array systems and cartridge array systems available from Affymetrix (Santa Clara, Calif. 95051) AutoLoader, Complete GeneChip® Instrument System, Fluidics Station 450, Hybridization Oven 645, QC Toolbox Software Kit, Scanner 3000 7G, Scanner 3000 7G plus Targeted Genotyping System, Scanner 3000 7G Whole-Genome Association System, GeneTitan™ Instrument, GeneChip® Array Station, HT Array.

In some embodiments of the present invention, an automated gene expression analysis system can record the data electronically or digitally, annotated and retrieved from databases including, but not limited to GenBank (NCBI) protein and DNA databases such as genome, ESTs, SNPS, Traces, Celara, Ventor Reads, Watson reads, HGTS, etc.; Swiss Institute of Bioinformatics databases, such as ENZYME, PROSITE, SWISS-2DPAGE, Swiss-Prot and TrEMBL databases; the Melanie software package or the ExPASy WWW server, etc., the SWISS-MODEL, Swiss-Shop and other network-based computational tools; the Comprehensive Microbial Resource database (The institute of Genomic Research). The resulting information can be stored in a relational data base that may be employed to determine homologies between the reference data or genes or proteins within and among genomes.

In some embodiments, a system as disclosed herein, can receive data can receive data from an allele-specific PCR. The term “allele-specific PCR” refers to PCR techniques where the primer pairs are chosen such that amplification is dependent upon the input template nucleic acid containing the polymorphism of interest. In such embodiments, primer pairs are chosen such that at least one primer is an allele-specific oligonucleotide primer. In some sub-embodiments of the present invention, allele-specific primers are chosen so that amplification creates a restriction site, facilitating identification of a polymorphic site. In other embodiments of the present invention, amplification of the target polynucleotide is by multiplex PCR (Wallace et al. (PCT Application WO89/10414)). Through the use of multiplex PCR, a multiplicity of regions of a target polynucleotide can be amplified simultaneously. This is particularly advantageous in embodiments where more than one SNP is to be detected.

In another embodiment, multiplex PCR procedures using allele-specific primers can be used to simultaneously amplify multiple regions of a target nucleic acid (PCT Application WO89/10414), enabling amplification only if a particular allele is present in a sample. Other embodiments using alternative primer-guided nucleotide incorporation procedures for assaying polymorphic sites in DNA can be used, and have been described (Komher, J. S. et al., Nucl. Acids. Res. 17:7779-7784 (1989); Sokolov, B. P., Nucl. Acids Res. 18:3671 (1990); Syvanen, A.-C., et al., Genomics 8:684-692 (1990); Kuppuswamy, M. N. et al., Proc. Nat. Acad. Sci. (U.S.A.) 88:1143-1147 (1991); Bajaj et al. (U.S. Pat. No. 5,846,710); Prezant, T. R. et al., Hum Mutat. 1: 159-164 (1992); Ugozzoli, L. et al., GATA 9:107-112 47 (1992); Nyr6n, P. et al., Anal. Biochem. 208:171-175 (1993)).

Other known nucleic acid amplification procedures include transcription-based amplification systems (Malek, L. T. et al., U.S. Pat. No. 5,130,238; Davey, C. et al., European Patent Application 329,822; Schuster et al.) U.S. Pat. No. 5,169,766; Miller, H. I. et al., PCT-Application WO89/06700; Kwoh, D. et al., Proc. Natl. Acad. Sci. (U.S.A.) 86:1173 Z1989); Gingeras, T. R. et al., PCT Application WO88/10315)), or isothermal amplification methods (Walker, G. T. et al., Proc. Natl. 4cad Sci. (U.S.A) 89:392-396 (1992)) can also be used.

In some embodiments, a system as disclosed herein, can receive data from any genotyping assay known by persons of ordinary skill in the art, including, but not limited to, those disclosed in U.S. Pat. No. 6,472,157; U.S. Patent Application Publications 20020016293, 20030099960, 20040203034; WO 0180896, all of which are hereby incorporated by reference, or by linkage disequlibrium, restriction fragment length polymorphism” (RFLP) analysis, single strand conformational polymorphism (SSCP), RNasel for mismatch detection, SNP mapping (Davis et al, Methods Mol Biology, 2006; 351; 75-92); Nanogen Nano Chip, (keen-Kim et al, 2006; Expert Rev Mol Diagnostic, 6; 287-294); Rolling circle amplification (RCA) combined with circularable oligonucleotide probes (c-probes) for the detection of nucleic acids (Zhang et al, 2006: 363; 61-70), luminex XMAP system for detecting multiple SNPs in a single reaction vessel (Dunbar S A, Clin Chim Acta, 2006; 363; 71-82; Dunbar et al, Methods Mol Med, 2005; 114:147-1471), enzymatic mutation detection methods (Yeung et al, Biotechniques, 2005; 38; 749-758), matrix-assisted laser desorption ionization/time-of-flight (MALDI-TOF) mass spectrometric (MS) analysis, long-range PCR (LR-PCR), genotype assays disclosed in Kwok, Hum Mut 2002; 9; 315-323 and Kwok, Annu Rev Genomic Hum Genetics, 2001; 2; 235-58, (which are incorporated herein in their entirety by reference), INVADER® Assay (Gut et al, Hum Mutat, 2001; 17:475-92, Shi et al, Clin Chem, 2001, 47,164-92, and Olivier et al, Mutat Res, 2005; 573:103-110), the method utilizing FLAP endonucleases (U.S. Pat. No. 6,706,476) and the SNPlex genoptyping systems (Tobler et al, J. Biomol Tech, 2005; 16; 398-406) and other such genotyping assays known to one of ordinary skill in the art.

In some embodiments, the data of (i) the level of expression of NT-proANP or ANP mRNA; and/or (ii) miR-103, or miR-105, or miR-107 and/or miR-425 expression level can be received from a memory, a storage device, or a database. The memory, storage device or database can be directly connected to the computer system retrieving the data, or connected to the computer through a wired or wireless connection technology and retrieved from a remote device or system over the wired or wireless connection. Further, the memory, storage device or database, can be located remotely from the computer system from which it is retrieved.

Examples of suitable connection technologies for use with the present invention include, for example parallel interfaces (e.g., PATA), serial interfaces (e.g., SATA, USB, Firewire), local area networks (LAN), wide area networks (WAN), Internet, Intranet, and Extranet, and wireless (e.g., Blue Tooth, Zigbee, WiFi, WiMAX, 3G, 4G) communication technologies

Storage devices are also commonly referred to in the art as “computer-readable physical storage media” which is useful in various embodiments, and can include any physical computer-readable storage medium, e.g., magnetic and optical computer-readable storage media, among others. Carrier waves and other signal-based storage or transmission media are not included within the scope of storage devices or physical computer-readable storage media encompassed by the term and useful according to the invention. The storage device is adapted or configured for having recorded thereon cytokine level information. Such information can be provided in digital form that can be transmitted and read electronically, e.g., via the Internet, on diskette, via USB (universal serial bus) or via any other suitable mode of communication.

As used herein, “stored” refers to a process for recording information, e.g., data, programs and instructions, on the storage device that can be read back at a later time. Those skilled in the art can readily adopt any of the presently known methods for recording information on known media to contribute to the data of (i) the level of expression of NT-proANP or NPPA mRNA; (ii) miR-103, or miR-105, or miR-107 and/or miR-425 expression level.

A variety of software programs and formats can be used to store information on the storage device. Any number of data processor structuring formats (e.g., text file or database) can be employed to obtain or create a medium having recorded scorecard thereon.

In some embodiment, the system has a processor for running one or more programs, e.g., where the programs can include an operating system (e.g., UNIX, Windows), a relational database management system, an application program, and a World Wide Web server program. The application program can be a World Wide Web application that includes the executable code necessary for generation of database language statements (e.g., Structured Query Language (SQL) statements). The executables can include embedded SQL statements. In addition, the World Wide Web application can include a configuration file which contains pointers and addresses to the various software entities that provide the World Wide Web server functions as well as the various external and internal databases which can be accessed to service user requests. The Configuration file can also direct requests for server resources to the appropriate hardware devices, as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GenBank or Swiss Pro World Wide Web site). Thus, in a particular preferred embodiment of the present invention, users can directly access data (via Hypertext links for example) residing on Internet databases using a HTML interface provided by Web browsers and Web servers.

In one embodiment, the system as disclosed herein can be used to compare the data of intensity of one or more of (i) the level of expression of NT-proANP or NPPA mRNA; (ii) miR-103, and/or miR-105, and/or miR-107 and/or miR-155 and/or miR-425 expression level and generate a report of the presence or absence, or amount of (i) the expression of NT-proANP or ANP mRNA; (ii) miR-103, and/or miR-105, and/or miR-107 and/or miR-155 and/or miR-425 expression level. By way of an example, but not as a limitation, reference data for NT-proANP can be the level of NT-proANP or ANP expression from a subject, or a representative pool of subjects who have normal blood pressure. In some embodiments, reference data for expression of miR-103, and/or miR-105, and/or miR-107 and/or miR-155 and/or miR-425 expression level is the level of the respective miR expression level from a subject or a representative pool or cohort of subjects who have normal blood pressure.

In some embodiments of this aspect and all other aspects of the present invention, the system can compare the data in a “comparison module” which can use a variety of available software programs and formats for the comparison operative to compare sequence information determined in the determination module to reference data. In one embodiment, the comparison module is configured to use pattern recognition techniques to compare levels of expression (e.g., mRNA levels and/or protein levels) as well as compare sequence information (e.g., identify the presence of an miR-103, and/or miR-105, and/or miR-107 and/or miR-155 and/or miR-425 expression level) from one or more entries to one or more reference data patterns. The comparison module may be configured using existing commercially-available or freely-available software for comparing patterns, and may be optimized for particular data comparisons that are conducted. The comparison module can also provide computer readable information related to the level or amount of intensity of expression of the level of expression of NT-proANP or ANP mRNA; or miR-103, and/or miR-105, and/or miR-107 and/or miR-425 expression level and the like as disclosed herein.

By providing data of the intensity of expression of (i) the level of expression of NT-proANP or ANP mRNA; and/or (ii) miR-103, and/or miR-105, and/or miR-107 and/or miR-155 and/or miR-425 expression level in computer-readable form, one can use the data to compare with data within the storage device. For example, search programs can be used to identify relevant reference data (i.e. data of appropriate reference cancer cell lines) that match the same type of cancer as the cancer of the test cancer cell population. The comparison made in computer-readable form provides computer readable content which can be processed by a variety of means. The content can be retrieved from the comparison module, the retrieved content.

In some embodiments, the comparison module provides computer readable comparison result that can be processed in computer readable form by predefined criteria, or criteria defined by a user, to provide a report which comprises content based in part on the comparison result that may be stored and output as requested by a user using a display module. In some embodiments, a display module enables display of a content based in part on the comparison result for the user, wherein the content is a report indicative of the results of the comparison of the intensity of expression of (i) the level of expression of NT-proANP or ANP mRNA; and/or (ii) miR-103, and/or miR-105, and/or miR-107 and/or miR-155 and/or miR-425 expression levels. For example, the data can be compared to representative reference values obtained from a cohort of subjects who have normal blood pressure (e.g., negative controls), or have high blood pressure (e.g., positive controls).

In some embodiments, the display module enables display of a report or content based in part on the comparison result for the end user, wherein the content is a report indicative of the results of the comparison of the intensity of expression of any one or more of (i) the level of expression of NT-proANP or ANP mRNA; and/or (ii) miR-103, and/or miR-105, and/or miR-107 and/or miR-155 and/or miR-425 expression levels.

In some embodiments of this aspect and all other aspects of the present invention, the comparison module, or any other module of the invention, can include an operating system (e.g., UNIX, Windows) on which runs a relational database management system, a World Wide Web application, and a World Wide Web server. World Wide Web application can includes the executable code necessary for generation of database language statements [e.g., Standard Query Language (SQL) statements]. The executables can include embedded SQL statements. In addition, the World Wide Web application may include a configuration file which contains pointers and addresses to the various software entities that comprise the server as well as the various external and internal databases which must be accessed to service user requests. The Configuration file also directs requests for server resources to the appropriate hardware—as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GenBank or Swiss Pro World Wide Web site). Thus, in a particular preferred embodiment of the present invention, users can directly access data (via Hypertext links for example) residing on Internet databases using an HTML interface provided by Web browsers and Web servers. In other embodiments of the invention, other interfaces, such as HTTP, FTP, SSH and VPN based interfaces can be used to connect to the Internet databases.

In some embodiments of this aspect and all other aspects of the present invention, a computer-readable media can be transportable such that the instructions stored thereon, such as computer programs and software, can be loaded onto any computer resource to implement the aspects of the present invention discussed herein. In addition, it should be appreciated that the instructions stored on the computer-readable medium, described above, are not limited to instructions embodied as part of an application program running on a host computer. Rather, the instructions may be embodied as any type of computer code (e.g., software or microcode) that can be employed to program a processor to implement aspects of the present invention. The computer executable instructions can be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001).

The computer instructions can be implemented in software, firmware or hardware and include any type of programmed step undertaken by modules of the information processing system. The computer system can be connected to a local area network (LAN) or a wide area network (WAN). One example of the local area network can be a corporate computing network, including access to the Internet, to which computers and computing devices comprising the data processing system are connected. In one embodiment, the LAN uses the industry standard Transmission Control Protocol/Internet Protocol (TCP/IP) network protocols for communication. Transmission Control Protocol Transmission Control Protocol (TCP) can be used as a transport layer protocol to provide a reliable, connection-oriented, transport layer link among computer systems. The network layer provides services to the transport layer. Using a two-way handshaking scheme, TCP provides the mechanism for establishing, maintaining, and terminating logical connections among computer systems. TCP transport layer uses IP as its network layer protocol. Additionally, TCP provides protocol ports to distinguish multiple programs executing on a single device by including the destination and source port number with each message. TCP performs functions such as transmission of byte streams, data flow definitions, data acknowledgments, lost or corrupt data re-transmissions, and multiplexing multiple connections through a single network connection. Finally, TCP is responsible for encapsulating information into a datagram structure. In alternative embodiments, the LAN can conform to other network standards, including, but not limited to, the International Standards Organization's Open Systems Interconnection, IBM's SNA, Novell's Netware, and Banyan VINES.

In some embodiments, the computer system as described herein can include any type of electronically connected group of computers including, for instance, the following networks: Internet, Intranet, Local Area Networks (LAN) or Wide Area Networks (WAN). In addition, the connectivity to the network may be, for example, remote modem, Ethernet (IEEE 802.3), Token Ring (IEEE 802.5), Fiber Distributed Datalink Interface (FDDI) or Asynchronous Transfer Mode (ATM). The computing devices can be desktop devices, servers, portable computers, hand-held computing devices, smart phones, set-top devices, or any other desired type or configuration. As used herein, a network includes one or more of the following, including a public internet, a private internet, a secure internet, a private network, a public network, a value-added network, an intranet, an extranet and combinations of the foregoing.

In one embodiment of the invention, the computer system can comprise a pattern comparison software can be used to determine whether the patterns of data of the intensity of expression of any one of (i) the level of expression of NT-proANP or ANP mRNA; and/or (ii) miR-103, and/or miR-105, and/or miR-107, and/or miR-155 and/or miR-425 expression levels are indicative of that the subject is at risk of hypertension, or high blood pressure and/or a cardiovascular disease or disorder as disclosed herein. In this embodiment, the pattern comparison software can compare at least some of the data (e.g., one or more of data of (i) the level of expression of NT-proANP or ANP mRNA; and/or (ii) miR-103, and/or miR-105, and/or miR-107, and/or miR-155 and/or miR-425 expression levels with reference data (e.g., data of (i) the level of expression of NT-proANP or ANP mRNA from subjects with normal blood pressure; and/or (ii) miR-103, and/or miR-105, and/or miR-107, and/or miR-155 and/or miR-425 expression levels for subjects with normal blood pressure to determine how closely they match. The matching can be evaluated and reported in portions or degrees indicating the extent to which all or some of the pattern matches.

In some embodiments of this aspect and all other aspects of the present invention, a comparison module provides computer readable data that can be processed in computer readable form by predefined criteria, or criteria defined by a user, to provide a retrieved content that may be stored and output as requested by a user using a display module.

Output Module

In accordance with some embodiments of the invention, the computerized system can include or be operatively connected to an output module. In some embodiments, the output module is a display module, such as computer monitor, touch screen or video display system. The display module allows user instructions to be presented to the user of the system, to view inputs to the system and for the system to display the results to the user as part of a user interface. Optionally, the computerized system can include or be operative connected to a printing device for producing printed copies of information output by the system.

In some embodiments, the results can be displayed on a display module or printed in a report, e.g., a to indicate any one or more of (i) the level of expression of NT-proANP or ANP mRNA; and/or (ii) miR-103, and/or miR-105, and/or miR-107, and/or miR-155 and/or miR-425 expression levels or any other report envisioned by the end user.

In some embodiments, the report is a hard copy printed from a printer. In alternative embodiments, the computerized system can use light or sound to report the result, e.g., to indicate any one or more of (i) the level of expression of NT-proANP or ANP mRNA; and/or (ii) miR-103, and/or miR-105, and/or miR-107, and/or miR-155 and/or miR-425 expression levels. For example, in all aspects of the invention, the report produced by the methods, assays, systems and kits as disclosed herein can comprise a report which is color coded to signal or indicate any one or more of (i) the level of expression of NT-proANP or ANP mRNA; and/or (ii) miR-103, and/or miR-105, and/or miR-107, and/or miR-155 and/or miR-425 expression levels, or compared another “gold” standard of, for example, the level of NT-proANP or ANP, or miR-103, and/or miR-105, and/or miR-107, and/or miR-155 and/or miR-425 expression levels for a subject who has normal blood pressure(e.g., negative standard).

For example, a red color or other predefined signal can indicate that the subject has NT-proANP, or ANP or miR-103, and/or miR-105, and/or miR-107 and/or miR-425 expression levels indicative of the subject having a high risk of hypertension as disclosed herein, and is a suitable subject for treatment with anti-miR agents as disclosed herein. In another embodiment, a green color or other predefined signal can indicate that the subject has NT-proANP, or ANP or miR-103, and/or miR-105, and/or miR-107, and/or miR-155 and/or miR-425 expression levels indicative of the subject having a low risk of hypertension as disclosed herein, and not a suitable subject for treatment with anti-miR agents as disclosed herein. Other color schemes and gradient schemes in the report are also encompassed.

In some embodiments, the report can display the normalized values of any one or more of the (i) the level of expression of NT-proANP or NPPA mRNA; and/or (ii) miR-103, and/or miR-105, and/or miR-107, and/or miR-155 and/or miR-425 expression levels, which can be normalized to the levels of a subject who is has normal blood pressure (e.g., a selected “gold” standard of subjects at “low risk of hypertension” or the investigators choice). Alternatively, the report can display the normalized values which are normalized to the levels of a subject who has high blood pressure (e.g., a reference of subjects at “high risk for hypertension” as disclosed herein). Accordingly, a report can display the % difference, and/or the change in absolute number of the values measured as compared to values for a subject with high blood pressure (“high risk” category) and/or a subject with low blood pressure (“low risk” category).

In some embodiments, the report can also present text, either verbally or written, giving a recommendation of if a subject is amenable to treatment with an anti-miR agent as disclosed herein. In other embodiments, the report provides just values or numerical scores for the presence of any one or more of the (i) the level of expression of NT-proANP or NPPA mRNA; and/or (ii) miR-103, and/or miR-105, and/or miR-107, and/or miR-155 and/or miR-425 expression levels which can be readily compared by a physician with reference values as disclosed herein.

In some embodiments of this aspect and all other aspects of the present invention, the report data from the comparison module can be displayed on a computer monitor as one or more pages of the printed report. In one embodiment of the invention, a page of the retrieved content can be displayed through printable media. The display module can be any device or system adapted for display of computer readable information to a user. The display module can include speakers, cathode ray tubes (CRTs), plasma displays, light-emitting diode (LED) displays, liquid crystal displays (LCDs), printers, vacuum florescent displays (VFDs), surface-conduction electron-emitter displays (SEDs), field emission displays (FEDs), etc.

In some embodiments of the present invention, a World Wide Web browser can be used to provide a user interface to allow the user to interact with the system to input information, construct requests and to display retrieved content. In addition, the various functional modules of the system can be adapted to use a web browser to provide a user interface. Using a Web browser, a user can construct requests for retrieving data from data sources, such as data bases and interact with the comparison module to perform comparisons and pattern matching. The user can point to and click on user interface elements such as buttons, pull down menus, scroll bars, etc. conventionally employed in graphical user interfaces to interact with the system and cause the system to perform the methods of the invention. The requests formulated with the user's Web browser can be transmitted over a network to a Web application that can process or format the request to produce a query of one or more database that can be employed to provide the pertinent information related to the tumor type, the retrieved content, process this information and output the results.

### Kits

In another embodiment, this invention provides kits for the practice of the methods of this invention. The kits preferably include one or more containers containing at least one anti-miR agent, e.g., an anti-miR which inhibits at least one or a combination of miR-103, and/or miR-105, and/or miR-107, and/or miR-155 and/or miR-425 as disclosed herein and a pharmaceutically acceptable excipient. The kit may optionally contain additional therapeutics to be co-administered with the one or more anti-miR agents. The kit may comprise instructions for administration of an anti-miR agent to a subject with a cardiovascular disease or condition, e.g., but not limited to, hypertension, congestive heart failure (CHF), myocardial infarction etc. or a subject in need of elevating or increasing plasma ANP levels.

In another embodiment, a kits can preferably include one or more containers containing at least one miR agent, e.g., at least one or a combination of miR-103, and/or miR-105, and/or miR-107, and/or miR-155 and/or miR-425 as disclosed herein and a pharmaceutically acceptable excipient. The kit may optionally contain additional therapeutics to be co-administered with the one or more miR agents. The kit may comprise instructions for administration of an miR agent as disclosed herein to a subject with low blood pressure, or where the subject is in need of increased blood pressure, e.g., a subject in a state of shock etc. or a subject in need of decreasing plasma ANP levels

The kits may also optionally include appropriate systems (e.g. opaque containers) or stabilizers (e.g. antioxidants) to prevent degradation of the anti-miR agent (e.g., that inhibits at least one of miR-103, miR-105, miR-107, and/or miR-155) or miR agent (e.g., miR-103, miR-105, miR-107, and/or miR-155 agent) by light or other adverse conditions.

In another aspect of the invention provides kits including one or more containers containing anti-miR agent (e.g., that inhibits at least one of miR-103, miR-105, miR-107, and/or miR-155 and/or miR-425) as disclosed herein and a pharmaceutically acceptable excipient. The kit may optionally contain additional therapeutics to be co-administered with the anti-miR agent (e.g., that inhibits at least one of miR-103, miR-105, miR-107, and/or miR-155), e.g., including but not limited to an anti-miR-425 agent as disclosed herein or in WO2013/0188787.

In some embodiments, the kit can contain at least two anti-miR agents (e.g., at least 4, 6, 8, 10, 12, 14, 16, 20, 22, 24, 26, 28, 30, 32, 34, 36, 38, or 40), e.g., wherein the kit comprises at least one anti-miR-155 agent and at least one anti-miR-425 agent. In some embodiments, a kit comprises at least one anti-miR-105 agent and at least one anti-miR-425 agent, and in some embodiments, the kit comprises at least one anti-miR-105 agent and at least one anti-miR-155 agent.

In some embodiments, the kits can contain at least one inhibitory nucleic acid and/or at least one sense nucleic acid (e.g., any of the inhibitory nucleic acids or sense nucleic acids described herein). In some embodiments, the kit contains at least one inhibitory nucleic acid (e.g., at least one inhibitory nucleic acid targeting any one of miR-103, miR-105, miR-107, and/or miR-155) formulated for intrathecal or intracranial injection or infusion.

The kits may optionally include instructional materials containing directions (i.e., protocols) providing for the use of anti-miR agent (e.g., that inhibits at least one of miR-103, miR-105, miR-107 and/or miR-155, and optionally an anti-miR-425 agent) for the treatment of a disease in a mammal, e.g., for the treatment of hypertension or low blood pressure respectively. In particular the diseases related to hypertension can include any one or more of the disorders described herein including, but not limited to hypertension (end stage renal hypertension, pregnancy-related hypertension (e.g., preeclampsia), salt sensitivity hypertension, type II diabetes hypertension, alcohol abuse or obesity related hypertension, systolic hypertension in the elderly, and essential hypertension), ischemic and hemorrhagic stroke.

Additionally, another aspect of the present invention relates to a kit comprising at least two or more primers (e.g., at least 4, 6, 8, 10, 12, 14, 16, 18, 20, 22, 24, 26, 28, 30, 32, 36, 38, or 40) for amplifying a sequence present within any of the miRs disclosed herein (e.g., mature miR or precursor miR of miR-103, miR-105, miR-107, miR-155 and/or miR-425) or for amplifying the sequence of any of the miRs of miR-103, miR-105, miR-107, miR-155 and/or miR-425 present within the plasma of a subject. In another embodiment,

While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

In some embodiments of the present invention may be defined in any of the following numbered paragraphs:
1. A method for increasing ANP levels (e.g., levels of pro-nt-ANP or ANP in the plasma) in a subject, comprising administering to the subject an anti-miR agent (e.g., miR antagonist) that inhibits the function and/or inhibits the expression of at least one of; miR-103, miR-105, miR-107 or miR-155, wherein the anti-miR agent prevents miR-103, miR-105, miR-107 or miR-155 mediated repression of the 3′UTR of the NPPA gene.
2. The method of paragraph 1, wherein the anti-miR agent is at least 80%, or at least 85%, or at least 90% complementary to the nucleobase sequence of any one of miR-103 (e.g, hsa-miR-103), miR-105 (e.g, hsa-miR-105), miR-107 (e.g, hsa-miR-107) or miR-155(e.g, hsa-miR-155).
3. The method of any of paragraphs 1-2, wherein the anti-miR agent specifically binds to the seed sequence of any one or a combination of miR-103, miR-105, miR-107 or miR-155, wherein the seed sequence of miR-103 or miR-107 is SEQ ID NO: 4, the seed sequence of miR-105 is SEQ ID NO: 9, and the seed sequence of miR-155 is SEQ ID NO: 14.
4. The method of any of paragraphs 1-3, wherein the anti-miR agent binds to the target sequence of miR-103, miR-105, miR-107 or miR-155 in the 3′UTR of the NPPA gene.
5. The method of any of paragraphs 1-4, wherein the subject has been identified to have any one of; hypertension, high blood pressure, low plasma ANP levels, or high levels of any one of miR-103, miR-105, miR-107 and/or miR-155, and wherein the subject has been selected for administration with the anti-miR agent.
6. The method of any of paragraphs 1-5, wherein the composition comprises at least two anti-miR agents selected from the groups of; anti-miR-103 and anti-miR-107; anti-miR-103 and anti-miR-105, anti-miR-105 and anti-miR-107, anti-miR-103 and anti-miR-155, anti-miR-107 and anti-miR-155 and anti-miR-105 and anti-miR-155.
7. The method of any of paragraphs 1-6, wherein the composition comprises at least an anti-miR-155 agent.
8. The method of any of paragraphs 1-7, wherein the composition comprises at an anti-miR-105 agent.
9. The method of any of paragraphs 1-8, wherein the composition futher comprises an anti-miR agent that inhibits the function of miR-425.
10. The method of any of paragraphs 1-9, wherein the composition comprises an anti-miR-105 agent and an anti-miR-425 agent, or an anti-mR-155 agent and anti-miR-425 agent.
11. The method of any of paragraphs 1-10, wherein the composition comprises an anti-miR-103 agent and anti-miR-425 agent, or an anti-miR-107 agent and an anti-miR-425 agent, or an anti-miR-103 agent and anti-miR-107 agent and an anti-miR-425 agent.
12. The method of any of paragraphs 1-11, wherein the anti-miR that inhibits the function of miR-103 inhibits the function of hsa-miR 103a-3p.
13. The method of any of paragraphs 1-12, wherein the anti-miR that inhibits the function of miR-105 inhibits the function of hsa-miR 105-5p.
14. The method of any of paragraphs 1-13, further comprising administering an additional therapeutic agent for the treatment of hypertension or high blood pressure.
15. The method of any of paragraph 1-14 for treating a subject with, or at risk of hypertension or a cardiovascular disease or disorder.
16. An isolated oligonucleotide at least 80%, or at least 85%, or at least 90% complementary to the nucleobase sequence of any one of miR-103 (e.g, hsa-miR-103), miR-105 (e.g, hsa-miR-105), miR-107 (e.g, hsa-miR-107) or miR-155(e.g, hsa-miR-155) for increasing ANP levels (e.g., plasma ANP levels) in a subject.
17. An isolated oligonucleotide at least 80%, or at least 85%, or at least 90% complementary to the nucleobase sequence of miR-105 or miR-155 for increasing plasma ANP levels in a subject.
18. An isolated oligonucleotide comprising a nucleotide sequence that is complementary to, and specifically binds to the seed sequence of any one or a combination of miR-103, miR-105, miR-107 or miR-155, wherein the seed sequence of miR-103 or miR-107 is SEQ ID NO: 4 and the seed sequence of miR-105 is SEQ ID NO: 9, and the seed sequence for miR-155 is SEQ ID NO: 14.
19. An isolated oligonucleotide comprising a nucleotide sequence that is at least 80%, or at least 85%, or at least 90% complementary to, and specifically binds to the target sequence of miR-103, miR-105, miR-107 or miR-155 in the 3′UTR of the NPPA gene, wherein the target sequence of miR-103 or miR-107 is SEQ ID NO: 6, and the target sequence of miR-105 is SEQ ID NO: 10 and the target sequence for miR-155 is SEQ ID NO: 199 or SEQ ID NO: 200.
20. A method for decreasing ANP levels (e.g., levels of pro-nt-ANP or ANP in the plasma) in a subject, comprising administering to the subject an miR agent (e.g, miR agonist) of miR-103 (e.g, hsa-miR-103), miR-105 (e.g, hsa-miR-105), miR-107 (e.g, hsa-miR-107) or miR-155 (e.g, hsa-miR-155), or is a mimetic thereof, or where the miR-agent increases the function of, at least one of; miR-103, miR-105, miR-107 or miR-155, wherein the miR agent increases miR-103, miR-105, miR-107 or miR-155 mediated repression of the 3′UTR of the NPPA gene.
21. The method of paragraph 20, wherein the miR agent is at least 80%, or at least 85%, or at least 90% identical to the nucleobase sequence of any one of miR-103, miR-105, miR-107 or miR-155.
22. The method of any of paragraphs 20-21, wherein the subject has been identified to have any of: hypotension, low blood pressure, high plasma ANP levels, or low levels of any one of miR-103, miR-105, miR-107 and/or miR-155, and has been selected for administration with the miR agent.
23. The method of any of paragraphs 20-22, wherein the composition comprises at least two miR agents selected from the groups of; miR-103 and miR-107; miR-103 and miR-105, miR-105 and miR-107, miR-103 and miR-155, miR-107 and miR-155, and miR-1-5 and miR-155.
24. The method of any of paragraphs 20-23, wherein the composition comprises miR-155 or a mimetic or modified miR-155 agent thereof, where the modified miR-155 agent binds to the G allele of rs61764044.
25. The method of any of paragraphs 20-23, wherein the composition comprises miR-105 or a mimetic or modified miR-105 agent thereof.
26. The method of any of paragraphs 20-25, wherein the composition futher comprises a miR agent that is a mimetic of, or increases the function of, miR-425.
27. The method of any of paragraphs 20-26, wherein the composition comprises a miR-105 agent and an a miR-425 agent, or miR-155 agent and miR-425 agent.
28. The method of any of paragraphs 20-27, wherein the composition comprises a miR-103 agent and a miR-425 agent, or a miR-107 agent and a miR-425 agent, or a miR-103 agent and a miR-107 agent and a miR-425 agent.
29. The method of any of paragraphs 20-28, wherein the miR agent that increases the function of miR-103 increases the function of hsa-miR 103a-3p.
30. The method of any of paragraphs 20-29, wherein the miR agent that increases the function of miR-105 increases the function of hsa-miR 105-5p.
31. The method of any of paragraphs 20-30, further comprising administering an additional therapeutic agent for the treatment of hypotension or low blood pressure.
32. The method of any of paragraphs 20-31 for treating a subject with hypotension or shock.
33. An assay comprising;
   a. measuring the levels of at least one or more of: miR-103, miR-105, miR-107 or miR-155 in a biological sample obtained from the subject;
   b. comparing the measured levels of one or more of miR-103, miR-105, miR-107 or miR-155 with a reference value, and
   c. characterizing the subject has being at risk of having high blood pressure, hypertension or a cardiovascular disease or disorder at if the amount of miR-103, miR-105, miR-107 or miR-155 is increased relative to the reference value; and not identifying the subject has being at risk of having high blood pressure, hypertension or a cardiovascular disease or disorder at if the amount of miR-103, miR-105, miR-107 or miR-155 is the same or lower relative to the reference value.
34. The assay of paragraph 33, further comprising administering to the subject characterized at risk of high blood pressure, hypertension or a cardiovascular disease or disorder a composition comprising an anti-miR agent (e.g., miR antagonist) that inhibits the function and/or inhibits the expression of at least one of; miR-103, miR-105, miR-107 or miR-155, wherein the anti-miR agent prevents miR-103, miR-105, miR-107 or miR-155 mediated repression of the 3′UTR of the NPPA gene.
35. An assay comprising;
   a. measuring the levels of at least one or more of: miR-103, miR-105, miR-107 or miR-155 in a biological sample obtained from the subject;
   b. comparing the measured levels of one or more of miR-103, miR-105, miR-107 or miR-155 with a reference value, and
   c. characterizing the subject has being at risk of having low blood pressure, hypotension or related condition if the amount of miR-103, miR-105, miR-107 or miR-155 is decreased relative to the reference value; and not identifying the subject being at risk of low blood pressure, hypotension or related condition at if the amount of miR-103, miR-105, miR-107 or miR-155 is the same or increased relative to the reference value.
36. The assay of paragraph 35, further comprising administering to the subject characterized at risk of low blood pressure, hypotension or related condition a composition comprising a miR agent (e.g, miR agonist) of miR-103, miR-105, miR-107 or miR-155, or is a mimetic thereof, or where the miR-agent increases the function of, at least one of; miR-103, miR-105, miR-107 or miR-155, wherein the miR agent increases miR-103, miR-105, miR-107 or miR-155 mediated repression of the 3′UTR of the NPPA gene.
37. A pharmaceutical composition comprising at least one anti-miR-155 agent and at least one anti-miR-425 agent, and a pharmaceutically acceptable carrier.
38. A pharmaceutical composition comprising at least one anti-miR-105 agent and at least one anti-miR-425 agent, and a pharmaceutically acceptable carrier.
39. The pharmaceutical composition of paragraph 37, wherein the anti-miR-155 agent is at least 80%, or at least 85%, or at least 90% complementary to the nucleobase sequence of miR-155, or the seed sequence of miR-155 of SEQ ID NO: 14.
40. The pharmaceutical composition of paragraph 37, wherein the anti-miR-155 agent is an inhibitory nucleic acid sequence comprising a sequence that is complementary to a contiguous sequence of at least 5, or at least 6, or at least 7 or at least 8, or at least 9 or at least 10, nucleotides present in hsa-miR-155.
41. The pharmaceutical composition of paragraph 37, wherein the anti-miR-155 agent comprises a nucleotide sequence that is complementary to, and specifically binds to the seed sequence of SEQ ID NO: 14, or is at least 80%, or at least 85%, or at least 90% complementary to, and specifically binds to the target sequence of miR-155 in the 3′UTR of the NPPA gene of SEQ ID NO: 199 or SEQ ID NO: 200.
42. The pharmaceutical composition of paragraph 37, wherein the anti-miR-155 has a sequence of SEQ ID NO: 240.
43. The pharmaceutical composition of paragraph 38, wherein the anti-miR-105 agent is at least 80%, or at least 85%, or at least 90% complementary to the nucleobase sequence of miR-105, or the seed sequence of SEQ ID NO: 9.
44. The pharmaceutical composition of paragraph 38, wherein the anti-miR-105 agent is an inhibitory nucleic acid sequence comprising a sequence that is complementary to a contiguous sequence of of at least 5, or at least 6, or at least 7 or at least 8, or at least 9 or at least 10 nucleotides present in hsa-miR-105.
45. The pharmaceutical composition of paragraph 38, wherein the anti-miR-105 agent comprises a nucleotide sequence that is complementary to, and specifically binds to the seed sequence of SEQ ID NO: 9, or is at least 80%, or at least 85%, or at least 90% complementary to, and specifically binds to the target sequence of miR-105 in the 3′UTR of the NPPA gene of SEQ ID NO: 10.
46. The pharmaceutical composition of paragraphs 37 or 38, wherein the anti-miR-425 agent is at least 80%, or at least 85%, or at least 90% complementary to the nucleobase sequence of miR-425, or the seed sequence of SEQ ID NO: 16.
47. The pharmaceutical composition of paragraphs 37 or 38, wherein the anti-miR-425 agent comprises a nucleotide sequence that is complementary to, and specifically binds to the seed sequence of SEQ ID NO: 16, or is at least 80%, or at least 85%, or at least 90% complementary to, and specifically binds to the target sequence of miR-425 in the 3′UTR of the NPPA gene of SEQ ID NO: 18.
48. The pharmaceutical composition of paragraphs 37 or 38, wherein the anti-miR-425 agent is an inhibitory nucleic acid sequence comprising a sequence that is complementary to a contiguous sequence of at least 5, or at least 6, or at least 7 or at least 8, or at least 9 or at least 10, nucleotides present in hsa-miR-425.
49. The pharmaceutical composition of any of paragraphs 37-48, wherein the at least one inhibitory nucleic acid is an antagomir.
50. The pharmaceutical composition of any of paragraphs 37-48, wherein the at least one inhibitory nucleic acid is an antisense oligonucleotide.
51. The use of the pharmaceutical composition of any of paragraphs 37-50 for the treatment of a subject with any one of: hypertension, high blood pressure or a cardiovascular disease or disorder.
52. A pharmaceutical composition comprising at least one miR-155 agent (e.g., miR-155 agonist) and at least one miR-425 agent (e.g., miR-425 agonist), and a pharmaceutically acceptable carrier.
53. A pharmaceutical composition comprising at least one miR-105 agent (e.g., miR-155 agonist) and at least one miR-425 agent (e.g., miR-425 agonist), and a pharmaceutically acceptable carrier.
54. A pharmaceutical composition of paragraph 52, wherein the miR-155 agent is at least 80%, or at least 85%, or at least 90% identical to the nucleobase sequence of miR-155 (e.g. the mature miR-155 or pre-miR-155).
55. The pharmaceutical composition of paragraph 52, wherein the miR-155 agent comprises a sequence a contiguous sequence of at least 5 nucleotides present in hsa-miR-155.
56. The pharmaceutical composition of paragraph 53, wherein the miR-105 agent is at least 80%, or at least 85%, or at least 90% identical to the nucleobase sequence of miR-105 (e.g. the mature miR-105 or pre-miR-105).
57. The pharmaceutical composition of paragraph 53, wherein the miR-105 agent comprises a sequence that is a contiguous sequence of at least 5 nucleotides present in hsa-miR-105.
58. The pharmaceutical composition of paragraphs 52 or 53, wherein the miR-425 agent is at least 80%, or at least 85%, or at least 90% identical to the nucleobase sequence of miR-425 (e.g. the mature miR-425 or pre-miR-425).
59. The pharmaceutical composition of paragraphs 52 or 53, wherein the miR-425 agent is a nucleic acid sequence comprising a contiguous sequence of at least 5 nucleotides present in hsa-miR-425.
60. The use of the pharmaceutical composition of any of paragraphs 52-59 for the treatment of a subject with any one of: hypotension or low blood blood pressure or a related disease or disorder.
61. A method of treating a subject with high blood pressure, hypertension or cardiovascular disease or disorder comprising administering to a subject an anti-miR agent that inhibits the function of at least one of; miR-103, miR-105, miR-107 or miR-155, wherein the anti-miR agent prevents miR-103, miR-105, miR-107 or miR-155 mediated repression of the 3′UTR of the NPPA gene, and wherein the subject has been identified to have the plasma expression of any one of; miR-103, miR-105, miR-107 or miR-155 above a pre-defined reference control level for that miRNA.

The technology described herein is further illustrated by the following examples which in no way should be construed as being further limiting, The contents of all cited references, including literature references, issued patents, published patent applications, and co-pending patent applications, cited throughout this application are hereby expressly incorporated by reference.

The technology described herein has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### EXAMPLES

Throughout this application, various publications are referenced. The disclosures of all of the publications and those references cited within those publications in their entireties are hereby incorporated by reference into this application in order to more fully describe the state of the art to which this invention pertains. The following examples are not intended to limit the scope of the claims to the invention, but are rather intended to be exemplary of certain embodiments. Any variations in the exemplified methods which occur to the skilled artisan are intended to fall within the scope of the present invention.

Materials and Methods:

Computational algorithms were used to generate a list of miRNAs predicted to interact with NPPA mRNA. miRNAs were prioritized based on miRNA expression data in human atrial tissues and/or presence of common genetic variants within the predicted miRNA binding site that are associated with circulating ANP levels in human population genetics studies. Candidate NPPA-targeting miRNAs were validated using luciferase reporter assays and human embryonic stem cell-derived cardiomyocytes.

In Silico Analysis—

The microRNA Data Integration Portal (mirDIP) (Shirdel E A, et al., PLoS One 2011; 6:e17429) was used to generate a list of miRNAs that are predicted in silico to interact with the NPPA 3′UTR (NM_006172; mirDIP accessed Jan. 18, 2015). The mirDIP (version 1.1.2) was run with the default settings.

Cell Culture—

COS-7 cells were purchased from ATCC (Manassas, Va.). Human cardiomyocytes were differentiated from human embryonic stem cells (hESCs) as previously described (Atmanli A, Adv Healthc Mater 2014; 3:1759-64). Specifically, hESCs from the WA07 cell line were used for differentiation, and cardiomyocytes were harvested 5-10 days after onset of beating, typically from day 15 to day 20 of differentiation. Expression of cardiac troponin T was confirmed in all experiments. Cardiomyocytes were confirmed by sequencing to be homozygous for the rs5068, rs61764044, and rs5067 major alleles.

Generation of Luciferase-NPPA 3′UTR Reporter Constructs—

The NPPA 3′UTR sequence (299 bp) including the major alleles of rs5068, rs5067, and rs61764044 was PCR amplified from a de-identified human HapMap genomic DNA sample obtained from Coriell Repositories (Coriell Institute for Medical Research, Camden, N.J.). The PCR product was cloned into the pISO vector (Addgene, Cambridge, Mass.) 3′ of the sequence encoding firefly luciferase to generate the wild-type luciferase construct (WT-Luc). In addition, mutated NPPA 3′UTR luciferase constructs were created by site-directed mutagenesis using QuickChange II XL site-directed mutagenesis kit (Stratagene, Santa Clara, Calif.) and synthetic oligonucleotides (Life Technologies, Grand Island, N.Y.) according to the manufacturers' protocols. Sequences of the synthetic oligonucleotides are provided in the Table 5.

TABLE 5
Sequences of the synthetic oligonucleotides used in site-directed mutagenesis
to generate the Luc-Mut NPPA 3′UTR constructs:

| Construct | Sequence (5′ to 3′) |
|---|---|
| 155 Mut-Luc | Forward: CTCGCCTCTCCCACCCCATCGTGGCAATTTTAAGGTAGAACCTC |
| | (SEQ ID NO: 241) |
| | Reverse: |
| | GAGGTTCTACCTTAAAATTGCCACGATGGGGTGGGAGAGGCGAGG (SEQ |
| | ID NO: 242) |
| rs61764044 | Forward: GCCTCTCCCACCCCACGCATTAAATTTTAAG (SEQ ID NO: 243) |
| Minor-Luc | Reverse: CTTAAAATTTAATGCGTGGGGTGGGAGAGGC (SEQ ID NO: 244) |
| rs5067 Minor- | Forward: GGTCTCTGCTGCATTCGTGTCATCTTGTTGC (SEQ ID NO: 245) |
| Luc | Reverse: GCAACAAGATGACACGAATGCAGCAGAGACC (SEQ ID NO: 246) |
| 103/107 | Forward: CTCCTGTCCCCTGGGGTCTCAACGCGATTTGTGTCATCTTGTTGC |
| Mut-Luc | (SEQ ID NO: 247) |
| | Reverse: |
| | GCAACAAGATGACACAAATCGCGTTGAGACCCCAGGGGACAGGAG (SEQ |
| | ID NO: 248) |

Transient Transfection of Luciferase Constructs and miRNAs/Anti-miRs into Heterologous Cells—

COS-7 cells were transfected with wild-type or mutated luciferase-NPPA 3′UTR constructs using X-tremeGENE HP DNA transfection reagent (Roche, Indianapolis, Ind.) according to the manufacturer's protocol. Twenty-four hours after transfection of the constructs, mirVana miRNA mimics (5 nM) or anti-miR inhibitors (100 nM) were transfected using Lipofectamine RNAiMax (Life Technologies). For miRNA transfection using two miRNA mimics, 5 nM of each miRNA mimic was transfected to obtain a final concentration of 10 nM. For anti-miR co-transfection, 100 nM of each anti-miR was transfected to obtain a final concentration of 200 nM. After an additional 48 hours, firefly and renilla luciferase activities in cell extracts were measured using the Dual Luciferase Reporter Assay System (Promega, Madison, Wis.). Each experiment was repeated 3 times (at least 6 replicates per experiment).

Transient Transfection of miRNAs/Anti-miRs into hESC-Derived Cardiomyocytes (hESC-CMs)—

hESC-CMs were transfected with either miRNA mimic (50 nM) or anti-miR (100 nM) using Lipofectamine RNAiMax. For miRNA co-transfection, 50 nM of each miRNA mimic was transfected to obtain a final concentration of 100 nM. After 24 hours, cells were washed and incubated in serum-free medium for an additional 24 hours. NPPA gene expression was measured using qRT-PCR and ANP protein production and secretion were assessed using an ELISA assay (proANP 1-98, Biomedica Medizinprodukte GmbH&Co KG, Austria) to detect N-terminal-proANP (Nt-proANP) levels in culture media. Each experiment was repeated 3 times (at least 6 replicates per experiment).

Transient Transfection of NPPA cDNA Expression Plasmid and miR-105/Anti-miR-105 into Heterologous Cells—

A full-length human NPPA cDNA expression plasmid containing the rs5067 major allele (OriGene, Rockville, Md., catalog # SC122740) and a plasmid specifying renilla luciferase (as a transfection efficiency control) were transfected into COS-7 cells using X-tremeGENE HP DNA transfection reagent. Twenty-four hours after transfection of the constructs, miR-105 (5 nM) or anti-miR-105 (100 nM) was transfected using Lipofectamine RNAiMax. Cells and culture media were collected 24 hours after miRNA/anti-miR transfection for measurement of renilla luciferase activity and secreted Nt-proANP levels by ELISA, respectively. The experiment was repeated 3 times (at least 6 replicates per experiment).

Measurement of mRNA and miRNA Levels—

Total RNA was extracted from cultured cells using TRIzol (Life Technologies), and cDNA was synthesized using High Capacity cDNA Reverse Transcription kit (Applied Biosystems, Foster City, Calif.) according to the manufacturer's protocol. NPPA and GAPDH mRNA levels were measured by qRT-PCR using Taqman assays (Applied Biosystems) in a 7500 Fast Real-Time PCR System (Life Technologies) according to the manufacturer's protocol. Relative changes in NPPA mRNA levels normalized to GAPDH mRNA levels were determined using the relative cycle threshold (Ct) method. TaqMan miRNA reverse transcription and real-time assay kits (Applied Biosystems) were used to detect mature miRNAs and U6 small nuclear RNA (U6 snRNA). Relative changes in miRNA levels normalized to U6 snRNA levels were determined using the relative Ct method.

Human Population Genetic Analysis—

Nt-proANP was measured in plasma samples from the Malmo Diet and Cancer Study using an immunoluminometric assay (BRAHMS, Berlin, Germany), as previously described (Smith J G, et al., J Am Coll Cardiol 2010; 56:1712-9). Genotypes were generated using a genome-wide SNP microarray (Illumina Omni Express Exome, Illumina, San Diego, Calif.). Genetic association was tested using linear regression with an additive genetic model adjusted for age and sex.

Statistics—

Data are presented as mean±SEM, and statistical significance was assessed by 2-tailed independent t test and one-way ANOVA with Bonferroni post-hoc testing, as appropriate. In all cases, a p value <0.05 was considered statistically significant.

### Example 1

The inventors previously discovered that miR-425 targets the NPPA mRNA rs5068 major allele and revealed a novel mechanism regulating ANP biosynthesis. These findings demonstrated that miR-425 antagonists could be used to increase ANP biosynthesis with the goal of reducing blood pressure and treating volume overload. The inventors has filed a patent application relating to the use of miR-425 antagonists (anti-miR-425) for the treatment of hypertension and other disorders associated with salt and water accumulation, as disclosed in International Application WO2013/188787, which is incorporated herein in its entirety by reference.

Multiple miRs are Predicted to Target the NPPA mRNA—

mRNAs typically contain multiple miR-binding sites, and the function of any given mRNA can be coordinately regulated by binding of different miRs (Balaga et al. Nucleic Acids Research 2012. 40:9404-9416). It is conceivable that other miRs, in addition to miR-425, can target the NPPA mRNA and that these miRs have an important role in the regulation of ANP biosynthesis. Identifying these other miRs could be crucial for the development of anti-miR therapies designed to augment ANP synthesis. Moreover, utilizing multiple anti-miRs may permit the use of low doses of individual anti-miRs, thereby minimizing adverse “off-target” effects induced by interfering with miR interactions with other mRNAs.

miR-103, miR-105, and miR-107 Target the NPPA 3′UTR—

Using bioinformatic methods, the inventors identified 130 miRs that are predicted to bind to the NPPA 3′UTR by any of several databases (TargetScan, PicTar, miRanda, mirBase, PITA, RNA22, and DIANA-microT). Of these, 24 miRs (including miR-425) are predicted to interact with NPPA mRNA by ≥3 databases. By performing luciferase-3′UTR reporter assays, the inventors tested the ability of 10 of the remaining 23 miRs to interact with the NPPA 3′UTR. Interestingly, of the 10 miRs tested, the inventors were only able to validate the interaction of 3 of the miRs (miR-103, miR-105, and miR-107) with the NPPA 3′UTR using the luciferase activity discussed below. Interestingly, miR-4770, miR-196a-3p, miR-1301, miR-139-5p, miR-143-3p, miR-151-5p, miR-194, miR-224, and miR-769-5p were identified as not interacting with the 3′UTR of NPPA. The inventors further characterized the interaction between these 3 miRs (miR-103, miR-105, and miR-107) and NPPA mRNA by investigating whether the miRs can modulate NPPA mRNA function in human cardiomyocyte progenitor cells and in heterologous cells.

### Example 2

Luciferase-3′UTR Reporter Assays to Test the Ability of miR-105 to Interact with the NPPA 3′UTR:

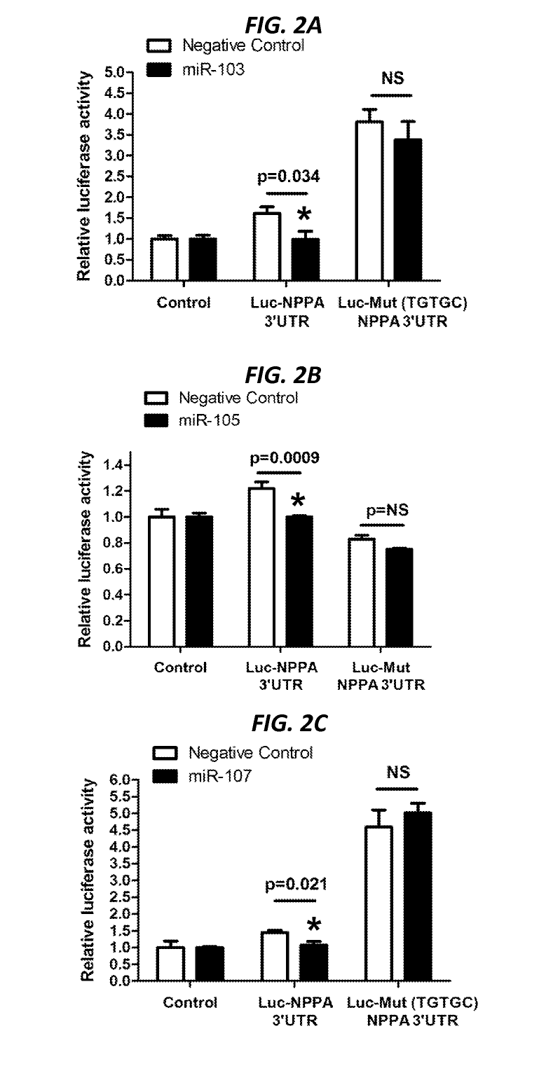

A plasmid containing a sequence specifying firefly luciferase ligated to the NPPA 3′UTR sequence was constructed (Luc-NPPA 3′UTR). Luc-NPPA 3′UTR or a plasmid expressing firefly luciferase without the NPPA 3′UTR (Control) were transfected into COS-7 cells together with a plasmid directing expression of renilla luciferase (as a control for transfection efficiency). Twenty-four hours later, COS-7 cells were transfected with miR-105 or a negative control miR. Forty-eight hours later, cells were harvested, and firefly and renilla luciferase activities in cell extracts were measured. The ratio of firefly luciferase activity to renilla luciferase activity was calculated and was normalized to that in cells transfected with a control plasmid directing expression of luciferase without the NPPA 3′UTR (relative luciferase activity). Transfection of miR-105 reduced the relative luciferase activity in cells transfected with Luc-NPPA 3′UTR (FIG. 2B). However, transfection of miR-105 had no significant effect on the relative luciferase activity in cells transfected with a plasmid expressing firefly luciferase ligated to the NPPA 3′UTR sequence in which one nucleotide in the region complementary to the miR-105 seed sequence was mutated (Luc-Mut NPPA 3′UTR) (FIG. 2B). Thus, the inventors discovered that the NPPA 3′UTR is a direct target of miR-105.

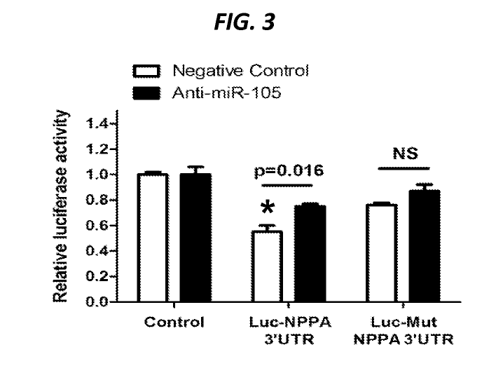

To further characterize the interaction of miR-105 with the NPPA 3′UTR, the inventors transfected COS-7 cells with plasmids directing expression of Luc-NPPA 3′UTR or Luc-Mut NPPA 3′UTR and an anti-miR-105. Transfection of anti-miR-105 increased the relative luciferase activity in cells transfected with Luc-NPPA 3′UTR, but there was no significant effect in cells transfected with Luc-Mut NPPA 3′UTR (FIG. 3).

Luciferase-3′UTR Reporter Assays to Test the Ability of miR-103 and miR-107 to Interact with the NPPA 3′UTR:

By performing similar luciferase-3′UTR reporter assays as described above, the inventors discovered that transfection of either miR-103 or miR-107 reduced the relative luciferase activity in COS-7 cells transfected with Luc-NPPA 3′UTR (FIG. 2A, 2C). To confirm that the reduction in luciferase activity is due to the direct interaction of miR-103 and miR-107 with the NPPA 3′UTR, the inventors constructed a plasmid expressing firefly luciferase ligated to the NPPA 3′UTR sequence in which 5 nucleotides in the region complementary to the seed sequence shared by miR-103 and miR-107 was mutated (Luc-Mut (TGTGC) (SEQ ID NO: 5) NPPA 3′UTR). Transfection of either miR-103 or miR-107 had no significant effect on the relative luciferase activity in cells transfected with Luc-Mut (TGTGC) (SEQ ID NO: 5) NPPA 3′UTR (FIG. 2A, 2C). Thus, the inventors discovered that the NPPA 3′UTR is also a direct target of miR-103 and miR-107.

### Example 3

Anti-miR-105 Increases NPPA mRNA Levels and ANP Biosynthesis:

To validate the interaction of miR-105 with endogenous NPPA, the inventors investigated the ability of a miR-105 antagonist, anti-miR-105, to modulate NPPA mRNA levels in human cardiomyocyte progenitor cells isolated from adult human heart auricle tissue (Dr. Marie-Jose Goumans). The cells were transfected with anti-miR-105 (or a negative control anti-miR), and endogenous NPPA mRNA levels were measured 48 hours later. Transfection of anti-miR-105 increased NPPA mRNA levels (FIG. 4A) demonstrating a role for miR-105 in the regulation of NPPA gene expression in human cardiomyocytes. Similar results were discovered when HepG2 cells (which express the NPPA gene at low levels) were transfected with anti-miR-105 (FIG. 5B). The invnetors also discovered that transfection of HepG2 cells with miR-105 decreased endogenous NPPA mRNA levels (FIG. 5A).

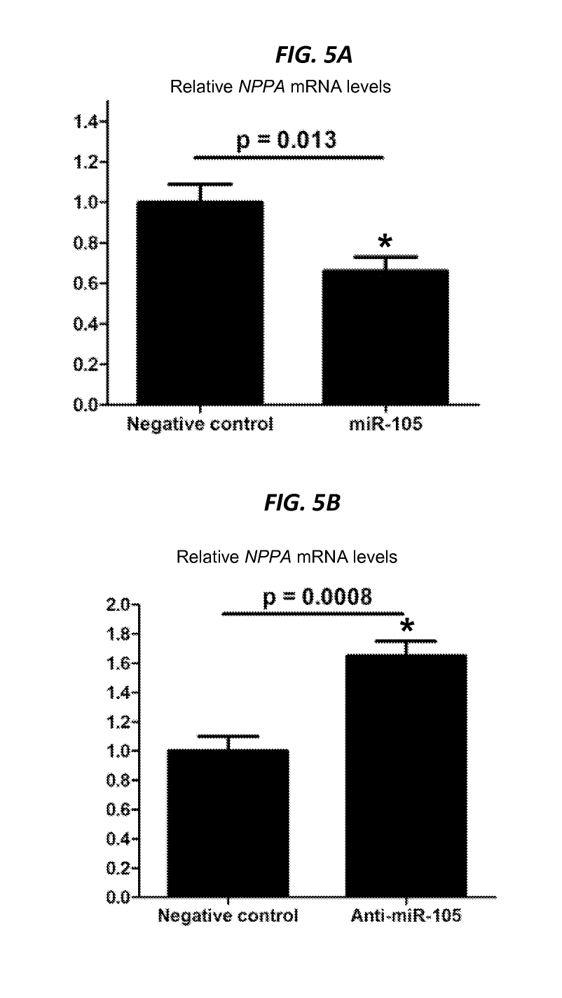

To test whether anti-miR-105 can increase ANP biosynthesis, the inventors transfected an expression plasmid directing expression of the human NPPA cDNA into COS-7 cells together with a plasmid directing expression of renilla luciferase (as a control for transfection efficiency). Twenty-four hours later, cells were transfected with anti-miR-105 or a negative control anti-miR. Twenty-four hours later, media was collected for measurement of NT-proANP, and cells were collected for measurement of renilla luciferase. Transfection of anti-miR-105 increased NT-proANP production (FIG. 6). These results demonstrate that anti-miR-105 can increase ANP biosynthesis.

Taken together, these results demonstrate that miR-105 directly targets NPPA mRNA and can regulate ANP biosynthesis.

Anti-miR-103 and Anti-miR-107 Increases NPPA mRNA Levels:

To validate the interaction of miR-103 and miR-107 with endogenous NPPA, the inventors investigated the ability of anti-miR-103 and anti-miR-107 to modulate NPPA mRNA levels in human cardiomyocyte progenitor cells using methods similar to those described above for miR-105. The inventors discovered that transfection of either anti-miR-103 or anti-miR-107 increased endogenous NPPA mRNA levels (FIG. 4B) demonstrating a direct role for miR-103 and miR-107 in the regulation of NPPA expression in human cardiomyocytes.

Taken together, the inventors have discovered that miR-105, as well as miR-103 and miR-107, are miRs, that in addition to miR-425, that can target NPPA mRNA.

### Example 4

miR-103, miR-105, and miR-107 Expression in Human Cardiac Tissues:

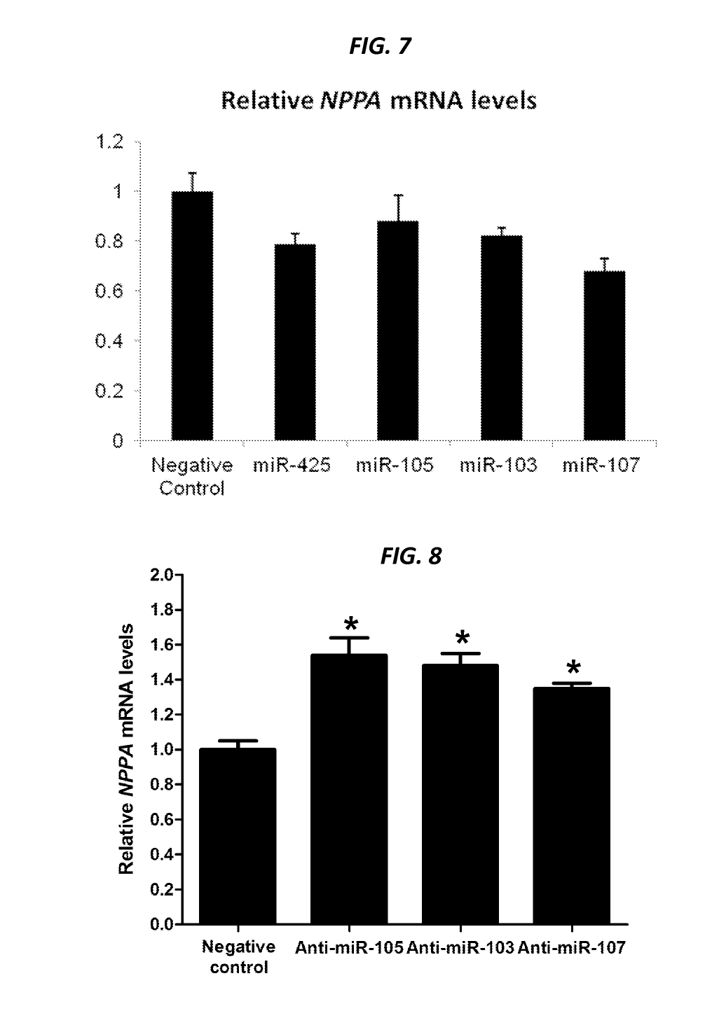

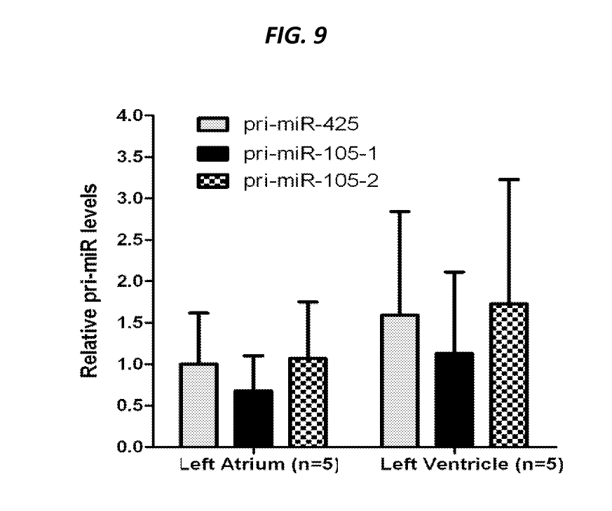

Atrial natriuretic peptide is predominantly expressed in the atrium with lower levels in the ventricle. To assess if miR-103, miR-105, and miR-107 play a role in the regulation of ANP production, the inventors assessed the miR expression in the heart. The inventors obtained left atrial and left ventricular tissue from donor hearts that were not suitable for transplantation but did not have known cardiac diseases (n=5 each). Using qRT-PCR, the inventors discovered that the expression of mature miR-103, miR-105, and miR-107 were detectable in the atrial and ventricular tissues, although the mature levels of miR-105 were much lower compared to that of miR-425. This discovery corroborated the finding reported by RNA sequencing data from human atrial tissues (J Hsu et al. Circulation: Cardiovascular Genetics 2012, 5:327-335). Despite the low expression levels of miR-105 in human cardiac tissues, the inventors demonstrate herein that anti-miR-105 in human cardiomyocyte progenitor cells (FIG. 4A) increases NPPA expression, and still plays an imporant role in regulating NPPA expression in human cardiomyocytes. Furthermore, to assess if anti-miR-103, anti-miR-105, and anti-miR-107 can increase production in human cardiomycocytes, the inventors assessed hESC-derived cardiomyocytes transfected with anti-miR-105, anti-miR-103 or anti-miR-107 and discovered that each of these anti-miRs could increase NPPA mRNA expression in human ESC-derived cardiomyocytes (FIG. 8). As mentioned above, biogenesis of a miR begins with the transcription of its gene into a primary transcript known as the pri-miR. Because of the low levels of expression of mature miR-105 in the atrial and ventricular tissues, the inventors assessed the expression of the primary transcripts of miR-105 to obtain further evidence for the expression of miR-105 in cardiac tissues. Two primary transcripts have been reported for miR-105: pri-miR-105-1 and pri-miR-105-2 (Z Mourelatos et al. Genes and Development 2002, 16:720-728). If miR-105 is expressed in the heart, there should be expression of its primary transcripts. By using qRT-PCR, the inventors discovered the expression of both pri-miR-105-1 and pri-miR-105-2 in all of the human atrial and ventricular tissues (FIG. 9). The levels of pri-miR-105-1 and pri-miR-105-2 in the tissues were comparable to that of the primary transcript of miR-425 (pri-miR-425) (FIG. 9). Accordingly, the inventors have discovered that miR-105 gene is transcribed and miR-105 is expressed in cardiac tissues.

### Example 5

Atrial natriuretic peptide (ANP) has a central role in regulating blood pressure in humans and is released in heart failure as a compensatory response to increased left ventricular wall tension. The inventors previously discovered that a cardiac-expressed microRNA, miR-425, regulated ANP production by binding to the mRNA of NPPA, the gene encoding ANP. mRNAs typically contain multiple predicted microRNA (miRNA)-binding sites, and binding of different miRNAs may independently or coordinately regulate the expression of any given mRNA.

Expanding on this, the inventors herein used a multifaceted screening strategy that integrates bioinformatics, next-generation sequencing data, human genetic association data, and cellular models to identify additional functional miRNAs that target NPPA mRNA. More specifically, computational algorithms were used to generate a list of miRNAs predicted to interact with NPPA mRNA. miRNAs were prioritized based on miRNA expression data in human atrial tissues and/or presence of common genetic variants within the predicted miRNA binding site that are associated with circulating ANP levels in human population genetics studies. Candidate NPPA-targeting miRNAs were validated using luciferase reporter assays and human embryonic stem cell-derived cardiomyocytes.

Using this approach, the inventors discovered that two novel miRNAs, miR-155 and miR-105 modulate ANP production in human cardiomyocytes and target genetic variants whose minor alleles are associated with higher human plasma ANP levels. Furthermore, the inventors discoverer herein that both miR-155 and miR-105 repressed NPPA mRNA in an allele-specific manner, with the minor allele of each respective variant conferring resistance to the miRNA either by disruption of miRNA base pairing or creation of wobble base pairing. Moreover, the inventors demonstrate that miR-155 can enhance (e.g., act synergistically) with the repressive effects of miR-425 on ANP production in human cardiomyocytes. Thus, the inventors herein have combined computational, genomic, and cellular tools to identify novel miRNA regulators of ANP production that are useful to be targeted (e.g., inhibited) to raise ANP levels in human subjects, for example, for the treatment of humans with hypertension or heart failure.

miRNAs Predicted to Target the NPPA 3′UTR

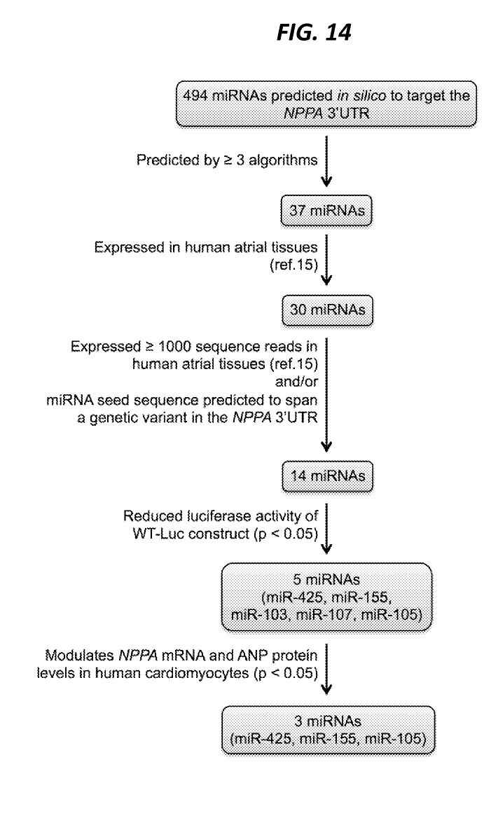

The mirDIP (Shirdel et al., PLoS One 2011; 6:e17429) was used to generate a list of miRNAs predicted in silico to interact with the NPPA 3′UTR. The 7 algorithms in mirDIP use a number of criteria to identify potential miRNA-mRNA interactions, including base pairing with the seed sequence (i.e. nucleotides 2-8 at the miRNA 5′-end), evolutionary conservation of target sites, thermal stability of the miRNA-mRNA duplex, and secondary structure surrounding the miRNA binding site (van Rooij E. et al. Circ Res 2011; 108:219-344). Four hundred ninety-four miRNAs were predicted to target the NPPA 3′UTR by at least one of the 7 mirDIP algorithms and 37 miRNAs were predicted to interact with the NPPA 3′UTR by 3 or more of the algorithms (FIG. 14). Thirty of the 37 miRNAs were expressed in human atrial tissues (Hsu J, et al., Circ Cardiovasc Genet 2012; 5:327-35.) As expected, miR-425 was among the 30 miRNAs predicted to target NPPA. The inventors next focused our attention on 12 of the remaining 29 miRNAs because they were expressed at high levels in human right/left atrial tissues (Hsu J, et al., Circ Cardiovasc Genet 2012; 5:327-35). Seed binding sites of 3 of these 12 miRNAs were predicted to include common genetic variants, either SNPs (miR-155 and miR-769-5p) or an insertion-deletion (miR-497-3p), in the NPPA 3′UTR (Table 6)).

TABLE 6
Summary of human atrial expression data, interaction
with genetic variants, and luciferase assay data for
the miRNAs predicted by at least 3 algorithms to target the
NPPA 3'UTR. Evidence of expression
(≥1000 sequence reads) in human atrial tissues were based
on RNA sequencing data in Hsu et al.,
Circ Cardiovasc Genet 2012;5:327-35. (

| | Predicted | Expressed | Seed | Reduces |
|---|---|---|---|---|
| | by ≥3 | ≥1000 | binding | luciferase |
| | algorithms | reads | site includes a | activity |
| | to target | in human | genetic variant | of WT-Luc |
| | NPPA | atrial | in the NPPA | construct |
| miRNA | 3'UTR? | tissues | 3'UTR? | (p < 0.05)? |
| miR-103a-3p | Yes | Yes | No | Yes |
| miR-105-5p | Yes | No | Yes | Yes |
| miR-107 | Yes | Yes | No | Yes |
| miR-1184-1 | Yes | No | No | Not tested |
| miR-1253 | Yes | No | No | Not tested |
| miR-125a-3p | Yes | Yes | No | No |
| miR-1294 | Yes | No | No | Not tested |
| miR-1301 | Yes | No | No | Not tested |
| miR-136-5p | Yes | No | No | Not tested |
| miR-139-5p | Yes | Yes | No | No |
| miR-143-3p | Yes | Yes | No | No |
| miR-151a-5p | Yes | Yes | No | No |
| miR-155-5p | Yes | Yes | Yes | Yes |
| miR-183-5p | Yes | No | No | Not tested |
| miR-194-1 | Yes | No | No | Not tested |
| miR-22-3p | Yes | Yes | No | No |
| miR-224-5p | Yes | No | No | Not tested |
| miR-24-3p | Yes | Yes | No | No |
| miR-342-5p | Yes | Yes | No | No |
| miR-425-5p | Yes | Yes | Yes | Yes |
| miR-497-3p | Yes | Yes | Yes | No |
| miR-498 | Yes | No | No | Not tested |
| miR-508-5p | Yes | No | No | Not tested |
| miR-511 | Yes | No | No | Not tested |
| miR-552 | Yes | No | No | Not tested |
| miR-573 | Yes | No | No | Not tested |
| miR-576-3p | Yes | No | No | Not tested |
| miR-582-5p | Yes | No | No | Not tested |
| miR-595 | Yes | No | No | Not tested |
| miR-607 | Yes | No | No | Not tested |
| miR-608 | Yes | No | No | Not tested |
| miR-622 | Yes | No | No | Not tested |
| miR-645 | Yes | No | No | Not tested |
| miR-769-5p | Yes | Yes | Yes | No |
| miR-802 | Yes | No | No | Not tested |
| miR-920 | Yes | No | No | Not tested |
| miR-922 | Yes | No | No | Not tested |

Despite lower expression of miR-105 in adult human atrial tissue (Hsu et al., Circ Cardiovasc Genet 2012; 5:327-35), this miRNA was included as a 13^{th} potential candidate NPPA-modulating miRNA because the seed sequence of miR-105 spans a genetic variant in the NPPA 3′UTR (FIGS. 22A and 22C) that is associated with plasma Nt-proANP levels in humans, as disclosed herein.

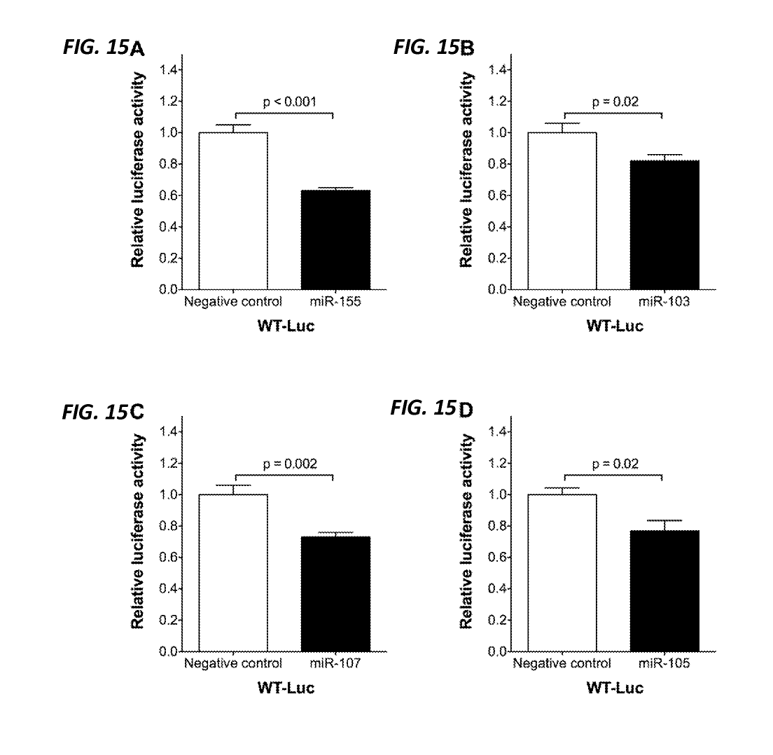

To test the predicted interaction of each of the 13 candidate miRNAs with the NPPA 3′UTR, we generated a reporter construct, in which the firefly luciferase gene was ligated to the wild-type NPPA 3′UTR (WT-Luc). The WT-Luc construct and a second plasmid directing constitutive expression of renilla luciferase to control for transfection efficiency were co-transfected with each of the candidate miRNAs into COS-7 cells. Of the 13 candidate miRNAs, miR-155, miR-103, miR-107, and miR-105, each reduced the luciferase activity in cells containing the WT-Luc construct (FIGS. 15A-15D), suggesting that these 4 miRNAs could interact with the NPPA 3′UTR. Transfection of the other 9 candidate miRNAs failed to alter luciferase activity (Table 6). Therefore, we chose to further investigate the potential roles of miR-155, miR-103, miR-107, and miR-105 in regulation of NPPA expression.

### Example 6

Validation of Predicted Binding of miR-155 with the NPPA 3′UTR

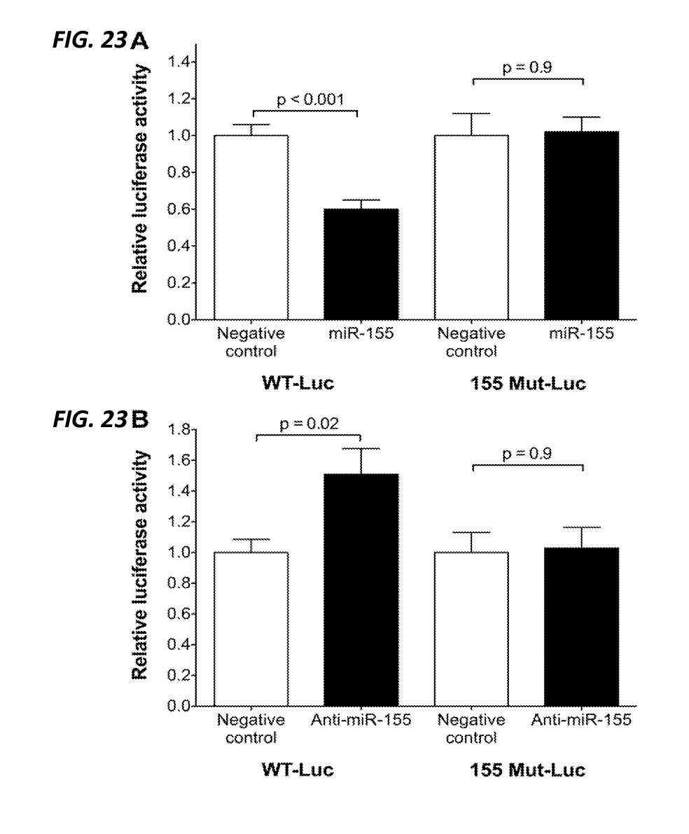

To confirm that the predicted seed binding sequence for miR-155 was required for interaction between miR-155 and the NPPA 3′UTR, a reporter construct (155 Mut-Luc), in which mutations were introduced in 6 of the NPPA 3′UTR seed binding nucleotides, was generated. Transfection of miR-155 reduced the luciferase activity in COS-7 cells containing the wild-type but not the mutant construct (FIG. 23A). The inventors confirmed that COS-7 cells express miR-155 (Table 7). To further establish that miR-155 interacts with the NPPA 3′UTR, we transfected an anti-miR directed against miR-155 (anti-miR-155) together with wild-type or mutated constructs into COS-7 cells. Inhibition of endogenous miR-155 by anti-miR-155 increased the luciferase activity in cells containing the wild-type but not the mutant 3′UTR (FIG. 23B). These results suggest that the NPPA 3′UTR is a direct target of miR-155 and that the effect is mediated by the sequence complementary to the seed sequence.

TABLE 7
Summary of data relating to miR-425, miR-155, miR-105, miR-103, and miR-107.
Expression levels in human atrial tissues are based on RNA sequencing data in
Hsu et al. (Hsu et al., Circ Cardiovasc Genet 2012; 5:327-35). Expression levels
in COS-7 and hESC-CMs were determined by qRT-PCR (↑ = low, ↑↑ =
intermediate, ↑↑↑ = high). For effect of miRNA/anti-miR in
luciferase assays, ↑ indicates an increase and ↓ indicates a decrease
in luciferase activity in cells containing the WT-Luc construct. For effect of
miRNA/anti-miR in hESC-CMs, ↑ indicates an increase and ↓ indicates
a decrease in NPPA mRNA and secreted Nt-proANP protein levels.

| | | | | | | NPPA mRNA and | | human |
|---|---|---|---|---|---|---|---|---|
| | | | | WT-Luc | | Nt-proANP | | population |
| | endogenous miRNA | | | luciferase activity | | protein levels | | genetics |
| | expression levels | | | (COS-7) | | (hESC-CMs) | | studies |
| | human | COS-7 | hESC- | effect of | effect | effect of | effect | minor |
| | atrial | | CMs | miRNA | of | miRNA | of | allele |
| | tissues | | | | anti- | | anti- | associated |
| | (ref.(15)) | | | | miR | | miR | with higher |
| | | | | | | | | plasma Nt- |
| | | | | | | | | proANP |
| | | | | | | | | levels |
| miR-425 | ↑↑↑ | ↑↑ | ↑↑ | ↓ | ↑ | ↓ | ↑ | rs5068 |
| miR-155 | ↑↑ | ↑↑↑ | ↑↑ | ↓ | ↑ | ↓ | ↑ | rs61764044 |
| miR-105 | ↑ | ↑ | ↑ | ↓ | ↑ | — | ↑ | rs5067 |
| miR-103 | ↑↑↑ | ↑↑↑ | ↑↑↑ | ↓ | ↑ | — | — | — |
| miR-107 | ↑↑ | ↑↑ | ↑ | ↓ | ↑ | — | — | — |

miR-155 Regulates Endogenous NPPA mRNA and ANP Protein Levels in Human Cardiomyocytes

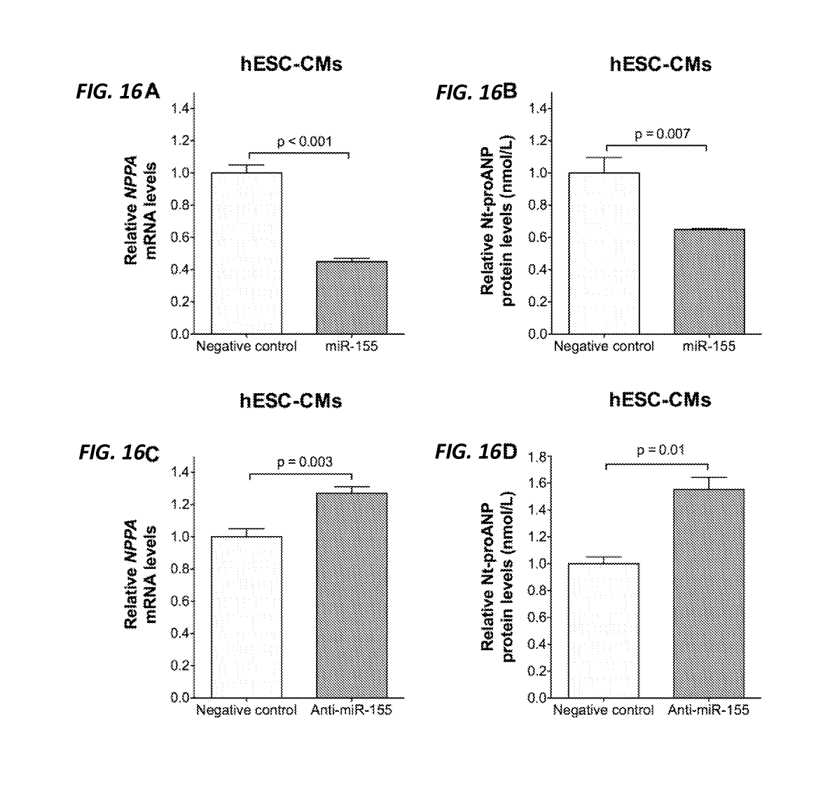

To determine whether miR-155 regulates endogenous NPPA mRNA expression and ANP protein production in cardiomyocytes, the inventors transfected miR-155 into human embryonic stem cell-derived cardiomyocytes (hESC-CMs) and evaluated its effect on NPPA mRNA levels by qRT-PCR and production of the secreted Nt-proANP protein by ELISA. Transfection of miR-155 into hESC-CMs reduced endogenous NPPA mRNA (FIG. 16A), as well as secreted Nt-proANP protein levels (FIG. 16B), while the opposite effects were obtained with the transfection of anti-miR-155 (FIGS. 16C and 16D). These findings demonstrate the biological relevance of miR-155 in regulating NPPA mRNA and ANP protein levels in human cardiomyocytes.

Genetic Variant Rs61764044 is Associated with Plasma ANP Levels and BP in Humans and Disrupts miR-155 Binding of NPPA mRNA

During the course of studies to identify additional variants in the NPPA locus, the inventors performed deep sequencing analyses on 268 samples from individuals of European descent and identified SNP rs61764044 which lies in the miR-155 binding site of the NPPA 3′UTR (FIG. 22B); this SNP rs61764044 has been reported in the 1000 Genomes Project database. This variant is perfectly correlated with rs5068, which lies 123 nucleotides upstream in the 3′UTR (FIG. 22A), indicating that the minor alleles of the 2 variants are always inherited together (perfect linkage disequilibrium, r²=1). Previous studies reported that the rs5068 minor allele disrupts miR-425 binding and is strongly associated with higher plasma ANP levels and lower BP (Newton-Cheh C, et al. Nat Genet 2009; 41:348-53). Because of perfect correlation of the rs61764044 and rs5068 minor alleles, the rs61764044 minor allele is associated with increased plasma ANP levels, lower BP, and reduced risk of hypertension in humans.

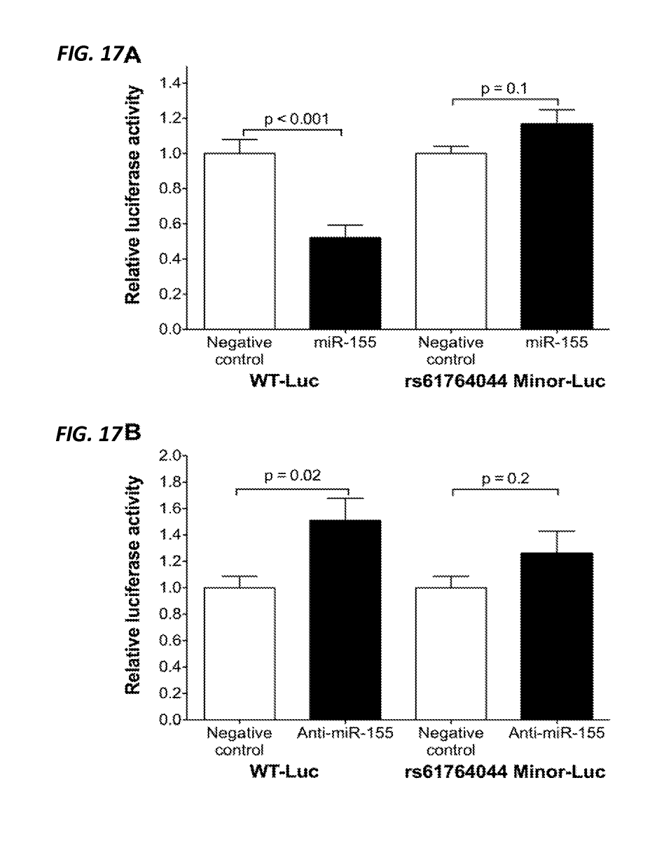

The miR-155 seed sequence spans the major allele of rs61764044, and the minor allele of rs61764044 is predicted to introduce G:U wobble base pairing within the miR-155 seed binding region (FIG. 22B). G:U wobbles in the miRNA seed binding region impair the ability of a miRNA to repress the mRNA target (16-18). The inventors generated a reporter construct (rs61764044 Minor-Luc) in which the firefly luciferase gene was ligated to the NPPA 3′UTR sequence containing the minor alleles of both rs61764044 and rs5068. Transfection of miR-155 reduced the luciferase activity in COS-7 cells containing the major allele (WT-Luc) but not the minor allele (FIG. 17A). Anti-miR-155 increased the luciferase activity in COS-7 cells containing the major but not minor allele (FIG. 17B). Taken together, these results indicate that the NPPA 3′UTR containing the rs61764044 major allele is a direct target of miR-155 and that presence of the rs61764044 minor allele confers resistance to miR-155-mediated repression.

miR-155 Augments miR-425-Mediated Decreases in Endogenous NPPA mRNA and ANP Protein Levels in Human Cardiomyocytes.

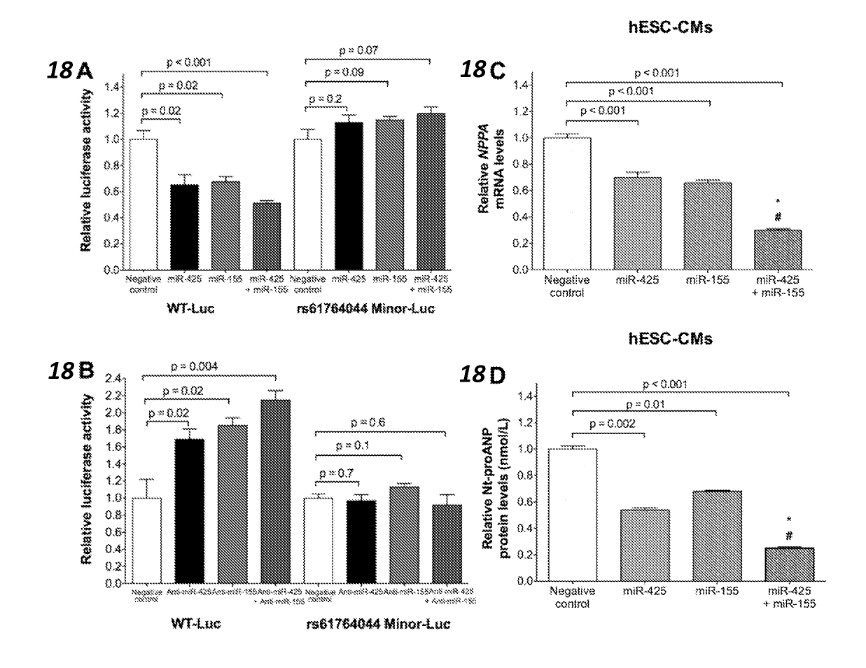

Because the inventors have previously demonstrated that miR-425 targets the NPPA 3′UTR with the rs5068 major allele and inhibits ANP biosynthesis in human cardiomyocytes (Arora P et al., J Clin Invest 2013; 123:3378-82), the inventors next assessed whether miR-155 can enhance the repressive effects of miR-425 on NPPA mRNA. Transfection of either miRNA alone reduced the luciferase activity in the presence of the major alleles but not minor alleles of rs61764044 and rs5068 (FIG. 18A). Co-transfection of miR-425 and miR-155 decreased luciferase activity in the presence of the major but not minor alleles (FIG. 18A). Co-transfection of anti-miR-425 and anti-miR-155 increased the luciferase activity in the presence of the major but not minor alleles (FIG. 18B). Moreover, co-transfection of miR-425 and miR-155 into hESC-CMs decreased endogenous NPPA mRNA levels and secreted Nt-proANP protein levels to a greater extent than either miRNA alone (FIGS. 18C and 18D). Taken together, the inventors have demonstrated that miR-155 regulates NPPA expression and ANP protein production through its interaction with the rs61764044 major allele and can act together with miR-425 to further suppress ANP levels. Thus, association of the rs5068 and rs61764044 minor alleles with higher plasma ANP and Nt-proANP levels, as well as lower BP and reduced risk of hypertension in humans, is likely due to co-inheritance of resistance to two independent miRNAs (Newton-Cheh C et al., Nat Genet 2009; 41:348-53, and Arora P, et al., J Clin Invest 2013; 123:3378-82.)

### Example 7

miR-103 and miR-107 do not Appear to Regulate NPPA mRNA and ANP Protein Levels in Human Cardiomyocytes

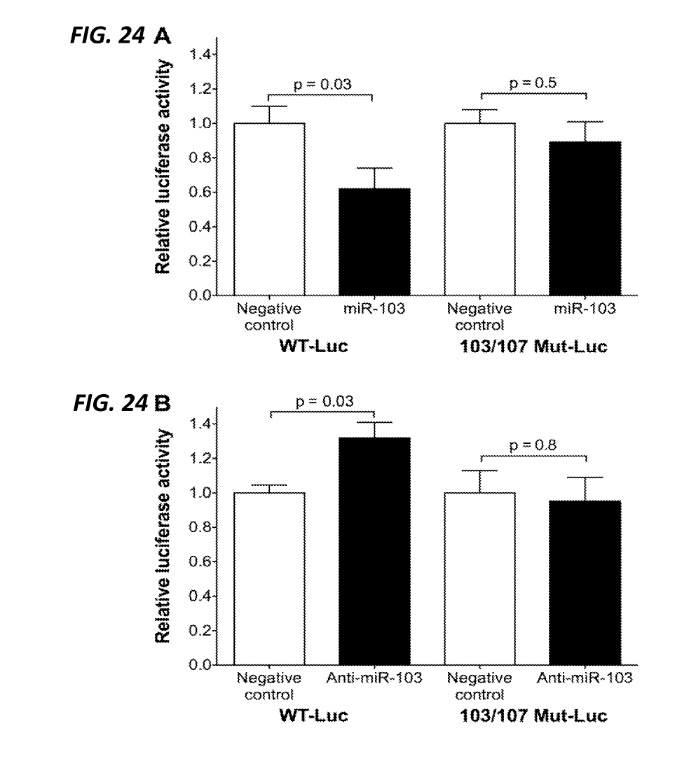

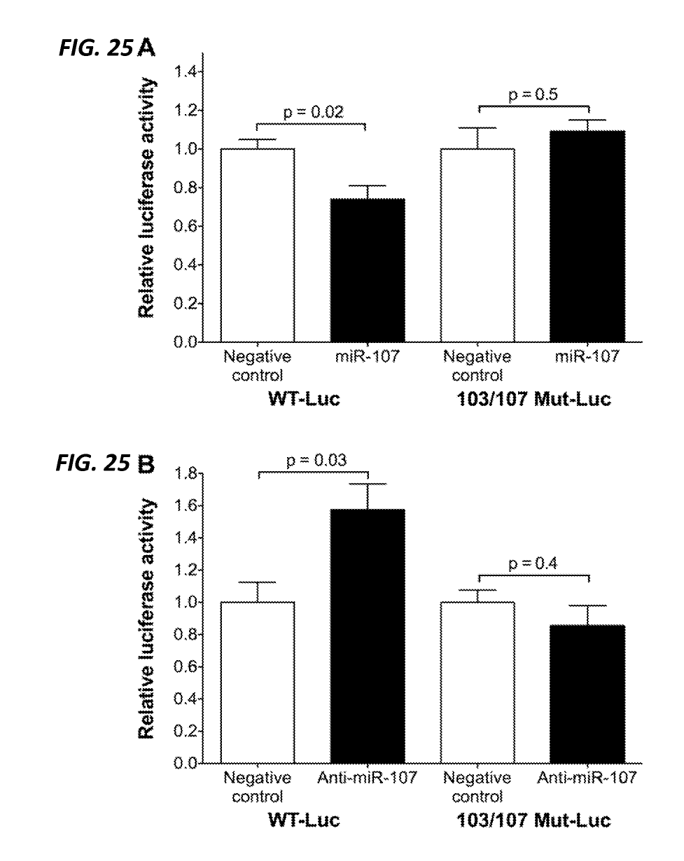

Of the 13 candidate miRNAs, miR-103 and miR-107 share the same seed sequence and thus are predicted to target the same complementary sequence in the NPPA 3′UTR. Exogenous administration of either miRNA reduced the luciferase activity in COS-7 cells containing the wild-type construct but not a construct (103/107 Mut-Luc) with a mutated seed binding site (FIGS. 24A and 25A), confirming that the seed binding site is required for interaction of miR-103 and miR-107 with the NPPA 3′UTR. Inhibition of endogenous miR-103 or miR-107 with their respective anti-miRs increased the luciferase activity in COS-7 cells containing the wild-type but not mutant 3′UTR (FIGS. 24B and 25B). These findings demonstrate that the NPPA 3′UTR is a direct target of miR-103 and miR-107.

Human ESC-CMs express both miR-103 and miR-107 (Table 7). The effects of over-expressing miR-103 and miR-107, as well as each of their anti-miRs, on NPPA mRNA and ANP protein levels were examined. Transfection of the miRNAs or their anti-miRs had no effect on NPPA mRNA or secreted Nt-proANP protein levels in hESC-CMs (data not shown). Accordingly, these findings demonstrate that these miRNAs (miR-103 and miR-107) may not have a role in regulating endogenous ANP levels in human cardiomyocytes, or in human cardiomyocytes derived from hESCs.

### Example 8

Genetic Variant Rs5067 is Associated with Plasma Nt-proANP Levels in Humans and Disrupts miR-105 Binding of NPPA mRNA

MicroRNA-105 (miR-105) was considered by the inventors as a candidate NPPA-targeting miRNA for further investigation because a common polymorphism (rs5067) lies in the miR-105 binding site (FIG. 14C). The miR-105 seed sequence binds to the rs5067 major allele, but the rs5067 minor allele is predicted to disrupt base pairing of miR-105 with the NPPA 3′UTR. In a study of 4,502 Swedish individuals in the Malmo Diet and Cancer Study, the rs5067 minor allele was found to be associated with higher plasma Nt-proANP levels (P=0.004). The correlation of rs5067 with rs5068 is low (r²=0.012, HapMap CEU), although with high linkage disequilibrium (D′=1) due to the fact that the haplotypes containing the minor alleles of the two variants are mutually exclusive (i.e. the minor alleles are never co-inherited). The variants rs5067 and rs5068 are thus independent genetic association signals with plasma Nt-proANP levels. The inventors hypothesized that disruption of the binding of miR-105 to the NPPA 3′UTR by the rs5067 minor allele could represent a mechanism by which rs5067 is associated with plasma Nt-proANP levels in humans.

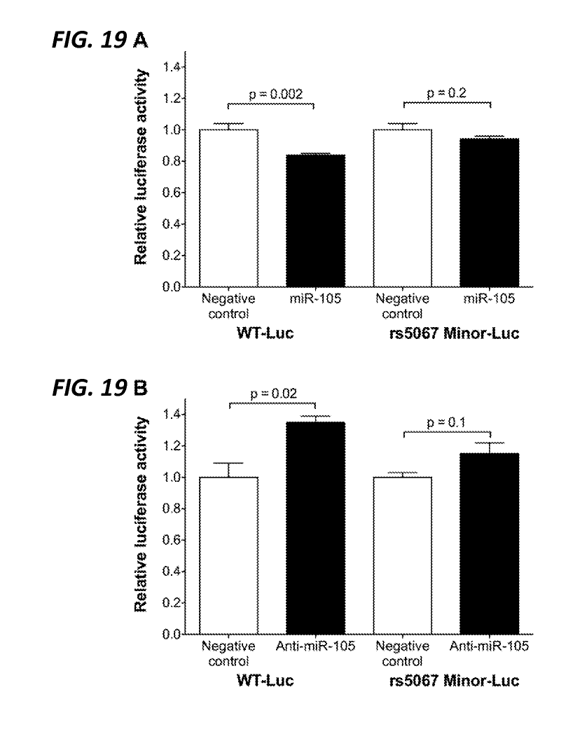

To test whether presence of the rs5067 minor allele alters interaction of miR-105 with the NPPA 3′UTR, the inventors performed luciferase reporter assays using constructs including either the major or the minor rs5067 allele in COS-7 cells. Transfection of miR-105 reduced the luciferase activity in the presence of the major but not minor allele (FIG. 19A). The inventors confirmed that COS-7 cells express miR-105 (Table 7). Anti-miR-105 increased the luciferase activity in the presence of the major but not minor allele (FIG. 19B). Taken together, the inventors have discovered that the NPPA 3′UTR containing the rs5067 major allele is a direct target of miR-105. Presence of the rs5067 minor allele confers resistance to miR-105-mediated repression, which could be a mechanism by which rs5067 alters ANP levels in humans.

miR-105 Regulates Endogenous NPPA mRNA and ANP Protein Levels in Human Cardiomyocytes

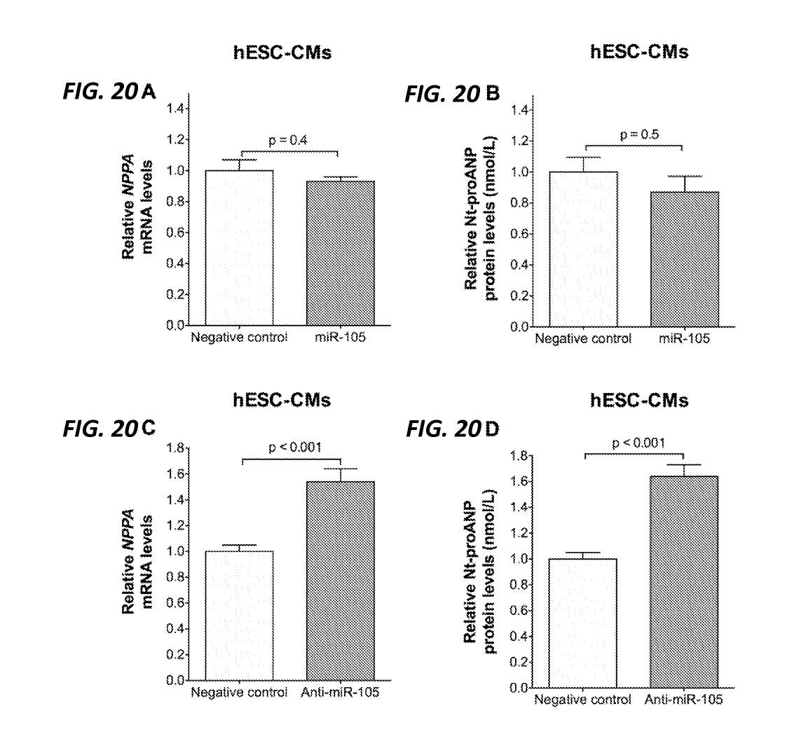

Human ESC-CMs, homozygous for the rs5067 major allele, were transfected with miR-105 to determine whether miR-105 can regulate endogenous NPPA mRNA expression and ANP production. Transfection of miR-105 had no effect on NPPA mRNA (FIG. 20A) or secreted Nt-proANP protein levels (FIG. 20B) in hESC-CMs. As with human atrial tissues (Hsu J et al., Circ Cardiovasc Genet 2012; 5:327-3), hESC-CMs express miR-105 (Table 7). Inhibition of endogenous miR-105 with anti-miR-105 did increase NPPA mRNA (FIG. 20C), as well as secreted Nt-proANP protein levels (FIG. 20D) in hESC-CMs. Although over-expression of miR-105 did not have an effect on NPPA mRNA or Nt-proANP protein levels in hESC-CMs, inhibition of endogenous miR-105 with anti-miR-105 increased NPPA mRNA and secreted Nt-proANP protein levels, suggesting that endogenous levels of miR-105 could modulate NPPA mRNA and protein levels in human cardiomyocytes.

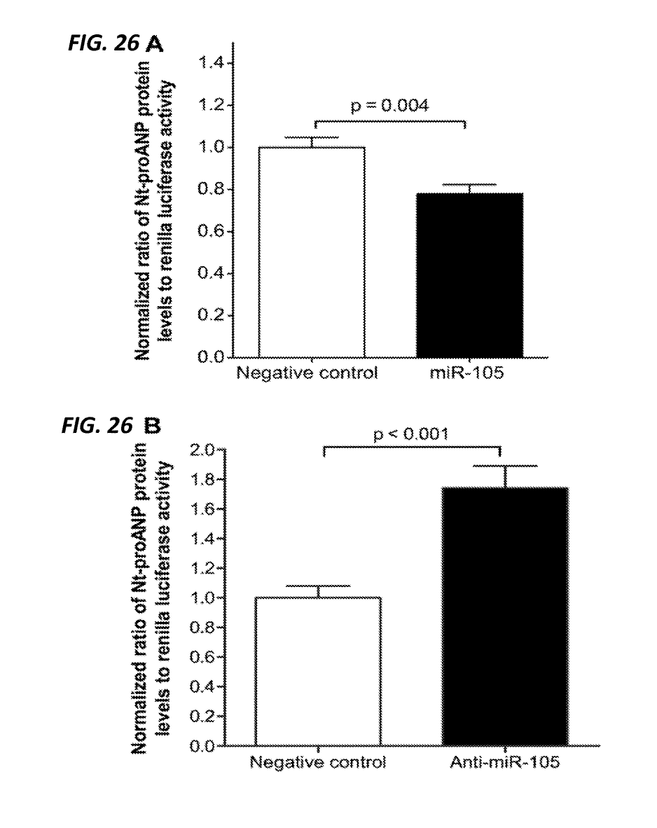

Although the cause of the failure of miR-105 to decrease NPPA mRNA and protein levels in hESC-CMs is unclear, it is likely that the levels of miR-105 in cardiomyocytes, albeit low (Table 7), may at endogenous levels already exert a maximal effect on the target NPPA mRNA. It has been reported that the threshold for saturation of miRNA activity varies for different miRNAs, independent of the expression levels of the miRNA (Gentner B. et al., Nat Methods 2009; 6:63-6, and Brown B D, et al., Nat Rev Genet 2009; 10:578-85). Therefore, in this model, introducing more miR-105 into the hESC-CMs would not have any further effect, but inhibiting endogenous miR-105 would have an effect on NPPA expression by relieving the repression mediated by endogenous miR-105. In COS-7 cells transfected with an NPPA cDNA expression plasmid containing the rs5067 major allele, the inventors determined that co-transfection of miR-105 reduced secreted Nt-proANP protein levels (FIG. 26A), while the opposite effect was obtained with co-transfection of anti-miR-105 (FIG. 26B). These results demonstrate that the repressive effect of over-expressing miR-105 on ANP production can be revealed when the target NPPA mRNA is also over-expressed, providing support for the possibility of saturation.

### Example 9

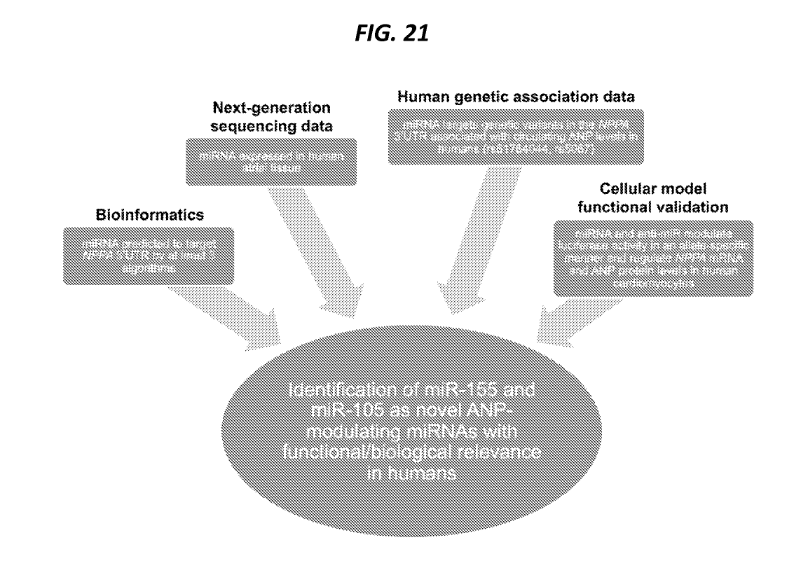

In this study, the inventors used a multifaceted screening strategy that combined in silico miRNA target prediction, miRNA expression data, human genetic association data, and cellular models to identify four miRNAs, in addition to miR-425, that target the NPPA 3′UTR. Two out of the four miRNAs (miR-155 and miR-105) were demonstrated to have a functional role in modulating endogenous NPPA mRNA levels and secreted Nt-proANP levels in human cardiomyocytes (Table 6, FIG. 21). In particular, the inventors have surprisingly discovered that a genetic variant (rs61764044), which disrupts the miR-155 binding site, contributes to the ANP-raising and BP-lowering effects of the previously published rs5068 variant (Arora P et al., J Clin Invest 2013; 123:3378-82, and Balaga O, Nucleic Acids Res 2012; 40:9404-16) by virtue of perfect linkage disequilibrium with rs5068. Similar to miR-425 and miR-155, miR-105 interacts with a genetic variant (rs5067), specifically binding to the rs5067 major but not minor allele, consistent with the finding that the rs5067 minor allele is associated with increased plasma Nt-proANP levels in humans. Thus, by integrating computational, genomic, and cellular approaches, the inventors have discovered novel regulatory mechanisms controlling ANP levels and highlighted the potential of applying a similar integrated screening strategy to uncover miRNA regulators of other genes of interest.

In silico analysis revealed that as many as 494 miRNAs are predicted to target the NPPA 3′UTR. Because miRNA:mRNA interactions predicted by multiple algorithms appear to provide greater specificity without excessive loss of sensitivity (14,21), miRNAs predicted to interact with the NPPA 3′UTR by at least 3 of the algorithms were given priority. To further narrow down the list of miRNAs for subsequent experimental validation, the inventors focused on miRNAs that are expressed at abundant levels in human atrial tissues (Hsu et al., Circ Cardiovasc Genet 2012; 5:327-35), where the target gene NPPA is highly expressed. Using this strategy, the inventors discovered miR-155 as a novel miRNA that interacted with the NPPA 3′UTR, which was confirmed by luciferase reporter assays, and discovered to decrease NPPA mRNA and increase secretion of Nt-proANP protein levels in hESC-CMs. Although miR-103 and miR-107 were also identified as miRNAs that can target the NPPA 3′UTR based on luciferase reporter assays, neither the miRNAs nor their anti-miRs altered NPPA expression in cardiac stem cells, e.g., hESC-CMs, a particular the cell type of interest (Table 6) with respect to modulation of NPPA mRNA expression and production of Nt-proANP protein. Others have also reported instances in which a miRNA was shown to target a 3′UTR of interest in luciferase reporter assays, but had no effects on endogenous target mRNA/protein levels in the cell type of interest (Jiang Q, et al., BMC Cancer 2009; 9:194). These differences may be due to the effect of target secondary structure (Long D, et al., Nat Struct Mol Biol 2007; 14:287-94, and Li F et al., Cell Rep 2012; 1:69-82) and/or the presence of RNA-binding protein levels (Kedde M, et al., Cell 2007; 131:1273-86; Beillard E et al., Rna 2012; 18:1091-100), which may vary between different cell types, and can influence the ability of miRNAs to regulate target mRNAs.

In addition to prioritizing miRNAs that are highly expressed in human atria, the inventors leveraged human population genetic data to investigate whether any of the candidate miRNAs were predicted to target variants in the NPPA 3′UTR that are associated with plasma ANP levels in humans. This approach led to the discovery of miR-105, which was validated to interact with rs5067 in an allele-dependent manner. The rs5067 minor allele disrupted base pairing between miR-105 and the NPPA 3′UTR, which could underlie the observed association of the rs5067 minor allele with higher plasma Nt-proANP levels in humans.

While miR-155 was predicted to target the NPPA 3′UTR and was expressed in human atria, the inventors only suprizingly discovered the rs61764044 variant in the miR-155 binding site when additional sequencing was performed to identify the variant. Prior to the present invention, the perfect correlation between rs5068 and rs61764044 was not known at the time of the inventors prior studies of rs5068 (Newton-Cheh C et al., Nat Genet 2009; 41:348-53, and Arora P, J Clin Invest 2013; 123:3378-82).

The discovery of an allele-specific negative regulatory effect of miR-155 as disclosed herein is a suprizing discovery that demonstrates that rs61764044 contributes to the association between rs5068 and plasma ANP levels, as well as blood pressure (BP). Furthermore, the inventors results highlight the importance of having a complete catalogue of genetic variation. More specifically, the presence of the co-inherited minor alleles G (rs5068) and G (rs61764044) prevents both miR-425 and miR-155, respectively, from binding to the NPPA 3′UTR, resulting in increased ANP production, lower systolic and diastolic BPs, and reduced risk of hypertension in humans. As discovered herein, the rs61764044 minor allele creates a G:U wobble base pairing in the miR-155 seed binding site. Studies have shown that the presence of even a single G:U wobble in the seed binding region impairs miRNA-mediated repression of the mRNA target (Doench J G, et al., Genes Dev 2004; 18:504-11; Yue D, et al., Curr Genomics 2009; 10:478-92; and Brennecke J, et al., PLoS Biol 2005; 3:e85). In the case of the FGF20 3′UTR, for example, a similar disruption of a seed binding site was reported for the T allele of rs12720208, which introduces a G:U wobble base pairing in the miR-433 seed binding site and disrupts ability of miR-433 to repress FGF20 (Wang G, et al., Am J Hum Genet 2008; 82:283-9.

The suprizing discovery that co-transfection of miR-425 and miR-155 resulted in greater NPPA gene repression in hESC-CMs than either miRNA alone, indicates that miR-425 and miR-155 function synergistically, and is has important implications for the therapeutic potential of using combinations of anti-miRs (e.g., anti-miR-425 and anti-miR-155 together) to increase ANP levels. In fact, co-inheritance of the rs5068 (G) and rs61764044 (G) minor alleles in humans demonstrates the physiologic impact of these ANP-raising mechanisms on blood pressure (BP), and demonstrates therapeutic opportunities to increase ANP levels with potential benefits in hypertension or heart failure. Previous reports have implicated the therapeutic potential of miR-155 inhibition in suppressing cardiac hypertrophy and heart failure (Seok H Y, et al., Circ Res 2014; 114:1585-95; Heymans S, et al., Circulation 2013; 128:1420-32). Moreover, miR-155 levels were eported to be increased in human hearts with hypertrophy compared to non-hypertrophic controls and increased miR-155 levels were correlated with depressed cardiac function and increased wall thickness (Heymans S, et al., Circulation 2013; 128:1420-32. However, the inventors herein are the first to discover and demonstrate that miR-155 can target the NPPA 3′UTR to directly regulate ANP levels in human cardiomyocytes. While miR-155 is a multi-functional miRNA with multiple targets (Faraoni I, et al., Biochim Biophys Acta 2009; 1792:497-505), the inventors discovery of its effects on human NPPA expression is important in developing therapies in the treatment or diagnosis of cardiac hypertrophy and heart failure.

The inventors herein chose to experimentally validate a subset of predicted NPPA-targeting miRNAs based on their expression levels in human atrial tissues. The inventors have assessed miR-155, miR-105 and miR103 and miR-107 in hESC-CMs, which are a well-characterized cell type and exhibit structural and functional properties of native human cardiomyocytes (Harding S E, et al., Pharmacol Ther 2007; 113:341-53; and Dick E, et al., Biochem Soc Trans 2010; 38:1037-45). Gene expression profiling studies have also shown that gene expression patterns of cardiac transcription factors and cardiac structural markers (including NPPA) in hESC-CMs are consistent with gene expression patterns in human cardiac tissues (Synnergren J, et al., Physiol Genomics 2010; 43:581-94; Puppala D, et al., Toxicological sciences: an official journal of the Society of Toxicology 2013; 131:292-301; Babiarz J E, et al., Stem cells and development 2012; 21:1956-65). Moreover, miRNA profiling has demonstrated that known cardiomyocyte-specific miRNAs are expressed in hESC-CMs and exhibit the expected expression pattern (Synnergren J, et al., Physiol Genomics 2010; 43:581-94). Thus hESC-CMs are a biologically relevant model for studying the effect of miRNAs on NPPA mRNA levels and ANP production in the human heart.

To conclude, the inventors have discovered herein miR-155 and miR-105 as novel regulators of ANP production. These miRs target (e.g., bind to) sequences including genetic variants in the 3′UTR of the human NPPA gene that are associated with plasma ANP levels in humans. Importantly, the inventors herein have discovered numerous miRs, such as miR-105 and miR-155, which can be used alone, or in combination with miR-425, for miRNA-targeted therapies (e.g., anti-miR-105, anti-miR-155 and/or anti-miR425) to increase ANP biosynthesis, which could supplement current therapy that reduces NP degradation.

### REFERENCES

The cited references and publications in the specification and Examples section are incorporated herein in their entirety by reference.
1. McGuire S. Institute of Medicine. 2010. Strategies to Reduce Sodium Intake in the United States. Washington, D.C.: The National Academies Press. Adv Nutr; 1:49-50.
2. Application of lower sodium intake recommendations to adults—United States, 1999-2006. MMWR Morb Mortal Wkly Rep 2009; 58:281-3.
3. Vollmer W M, Sacks F M, Ard J, et al. Effects of diet and sodium intake on blood pressure: subgroup analysis of the DASH-sodium trial. Ann Intern Med 2001; 135:1019-28.
4. He F J, MacGregor G A. Salt reduction lowers cardiovascular risk: meta-analysis of outcome trials. Lancet; 378:380-2.
5. Sacks F M, Svetkey L P, Vollmer W M, et al. Effects on blood pressure of reduced dietary sodium and the Dietary Approaches to Stop Hypertension (DASH) diet. DASH-Sodium Collaborative Research Group. N Engl J Med 2001; 344:3-10.
6. Bibbins-Domingo K, Chertow G M, Coxson P G, et al. Projected effect of dietary salt reductions on future cardiovascular disease. N Engl J Med 2010; 362:590-9.
7. John S W, Krege J H, Oliver P M, et al. Genetic decreases in atrial natriuretic peptide and salt-sensitive hypertension. [erratum appears in Science 1995 Mar. 24; 267(5205):1753]. Science 1995; 267:679-81.
8. Oliver P M, Fox J E, Kim R, et al. Hypertension, cardiac hypertrophy, and sudden death in mice lacking natriuretic peptide receptor A. Proceedings of the National Academy of Sciences 1997; 94:14730-5.
9. Newton-Cheh C, Larson M G, Vasan R S, et al. Association of common variants in NPPA and NPPB with circulating natriuretic peptides and blood pressure. Nat Genet 2009; 41:348-53.
10. Newton-Cheh C, Johnson T, Gateva V, et al. Genome-wide association study identifies eight loci associated with blood pressure. Nat Genet 2009; 41:676.
11. Go, et al., Circulation 127:143-152.5
12. Roger et al., Circulation 125:e2-e2206
13. Heidenreich, et al., Circulation 123:933-944
1. Volpe M, Rubattu S, Burnett J, Jr. Natriuretic peptides in cardiovascular diseases: current use and perspectives. Eur Heart J 2013; 35:419-25.
2. McMurray J J, Packer M, Desai A S et al. Angiotensin-neprilysin inhibition versus enalapril in heart failure. N Engl J Med 2014; 371:993-1004.
3. Potter L R. Regulation and therapeutic targeting of peptide-activated receptor guanylyl cyclases. Pharmacol Ther 2011; 130:71-82.
4. Steinhelper M E, Cochrane K L, Field U. Hypotension in transgenic mice expressing atrial natriuretic factor fusion genes. Hypertension 1990; 16:301-7.
5. John S W, Krege J H, Oliver P M et al. Genetic decreases in atrial natriuretic peptide and salt-sensitive hypertension. Science 1995; 267:679-81.
6. Newton-Cheh C, Larson M G, Vasan R S et al. Association of common variants in NPPA and NPPB with circulating natriuretic peptides and blood pressure. Nat Genet 2009; 41:348-53.
7. Arora P, Wu C, Khan A M et al. Atrial natriuretic peptide is negatively regulated by microRNA-425. J Clin Invest 2013; 123:3378-82.
8. Balaga O, Friedman Y, Linial M. Toward a combinatorial nature of microRNA regulation in human cells. Nucleic Acids Res 2012; 40:9404-16.
9. Krek A, Grun D, Poy M N et al. Combinatorial microRNA target predictions. Nat Genet 2005; 37:495-500.
10. Akbari Moqadam F, Pieters R, den Boer M L. The hunting of targets: challenge in miRNA research. Leukemia 2013; 27:16-23.
11. Shirdel E A, Xie W, Mak T W, Jurisica I. NAViGaTing the micronome—using multiple microRNA prediction databases to identify signalling pathway-associated microRNAs. PLoS One 2011; 6:e17429.
12. Atmanli A, Hu D, Domian I J. Molecular etching: a novel methodology for the generation of complex micropatterned growth surfaces for human cellular assays. Adv Healthc Mater 2014; 3:1759-64.
13. Smith J G, Newton-Cheh C, Almgren P et al. Assessment of conventional cardiovascular risk factors and multiple biomarkers for the prediction of incident heart failure and atrial fibrillation. J Am Coll Cardiol 2010; 56:1712-9.
14. van Rooij E. The art of microRNA research. Circ Res 2011; 108:219-34.
15. Hsu J, Hanna P, Van Wagoner D R et al. Whole genome expression differences in human left and right atria ascertained by RNA sequencing. Circ Cardiovasc Genet 2012; 5:327-35.
16. Doench J G, Sharp P A. Specificity of microRNA target selection in translational repression. Genes Dev 2004; 18:504-11.
17. Yue D, Liu H, Huang Y. Survey of Computational Algorithms for MicroRNA Target Prediction. Curr Genomics 2009; 10:478-92.
18. Brennecke J, Stark A, Russell R B, Cohen S M. Principles of microRNA-target recognition. PLoS Biol 2005; 3:e85.
19. Gentner B, Schira G, Giustacchini A et al. Stable knockdown of microRNA in vivo by lentiviral vectors. Nat Methods 2009; 6:63-6.
20. Brown B D, Naldini L. Exploiting and antagonizing microRNA regulation for therapeutic and experimental applications. Nat Rev Genet 2009; 10:578-85.
21. Karbiener M, Glantschnig C, Scheideler M. Hunting the needle in the haystack: a guide to obtain biologically meaningful microRNA targets. Int J Mol Sci 2014; 15:20266-89.
22. Jiang Q, Feng M G, Mo Y Y. Systematic validation of predicted microRNAs for cyclin D1. BMC Cancer 2009; 9:194.
23. Long D, Lee R, Williams P, Chan C Y, Ambros V, Ding Y. Potent effect of target structure on microRNA function. Nat Struct Mol Biol 2007; 14:287-94.
24. Li F, Zheng Q, Ryvkin P et al. Global analysis of RNA secondary structure in two metazoans. Cell Rep 2012; 1:69-82.
25. Kedde M, Strasser M J, Boldajipour B et al. RNA-binding protein Dnd1 inhibits microRNA access to target mRNA. Cell 2007; 131:1273-86.
26. Beillard E, Ong S C, Giannakakis A, Guccione E, Vardy L A, Voorhoeve P M. miR-Sens—a retroviral dual-luciferase reporter to detect microRNA activity in primary cells. Rna 2012; 18:1091-100.
27. Wang G, van der Walt J M, Mayhew G et al. Variation in the miRNA-433 binding site of FGF20 confers risk for Parkinson disease by overexpression of alpha-synuclein. Am J Hum Genet 2008; 82:283-9.
28. Seok H Y, Chen J, Kataoka M et al. Loss of MicroRNA-155 protects the heart from pathological cardiac hypertrophy. Circ Res 2014; 114:1585-95.
29. Heymans S, Corsten M F, Verhesen W et al. Macrophage microRNA-155 promotes cardiac hypertrophy and failure. Circulation 2013; 128:1420-32.
30. Faraoni I, Antonetti F R, Cardone J, Bonmassar E. miR-155 gene: a typical multifunctional microRNA. Biochim Biophys Acta 2009; 1792:497-505.
31. Mitcheson J S, Hancox J C, Levi A J. Cultured adult cardiac myocytes: future applications, culture methods, morphological and electrophysiological properties. Cardiovasc Res 1998; 39:280-300.
32. Harding S E, Ali N N, Brito-Martins M, Gorelik J. The human embryonic stem cell-derived cardiomyocyte as a pharmacological model. Pharmacol Ther 2007; 113:341-53.
33. Dick E, Rajamohan D, Ronksley J, Denning C. Evaluating the utility of cardiomyocytes from human pluripotent stem cells for drug screening. Biochem Soc Trans 2010; 38:1037-45.
34. Synnergren J, Ameen C, Lindahl A, Olsson B, Sartipy P. Expression of microRNAs and their target mRNAs in human stem cell-derived cardiomyocyte clusters and in heart tissue. Physiol Genomics 2010; 43:581-94.
35. Puppala D, Collis L P, Sun S Z et al. Comparative gene expression profiling in human-induced pluripotent stem cell—derived cardiocytes and human and cynomolgus heart tissue. Toxicological sciences: an official journal of the Society of Toxicology 2013; 131:292-301.
36. Babiarz J E, Rayon M, Sridhar S et al. Determination of the human cardiomyocyte mRNA and miRNA differentiation network by fine-scale profiling. Stem cells and development 2012; 21:1956-65.

1. A method for increasing ANP levels in a subject, comprising administering to the subject a composition that comprises an anti-miR agent that inhibits the function of at least one of; miR-103, miR-105, miR-107 or miR-155, wherein the anti-miR agent prevents miR-103, miR-105, miR-107 or miR-155 mediated repression of the 3′UTR of the NPPA gene.

2. The method of claim 1, wherein the composition comprises an anti-miR agent selected from any of:
(i) an anti-miR agent that is at least 90% complementary to the nucleobase sequence of any one of miR-103, miR-105, miR-107 or miR-155;
(ii) an anti-miR agent that specifically binds to the seed sequence of any one or a combination of miR-103, miR-105, miR-107 or miR-155, wherein the seed sequence of miR-103 or miR-107 is SEQ ID NO: 4, the seed sequence of miR-105 is SEQ ID NO: 9, and the seed sequence of miR-155 is SEQ ID NO: 14; or
(iii) an anti-miR agent binds to the target sequence of miR-103, miR-105, miR-107 or miR-155 in the 3′UTR of the NPPA gene.

3. (canceled)

4. (canceled)

5. The method of claim 1, wherein the subject has been identified to have hypertension and has been selected for administration with the anti-miR agent or is a subject with, or at risk of hypertension or a cardiovascular disease or disorder.

6. The method of claim 1, wherein the composition comprises at least two anti-miR agents selected from the groups of; anti-miR-103 and anti-miR-107; anti-miR-103 and anti-miR-105, anti-miR-105 and anti-miR-107, anti-miR-103 and anti-miR-155, anti-miR-107 and anti-miR-155 and anti-miR-105 and anti-miR-155.

7. The method of claim 1, wherein the composition comprises at least an anti-miR-155 agent or at least an anti-miR-105 agent, or both.

8. (canceled)

9. The method of claim 1, wherein the composition further comprises an anti-miR agent that inhibits the function of miR-425.

10. The method of claim 9, wherein the composition comprises any of the following combinations:
(i) an anti-miR-105 agent and an anti-miR-425 agent,
(ii) an anti-mR-155 agent and an anti-miR-425 agent,
(iii) an anti-miR-103 agent and an anti-miR-425 an agent,
(iv) an anti-miR-107 agent and an anti-miR-425 agent, or
(v) an anti-miR-103 agent and anti-miR-107 agent and an anti-miR-425 agent.

11. (canceled)

12. The method of claim 1, wherein the anti-miR that inhibits the function of miR-103 inhibits the function of hsa-miR 103a-3p and wherein the anti-miR that inhibits the function of miR-105 inhibits the function of hsa-miR 105-5p.

14.-17. (canceled)

18. An isolated oligonucleotide comprising a nucleotide sequence that is:
at least 90% complementary to, and specifically binds to the seed sequence of any one or a combination of miR-103, miR-105, miR-107 or miR-155, wherein the seed sequence of miR-103 or miR-107 is SEQ ID NO: 4 and the seed sequence of miR-105 is SEQ ID NO: 9, and the seed sequence for miR-155 is SEQ ID NO: 14 or
at least 90% complementary to, and specifically binds to the target sequence of miR-103, miR-105, miR-107 or miR-155 in the 3′UTR of the NPPA gene, wherein the target sequence of miR-103 or miR-107 is SEQ ID NO: 6, and the target sequence of miR-105 is SEQ ID NO: 10 and the target sequence for miR-155 is SEQ ID NO: 199 or SEQ ID NO: 200.

19. (canceled)

20. A method for decreasing ANP levels in a subject, or for treating a subject with hypotension or shock, the method comprising administering to the subject a composition comprising an miR agent that is a mimetic of, or increases the function of, at least one of; miR-103, miR-105, miR-107 or miR-155, wherein the miR agent increases miR-103, miR-105, miR-107 or miR-155 mediated repression of the 3′UTR of the NPPA gene.

21. The method of claim 20, wherein the composition comprises an miR agent is selected from any of:
(i) a miR agent that is at least 90% identical to the nucleobase sequence of any one of miR-103, miR-105, miR-107 or miR-155;
(ii) a miR agent that is miR-155 or a mimetic or modified miR-155 agent thereof, where the modified miR-155 agent binds to the G allele of rs61764044;
(iii) a miR agent that is miR-105 or a mimetic or modified miR-105 agent thereof.

22. The method of claim 20, wherein the subject has been identified to have hypotension and has been selected for administration with the miR agent.

23. The method of claim 20, wherein the composition comprises at least two miR agents selected from the groups of;
(i) miR-103 and miR-107,
(ii) miR-103 and miR-105,
(iii) miR-105 and miR-107,
(iv) miR-103 and miR-155,
(v) miR-107 and miR-155, and
(vi) miR-1-5 and miR-155.

24.-25. (canceled)

26. The method of claim 20, wherein the composition further comprises a miR agent that is a mimetic of, or increases the function of, miR-425.

27. The method of claim 26, wherein the composition comprises any one of:
(i) a miR-105 agent and an a miR-425 agent,
(ii) miR-155 agent and miR-425 agent,
(iii) a miR-103 agent and a miR-425 agent,
(iv) a miR-107 agent and a miR-425 agent, or
(v) a miR-103 agent and a miR-107 agent and a miR-425 agent.

28. (canceled)

29. The method of claim 20, wherein the miR agent that increases the function of miR-103 increases the function of hsa-miR 103a-3p, or wherein the miR agent that increases the function of miR-105 increases the function of hsa-miR 105-5p.

30.-36. (canceled)

37. A pharmaceutical composition comprising:
at least one anti-miR-155 agent and at least one anti-miR-425 agent, or
at least one anti-miR-105 agent and at least one anti-miR-425 agent, or
at least one anti-miR-155 agent, at least one anti-miR105 agent and at least one anti-miR-425 agent,
and a pharmaceutically acceptable carrier.

38. (canceled)

39. The pharmaceutical composition of claim 37, wherein the anti-miR-155 agent is selected from the group consisting of:
(i) an anti-miR-155 agent that is at least 90% complementary to the nucleobase sequence of miR-155, or the seed sequence of miR-155 of SEQ ID NO: 14;
(ii) an anti-miR-155 agent that is an inhibitory nucleic acid sequence comprising a sequence that is complementary to a contiguous sequence of at least 5 nucleotides present in hsa-miR-155;
(iii) an anti-miR-155 agent that comprises a nucleotide sequence that is complementary to, and specifically binds to the seed sequence of SEQ ID NO: 14, or is at least 80% complementary to, and specifically binds to the target sequence of miR-155 in the 3′UTR of the NPPA gene of SEQ ID NO: 199 or SEQ ID NO: 200, and
(iv) an anti-miR-155 agent having a sequence of SEQ ID NO: 240.

40.-42. (canceled)

43. The pharmaceutical composition of claim 37, wherein the anti-miR-105 agent is selected from any of the group consisting of:
(i) an anti-miR-105 agent that is at least 80% complementary to the nucleobase sequence of miR-105, or the seed sequence of SEQ ID NO: 9;
(ii) an anti-miR-105 agent that is an inhibitory nucleic acid sequence comprising a sequence that is complementary to a contiguous sequence of at least 5 nucleotides present in hsa-miR-105;
(iii) an anti-miR-105 agent comprises a nucleotide sequence that is complementary to, and specifically binds to the seed sequence of SEQ ID NO: 9, or is at least 90% complementary to, and specifically binds to the target sequence of miR-105 in the 3′UTR of the NPPA gene of SEQ ID NO: 10.

44. (canceled)

45. (canceled)

46. The pharmaceutical composition of claim 37, wherein the anti-miR-425 agent is selected from the group of:
(i) an anti-miR-425 agent that is at least 90% complementary to the nucleobase sequence of miR-425, or the seed sequence of SEQ ID NO: 16,
(ii) an anti-miR-425 agent that comprises a nucleotide sequence that is complementary to, and specifically binds to the seed sequence of SEQ ID NO: 16, or is at least 90% complementary to, and specifically binds to the target sequence of miR-425 in the 3′UTR of the NPPA gene of SEQ ID NO: 18,
(iii) an anti-miR-425 agent that is an inhibitory nucleic acid sequence comprising a sequence that is complementary to a contiguous sequence of at least 5 nucleotides present in hsa-miR-425.

47.-51. (canceled)

52. A pharmaceutical composition comprising:
at least one miR-155 agent and at least one miR-425 agent, or
at least one miR-105 agent and at least one miR-425 agent, or
at least one miR-155 agent, at least one miR-105 agent and at least one miR-425 agent,
and a pharmaceutically acceptable carrier.

53. (canceled)

54. A pharmaceutical composition of claim 52, wherein the composition comprises any of:
(i) a miR-155 agent comprising a nucleic acid sequence which is at least 80% identical to the nucleobase sequence of miR-155 (e.g. the mature miR-155 or pre-miR-155), or a miR-155 agent comprising a sequence a contiguous sequence of at least 5 nucleotides present in hsa-miR-155, or
(ii) a miR-105 agent comprising a nucleic acid sequence which is at least 80% identical to the nucleobase sequence of miR-105 (e.g. the mature miR-105 or pre-miR-105), or a miR-105 agent comprising a sequence that is a contiguous sequence of at least 5 nucleotides present in hsa-miR-105, or
(iii) a the miR-425 agent comprising a nucleic acid sequence which is at least 80% identical to the nucleobase sequence of miR-425 (e.g. the mature miR-425 or pre-miR-425), or a miR-425 agent comprising a nucleic acid sequence comprising a contiguous sequence of at least 5 nucleotides present in hsa-miR-425.

55.-61. (canceled)
